(19) United States
(12) Patent Application Publication
(10) Pub. No.: US 2018/0071272 A1
(43) Pub. Date: Mar. 15, 2018

(54) NOVEL CHRONOTHERAPY BASED ON CIRCADIAN RHYTHMS

(71) Applicant: THE TRUSTEES OF THE UNIVERSITY OF PENNSYLVANIA, PHILADELPHIA, PA (US)

(72) Inventors: JOHN B. HOGENESCH, ROSE VALLEY, PA (US); GARRET A. FITZGERALD, WAYNE, PA (US)

(21) Appl. No.: 15/520,317

(22) PCT Filed: Oct. 19, 2015
(86) PCT No.: PCT/US15/56232

§ 371 Date: Apr. 19, 2017

Related U.S. Application Data
(60) Provisional application No. 62/122,525, filed on Oct. 23, 2014.

(51) Int. Cl. A61K 31/455; A61K 31/41; A61K 31/4178; A61K 31/138; A61K 31/5377; A61K 31/549; A61K 38/05; A61K 31/192; A61K 31/405

(52) U.S. Cl. CPC A61K 31/455; A61K 31/41; A61K 31/4178; A61K 31/138; A61K 31/405; A61K 31/549; A61K 38/05; A61K 31/192; A61K 31/5377

(57) ABSTRACT

The invention includes a formulation of a therapeutic compound, wherein release of the therapeutic compound from the formulation coincides with peak or trough expression of at least one target gene of the therapeutic compound. The invention also includes a method of developing such formulations and a method of treating a disorder in a subject using such formulations.

### CROSS-REFERENCE TO RELATED APPLICATIONS

The present application claims priority under 35 U.S.C. § 119(e) to U.S. Provisional Patent Application No. 62/122,525, filed Oct. 23, 2014, which application is incorporated herein by reference in its entirety.

### STATEMENT REGARDING FEDERALLY SPONSORED RESEARCH OR DEVELOPMENT

This invention was made with government support under grant number 5-R01-HL097800 awarded by the National Heart, Lung, and Blood Institute and under grant number 12-DARPA-1068 awarded by the Defense Advanced Research Planning Agency. The government has certain rights in the invention.

### BACKGROUND OF THE INVENTION

Circadian rhythms are endogenous 24-hour oscillations in behavior and biological processes found in all lives. This internal clock allows an organism to adapt its physiology in anticipation of transitions between night and day. The circadian clock drives oscillations in a diverse set of biological processes, including sleep, locomotor activity, blood pressure, body temperature, and blood hormone levels (Levi, et al., 2007, Annu. Rev. Pharmacol. Toxicol., 47:593-628; Curtis et al, 2006, Ann. Med., 38:552-9). Disruption of normal circadian rhythms leads to clinically relevant disorders including neurodegeneration and metabolic disorders (Hastings, et al., 2013, Curr. Opin. Neurobiol., 23:880-7; Marcheva, et al., 2010, Nature, 466:627-631). In mammals, the molecular basis for these physiological rhythms arises from the interactions between two transcriptional/translational feedback loops (Lowrey, 2011, Adv. Genet., 74:175-230). Many members of the core clock regulate the expression of other transcripts. These clock-controlled genes mediate the molecular clock's effect on downstream rhythms in physiology.

There is a need in the art for a novel formulation of a therapeutic compound to improve its efficacy and safety according to the circadian rhythms. The present invention satisfies this need.

### BRIEF SUMMARY OF THE INVENTION

In one aspect, the present invention includes a formulation providing coordinated release of a therapeutic compound selected from Table 1, wherein release of the therapeutic compound from the formulation coincides with peak or trough expression of at least one target gene of the therapeutic compound. In certain embodiments, the at least one target gene is PPARα. In other embodiments, the target gene of the therapeutic compound is a niacin receptor, Niacr1. In yet other embodiments, the therapeutic compound is niacin. In yet other embodiments, the niacin is released zero to six hours after contact with a solution having a pH of between 1 and 5 and a temperature of between 35 and 42° C. In yet other embodiments, the therapeutic compound is dosed within one hour of a final meal before bedtime.

In another aspect, the formulation of the invention provides coordinated release of a first portion of the therapeutic compound and a second portion of the therapeutic compound such that release of the first portion of the therapeutic compound coincides with peak or trough expression of the at least one target gene and release of the second portion of the therapeutic compound occurs after peak or trough expression of the at least one target gene. In certain embodiments, release of the second portion of the therapeutic compound occurs prior to one half-life of the therapeutic compound following the first portion release. In other embodiments, release of the second portion of the therapeutic compound occurs after one half-life of the therapeutic compound following the first portion release. In yet other embodiments, release of the second portion of the therapeutic compound occurs after the release of substantially the entire first portion and prior to one half-life of the therapeutic compound following the release of the first portion. In yet other embodiments, release of the second portion of the therapeutic compound occurs prior to the release of substantially the entire first portion. In yet other embodiment, release of a second portion of the therapeutic compound contained in the formulation occurs at a time independent of an expression peak or trough of its target gene in a tissue type and wherein the release of the second portion avoids an undesirable side effect. In yet other embodiments, the formulation further provides release of at least a third portion of the therapeutic compound.

In yet another aspect, the therapeutic compound of the formulation inhibits at least two target genes and wherein the formulation provides coordinated release such that release of a first portion of the therapeutic compound contained in the formulation coincides with peak or trough expression of a first target gene and release of a second portion of the therapeutic compound contained in the formulation coincides with peak or trough expression of a second target gene. In certain embodiments, the formulation further provides release of at least a third portion of the therapeutic compound contained in the formulation such that release of the at least third portion coincides with peak or trough expression of at least a third target gene and wherein peak or trough expression of the at least third target gene is defined in Table 2. In other embodiments, the first target gene and the second target gene are each selected from Table 1. In yet other embodiments, peak or trough expression of the target gene in each tissue type is defined in Table 2. In yet other embodiments, each of the at least two target genes is selected from the group consisting of PPARα, PPARδ, and PPARγ. In yet other embodiments, the therapeutic compound is a fibrate having a half-life of less than six hours. In yet other embodiments, the fibrate is released two to four hours after contact with a solution having a pH of between 1 and 5 and a temperature of between 35 and 42° C. In yet other embodiments, the at least two target genes are expressed in at least two tissue types and wherein the formulation provides coordinated release of the therapeutic compound such that release of the first portion of the therapeutic compound contained in the formulation coincides with peak or trough expression of the first target gene in the first tissue type and release of the second portion of the therapeutic compound contained in the formulation coincides with peak or trough expression of the second target gene in the second tissue type.

In yet other aspect, the formulation provides coordinated release of the therapeutic compound such that release of a first portion of the therapeutic compound contained in the formulation coincides with peak or trough expression of the at least one target gene in a first tissue type and release of a second portion of the therapeutic compound contained in the formulation coincides with peak or trough expression of the at least one target gene in a second tissue type, and the at least one target gene is expressed in at least two tissue types. In certain embodiments, the first tissue type and the second tissue type are each selected from Table 1. In other embodiments, the first tissue type is liver and the second tissue type is kidney. In yet other embodiments, the therapeutic compound is Gemfibrozil or Bezafibrate. In yet other embodiments, the formulation further provides release of at least a third portion of the therapeutic compound contained in the formulation such that the release of the at least third portion coincides with peak or trough expression of the at least on target gene in an at least third tissue type and wherein peak or trough expression of the at least one target gene in the at least third tissue type is defined in Table 2. In yet other embodiments, the first target gene is PPARα and the first tissue type is liver. In yet other embodiments, the second target gene is PPARγ and the second tissue type is kidney. In yet other embodiments, the formulation provides release of at least a third portion of the therapeutic compound contained in the formulation such that the release of the at least third portion coincides with peak or trough expression of at least a third target gene and wherein peak or trough expression of the at least third target gene is defined in Table 2, optionally, wherein the at least a third target gene is expressed in a third tissue type.

In yet another aspect, the invention includes a formulation providing coordinated release of at least two therapeutic compounds selected from Table 1, wherein each therapeutic compound inhibits at least one different target gene wherein release of a first therapeutic compound from the formulation coincides with peak or trough expression of at least one target gene of the first therapeutic compound and wherein release of a second therapeutic compound from the formulation coincides with peak or trough expression of at least one target gene of the second therapeutic compound. In certain embodiments, release of the second therapeutic compound occurs at a specified time following release of the first therapeutic compound wherein the specified time correlates with a differential between peak or trough expression of at least one target gene of the first therapeutic compound and peak or trough expression of at least one target gene of the second therapeutic compound and wherein peak or trough expression of each target gene is defined in Table 2. In other embodiments, release of the second therapeutic compound occurs at a specified time following release of the first therapeutic compound wherein the specified time correlates with a differential in peak or trough expression of the target gene of the first therapeutic compound and the peak or trough expression of the target gene of the second therapeutic compound as defined in Table 2. In yet other embodiments, the target gene of the first therapeutic compound is Agtr1a and the target gene of the second therapeutic compound is Adrb2 or Adrb1. In yet other embodiments, the first therapeutic compound is an angiotensin receptor blocker (ARB) having a half-life of less than six hours and wherein the second therapeutic compound is a beta blocker having a half-life of less than three hours. In yet other embodiments, the ARB is released zero to two hours after contact with a solution having a pH of between 1 and 5 and a temperature of between 35 and 42° C. and the beta blocker is released two to four hours after contact with a solution having a pH of between 1 and 5 and a temperature of between 35 and 42° C. In yet other embodiments, the ARB is Valsartan or Losartan and the beta blocker is Metoprolol or Timolol. In yet other embodiments, the target gene of the first therapeutic compound is Agtr1a and the target gene of the second therapeutic compound is Car4, Cart, Car12, or Car9. In yet other embodiments, the first therapeutic compound is an angiotensin receptor blocker (ARB) having a half-life of less than six hours and wherein the second therapeutic compound is a diuretic. In one embodiment, the ARB is released zero to two hours after contact with a solution having a pH of between 1 and 5 and a temperature of between 35 and 42° C. and the diuretic is released six to eight hours after contact with a solution having a pH of between 1 and 5 and a temperature of between 35 and 42° C. In another embodiment, the ARB is Valsartan or Losartan and diuretic is Hydrochlorothiazide. In yet another embodiment, the target gene of the first therapeutic compound is Ace and the target gene of the second therapeutic compound is Adrb2 or Adrb1. In yet other embodiments, the first therapeutic compound is an acetylcholinesterase (ACE) inhibitor having a half-life of less than six hours and wherein the second therapeutic compound is a beta blocker having a half-life of less than three hours. In one embodiment, the ACE inhibitor is released zero to two hours after contact with a solution having a pH of between 1 and 5 and a temperature of between 35 and 42° C. and the beta blocker is released two to four hours after contact with a solution having a pH of between 1 and 5 and a temperature of between 35 and 42° C. In another embodiment, the ACE inhibitor is Enalapril or Ramipril and the beta blocker is Metoprolol or Timolol. In yet other embodiments, the target gene of the first therapeutic compound is Ace and the target gene of the second therapeutic compound is Car4, Car2, Car12, or Car9. In one embodiment, wherein the first therapeutic compound is an acetylcholinesterase (ACE) inhibitor having a half-life of less than six hours and wherein the second therapeutic compound is a diuretic. In another embodiment, the ACE inhibitor is released zero to two hours after contact with a solution having a pH of between 1 and 5 and a temperature of between 35 and 42° C. and the diuretic is released six to eight hours after contact with a solution having a pH of between 1 and 5 and a temperature of between 35 and 42° C. In yet another embodiment, the ACE inhibitor is Enalapril or Ramipril and diuretic is Hydrochlorothiazide. In yet other embodiments, the target gene of the first therapeutic compound is PPARα and the target gene of the second therapeutic compound is Hmgcr. In certain embodiments, the first therapeutic compound is a fibrate having a half-life of less than two hours and wherein the second therapeutic compound is a statin having a half-life of less than two hours. In one embodiment, the fibrate is released zero to two hours after contact with a solution having a pH of between 1 and 5 and a temperature of between 35 and 42° C. and the statin is released four to six hours after contact with a solution having a pH of between 1 and 5 and a temperature of between 35 and 42° C. In another embodiment, the fibrate is principally metabolized by CYP3A4 and the statin is principally metabolized by CYP2C9. In yet another embodiment, the fibrate is Gemfibrozil and the statin is Fluvastatin. In other embodiments, the first therapeutic compound and the second therapeutic compound are dosed before bedtime and each exhibits normal pharmacokinetics once released from the formulation. In yet other embodiments, the formulation of the invention further provides release of at least a third therapeutic compound contained in the formulation such that release of the at least third therapeutic compound coincides with peak or trough expression of at least a third target gene and wherein peak or trough expression of the at least third target gene is defined in Table 2.

In yet another aspect, the formulation of the invention provides coordinated release of at least two different therapeutic compounds selected from Table 1, wherein the at least two therapeutic compounds have at least one common target gene, wherein release of a first therapeutic compound coincides with peak or trough expression of the common target gene and release of a second therapeutic compound coincides with peak or trough expression of the common target gene.

In yet another aspect, the invention includes a method for treating a disease in a subject in need thereof. The method comprises administering an effective amount of a formulation of the invention at a specified time, such that release of a therapeutic compound from the formulation coincides with peak or trough expression of at least one target gene of the therapeutic compound.

In yet another aspect, the invention includes a kit comprising a formulation of the invention and instructions for use. In certain embodiments, the instructions specify that the formulation is provided such that release of a first therapeutic compound or a first portion of the first therapeutic compound from the formulation coincides with peak or trough expression of at least one target gene of the first therapeutic compound.

In yet another aspect, the invention includes a method of developing an improved formulation for a therapeutic compound. The method comprises: identifying the circadian phase of gene expression of a target for the therapeutic compound; identifying a desired administration time; and calculating a difference between the circadian phase of the target gene expression and the desired administration time; and developing a delayed-release formulation corresponding to the calculated difference.

In yet another aspect, the invention includes a method of developing an improved formulation to reduce an undesired side effect of a therapeutic compound. The method comprises: identifying a circadian phase of gene expression of a target associated with the undesired side effect of the therapeutic compound; identifying a desired administration time to minimize the undesired side effect; calculating a difference between circadian phase of gene expression of the target and the desired administration time; and developing a delayed-release formulation corresponding to the calculated difference.

In yet another aspect, the invention includes a method of developing an improved formulation to reduce the metabolism of a therapeutic compound. The method comprises: identifying a circadian phase of expression of a metabolic enzyme involved in the metabolism of the therapeutic compound; identifying a desired administration time to minimize the metabolism of the therapeutic compound; calculating a difference between the circadian phase of expression of the metabolic enzyme and the desired administration time; and developing a delayed-release formulation corresponding to the calculated difference.

In yet another aspect, the invention includes a method of developing an improved formulation to increase the metabolism of a prodrug. The method comprises: identifying a circadian phase of expression of a metabolic enzyme involved in converting the prodrug to a drug; identifying a desired administration time to maximize the metabolism of the prodrug; calculating a difference between circadian phase of expression of the metabolic enzyme and the desired administration time; and developing a delayed-release formulation corresponding to the calculated difference.

In yet another aspect, the invention includes a method of developing an improved formulation to increase the transportation of a therapeutic compound to its desired target. The method comprises: identifying a circadian phase of expression of a transporter involved in the transportation of the therapeutic compound to its desired target; identifying a desired administration time to increase the transportation of the therapeutic compound to its desired target; calculating a difference between circadian phase of expression of the transporter and the desired administration time; and developing a delayed-release formulation corresponding to the calculated difference.

In yet another aspect, the invention includes a method of developing an improved formulation to decrease the transportation of a therapeutic compound to its undesired target. The method comprises: identifying a circadian phase of expression of a transporter involved in the transportation of the therapeutic compound to its undesired target; identifying a desired administration time to decrease the transportation of the therapeutic compound to its undesired target; calculating a difference between circadian phase of expression of the transporter and the desired administration time; and developing a delayed-release formulation corresponding to the calculated difference.

In certain embodiments, the therapeutic compound is selected from the group consisting of esomeprazole, valsartan, rituximab, fluticasone, lisdexamfetamine dimesylate, oseltamivir, methylphenidate, testosterone, lidocaine, quetiapine, sildenafil, niacin, insulin lispro, pemetrexed, ipratropium bromide/albuterol, albuterol sulfate, sitagliptin/metformin, metoprolol succinate, ezetimibe/simvastatin, rabeprazole, eszopiclone, omeprazole, dexmethylphenidate, enalapril, neostigmine, ephedrine, pyridostigmine, lisdexamfetamine, salmeterol, salbutamol, timolol, metoprolol, epinephrine, propranolol, hydralazine, acetazolamide, fludrocortisone, spironolactone, docetaxel, paclitaxel, nifedipine, pilocarpine, atropine, levamisole, carbidopa, flucytosine, levodopa, dopamine, naloxone, propofol, midazolam, ondansetron, ethionamide, vinblastine, hydrochlorothiazide, primaquine, gentamicin, dacarbazine, didanosine, cytarabine, cefazolin, metformin, tetracycline, misoprostol, sulfasalazine, ibuprofen, acetylsalicylic acid, riboflavin, verapamil, ketamine, ciprofloxacin, etoposide, propylthiouracil, mebendazole, fluorouracil, and allopurino. In one embodiment, the therapeutic compound is valsartan. In another embodiment, the desired administration time is between 5 pm and 9 pm.

In yet another aspect, the invention includes to a delayed-release formulation comprising a pharmaceutically effective amount of valsartan, wherein the valsartan is delayed to be released to gastrointestinal tract from the time when the valsartan is orally administered. In certain embodiments, the delay is about 6 hours. In other embodiments, the delayed-release formulation further comprises an erodible plug, an impermeable capsule body, and soluble cap.

In yet another aspect, the invention includes a method of maximizing the efficacy of a therapeutic compound in a subject. The method comprises identifying the circadian phase of the subject using a measuring device; identifying the target gene of the therapeutic compound; and administering the therapeutic compound to the subject at the circadian phase when the target gene for the therapeutic compound is maximally or minimally expressed; wherein the measuring device is installed with a suitable application that identifies or tracks the circadian phases of the subject. In one embodiment, the therapeutic compound is streptozocin.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the breakdown of circadian genes and non-coding RNAs. Panel A illustrates the number of protein-coding genes in each organ that exhibit circadian expression. Blue marks indicate the number of genes with at least 1 spliceform detected by RNA-seq. Orange marks indicate the number of genes with at least 2 spliceforms detected by RNA-seq. Blue numbers to the right of each bar list the percentage of protein coding genes with rhythmic expression in each tissue. Panel B is a graph illustrating the distribution of the number of organs in which a protein-coding gene oscillated according to the circadian cycle. Panel C is a graph illustrating average total number of circadian genes detected as a function of the number of organs sampled. Panel D is a graph illustrating the percentages of each transcript class that did vs. did-not oscillate in at least one organ.

FIG. 2 illustrates parameters of circadian gene expression across organs. Panel A is a graph illustrating the relationship between organ, oscillation amplitude and oscillation phase of circadian gene expression. Upper-left quadrant illustrates histograms of amplitudes within each organ (number of circadian genes expressed within each amplitude bin is shown on the horizontal axis, grouped by organ). Upper-right quadrant illustrates histograms of amplitudes of expression within each phase, across all organs. Lower-right quadrant illustrates histograms of phases of expression within each organ, with summary radial diagrams (number of circadian genes within each phase bin is shown on the vertical axis, grouped by organ). Lower-left quadrant illustrates Venn diagrams of the identities of the genes whose expression oscillated within a given pair of organs. Panel B is schematic ontogenic tree constructed using the average phase differences between each organ pair's shared circadian gene expression as the distance metric. Shared gene expression corresponds to the overlapping regions from Venn diagrams in panel A.

FIG. 3 illustrates pathways of gene expression across biological space and time. Panel A illustrates a superimposed circadian graph of the deltex gene Dtx4 expression in all organs tested. Panel B illustrates an example of pathway components' timing of gene expression reflecting function: expression profiles from the heart, for Vegfa and its two receptors Kdr and Flt1. Black arrows highlight times at which Flt1 and Kdr are anti-phased. Panel C illustrates an example of systemic pathway of gene expression orchestration segregating in time and space: expression profile of Igf1 in the liver, as compared to its downstream target Pik3 in several organs. Panel D illustrates an example of widespread pathway gene expression component synchronization within the same space (organ): expression profiles from the kidney for multiple signaling receptors that activate the PIK3-AKT-MTOR pathway.

FIG. 4 illustrates the overlap of circadian disease gene expression and drug targets. Panel A is a schematic diagram illustrating overlap between expression of circadian genes, expression of known disease-associated genes, and expression of drug targets. Panel B illustrates an example of a common drug having an oscillatory target gene expression: expression profiles for the aspirin target Ptgs1 from heart, lung, and kidney. Traces of expression from these organs of the mir22 host gene, predicted to target Ptgs1, are also shown. Panel C illustrates the number of PubMed references disclosing circadian vs. non-circadian genes.

FIG. 5 illustrates oscillating transcripts from expression of genes across different organs. Panel A is a graph illustrating the effect of 5% false-discovery rate for detection. Panel B is a graph illustrating the average total number of oscillating genes expressed and detected as a function of the number of organs sampled. Panel C is a set of radial diagrams illustrating the phase distribution of oscillating gene expression in each organ.

FIG. 6 illustrates conserved circadian non-coding RNAs (ncRNAs). Panel A is a schematic diagram illustrating method overview for identifying conserved ncRNAs. Panel B is a diagram illustrating functional types of circadian conserved ncRNAs. Types were defined by GENCODE and Ensembl biotypes, assigned by using Ensembl and manual annotation.

FIG. 7 illustrates representative examples of conserved circadian ncRNAs and anti-sense transcripts. Panel A is a RNA-seq coverage plot for Galt (red) and its antisense transcript (blue). The gene model for Galt is displayed above the coverage plots. Panel B comprises two graphs illustrating expression profiles for Galt (red; data from microarrays) and the antisense transcripts (blue; data from RNA-seq). Gray regions indicate subjective night. Panel C is a RNA-seq coverage plot for Snhg12. The gene model is displayed below the coverage plot. Note the locations of the mature small nucleolar RNA (snoRNA) sequences located in the introns of Snhg12. Panel D comprises two graphs illustrating RNA-seq expression profiles for Snhg12 in brown adipose and hypothalamus. Panel E is a RNA-seq coverage plot for Arntl (red) and its antisense transcript (blue), from white adipose tissue. The gene model for Arntl is displayed above the coverage plots. Panel F comprises two graphs illustrating expression profiles for Arntl (red; data from microarrays) and the antisense transcripts (blue; data from RNA-seq), from white adipose tissue and liver. Panel G is a RNA-seq coverage plot for Per2 (red) and its antisense transcript (blue), from white adipose tissue. The gene model for Per2 is displayed above the coverage plots. Panel H comprises four graphs illustrating expression profiles for Per2 (red) and the antisense transcript (blue) from liver, adrenal gland, lung, and kidney.

FIG. 8 illustrates genomic characteristics common to rhythmically-expressed genes. Panel A comprises a plot and a gene map illustrating genomic clustering of each organ's oscillatory gene expression. The test-statistic used was the sum of the squared number of oscillatory genes expressed within a sliding nine-gene window (intergenic distance disregarded). Significance values were derived using null distributions determined by randomly shuffling gene positions 1-million times for each organ-chromosome pair. Panel B is a graph illustrating the total length of circadian vs. non-circadian genes. Panel C is a graph illustrating length of circadian vs. non-circadian genes across 5′UTRs. Panel D is a graph illustrating length of circadian vs. non-circadian genes across CDS length. Panel E is a graph illustrating length of circadian vs. non-circadian genes across 3′UTRs. Panel F is a graph illustrating spliceforms counts of circadian vs. non-circadian gene expression for detected spliceforms. Panel G is a graph illustrating spliceforms counts of circadian vs. non-circadian gene expression for unique sets of spliceforms expressed across organs. Panel H is a graph illustrating spliceforms counts of circadian vs. non-circadian gene expression for unique, dominant spliceforms expressed across organs. Panel I is a graph illustrating number of genes having the given maximum phase difference in expression between any two organs. Vegfa is shown as an example.

FIG. 9 illustrating expression of core circadian oscillator genes across organs. Panel A is a scheme illustrating expression of each gene in all organs superimposed. Panel B is a heatmap representation of expression of the circadian genes described in Panel A.

FIG. 10 is a scheme illustrating the method of discovering oscillation influence on pathways. Nodes represent Reactome pathways, with size corresponding to total number of genes in a pathway and color corresponding to percent of genes with rhythmic expression at the organism level. Edges convey pathway hierarchy. Heatmap depicts significance of pathways' oscillatory fractions by Fisher's exact test at the organ level.

FIG. 11 illustrates that Mir22 expression reduced endogenous PTGS1 in NIH3T3 cells. Panel A is a graph illustrating the representative Western blot analysis of lysates from NIH3T3 cells transfected with mirNeg, mir-22-3p, or mir-22-5p. Panel B is a graph illustrating densitometric quantification of PTGS1 protein expression from Western blots, normalized to GAPDH protein expression. Values are mean intensities relative to the mirNeg condition, ±SD. Panel C is a graph illustrating the quantification of Ptgs1 mRNA by qPCR from the same samples assayed in FIG. 11, Panel B.

FIG. 12 is a set of graphs illustrating circadian expression of core clock genes and drug targets in human lung. Data from human lung samples were downloaded from the NCBI GEO database (GSE23546). Using CYCLOPS and a set of ˜1000 homologs of clock-regulated genes in the mouse, 1349 human lung samples were re-ordered in periodic space. Each blue dot represents data from a single sample, while the red line indicates the best fit to the cosine trend. Plotted are expression levels of 33 core clock gene and drug target transcripts. If a gene had multiple clock-regulated transcripts, they were plotted. For example, CLOCK and CRY1, core clock genes, and DBP and TEF, output regulators, are expressed with high amplitude circadian rhythms as evaluated by cosinor regression. As seen in animal models, CRY1 (RORE regulated) and DBP/TEF (E-box) regulated are opposite phase. Several drug targets were also found to be clock regulated in human lung samples. For example, DDC, PDE4A, PDE4B, PDESA, PPARA, and XDH were all found to be clock-regulated.

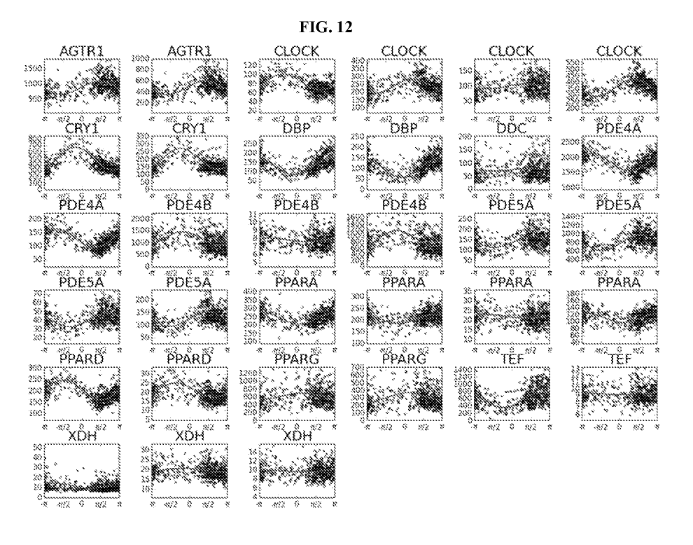

FIG. 13 is a set of graphs illustrating circadian expression of core clock genes and drug targets in human liver. Data from human lung samples were downloaded from the NCBI GEO database (GSE9588). Using CYCLOPS, 427 human liver samples were re-ordered in periodic space. Each blue dot represents data from a single sample, while the red line indicates the best fit to the cosine trend. Plotted are 20 core clock genes and drug target transcripts. If a gene had multiple clock-regulated transcripts, they were plotted. For example, CLOCK and CRY1, core clock genes, and DBP and TEF, output regulators, are expressed with high amplitude circadian rhythms as evaluated by cosinor regression. As seen in animal models, CRY1 (RORE regulated) and DBP (E-box) regulated are opposite phase. Several drug targets were also found to be clock regulated in human liver samples. For example, AGTR1, DDC, PDE4A, PDE4B, PDESA, PPARA, and XDH were all found to be clock-regulated.

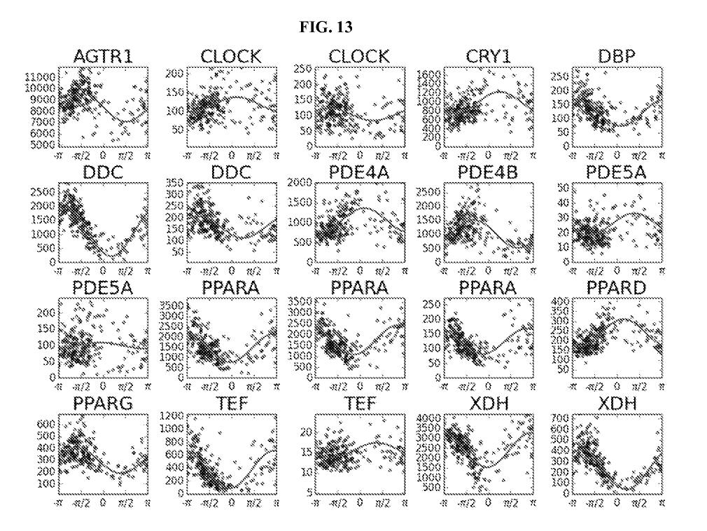

### DETAILED DESCRIPTION OF THE INVENTION

The present invention relates to the unexpected discovery of patterns of circadian gene expression within various organs and tissues of a human. The invention further relates to a method of developing an improved formulation of a therapeutic substance to improve its efficacy and reduce its side effects according to the expression of these circadian genes.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the terms “adverse effect” and “side effect” are used interchangeably. Both refer to an undesired harmful effect resulting from a medication.

As used herein, the phrase “before bedtime” means up to 6 hours prior to bedtime, e.g., 1 hour, 2 hours, three hours, four hours, five hours, and 6 hours prior. Before bedtime also means at or about bedtime. In certain embodiments, it includes at the time of a final meal prior to bedtime. Bedtime is relative to a subject. For example, a subject who sleeps during the day will have a bedtime in the morning and a standard subject who sleeps at night bill have a bedtime in the evening.

The terms “carrier” or “carrier system” means one or more compatible substances that are suitable for delivering, containing, or “carrying” therapeutic compound ingredient(s) for administration to a patient or subject.

As used herein, the term “chronotherapy” refers to the use of circadian time in determining optimal formulation and dosage of therapeutic compounds to be administered.

As used herein, the term “circadian gene” refers to any gene identified whose expression cycles with a 24-hour period.

As used herein, the term “circadian hour” is defined as the unit of time corresponding to 1/24 of the duration of a circadian cycle. By convention, the onset of locomotor activity of diurnal organisms defines circadian time zero (CT 0). Thus, the onset of activity of nocturnal organisms defines circadian time twelve (CT 12).

As used herein, the terms “circadian phase” and “circadian cycle” are used interchangeably. Both refer to the phase of a circadian rhythm where its peak and trough occur within 24 hours.

As used herein, the term “circadian time” refers to a standard of time based on the free-running period of a rhythm (oscillation).

As used herein, the term “coordinated release” refers to release of at least one therapeutic compound such that the release of the therapeutic compound coincides with peak or trough expression of one or more target genes of the therapeutic compound.

As used herein, the term “drug target” refers to genes whose expression products are bound by or are otherwise functionally affected by a given drug.

As used herein, the term “delayed-release” refers to a medication that does not immediately disintegrate and release the active ingredient into the body of a mammal when administered thereto.

As used herein, the term “delayed-release formulation” refers to a formulation delaying the active ingredient's release to the body of a mammal.

As used herein, the term “enteric coating” relates to a polymer barrier applied on an oral medication. In one instance, the enteric coating works by presenting a barrier wrapping around the active ingredient of an oral medication. Such barrier is stable at the highly acidic PH found in the stomach, but breaks down rapidly at a less acidic or basic environment.

The term “extended-release” is used herein with reference to a drug formulation that releases the therapeutic compound slowly into the bloodstream over time. The advantage of extended-release formulations is to take at less frequent intervals than immediate-release formulations of the same drug.

As used herein, the term “half-life” refers to the duration of time required for the concentration or amount of drug in the body to be reduced by one-half. Generally, the half-life of a drug relates to the amount of the drug in plasma.

The term “immediate-release” is used herein with reference to a drug formulation that does not contain a dissolution rate controlling material. There is substantially no delay in the release of the active ingredient following administration of an immediate-release formulation.

As used herein, the term “inhibit” as it relates to a gene refers to restraining or preventing the expression of the gene, including production of the corresponding RNA or protein.

As used herein, the terms “peak phase” and “peak expression” are used interchangeably. Both refer to the time when the circadian genes or protein expressed thereby are most active.

As used herein, the term “pharmaceutically-acceptable excipients” refers to any physiologically inert, pharmacological inactive material known to one skilled in the art, which is compatible with the physical and chemical characteristics of the active ingredient selected for use. Pharmaceutically-acceptable excipients include, but are not limited to, polymers, resins, plasticizers, fillers, lubricants, solvents, co-solvents, surfactants, preservatives, sweetener agents, flavoring agents, buffer systems, pharmaceutical-grade dyes or pigments, and viscosity agents. Flavoring agents among those useful herein include those described in Remington's Pharmaceutical Sciences, 18th Edition Mack Publishing Company, 1990, pp. 1288-1300, incorporated by reference herein. Dyes or pigments among those useful herein include those described in Handbook of Pharmaceutical Excipients pp. 81-90, 1986 by the American Pharmaceutical Association & the Pharmaceutical Society of Great Britain, incorporated by reference herein.

As used herein, “pharmaceutically acceptable salts” refer to derivatives of the therapeutic compound wherein the parent compound is modified by making an acid or base salt thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, and arginine.

As used herein, the term “pharmaceutical composition” means an oral dosage form comprised of a safe and effective amount of an active ingredient and a pharmaceutically-acceptable excipient.

As used herein, “preventing,” “prevent,” or “protecting against” describes reducing or eliminating the onset of the symptoms or complications of a disease or disorder.

The phrase “reducing the risk of”, as used herein, means to lower the likelihood or probability of a disease or disorder from occurring in a patient or subject, especially when the patient or subject is predisposed to such or at risk of contracting a disease or disorder.

One of ordinary skill in the art will appreciate that there is some overlap in the definitions of “treating”, “preventing”, and “reducing the risk of”.

As used herein, the term “prodrug” refers to a medication that is administered in an inactive or less than fully active form, and is then converted to its active form through a normal metabolic process, such as hydrolysis of an ester form of the drug.

As used herein, the terms “safe and effective amount”, “effective amount”, and “pharmaceutically effective amount” are used interchangeably. All refers to an amount of a compound or composition high enough to significantly positively modify the symptoms and/or condition to be treated, but low enough to avoid serious side effects (at a reasonable benefit/risk ratio), within the scope of sound medical judgment. The safe and effective amount of active ingredient for use in the method of the invention herein will vary with the particular condition being treated, the age and physical condition of the patient being treated, the severity of the condition, the duration of the treatment, the nature of concurrent therapy, the particular active ingredient being employed, the particular pharmaceutically-acceptable excipient utilized, and like factors within the knowledge and expertise of the attending physician.

As used herein, the phrase “pharmaceutically acceptable” refers to those therapeutic compounds, materials, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

As used herein, the phrase “release of a therapeutic compound” means that the therapeutic compound enters plasma and reaches at safe and effective amount.

As used herein, the phrase “regulated release” includes immediate-release, extended-release, delayed release, or combination thereof.

As used herein, the terms “synchronize” and “coincide” are used interchangeably. Both refers to an action matching the time when a therapeutic compound reaches safe and effective amount in plasma with the peak or trough of circadian genes or proteins.

A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, the term “tablet” is intended to encompass compressed formulations of all shapes and sizes whether coated or uncoated. As used herein, the term “capsule” or “caplet” is intended to encompass a powdered, pelleted, or beaded formulations enclosed in a shell, e.g., a gelatin shell such as a soft gelatin or hard gelatin capsule.

As used herein, the terms “therapeutic substance,” “drug,” “therapeutic compound,” and “active ingredient” are used interchangeably. All refer to a substance having or exhibiting healing power, curing or mitigating the symptoms of a disease.

As used herein, the phrase “time-release” includes extended-release, delayed release, or combination thereof.

As used herein, the term “transporter” refers to a transport protein that serves the function of moving other material within an organism.

The term “treating”, as used herein, means to cure an already present disease or disorder. Treating can also include inhibiting, i.e., arresting the development of a disease or disorder, and relieving or ameliorating, i.e., causing regression of the disease or disorder.

As used herein, the term “trough” or “trough expression” refers to the time when the target genes or proteins expressed thereby are least active.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6, and so on, as well as individual numbers within that range, for example, 1, 2, 2.7, 3, 4, 5, 5.3, and 6. This applies regardless of the breadth of the range.

### DESCRIPTION

The present invention relates to methods for developing formulations for treating one or more diseases, conditions, or disorders associated with genes that are expressed with circadian rhythms (i.e., genes that oscillate with circadian rhythm). Such formulations have regulated release of at least one therapeutic compound such that the compound's release coincides with peak or trough expression of one or more of the compound's target genes and in at least one tissue type.

The design of appropriate formulation(s) is within the routine level of skill in the art. Before formulations are designed, it is first necessary to identify the disorders and as well as the therapeutic compounds capable of treating the disorder. Then, target gene(s) for the therapeutic compounds are ascertained. Examples of suitable disorders, therapeutic compounds, target gene(s) for the various therapeutic compounds, and the half-lives of exemplary therapeutic compounds are listed in Table 1, infra.

Next, circadian oscillations in transcript expression (including peak and trough expressions) for the target genes in specific tissue types are determined, for example, by using the methods described herein. Data regarding circadian oscillations, including coding and non-coding genes, are available via the World Wide Web (www) bioinf dot itmat dot upenn dot edu/circa, a subset of which is summarized in Table 2, infra.

Using the information provided in Tables 1 and 2, as well as methods well known in the art for making appropriate immediate release and/or time-releases formulations, suitable formulation(s) can be devised that will be useful in treating disease(s), condition(s), or disorder(s) associated with genes that are expressed with circadian rhythms.

For example, formulations can be prepared for situations where a given therapeutic compound has one target gene in one tissue; where a given therapeutic compound has more than one target gene in one tissue; where therapeutic compound(s) have a target gene that is differentially expressed in more than one tissue type; and/or where therapeutic compound(s) have two (or more) target genes that are differentially expressed in more than one tissue type. Formulations can also be designed to include more than one therapeutic compound, wherein the more than one therapeutic compound may have two (or more) target genes that are differently expressed, in time and/or in tissue types. In addition, formulations can also be designed including more than two (e.g., three, four, five, or more) therapeutic compounds.

In other embodiments, formulations can also be designed such that one therapeutic compound is released coincidental with peak or trough expression of its target gene and a second therapeutic compound is released at times that may be independent of its target gene's peak or trough expression. It is often preferable to temporally segregate a therapeutic effect from unwanted side effects. For example, certain statins can cause rhabdomyolysis, which is breakdown of muscle fibers that leads to the release of muscle fiber contents (myoglobin) into the bloodstream. Thus, it is ideal if a statin's therapeutic effect of lipid lowering in the liver is temporally segregated from a side effect of muscle fiber breakdown.

The present invention also includes coordinated release of a therapeutic compound selected from Table 1, wherein release of the therapeutic compound from the formulation coincides with peak or trough expression of at least one target gene of the therapeutic compound. For example, the at least one target gene is selected from Table 1. In these formulations, the therapeutic compound is released at a defined time (in hours) after contact with a solution having a pH of between 1 and 5 and a temperature of between 35 and 42° C. Those skilled in the art will recognize that, while the exact time for release of the therapeutic compound from the formulation is application specific, the defined time will never be higher than 12 hours.

In one specific example, the at least one target gene is PPARα, and the therapeutic compound may be a fibrate having a half-life of less than six hours. In such formulations, the fibrate is released two to four hours after contact with a solution having a pH of between 1 and 5 and a temperature of between 35 and 42° C. Suitable fibrates for use in such formulations include, but are not limited to, Gemfibrozil or Bezafibrate. Ideally, the formulation is taken by a patient before bedtime (e.g., at bedtime or two to six hours before bedtime) and exhibits normal pharmacokinetics once released from the formulation.

In another specific example, the target gene is Niar1, a niacin receptor, and the therapeutic compound may be niacin (i.e., less than about 500 mg niacin per dose). In such formulations, the niacin is released zero to six hours (e.g., zero to two hours; two to four hours; or four to six hours) after contact with a solution having a pH of between 1 and 5 and a temperature of between 35 and 42° C. The therapeutic compound can be dosed before bedtime (e.g., at bedtime or two to six hours before bedtime) and exhibits normal pharmacokinetics once released from the formulation. The therapeutic compound may also be dosed within one hour of a final meal before bedtime. The niacin can be immediate-released once release from a formulation has begun.

Also included are formulations providing coordinated release of a therapeutic compound selected from Table 1, wherein release of the therapeutic compound from the formulation coincides with peak or trough expression of at least one target gene of the therapeutic compound. The formulation comprises two portions of the therapeutic compound: a first portion and a second portion, and provides coordinated release of the two portions of the therapeutic compound such that release of the first portion of the therapeutic compound coincides with peak or trough expression of the at least one target gene and release of the second portion of the therapeutic compound occurs after peak or trough expression of the at least one target gene.

In such formulations, the first portion of the therapeutic compound is immediate-released or is time-released.

In various embodiments, the release of the second portion of the therapeutic compound occurs prior to one half-life of the therapeutic compound following the first portion release; occurs after one half-life of the therapeutic compound following the first portion release; occurs after the release of substantially the entire first portion and prior to one half-life of the therapeutic compound following the release of the first portion; or occurs prior to the release of substantially the entire first portion.

In some formulations, release of a second portion of the therapeutic compound contained in the formulation occurs at a time independent of an expression of its target gene in a tissue type and avoids undesirable side effect(s).

Also included are formulations providing coordinated release of a therapeutic compound selected from Table 1, wherein the therapeutic compound inhibits at least two target genes and wherein the formulation provides coordinated release such that release of a first portion of the therapeutic compound contained in the formulation coincides with peak or trough expression of a first target gene and release of a second portion of the therapeutic compound contained in the formulation coincides with peak or trough expression of a second target gene. For example, the first target gene and the second target gene are each selected from Table 1, and the peak or trough expression of the first target gene and peak or trough expression of the second target gene are defined in Table 2.

The first portion of the therapeutic compound can be released 0 to 2 hours after contact with a solution having a pH of between 1 and 5 and a temperature of between 35 and 42° C.

The second portion of the therapeutic compound can be released 2-6 hours following the first portion is released, which correlates with a differential in peak or trough expression of the first and second target genes as defined in Table 2.

In such formulations, the release of a second portion of the therapeutic compound contained in the formulation occurs at a time independent of a differential in peak or trough expression of a first target gene and a second target gene as defined in Table 2 and avoids undesirable side effect(s).

The first portion of the therapeutic compound can be immediate-released or time-released.

These formulations further comprise at least a third portion of the therapeutic compound. The release of the at least third portion of the therapeutic compound contained in the formulation coincides with peak or trough expression of at least a third target gene and wherein peak or trough expression of the at least third target gene is defined in Table 2. In one specific example, the at least two target genes is selected from the group consisting of PPARα, PPARδ, and PPARγ. In such formulations, the therapeutic compound is a fibrate (e.g., Bezafibrate) having a half-life of less than six hours. For example, the fibrate is released two to four hours after contact with a solution having a pH of between 1 and 5 and a temperature of between 35 and 42° C. Ideally, in these formulations, the therapeutic compound is dosed before the patient's bedtime and exhibits normal pharmacokinetics once released from the formulation.

Also included are formulations providing coordinated release of a therapeutic compound selected from Table 1, wherein release of the therapeutic compound from the formulation coincides with peak or trough expression of at least one target gene of the therapeutic compound, wherein the target gene is expressed in at least two tissue types and wherein the formulation provides coordinated release of the therapeutic compound such that release of a first portion of the therapeutic compound contained in the formulation coincides with peak or trough expression of the target gene in a first tissue type and release of a second portion of the therapeutic compound contained in the formulation coincides with peak or trough expression of the target gene in a second tissue type. In such formulations, the target gene is selected from Table 1 and/or the peak or trough expression of the target gene in each tissue type is defined in Table 2. The first tissue type and the second tissue type are each selected from Table 1.

In these formulations, the first portion of the therapeutic compound is released 0-2 hours after contact with a solution having a pH of between 1 and 5 and a temperature of between 35 and 42° C. The second portion of the therapeutic compound is released 2-6 hours following the release of the first portion, which correlates with a differential in peak or trough expression of the target gene between the first and second tissue types as defined in Table 2.

In such formulations, the release of a second portion of the therapeutic compound contained in the formulation occurs at a time independent of a differential in peak or trough expression of a first target gene and a second target gene as defined in Table 2 and avoids undesirable side effect(s).

The first portion of the therapeutic compound can be immediate-released or time-released.

In one specific example, the target gene is PPARα, the first tissue type is liver and the second tissue type is kidney. In such formulations, the therapeutic compound is Gemfibrozil or Bezafibrate. The therapeutic compound can be dosed before bedtime.

Such formulations can also provide release of at least a third portion of the therapeutic compound contained in the formulation such that the release of the at least third portion coincides with peak or trough expression of the target gene in an at least third tissue type and wherein peak or trough expression of the target gene in the at least third tissue type is defined in Table 2.

Also included are formulations providing coordinated release of a therapeutic compound selected from Table 1, wherein the therapeutic compound inhibits at least two target genes, wherein the formulation provides coordinated release such that release of a first portion of the therapeutic compound contained in the formulation coincides with peak or trough expression of a first target gene and release of a second portion of the therapeutic compound contained in the formulation coincides with peak or trough expression of a second target gene, wherein the at least two target genes are expressed in at least two tissue types and wherein the formulation provides coordinated release of the therapeutic compound such that release of the first portion of the therapeutic compound contained in the formulation coincides with peak or trough expression of the first target gene in the first tissue type and release of the second portion of the therapeutic compound contained in the formulation coincides with peak or trough expression of the second target gene in the second tissue type. In such formulations, the first target gene and the second target gene are each selected from Table 1 and/or peak or trough expression of the first target gene and peak or trough expression of the second target gene are defined in Table 2.

The first portion of the therapeutic compound can be immediate-released or time-released.

In these formulations, the first portion of the therapeutic compound can be released 0-2 hours after contact with a solution having a pH of between 1 and 5 and a temperature of between 35 and 42° C. The second portion of the therapeutic compound can be released 2-6 hours following the release of the first portion, which correlates with a differential in peak or trough expression of the first and second target genes as defined in Table 2.

In one specific example, the first target gene is PPARα and the first tissue type is liver. In this example, the second target gene is PPARγ and the second tissue type is kidney. The therapeutic compound is Bezafibrate. In this formulation, the therapeutic compound is dosed before bedtime.

Such formulations may additionally provide release of at least a third portion of the therapeutic compound contained in the formulation such that the release of the at least third portion coincides with peak or trough expression of at least a third target gene and wherein peak or trough expression of the at least third target gene is defined in Table 2, optionally, wherein the at least a third target gene is expressed in a third tissue type.

Also included is a formulation comprising at least two therapeutic compounds selected from Table 1, wherein each therapeutic compound inhibits at least one different target gene wherein release of a first therapeutic compound from the formulation coincides with peak or trough expression of at least one target gene of the first therapeutic compound and wherein release of a second therapeutic compound from the formulation coincides with peak or trough expression of at least one target gene of the second therapeutic compound. Release of the second therapeutic compound occurs a specified time following release of the first therapeutic compound wherein the specified time correlates with a differential between peak or trough expression of at least one target gene of the first therapeutic compound and peak or trough expression of at least one target gene of the second therapeutic compound and wherein peak or trough expression of each target gene is defined in Table 2. Release of the second therapeutic compound can also occur at a specified time following release of the first therapeutic compound wherein the specified time correlates with a differential between peak or trough expression of the at least one target gene of the first therapeutic compound in a first tissue type and peak or trough expression of the at least one target gene of the second therapeutic compound in a second tissue type and wherein peak or trough expression of each target gene in each tissue type is defined in Table 2.

The first target gene and the second target gene can each be selected from Table 1.

For example, release of the second therapeutic compound occurs at a specified time following release of the first therapeutic compound wherein the specified time correlates with a differential in peak or trough expression of the target gene of the first therapeutic compound and the peak or trough expression of the target gene of the second therapeutic compound as defined in Table 2.

The first therapeutic compound may be immediate-released or time-released.

In these formulations, the first therapeutic compound is released 0-2 hours after contact with a solution having a pH of between 1 and 5 and a temperature of between 35 and 42° C. The second therapeutic compound can be released 2-4 hours following release of the first therapeutic compound, which correlates with a differential in peak or trough expression of the target gene of the first therapeutic compound and the target gene of the second therapeutic compound as defined in Table 2.

In one specific example, the target gene of the first therapeutic compound is Niacr1, or a niacin receptor and the target gene of the second therapeutic compound is Hmgcr. For example, when the first therapeutic compound is niacin (e.g., less than 500 mg per dose) and the second therapeutic compound is a statin (e.g., Cerivastatin, Fluvastatin, or Simvastatin) having a half-life of less than three hours, niacin is released two to four after contact with a solution having a pH of between 1 and 5 and a temperature of between 35 and 42° C. and the statin is released four to six after contact with a solution having a pH of between 1 and 5 and a temperature of between 35 and 42° C. In such formulations, the first therapeutic compound and the second therapeutic compound are dosed before bedtime (e.g., within 2 hours of bedtime or within one hour of a final meal before bedtime) and each exhibits normal pharmacokinetics once released from the formulation.

In one specific example of such a formulation, the target gene of the first therapeutic compound is Agtr1a and the target gene of the second therapeutic compound is Adrb2 or Adrb1. For example, when the first therapeutic compound is an angiotensin receptor blocker (ARB) having a half-life of less than six hours (e.g., Valsartan or Losartan) and wherein the second therapeutic compound is a beta blocker having a half-life of less than three hours (e.g., Metoprolol or Timolol), the ARB can be released zero to two hours after contact with a solution having a pH of between 1 and 5 and a temperature of between 35 and 42° C. and the beta blocker can be released two to four hours after contact with a solution having a pH of between 1 and 5 and a temperature of between 35 and 42° C. In these formulations, the first therapeutic compound and the second therapeutic compound are dosed before bedtime and each exhibits normal pharmacokinetics once released from the formulation.

In another specific example of such a formulation, the target gene of the first therapeutic compound is Agtr1a and the target gene of the second therapeutic compound is Car4, Car2, Car12, or Car9. For example, when the first therapeutic compound is an angiotensin receptor blocker (ARB) having a half-life of less than six hours (e.g., Valsartan or Losartan) and the second therapeutic compound is a diuretic (e.g., Hydrochlorothiazide), the ARB can be released zero to two hours after contact with a solution having a pH of between 1 and 5 and a temperature of between 35 and 42° C. and the diuretic can be released six to eight hours after contact with a solution having a pH of between 1 and 5 and a temperature of between 35 and 42° C. In these formulations, the first therapeutic compound and the second therapeutic compound each exhibit normal pharmacokinetics once released from the formulation.

In a further specific example of such a formulation, the target gene of the first therapeutic compound is Ace and the target gene of the second therapeutic compound is Adrb2 or Adrb1. For example, when the first therapeutic compound is an acetylcholinesterase (ACE) inhibitor having a half-life of less than six hours (e.g., Enalapril or Reamipril) and the second therapeutic compound is a beta blocker having a half-life of less than three hours (e.g., Metoprolol or Timolol), the ACE inhibitor can be released zero to two hours after contact with a solution having a pH of between 1 and 5 and a temperature of between 35 and 42° C. and the beta blocker can be released two to four hours after contact with a solution having a pH of between 1 and 5 and a temperature of between 35 and 42° C. In these formulations, the first therapeutic compound and the second therapeutic compound are dosed before bedtime and each exhibits normal pharmacokinetics once released from the formulation.

In yet another specific example of such a formulation, the target gene of the first therapeutic compound is Ace and the target gene of the second therapeutic compound is Car4, Car2, Car12, or Car9. For example, when the first therapeutic compound is an acetylcholinesterase (ACE) inhibitor having a half-life of less than six hours (e.g., Enalapril or Reamipril) and the second therapeutic compound is a diuretic (e.g., Hydrochlorothiazide), the ARB can be released zero to two hours after contact with a solution having a pH of between 1 and 5 and a temperature of between 35 and 42° C. and the diuretic can be released six to eight hours after contact with a solution having a pH of between 1 and 5 and a temperature of between 35 and 42° C. In these formulations, the first therapeutic compound and the second therapeutic compound each exhibit normal pharmacokinetics once released from the formulation.

In another embodiment, target gene of the first therapeutic compound is PPARα and the target gene of the second therapeutic compound is Hmgcr. For example, when the first therapeutic compound is a fibrate having a half-life of less than two hours and the second therapeutic compound is a statin having a half-life of less than two hours, the fibrate can be

released zero to two hours after contact with a solution having a pH of between 1 and 5 and a temperature of between 35 and 42° C. and the statin can released four to six hours after contact with a solution having a pH of between 1 and 5 and a temperature of between 35 and 42° C. In these formulations, the fibrate is principally metabolized by CYP3A4 (e.g., Gemfibrozil) and the statin is principally metabolized by CYP2C9 (e.g., Fluvastatin). In these formulations, the first therapeutic compound and the second therapeutic compound can be dosed before bedtime and are each exhibits normal pharmacokinetics once released from the formulation.

Any of these formulations can further provide release of at least a third therapeutic compound contained in the formulation such that release of the at least third therapeutic compound coincides with peak or trough expression of at least a third target gene and wherein peak or trough expression of the at least third target gene is defined in Table 2.

Also included are formulations providing coordinated release of at least two different therapeutic compounds selected from Table 1, wherein the at least two therapeutic compounds may independently inhibit more than two target genes, but have at least one common target gene, wherein release of a first therapeutic compound coincides with peak or trough expression of the common target gene at one time and release of a second therapeutic compound coincides with peak or trough expression of the common target gene at a different time. In such formulations, the first therapeutic compound has a half-life that differs from the half-life of the second therapeutic compound and wherein the half-lives of the first therapeutic compound and the second therapeutic compound are identified in Table 1. The first therapeutic compound has a half-life shorter than the half-life of the second therapeutic compound. Alternatively, the first therapeutic compound has a half-life longer than the half-life of the second therapeutic compound. In these formulations, the first therapeutic compound is immediate-release or time-released. Likewise, the second therapeutic compound is immediate-release or time-released.

In various embodiments, the first therapeutic compound is released before peak or trough expression of the common target gene and the second therapeutic compound is released after peak or trough expression of the common target gene or the first and second therapeutic compounds are both released before peak or trough expression of the common target gene.

In further embodiments, the release of the second therapeutic compound occurs a specified time following release of the first therapeutic compound and wherein the specified time correlates with a differential in half-lives between the first and second therapeutic compounds as defined in Table 2.

The common target gene of the first and second therapeutic compounds is selected from Table 1.

In these formulations, the first therapeutic compound is released at a defined time (in hours) following after contact with a solution having a pH of between 1 and 5 and a temperature of between 35 and 42° C. Determination of the defined time is within the routine level of skill in the art. Likewise, the second therapeutic compound is released at a defined time (in hours) following release of the first therapeutic compound, which correlates with a differential in half-lives between the first and second compounds as defined in Table 2. Determination of this defined time is within the routine level of skill in the art.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990) and Remington: The Science and Practice of Pharmacy, 22^{nd} Edition, Baltimore, Md.: Lippincott Williams & Wilkins, 2012, both of which are herein incorporated by reference.

Additionally, any of the therapeutic compounds of the present invention, for example, the salts of the compounds, can exist in either hydrated or unhydrated (the anhydrous) form or as solvates with other solvent molecules. Non-limiting examples of hydrates include monohydrates and dehydrates. Non-limiting examples of solvates include ethanol solvates and acetone solvates.

The therapeutic compounds of the present invention can also be prepared as esters, for example pharmaceutically acceptable esters. For example a carboxylic acid function group in a compound can be converted to its corresponding ester, e.g., a methyl, an ethyl, and another ester. Also, an alcohol group in a compound can be converted to its corresponding ester, e.g., an acetate, a propionate, and another ester.

The therapeutic compounds of the present invention can also be prepared as prodrugs, for example pharmaceutically acceptable prodrugs. Since prodrugs are known to enhance numerous desirable qualities of pharmaceuticals (e.g., solubility, bioavailability, manufacturing, etc.) the therapeutic compounds of the present invention can be delivered in prodrug form. Thus, the present invention is intended to cover prodrugs of the presently claimed therapeutic compounds, methods of delivering the same and compositions containing the same. “Prodrugs” are intended to include any covalently bonded carriers that release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to the parent compound. Prodrugs include therapeutic compounds of the present invention wherein a hydroxy, amino, or sulfhydryl group is bonded to any group that, when the prodrug of the present invention is administered to a mammalian subject, cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

The formulations disclosed herein may optionally contain an immediate release portion. An immediate release portion of the formulation may to release more than 50%, (e.g., 50%, 60%, 70%, 80%, 85%, 90%, 95%, 96%, 97%, 98%, 99%, or essentially all) of the therapeutic compound(s) in the at least one immediate release portion(s) within about one hour. In certain embodiments, more than 50% and up to essentially all the therapeutic compound(s) in the at least one immediate release portion(s) may be released in less than about 45 min. In other embodiments, more than 50% and up to essentially all the therapeutic compound(s) in the at least one immediate release portion(s) may be released in less than about 30 min. In yet other embodiments, more than 50% and up to essentially all the therapeutic compound(s) in the at least one immediate release portion(s) may be released in less than about 20 min. In yet other embodiments, more than 50% and up to essentially all the therapeutic compound(s) in the at least one immediate release portion(s) may be released in less than about 15 min. In yet other embodiments, more than 50% and up to essentially all the therapeutic compound(s) in the at least one immediate release portion(s) may be released in less than about 10 min. In yet other embodiments, more than 50% and up to essentially all the therapeutic compound(s) in the at least one immediate release portion(s) may be released in less than about 5 min.

### Formulation:

The formulation of the present invention includes one or more of the following essential and optional components. The formulation of the present invention also includes therapeutic compound(s).

Suitable carrier components are described in e.g., Eds. R. C. Rowe, et al., Handbook of Pharmaceutical Excipients, Fifth Edition, Pharmaceutical Press (2006); Remington's Pharmaceutical Sciences, 18th ed. (Mack Publishing Company, 1990); and Remington: The Science and Practice of Pharmacy, 22^{nd} Edition, Baltimore, Md.: Lippincott Williams & Wilkins, 2012. Even though a functional category can be provided for many of these carrier components, such a functional category is not intended to limit the function or scope of the component, as one of ordinary skill in the art will recognize that a component can belong to more than one functional category and that the level of a specific component and the presence of other components can affect the functional properties of a component.

a. Emulsifier

The formulations of the present invention may include at least one emulsifier. Useful emulsifiers include polyglycolized glycerides (also known as polyglycolysed glycerides). These materials are generally surface active and depending on their exact composition have a range of melting points and hydrophilic/lipophilic balance ranges (HLBs). These materials are often further combined with a polyhydric alcohol, such as glycerol. The polyglycolized glycerides are mixtures of glycerides of fatty acids and of esters of polyoxyethylene with fatty acids. In these mixtures, the fatty acids are generally saturated or unsaturated C₈-C₂₂, for example C₈-C₁₂ or C₁₆-C₂₀. The glycerides are generally monoglycerides, diglycerides, or triglycerides or mixtures thereof in any proportions. Polyglycolysed glycerides are marketed e.g., by Gattefosse under the trade names Labrafil, Labrosol, and Gelucire. The Gelucire polyglycolized glycerides are often designated with the melting point and HLB. For example, Gelucire 53/10 refers to a material having a melting point of 53° C. and an HLB of 10. Gelucire materials useful herein include Gelucire 44/14 and Gelucire 50/13. Other emulsifiers useful herein include vitamin E TPGS, ploxamers, and lecithin. Vitamin E TPGS is also known as d-α-tocopheryl polyethylene glycol 1000 succinate. Ploxamers are known by the trade name Pluronics, and are nonionic triblock copolymers composed of a central hydrophobic chain of polyoxypropylene (poly(propylene oxide)) flanked by two hydrophilic chains of polyoxyethylene (poly(ethylene oxide)).

The emulsifier can constitute from about 0.1% to about 99.9% of the formulation of the present invention. In embodiments, the emulsifier can constitute from about 1% to about 20%, from about 1% to about 15%, and from about 1% to about 10% of the formulation of the present invention.

b. Polymeric Dissolution Aid

The formulations of the present invention may include at least one polymeric dissolution aid. Such polymeric dissolution aids include polymers of 1-ethenyl-2-pyrrolidinone; polyamine N-oxide polymers; copolymers of N-vinylpyrrolidone and N-vinylimidazole; polyvinyloxazolidones and polyvinylimidazoles or mixtures thereof. Particularly useful are polymers of 1-ethenyl-2-pyrrolidinone, especially the homopolymer. Generally this homopolymer has a molecular weight range of about 2500 to 3,000,000. This homopolymer is known as polyvinylpyrollidone, PVP, or povidone and in other instances can function as a dissolution aid, disintegrant, suspending agent, or binder.

The polymeric dissolution aid can constitute from about 0.1% to about 99.9% of the formulations of the present invention. In certain embodiments, the polymeric dissolution aid can constitute from about 1% to about 10%, from about 1% to about 5%, and from about 1% to about 2.5% of the formulations of the present invention.

c. Binder

The formulations of the present invention can include at least one binder or binding agent. Examples of binders are cellulose; microcrystalline cellulose; low viscosity water soluble cellulose derivatives such as microcrystalline cellulose, hydroxypropyl cellulose, hydroxypropylmethyl cellulose (HPMC), hydroxyethyl cellulose, ethyl cellulose, methyl cellulose, and sodium carboxy-methyl cellulose; alginic acid derivatives; polyvinylpyrrolidone; magnesium aluminum silicate; starches such as corn starch and potato starch; gelatin; sugars (including sucrose, glucose, dextrose and lactose); waxes; gums (e.g., guar gum, arabic gum, acacia gum, and xanthan gum); and tragacanth. A preferred binder is HPMC. Preferably the binding agent constitutes from about 1 to about 10%. Preferably, the binder constitutes from about 1 to about 4% by weight of the formulation.

### d. pH Modifier

The formulations of the present invention can further include at least one pH modifier. Examples of pH modifiers are generally acidic or basic materials that can be used to modify or adjust the pH of the formulation or its environment. Non-limiting examples of pH modifiers useful herein include aspartic acid, citric acid, ethanesulfonic acid, fumaric acid, lactic acid, methanesulfonic acid, tartaric acid, and mixtures thereof.

e. Filler

The formulations of the present invention can further include at least one filler. Examples of fillers are microcrystalline cellulose; glucose; lactose; dextrose; mannitol; sorbitol; sucrose; starches; fumed silica; salts such as sodium carbonate and calcium carbonate; and polyols such as propylene glycol. Preferably, fillers are present in an amount of from 0% to about 50% by weight of the formulations, either alone or in combination. More preferably they are present from about 5% to about 20% of the weight of the formulation.

f. Dispersing or Wetting Agent

The formulations of the present invention can further include at least one dispersing or wetting agent. Examples of dispersing or wetting agents are polymers such as polyethylene-polypropylene, and surfactants such as sodium lauryl sulfate. Preferably the dispersing or wetting agent is present in an amount of from 0% to about 50% by weight, either alone or in combination. More preferably they are present from about 5% to about 20% of the weight of the formulation.

g. Disintegrant

The formulations of the present invention can further include at least one disintegrant. Examples of disintegrants are modified starches or modified cellulose polymers, e.g., sodium starch glycolate. Other disintegrants include agar; alginic acid and the sodium salt thereof; effervescent mixtures (e.g., the combination of an acid such as tartaric acid or citric acid and a basic salt such as sodium or potassium bicarbonate, which upon contact with an aqueous environment react to produce carbon dioxide bubbles which help to break up or disintegrate the composition); croscarmelose; crospovidone; sodium carboxymethyl starch; sodium starch glycolate; clays; and ion exchange resins. Preferably the disintegrant is present in an amount of from 0% to about 50% by weight of the formulation, either alone or in combination. More preferably the disintegrant is present from about 5% to about 20% by weight of the formulation.

h. Lubricant

The formulations of the present invention can further include at least one lubricant. Generally, the lubricant is selected from a long chain fatty acid or a salt of a long chain fatty acid. Suitable lubricants are exemplified by solid lubricants including silica; talc; stearic acid and its magnesium salts and calcium salts; calcium sulfate; and liquid lubricants such as polyethylene glycol; and vegetable oils such as peanut oil, cottonseed oil, sesame oil, olive oil, corn oil and oil of theobroma. Preferably the lubricant is present in an amount of from 0% to about 50% by weight of the formulation, either alone or in combination. More preferably it is present from about 5% to about 20% of the weight of the formulation.

i. Additional Components

The formulations of the present invention can further include one or more additional components selected from a wide variety of excipients known in the pharmaceutical formulation art. According to the desired properties of the tablet or capsule, any number of ingredients can be selected, alone or in combination, based upon their known uses in preparing the formulations of the present invention. Such ingredients include, but are not limited to, water, nonaqueous solvents (e.g., ethanol), coatings, capsule shells, colorants, waxes, gelatin, flavorings, preservatives (e.g., methyl paraben, sodium benzoate, and potassium benzoate), antioxidants (e.g., ascorbic acid, butylated hydroxyanisole (“BHA”), butylated hydroxytoluene (“BHT”), and vitamin E and vitamin E esters such as tocopherol acetate), flavor enhancers, sweeteners (e.g., aspartame and saccharin), compression aids, and surfactants. Exemplary coating agents include, but are not limited to: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

### Extended-Release Formulation:

In certain embodiments, the therapeutic compound described herein may have little side effect in treating the intended disease, and the desired administration time is not convenient, an extended-release formulation is desirable. In other embodiments, an extended-release formulation may be used in combination with a delayed-release formulation or an immediate-release formulation to exploit the circadian gene expression.

The formulations disclosed herein may include at least one extended-release portion containing the therapeutic compound(s) and an extended-release component. Suitable extended-release components include, for example, polymers, resins, hydrocolloids, hydrogels, and the like.

Suitable polymers for inclusion in an extended-release portion of the formulation may be linear, branched, dendrimeric, or star polymers, and include synthetic hydrophilic polymers as well as semi-synthetic and naturally occurring hydrophilic polymers. The polymers may be homopolymers or copolymers, such as random copolymers, block copolymers, and graft copolymers. Suitable hydrophilic polymers include, but are not limited to: polyalkylene oxides, particularly poly(ethylene oxide), polyethylene glycol and poly(ethylene oxide)-poly(propylene oxide) copolymers; cellulosic polymers, such as methylcellulose, hydroxymethylcellulose, hydroxyethylcellulose, hydroxypropylcellulose, hydroxypropylmethylcellulose, carboxymethylcellulose, microcrystalline cellulose, and polysaccharides and their derivatives; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); polyalkylene oxides; poly(olefinic alcohol)s such as polyvinyl alcohol); poly(N-vinyl lactams) such as polyvinyl pyrrolidone), poly(N-vinyl caprolactam), and copolymers thereof; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol and polyoxyethylated glucose; polyoxazolines, including poly(methyloxazoline) and poly(ethyloxazoline); polyvinylamines; polyvinylacetates, including polyvinylacetate per se as well as ethylene-vinyl acetate copolymers, polyvinyl acetate phthalate, and the like, polyimines, such as polyethyleneimine; starch and starch-based polymers; polyurethane hydrogels; chitosan; polysaccharide gums; xanthan gum; zein; shellac, ammoniated shellac, shellac-acetyl alcohol, and shellac n-butyl stearate. The polymers may be used individually or in combination. Certain combinations will often provide a more extended-release of certain therapeutic compounds than their components when used individually. Suitable combinations include cellulose-based polymers combined with gums, such as hydroxyethyl cellulose or hydroxypropyl cellulose combined with xanthan gum, and poly(ethylene oxide) combined with xanthan gum.

In certain embodiments, the extended-release polymer(s) may be a cellulosic polymer, such as an alkyl substituted cellulose derivative as detailed above. In terms of their viscosities, one class of exemplary alkyl substituted celluloses includes those whose viscosity is within the range of about 100 to about 110,000 centipoise as a 2% aqueous solution at 20 C. Another class includes those whose viscosity is within the range of about 1,000 to about 4,000 centipoise as a 1% aqueous solution at 20 C.

In certain embodiments, the extended-release polymer(s) may be a polyalkylene oxide. In other embodiments, the polyalkylene oxide may be poly(ethylene oxide). In yet other embodiments, the poly(ethylene oxide) may have an approximate molecular weight between 500,000 Daltons (Da) to about 10,000,000 Da or about 900,000 Da to about 7,000,000 Da. In yet a further embodiment, the poly(ethylene oxide) may have a molecular weight of approximately 600,000 Da, 700,000 Da, 800,000 Da, 900,000 Da, 1,000,000 Da, 2,000,000 Da, 3,000,000 Da, 4,000,000 Da, 5,000,000 Da, 6,000,000 Da, 7,000,000 Da, 8,000,000 Da 9,000,000 Da, or 10,000,000 Da. The poly(ethylene oxide) may be any desirable grade of POLYOX™ or any combination thereof. By way of example and without limitation, the POLYOX™ grade may be WSR N-10, WSR N-80, WSR N-750, WSR 205, WSR 1105, WSR N-12K, WSR N-60K, WSR-301, WSR Coagulant, WSR-303, WSR-308, WSR N-3000, UCARFLOC Polymer 300, UCARFLOC Polymer 302, UCARFLOC Polymer 304, and UCARFLOC Polymer 309. In still another embodiment, the poly(ethylene oxide) may have an average number of repeating ethylene oxide units (—CH₂CH₂O—) of about 2,000 to about 160,000. In yet another embodiment, the poly(ethylene oxide) may have an average number of repeating ethylene oxide units of about 2,275, about 4,500, about 6,800, about 9,100, about 14,000, about 20,000, about 23,000, about 45,000, about 90,000, about 114,000, or about 159,000.

Often extended-release formulations utilize an enteric coating. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques.

The choice of polymer will be determined by the nature of the therapeutic compound that is employed in the formulation as well as the desired rate of release. In particular, it will be appreciated by the skilled person, for example in the case of HPMC, that a higher molecular weight will, in general, provide a slower rate of release of therapeutic compound from the formulation. Furthermore, in the case of HPMC, different degrees of substitution of methoxy groups and hydroxypropoxyl groups will give rise to changes in the rate of release of therapeutic compound from the formulation. In this respect, and as stated above, it may be desirable to provide formulation of the disclosure in the form of coatings in which the polymer carrier is provided by way of a blend of two or more polymers of, for example, different molecular weights in order to produce a particular required or desired release profile. The coating can be any of a number of materials conventionally used such for extending drug release such as ethyl cellulose, the Eudragit™ polymers (manufactured by Degussa Rohm Pharma Polymers of Germany), Aquacoat™ (by FMC Biopolymer) and Surelease™ (by Colocon Inc.)

A therapeutic compound is said to be “encapsulated” or “embedded” within a polymer when it is not covalently bound to the polymer but is surrounded by material making up the polymer so that it cannot escape therefrom under physiological conditions unless the permeability of the polymer is enhanced.

This invention provides methods for controlled delivery of an amine-, alcohol-, or thiol-containing therapeutic compound to a patient by providing a therapeutic compound-delivery molecule. Here, the therapeutic compound's amine nitrogen, alcohol oxygen, or thiol sulfur is covalently attached via to a carbon atom of a drug-delivery molecule. The drug-delivery molecule also includes a masked release-enhancing moiety. When the therapeutic compound-delivery molecule is exposed to selected conditions under which an unmasking reaction occurs a release-enhancing moiety facilitates breaking of the covalent bond attaching the therapeutic compound from the drug-delivery molecule, and the therapeutic compound is released. The release-enhancing moiety may be a nucleophilic moiety, an electron-donating moiety or an electron-withdrawing moiety, as more fully described below. The selected conditions may be any conditions inside a patient's body, such as acidic conditions within a patient's stomach or more basic conditions within a patient's intestine.

The covalent bond between the therapeutic compound and the drug-delivery molecule is preferably broken by an intramolecular reaction, such as between the release enhancing moiety and the carbon atom to which the therapeutic compound is covalently attached. To prevent the therapeutic compound from being active before the desired time and place of release inside a patient's body, another moiety, preferably a polymeric moiety, is covalently attached to the therapeutic compound-delivery molecule.

The rate of release of the therapeutic compound from the therapeutic compound-delivery molecule can be controlled by a number of means including controlling the unmasking reaction, or controlling the breaking of the covalent-bond attaching the therapeutic compound to the drug-delivery molecule. The unmasking reaction can be controlled by selecting a more easily hydrolyzable masking group for the therapeutic compound-delivery molecule when a faster rate is desired and a less easily hydrolyzable masking group when a slower reaction is desired. The release reaction can be used to control the release rate of the therapeutic compound by providing a more powerful release-enhancing moiety when a faster rate is desired, and a less powerful release-enhancing moiety when a slower rate is desired. When the release-enhancing moiety is an electron donor or an electron-withdrawing moiety, a more or less powerful electron donor or electron-withdrawing moiety can be used to control the release rate. When the release rate depends on a nucleophilic release-enhancing moiety, a more nucleophilic moiety can be used for a faster rate, and a less nucleophilic moiety can be used for a slower rate.

### Delayed-Release Formulation

Delayed-release formulation of a therapeutic compound can be developed in a number of ways, either using a device, or a capsule comprising a delayed release formulation, or by providing an enteric coating. Non-limiting examples of delayed-release formulations are disclosed herein. It should be noted that delayed release formulations are not limited solely to oral administration of therapeutic compounds, but rather the invention contemplates the use of delayed release formulations useful for delivery of a therapeutic compound via any route available for that compound, such as oral administration, topical administration, transdermal administration, rectal administration, inhalation, and injection.

Non-limiting examples of delayed release formulations for oral delivery are now described. Mahajan (Mahajan et al., 2010, Ars Pharm, 50:215-223), incorporated herein by reference in its entirety, discloses a timed delayed capsule device for chronotherapy. Such capsule device is prepared by sealing the drug tablet and the expulsion excipient inside the insoluble hard gelation capsule body with erodible tablet plug and a soluble cap. Once orally administered, the capsule cap dissolves, and the tablet plug slowly erodes away until a certain time to expose the active ingredient. Accordingly, there is lag time between when the capsule is administered and when the active ingredient is released into the body. The lag time (delayed-release) can be adjusted according to the desired administration time by adding or removing the amount of tablet plug.

PCT/US1992/009385, incorporated herein by reference in its entirety, discloses a delayed-released formulation comprising a core with an enteric coating material. The core includes a pharmaceutical composition. The enteric coating material is a pharmaceutically acceptable excipient that allows the therapeutic compound in the core to be released into the body after certain amount of time.

Alternatively, a delayed-release formulation can be developed by using a barrier coating that delays the release of the active ingredient. The barrier coating may consist of a variety of different materials, depending on the objective. In addition, a formulation may comprise a plurality of barrier coatings to facilitate release in a temporal manner. The barrier coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or a coating based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, the formulation may additionally include a time delay material such as, for example, glyceryl monostearate or glyceryl distearate.

A delayed-release formulation may further comprise a pharmaceutically acceptable excipient. A pharmaceutically acceptable excipient can be a disintegrator, a binder, a filler, a lubricant, or combination thereof used in formulating pharmaceutical products.

In a delayed-release formulation, the delay may be up to 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, or longer.

A delayed-release formulation may comprise 1-80% of a given therapeutic compound administered in a single unit dose. In certain embodiments, the delayed-release formulation comprises about 5, 10, 15, 20, 25, 30, 35, 40, 45, 50, 55, 60, 65, 70, 75, or 80 of the therapeutic compound to be delivered by the formulation.

In certain embodiments, a delayed-release formulation of a therapeutic compound may be administered concurrently with an immediate-release formulation of the same therapeutic compound. Alternatively, a delayed-release formulation of a therapeutic compound may be administered concurrently with an immediate-release formulation of a different therapeutic compound.

In certain embodiment, the delayed-release formulation mixes with the immediate-release formulation to form a pharmaceutical composition before administration.

Valsartan is a once daily drug for treatment of high blood pressure, congestive heart failure, or post-myocardial infarction. Its action mechanism is to block the action of angiotensin. That leads to dilation of blood vessels and hence reduces blood pressure. The drug target of valsartan is circadian gene Agtr1a expression. Its peak phase is about 6 hours after sleep and trough is about 8 hours after awakening. The concentration of Valsartan in plasma reaches the maximum 2-4 hours after administration. For a patient whose desired administration time is same as bedtime 10 pm, the delayed-release formulation of valsartan delays the release of valsartan 2-4 hours.

In one embodiment, the delayed-release formulation comprises a pharmaceutically effective amount of valsartan, wherein the release of valsartan to gastrointestinal tract is delayed about 1 hour, about 2 hours, about 3 hours, about 4 hours, about 5 hours, about 6 hours, about 7 hours, about 8 hours, about 9 hours, about 10 hours, about 11 hours, about 12 hours, or longer, and any and all whole or partial integers there between. The delayed-release formulation of valsartan further comprises an erodible plug, an impermeable capsule body, and soluble cap. These components of the delayed-release formulation of valsartan are configured in the same way as that described in Mahajan (Mahajan et al., 2010, Ars Pharm, 50:215-223).

In another embodiment, the delayed-release formulation of valsartan can be added or mixed with the immediate-release formulation of valsartan to form a pharmaceutical composition of valsartan, then the pharmaceutical composition of valsartan is orally administered. Alternatively, the delayed-release formulation of valsartan is separated from the immediate-release formulation of valsartan, but both are concurrently administered.

### Methods

The present invention also includes methods for treating a disease, disorder, or condition by administering an effective amount of any of the formulations described herein at a specified time such that release of a therapeutic compound from the formulation coincides with peak or trough expression of at least one target gene for the therapeutic compound. For example, the disease, disorder, or condition may be cancer, diabetes mellitus type 2, Alzheimer's disease, schizophrenia, Down's syndrome, obesity, coronary artery disease, and/or any other disease, disorder, or condition associated with circadian genes.

Also included is a method of developing an improved formulation for a therapeutic compound to improve its efficacy. The method comprises: identifying the circadian phase of a target gene for the therapeutic compound; identifying a desired administration time; and calculating a difference between the circadian phase of the target gene expression and the desired administration time. The method further comprises developing a delayed-release formulation based on the calculated difference to synchronize the therapeutic compound's safe and effective amount in plasma with the target's peak phase of gene expression.

In one aspect, the invention includes a method of developing an improved formulation to reduce an undesired side effect of a therapeutic compound. The method comprises: identifying a circadian phase of a target gene associated with the undesired side effect of the therapeutic compound; identifying a desired administration time to minimize the undesired side effect; and calculating a difference between circadian phase of target gene expression and the desired administration time. The method further comprises developing a delayed-release formulation based on the calculated difference to synchronize the therapeutic compound's safe and effective amount in plasma with the target gene's trough expression.

Another aspect of the present invention includes a method of developing an improved formulation to reduce the metabolism of a therapeutic compound. The method comprises: identifying the circadian phase of expression of a metabolic enzyme involved in the metabolism of the therapeutic compound; identifying a desired administration time to minimize the metabolism of the therapeutic compound; and calculating a difference between the circadian phase of expression of the metabolic enzyme and the desired administration time. The method further comprises developing a delayed-release formulation based on the calculated difference to synchronize the therapeutic compound's safe and effective amount in plasma with the metabolic enzyme's trough expression. This means by which the parameters herein are assessed and used are similar to those already described herein for determining the timing of expression and therefore administration of therapeutic compounds in general.

Another aspect of the present invention includes a method of developing an improved formulation to increase the metabolism of a prodrug. The method comprises: identifying the circadian phase of expression of a metabolic enzyme involved in the metabolism of the prodrug; identifying a desired administration time to maximize the metabolism of the prodrug; and calculating a difference between the circadian phase of expression of a metabolic enzyme that converts the prodrug to a drug and the desired administration time. The method further comprises developing a delayed-release formulation based on the calculated difference to synchronize the prodrug's safe and effective amount in plasma with the metabolic enzyme's peak phase of expression.

Another aspect of the present invention includes a method of developing an improved formulation to increase the transportation of a therapeutic compound to its desired target. The method comprises: identifying the circadian phase of expression of a transporter involved in the transportation of the therapeutic compound to its desired target; identifying a desired administration time to increase the transportation of the therapeutic compound to its desired target; and calculating a difference between the circadian phase of expression of the transporter and the desired administration time. The method further comprises developing a delayed-release formulation based on the calculated difference to synchronize the therapeutic compound's safe and effective amount in plasma with the transporter's peak phase of expression.

Another aspect of the present invention includes a method of developing an improved formulation to decrease the transportation of a therapeutic compound to its undesired target. The method comprises: identifying the circadian phase of expression of a transporter involved in the transportation of the therapeutic compound to its undesired target; identifying a desired administration time to decrease the transportation of the therapeutic compound to its undesired target; and calculating a difference between the circadian phase of expression of the transporter and the desired administration time. The method further comprises developing a delayed-release formulation based on the calculated difference to synchronize the therapeutic compound's safe and effective amount in plasma with the transporter's trough of expression.

In certain embodiments, a target associated with a therapeutic compound, also called drug target, can be a DNA, a RNA, a DNA expression, a RNA expression, a protein, a metabolic protein, a transporter, or combination thereof. For example, the target for esomeprazole, a drug for the treatment of dyspepsia, peptic ulcer disease, gastroesophageal reflux disease, and Zollinger-Ellison syndrome, is a protein encoded by Atp4a gene. Non-limiting examples of other drug targets are provided herein in Table 1 and Table 2.

In one embodiment, a non-limiting example of a therapeutic compound used in the methods of the invention is selected from Table 1.

In another embodiment, a non-limiting example of a therapeutic compound used herein in the methods of the invention is selected from the group consisting of esomeprazole, valsartan, rituximab, fluticasone, lisdexamfetamine dimesylate, oseltamivir, methylphenidate, testosterone, lidocaine, quetiapine, sildenafil, niacin, insulin lispro, pemetrexed, ipratropium bromide/albuterol, albuterol sulfate, sitagliptin/metformin, metoprolol succinate, ezetimibe/simvastatin, rabeprazole, eszopiclone, omeprazole, dexmethylphenidate, enalapril, neostigmine, ephedrine, pyridostigmine, lisdexamfetamine, salmeterol, salbutamol, timolol, metoprolol, epinephrine, propranolol, hydralazine, acetazolamide, fludrocortisone, spironolactone, docetaxel, paclitaxel, nifedipine, pilocarpine, atropine, levamisole, carbidopa, flucytosine, levodopa, dopamine, naloxone, propofol, midazolam, ondansetron, ethionamide, vinblastine, hydrochlorothiazide, primaquine, gentamicin, dacarbazine, didanosine, cytarabine, cefazolin, metformin, tetracycline, misoprostol, sulfasalazine, ibuprofen, acetylsalicylic acid, riboflavin, verapamil, ketamine, ciprofloxacin, etoposide, propylthiouracil, mebendazole, fluorouracil, and allopurinol.

In yet another embodiment, the therapeutic compound is valsartan.

The desired administration time varies according to expression of the therapeutic target, dosage of the therapeutic compound, the half-life of the therapeutic compound, and the disease associated with the therapeutic target. In certain embodiments, the desired administration time is between 6 am and 9 am or between 9 am and 12 am or 5 pm and 12 am. In one embodiment, the desired administration time is between 5 pm and 9 pm. In another embodiment, the desired administration time is between 6 pm and 8 pm. In yet another embodiment, the desired administration time is between 6 pm and 7 pm.

The half-life of a therapeutic compound is critical in determining the desired administration time. The half-life of the therapeutic compound can be found in the Orange Book of US Food and Drug Administration or can be measured by one skilled in the art. The half-lives of common therapeutic compounds, for example, are listed in Table 1.

Also included are methods for designing a formulation for treating a disorder in a subject in need thereof. Such methods may involve one or more of the steps of (1) identifying one or more therapeutic compounds that treat the disorder; (2) ascertaining at least one target gene for the one or more therapeutic compounds; (3) determining the peak or trough expression for the at least one target gene in one or more target tissues; and/or (4) devising or designing one or more formulation(s) such that release of the one or more therapeutic compounds coincides with the peak or trough expression for the at least one target gene in one or more target tissues. In some embodiments, the methods additionally include the step of determining the half-life of the one or more therapeutic compounds.

In yet another aspect of the invention, there is included a method of maximizing the efficacy of a therapeutic compound in a subject by administering the therapeutic compound at a time dictated by the circadian phase of the subject, where the circadian phase of the subject is monitored by a device. The method comprises identifying the circadian phase of a subject using any measuring device available in the art that can monitor a subject's circadian phase. The therapeutic compound is then administered to the subject at the precise circadian phase wherein the target gene is maximally or minimally expressed. In certain embodiments without limitation, the device is a smart phone, a smart watch, an activity tracker, or any other known or as yet unknown device installed with a suitable application that identifies or tracks the circadian phases of a subject's circadian phase. Measurement of a subject's circadian phase informs the timing of therapeutic compound delivery to the subject. The method is useful for timing the delivery of any therapeutic compound to the subject, whether formulated or unformulated, but may be particularly useful in situations where the therapeutic compound is administered by injection. In one non-limiting example, timing the delivery of the therapeutic compound streptozocin to a subject is included. Streptozocin is used for treating metastatic pancreatic islet cell carcinoma and is normally administered in a hospital setting by intravenous infusion. Streptozocin is a genotoxic agent and toxic to both the kidney and liver. In the method of the present invention, a subject's circadian cycle is monitored such that the circadian phase for minimal expression of the target gene for streptozocin, Slc2a2, is identified and the infusion of streptozocin is then timed to coincide with minimal expression of Slc2a2 in the subject. As many tumors have lost their circadian clock, timing streptozocin administration to the minimal phase of Slc2a2 expression will improve the therapeutic window and allow subjects to remain on streptozocin longer. The method of the invention should not be construed to be limited to any particular therapeutic compound or any particular measuring device, but should instead include any and all therapeutic compounds to be administered to a subject where the circadian cycle of the subject is measured so that the therapeutic compound is administered at a time when appropriate expression of the target gene is evident.

The circadian phase of the subject may also be measured physiologically, for example, by measuring melatonin levels in the subject.

### Kits

The invention also includes kits for performing any of these methods including the formulation and instructions for use which define when the formulation is provided to a subject in need. Likewise, kits include any of the formulations described herein along with instructions for use which define when the formulation is provided to a subject in need. For example, in such kits, the instructions may specify that the formulation is provided such that release of a first therapeutic compound or a first portion of the first therapeutic compound from the formulation coincides with peak or trough expression of at least one target gene of the first therapeutic compound.

The pharmaceutical formulations of the present invention can be included in a container, pack, or dispenser together with instructions for use and/or administration.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

The total amount of each therapeutic compound present in a formulation can and will vary. Depending on the therapeutic compound, the total amount of a therapeutic compound in a formulation can be between 1 μg to about 2000 mg per dose. In certain embodiments, the amount of therapeutic compound may be between about 1 μg to about 1 mg, e.g., 1 μg, 2, μg, 3 μg, 4 μg, 5 μg, 5.5 μg, 6.0 μg, 6.5 μg, 7.0 μg, 7.5 μg, 8.0 μg, 8.5 μg, 9.0 μg, 9.5 μg, 10 μg, 10.5 μg, 11 μg, 11.5 μg, 12 μg, 12.5 μg, 13 μg, 13.5 μg, 14 μg, 14.5 μg, 15 μg, 15.5 μg, 16 μg, 16.5 μg, 17 μg, 17.5 μg, 18 μg, 18.5 μg, 19 μg, 19.5 μg, 20 μg, 22.5 μg, 25 μg, 27.5 μg, 30 μg, 32.5 μg, 35 μg, 37.5 μg, 40 μg, 45 μg, 50 μg, 60 μg, 70 μg, 80 μg, 100 μg, 110 μg, 120 μg, 130 μg, 140 μg, 150 μg, 160 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 550 μg, 600 μg, 650 μg, 700 μg, 750 μg, 800 μg, 900, μg, and 1 mg. In other embodiments, the amount of therapeutic compound may be between about 1 mg to about 2000 mg, e.g., 1 mg, 2, mg, 3 mg, 4 mg, 5 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 45 mg, 50 mg, 60 mg, 70 mg, 80 mg, 100 mg, 110 mg, 120 mg, 130 mg, 140 mg, 150 mg, 160 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 600 mg, 650 mg, 700 mg, 750 mg, 800 mg, 900, mg, 1000 mg, 1100 mg, 1200 mg, 1300 mg, 1400 mg, 1500 mg, 1600 mg, 1700 mg, 1800 mg, 1900 mg, and 2000 mg.

Throughout the description, where compositions are described as having, including, or comprising specific components, it is contemplated that compositions also consist essentially of, or consist of, the recited components. Similarly, where methods or processes are described as having, including, or comprising specific process steps, the methods or processes also consist essentially of, or consist of, the recited processing steps. Further, it should be understood that the order of steps or order for performing certain actions is immaterial so long as the invention remains operable. Moreover, two or more steps or actions can be conducted simultaneously.

Described herein are RNA sequencing and DNA microarrays that characterize circadian oscillations in transcript expression across twelve mouse organs. It was found that the RNA abundance of 43% of mouse protein-coding genes cycle in at least one organ. Based on these results, it is estimated that over half of the mouse protein-coding genome is rhythmic somewhere in the body.

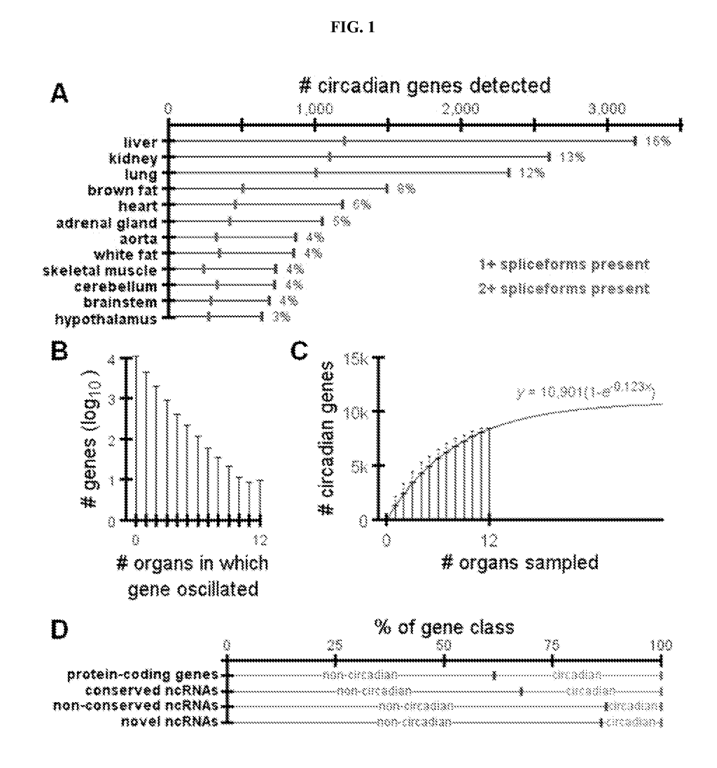

In most organs, expression of many oscillating genes peaked during transcriptional “rush hours” preceding dawn and dusk. A majority of these transcriptional rhythms were found to be organ-specific. The major exception to this finding is the set of core clock genes, which oscillated in phase across all twelve organs (see FIG. 1). Those skilled in the art will recognize that external cues such as restricted feeding or jet-lag could phase-shift these peripheral oscillators with respect to one another. However, these findings agree with the notion that peripheral clocks are largely synchronized in a healthy organism.

Additionally, oscillations in the expression of more than one thousand known and novel non-coding RNAs (ncRNAs) were also observed. ncRNAs conserved between human and mouse oscillated in the same proportion as protein coding genes, and this data supports ncRNAs believed role in mediating clock function. While some of these rhythmic ncRNAs have recognized functions, like snoRNA and miRNA host genes, little is known about the majority. The oscillations of these ncRNAs may prove advantageous for functional studies, e.g., linking a cycling miRNA to its predicted target genes by comparing their cycles.

Table 1 includes a list of top selling therapeutic compounds, their half-lives, the disease/disorder treated by the therapeutic compound, the target gene or gene product targeted by the therapeutic compound, and the organs in which the target gene is expressed.

TABLE 1
List of Top Selling Therapeutic Compounds

| Therapeutic | ½ Life | | | |
|---|---|---|---|---|
| Compound | in hours | Disorder(s) | Target gene(s) | Tissue Type |
| Abiraterone | 5 | Cancer, Prostate Cancer | Cyp17a1 | Liver |
| Acarbose | 2 | Diabetes Drugs, Diabetes | Gaa | Aorta, Kidney |
| | | Mellitus, Diarrhea, | | |
| | | Flatulence, Type 2 Diabetes | | |
| Acebutolol | 3 | Hypertension, Liver | Adrb2, Adrb1 | Adr, Kidney, Lung, Mus |
| Acepromazine | 3 | Hypotension, Priapism, | Adra1a, Htr2a, Adra1b | BFAT, BS, Heart, |
| | | Schizophrenia | | Kidney, Liver, Lung, |
| | | | | Mus, WFAT |
| Acetaminophen | 1 | | Ptgs2, Ptgs1 | Aorta, Heart, Kidney, |
| | | | | Lung |
| Acetazolamide | 3 | Cystinuria, Glaucoma, | Car14, Car4, Aqp1, | Adr, Aorta, BFAT, BS, |
| | | Hypertension, Idiopathic | Car3, Car2 | Cere, Heart, Kidney, |
| | | Intracranial Hypertension, | | Liver, Lung, Mus |
| | | Intracranial Hypertension, | | |
| | | Seizure, Sleep | | |
| Acetohexamide | 1.3 | Diabetes Mellitus | Kcnj1 | Kidney |
| Acetylcysteine | 5.6 | Autism, Cough, CP, Cystic | Grin3a, Slc7a11, | Adr, BS, Cere, Hypo, |
| | | Fibrosis, Inhalation, Liver, | Grin2b, Gss, Acy1, | Kidney, Liver, Lung |
| | | Pulmonary Fibrosis | Chuk, Ikbkb, Grin2d | |
| Aclidinium | 2.4 | COPD | Chrm3, Chrm2, Chrm4 | Adr, BS, Heart, Kidney, |
| | | | | Liver, Lung |
| Adinazolam | 3 | Liver | Gabra3, Gabrg3, | Adr, Aorta, BFAT, BS, |
| | | | Gabrb1, Gabra2, | Cere, Heart, Hypo, |
| | | | Gabrr2, Gabra5, | Kidney, Lung, Mus, |
| | | | Gabrb2, Gabrb3, | WFAT |
| | | | Gabrp, Gabrr1 | |
| Agomelatine | 2 | CP, Depressive Disorder, | Htr2c, Mtnr1a | Cere, Liver |
| | | Sleep | | |
| Aldesleukin | 0.216 | | Il2rg, Il2rb, Il2ra | Adr, Heart, Liver, Lung, |
| | | | | Mus |
| Alfentanil | 1.5 | Breathing, Depression, | Oprm1 | BS |
| | | Liver, Pain | | |
| Alglucosidase alfa | 2.3 | | M6pr | Kidney, Liver, WFAT |
| Allopurinol | 1 | Gout, Hyperuricemia | Xdh | Adr, Aorta, BFAT, BS, |
| | | | | Cere, Heart, Kidney, |
| | | | | Liver, Lung, Mus |
| Almotriptan | 3 | Headache, Liver, Migraine, | Htr1b, Htr1d | Adr, BS, Lung |
| | | Migraine Headache | | |
| Alprenolol | 2 | | Adrb2, Adrb1 | Adr, Kidney, Lung, Mus |
| Alprostadil | 5 | | Ptger2, Ptger1 | Aorta, Heart |
| Amifostine | 0.133 | Cancer, Chemotherapy, | Enpp1 | Adr, Liver |
| | | Radiotherapy | | |
| Aminocaproic Acid | 2 | | Plat | Hypo, Mus |
| Aminolevulinic acid | 0.7 | | Alad | Cere, Hypo, Lung |
| Amlexanox | 3.5 | | S100a13, Fgf1 | Aorta, BFAT, Kidney, |
| | | | | Liver, Lung |
| Amrinone | 5 | Heart Failure, Liver | Pde3a, Pde4b | BFAT, Cere, Heart, |
| | | | | Kidney, Mus, WFAT |
| Anagrelide | 0.5 | Chronic Myeloid Leukemia, | Pde3a | BFAT, Heart, Kidney, |
| | | ET, Leukemia | | Mus |
| Anakinra | 4 | Rheumatoid Arthritis | Il1r1 | Adr, Kidney, Lung, Mus |
| Ancrod | 3 | Clinical Trials | Fga | Liver |
| Aniracetam | 1 | Alzheimer's Disease | Gria2, Htr2a, Gria3 | Adr, BS, Heart, Lung |
| Apomorphine | 0.66 | Addiction, Anxiety, | Drd4, Htr1b, Htr2a, | Adr, BS, Cere, Heart, |
| | | Consumption, Parkinson's | Htr2c, Drd3, Caly, | Hypo, Kidney, Lung, |
| | | Disease, Erectile | Adra2b, Htr1d, Adra2a | WFAT |
| | | Dysfunction, Vomiting | | |
| Arbaclofen Placarbil | 0.1 | | Gabbr1, Gabbr2 | Kidney, Liver |
| Arbutamine | 0.133 | | Adrb2, Adrb3, Adrb1 | Adr, Aorta, BFAT, |
| | | | | Kidney, Lung, Mus |
| Ardeparin | 3.3 | Sodium | Serpind1, Serpinc1 | BS, Liver, Lung, WFAT |
| Aspartame | 4.3 | Consumption, Hearing, | Tas1r2 | Lung |
| | | Phenylketonuria, PKU | | |
| Atomoxetine | 5 | Hyperactivity, Hyperactivity | Slc6a4, Grin3a, | Adr, BS, Cere, Kidney, |
| | | Disorder | Grin2b, Grin2c, | Lung |
| | | | Grin3b, Grin2d | |
| Atracurium | 0.33 | | Chrna2 | BFAT |
| Atropine | 3 | | Chrm3, Chrm2, Chrm4 | Adr, BS, Heart, Kidney, |
| | | | | Liver, Lung |
| Avanafil | 5.36 | Erectile Dysfunction | Pde5a | Adr, Kidney |
| Axitinib | 2.5 | Breast, Breast Cancer, | Flt1, Kdr, Flt4 | Adr, BFAT, BS, Cere, |
| | | Cancer, Clinical Trials, | | Heart, Hypo, Kidney, |
| | | Magnetic Resonance | | Liver, Lung, Mus, |
| | | Imaging, MS | | WFAT |
| Azacitidine | 4 | Cancer, Chemotherapy | Dnmt1 | Adr, Lung |
| Baclofen | 2.5 | Alcohol Dependence, | Gabbr1, Gabbr2 | Kidney, Liver |
| | | Hiccups, Pain, Sleep | | |
| Banoxantrone | 0.64 | | Top2a | Hypo |
| Beclomethasone | 2.8 | Aphthous Ulcers, Colitis, | Nr3c1 | BFAT, Cere, Mus |
| | | Ulcerative Colitis, Hay | | |
| | | Fever, Inhalation, Psoriasis, | | |
| | | Rhinitis, Sinusitis | | |
| Benzonatate | 3 | Cough | Scn5a | Heart |
| Benzquinamide | 1 | | Chrm3, Chrm2, Chrm4 | Adr, BS, Heart, Kidney, |
| | | | | Liver, Lung |
| Betahistine | 3 | Balance | Hrh3 | Kidney |
| Betamethasone | 5.6 | Sodium | Nr3c1 | BFAT, Cere, Mus |
| Bezafibrate | 1 | LDL Cholesterol | Ppara, Ppard, Pparg | Adr, BFAT, Heart, |
| | | | | Kidney, Liver, Lung, |
| | | | | Mus, WFAT |
| Bifonazole | 1 | | Cyp2b10 | Aorta, BFAT, Liver, |
| | | | | Lung, WFAT |
| Bimatoprost | 0.75 | Glaucoma, Hypertension | Ptger3, Ptgfr, Ptger1, | Aorta, BFAT, Heart, |
| | | | Akr1c18 | Kidney, Lung, Mus, |
| | | | | WFAT |
| Binodenoson | 0.166 | | Adora2a | Heart, WFAT |
| Bleomycin | 1.916 | Cancer, Chemotherapy, | Lig3, Lig1 | Lung, WFAT |
| | | Testicular Cancer, Warts | | |
| Bosentan | 5 | Hypertension, Liver | Ednra, Ednrb | Adr, BFAT, BS, Heart, |
| | | | | Kidney, Lung, Mus |
| Brimonidine | 2 | Glaucoma, Hypertension, | Adra2b, Adra2a | Kidney, WFAT |
| | | Liver, Rosacea | | |
| Bromocriptine | 2 | Diabetes Mellitus, | Drd4, Htr1b, Adra1d, | Adr, BFAT, BS, Cere, |
| | | Parkinson's Disease, Liver, | Adra1a, Htr7, Htr2a, | Heart, Hypo, Kidney, |
| | | Type 2 Diabetes | Htr2c, Drd3, Adra2b, | Liver, Lung, Mus, |
| | | | Adra1b, Htr1d, Adra2a | WFAT |
| Budesonide | 2 | Allergies, Colitis, Ulcerative | Nr3c1 | BFAT, Cere, Mus |
| | | Colitis, COPD, Crohn's | | |
| | | Disease, Hay Fever, | | |
| | | Prevention, Rhinitis | | |
| Bumetanide | 1 | Chloride, Heart Failure, | Slc12a2, Slc12a4, Cftr | Aorta, BFAT, Cere, |
| | | Seizure | | Heart, Kidney, Liver, |
| | | | | Lung, Mus, WFAT |
| Bupivacaine | 2.7 | Dental | Ptger1 | Heart |
| Bupranolol | 2 | | Adrb2, Adrb3, Adrb1 | Adr, Aorta, BFAT, |
| | | | | Kidney, Lung, Mus |
| Buprenorphine | 0.3 | Addiction, Chronic Pain, | Oprm1, Oprd1 | BS, Lung |
| | | Liver, Pain | | |
| Cabazitaxel | 4 | Cancer, Prostate Cancer | Tuba4a, Tubb1 | BFAT, Cere, Hypo, |
| | | | | Kidney, Liver |
| Caffeine | 3 | Addiction, Cancer, | Pde1a, Pde1b, Pde2a, | Adr, Aorta, BFAT, BS, |
| | | Consumption, Dehydration, | Pde3b, Pde5a, Pde8b, | Cere, Heart, Hypo, |
| | | Parkinson's Disease, | Adora1, Pde4a, | Kidney, Liver, Lung, |
| | | Headache, Heart Disease, | Pik3cb, Pde4b, Ryr1, | Mus, WFAT |
| | | Insomnia, Pregnancy, Sleep, | Pde10a, Pde7b, Pde3a, | |
| | | Sodium | Pde7a, Pde8a, Prkdc, | |
| | | | Pde9a, Adora2a, | |
| | | | Pik3ca, Pde4c, Pik3cd | |
| Calcitriol | 5 | | Vdr | Adr, Aorta, BFAT |
| Capecitabine | 0.75 | | Tyms | Aorta |
| Captopril | 2 | Heart Failure, Hypertension | Mmp2, Ace, Lta4h | Adr, BS, Cere, Heart, |
| | | | | Hypo, Liver, Lung, |
| | | | | WFAT |
| Carbidopa | 1 | Parkinson's Disease | Ddc | Kidney, Liver |
| Carmustine | 0.25 | Chemotherapy, Crystals | Gsr | Adr, BFAT, BS, Mus |
| Cefazolin | 1.8 | Infusion | Pon1 | Adr, Mus |
| Cerivastatin | 2 | Renal Failure, | Hmgcr | Liver |
| | | Rhabdomyolysis | | |
| Cevimeline | 5 | Dry Mouth, Xerostomia | Chrm3 | Adr, BS, Kidney, Liver, |
| | | | | Lung |
| Chlorothiazide | 0.75 | Heart Failure, Intubation, | Car4, Car2 | Adr, BS, Heart, Kidney, |
| | | Pill, Sodium | | Liver, Lung |
| Ciclopirox | 1.7 | | Atp1a1 | BFAT, BS, Cere, |
| | | | | Kidney, Liver, Lung |
| Cinitapride | 3 | | Htr2a | BS, Heart, Lung |
| Ciprofloxacin | 4 | Clostridium Difficile, | Top2a | Hypo |
| | | Escherichia Coli, | | |
| | | Myasthenia Gravis, | | |
| | | Staphylococcus Aureus | | |
| Cisatracurium | 0.366 | | Chrna2 | BFAT |
| Besylate | | | | |
| Cladribine | 5.4 | Adenosine, Cancer, Hairy | Pola1, Pole4, Pnp, | Adr, Aorta, BS, Cere, |
| | | Cell Leukemia, Leukemia, | Pole2, Rrm2b, Pole3, | Hypo, Kidney, Liver, |
| | | Multiple Sclerosis | Rrm1, Rrm2, Pole | Lung, WFAT |
| Clevidipine | 0.0166 | Blood Pressure | Cacna1c, Cacna1s, | Adr, BFAT, Cere, |
| | | | Cacna1d | Kidney, Lung |
| Clofarabine | 5.2 | ALL, AML | Pola1, Rrm1 | Adr, Kidney |
| Clorazepate | 2 | Liver, Potassium | Gabra3, Gabrg3, | Adr, Aorta, BFAT, BS, |
| | | | Gabrq, Gabrb1, Tspo, | Cere, Hypo, Kidney, |
| | | | Gabra2, Gabra5, | Liver, Lung |
| | | | Gabrb2, Gabrb3, | |
| | | | Gabra6, Gabrp | |
| Clotiazepam | 4 | Anxiety, Liver, Sleep | Gabra3, Gabrg3, | Adr, Aorta, BFAT, BS, |
| | | | Gabrb1, Gabra2, | Cere, Heart, Hypo, |
| | | | Gabrr2, Gabra5, | Kidney, Lung, Mus, |
| | | | Gabrb2, Gabrb3, | WFAT |
| | | | Gabrp, Gabrr1 | |
| Clotrimazole | 2 | Athlete's Foot, Itch, Jock | Kcnn4 | Kidney |
| | | Itch, Oral Candidiasis, | | |
| | | Ringworm, Thrush, Yeast | | |
| | | Infections | | |
| Cocaine | 1 | Balance, Sudden Cardiac | Slc6a4, Chrm2, Scn5a | Adr, BS, Heart, Kidney |
| | | Death, Potassium, Smoking, | | |
| | | Sodium | | |
| Codeine | 3 | Cough, Liver, Myocardial | Oprm1, Oprd1 | BS, Lung |
| | | Infarction | | |
| Colchicine | 1 | Gout, Pericarditis | Tubb5, Tubb1 | Kidney, Liver, WFAT |
| Conivaptan | 5 | Diabetes Insipidus, Heart | Avpr1a, Avpr2 | BFAT, Kidney, Liver, |
| | | Failure, Hyponatremia, | | Lung |
| | | Insipidus, Sodium | | |
| Corticotropin | 0.25 | Adrenal Insufficiency, | Mc2r | Adr, BFAT, Mus, |
| | | Circadian Rhythm, | | WFAT |
| | | Cushing's Syndrome, | | |
| | | Hypercortisolism, Rhythm, | | |
| | | Stress | | |
| Cosyntropin | 0.25 | Cushing's Syndrome, | Mc2r | Adr, BFAT, Mus, |
| | | Infusion | | WFAT |
| Creatine | 3 | Adenosine, Crystals, | Slc6a8, Gamt, Ckm, | Adr, Aorta, BFAT, BS, |
| | | Equilibrium, Liver, | Ckb, Ckmt2, Ckmt1 | Heart, Kidney, Lung |
| | | Supplements | | |
| Cromoglicic acid | 1.3 | | Kcnma1 | Adr, Liver |
| Cytarabine | 0.166 | Acute Myeloid Leukemia, | Polb | Kidney |
| | | ALL, AML, Cancer, | | |
| | | Chemotherapy, Infusion, | | |
| | | Leukemia, Liver, | | |
| | | Malignancy, WS | | |
| Dacarbazine | 5 | Cancer, Chemotherapy, | Pola2, Pgd | Aorta, BFAT, Kidney, |
| | | Infusion, Liver, Malignant | | Liver, Lung, Mus, |
| | | Melanoma, Melanoma | | WFAT |
| Dalfampridine | 3.5 | Multiple Sclerosis, | Kcna1, Kcnd2, Kcna4 | Aorta, BFAT, Cere, |
| | | Potassium | | Heart, Kidney |
| Dantrolene | 4 | Cerebral Palsy, | Ryr1 | BFAT |
| | | Hyperthermia, Malignant | | |
| | | Hyperthermia, Liver, | | |
| | | Multiple Sclerosis, Sodium | | |
| Dapoxetine | 1 | | Htr1b, Htr2c | Adr, BS, Cere |
| Dasatinib | 3 | ALL, BMS, Cancer, CML, | Stat5b, Epha2, Abl1, | Aorta, BFAT, Heart, |
| | | Leukemia, Liver, Prostate | Src, Kit, Pdgfrb, Fyn, | Hypo, Kidney, Liver, |
| | | Cancer | Abl2, Lck | Lung, Mus |
| Decitabine | 0.51 | Acute Myeloid Leukemia, | Dnmt1 | Adr, Lung |
| | | AML, Leukemia | | |
| Defibrotide | 1 | | Adora1, Adora2a, | Aorta, BFAT, Heart, |
| | | | Adora2b | Liver, Lung, WFAT |
| Denileukin diftitox | 1.166 | Leukemia, Neuropathy, | Il2rg, Il2rb, Il2ra | Adr, Heart, Liver, Lung, |
| | | Optic Neuropathy | | Mus |
| Desmopressin | 1 | Bedwetting, Diabetes | Avpr1a, Avpr2 | BFAT, Kidney, Liver, |
| | | Insipidus, Insipidus | | Lung |
| Dexmedetomidine | 2 | Depression, Infusion | Adra2a | WFAT |
| Dexmethylphenidate | 2 | Hyperactivity, Hyperactivity | Slc6a4 | Adr, Kidney |
| | | Disorder, Psychosis | | |
| Dexrazoxane | 2.5 | | Top2a, Top2b | Hypo, Kidney, Mus |
| Dextromethorphan | 3 | CF, COLD, Cold, Common | Sigmar1, Grin3a, | Adr, Aorta, BFAT, BS, |
| | | Cold, Cough, Liver, Pain | Slc6a4, Chrnb4, | Cere, Hypo, Kidney, |
| | | | Chrna2, Chrna3, | Liver, Lung, WFAT |
| | | | Oprm1, Chrna4, | |
| | | | Chrnb2, Oprd1 | |
| Dezocine | 2.2 | | Oprm1 | BS |
| Diazepam | 1 | Anxiety, Depression, | Gabra3, Gabrg3, | Adr, Aorta, BFAT, BS, |
| | | Insomnia, Restless Legs, | Gabrq, Gabrb1, Tspo, | Cere, Heart, Hypo, |
| | | Liver, Muscle Spasms, | Gabra2, Gabrr2, | Kidney, Liver, Lung, |
| | | Seizure, Restless Legs | Gabra5, Gabrb2, | Mus, WFAT |
| | | Syndrome, Tetanus | Gabrb3, Gabra6, | |
| | | | Gabrp, Gabrr1 | |
| Diclofenac | 2 | Aches, Gallstones, NSAID, | Ptgs2, Scn4a, Kcnq2, | Aorta, BFAT, BS, Cere, |
| | | Pain, Potassium, Sodium | Kcnq3, Ptgs1 | Heart, Kidney, Lung |
| Diltiazem | 3 | Arrhythmia, Hypertension, | Cacng1 | BFAT |
| | | Mammary Gland, Migraine | | |
| Dinoprost | 3 | Amniotic Fluid, | Ptgfr | Heart, Lung, Mus |
| Tromethamine | | Endometriosis, Stress | | |
| Dinoprostone | 0.0833 | | Ptger2, Ptger3, Ptger1, | Aorta, BFAT, Heart, |
| | | | Ptger4 | Kidney, Lung, WFAT |
| Diphenidol | 4 | | Chrm3, Chrm2 | Adr, BS, Heart, Kidney, |
| | | | | Liver, Lung |
| Dipyridamole | 0.66 | | Pde5a, Pde4a, Pde10a, | Adr, Aorta, BFAT, |
| | | | Ada | Cere, Heart, Hypo, |
| | | | | Kidney, Liver, Lung, |
| | | | | Mus |
| Dobutamine | 0.033 | Dobutamine, Heart Failure, | Adrb2, Adrb1 | Adr, Kidney, Lung, Mus |
| | | Shock | | |
| Dopamine | 0.033 | Anti-nausea, Attention | Drd4, Slc6a4, Htr7, | Adr, BFAT, Cere, Hypo, |
| | | Deficit Hyperactivity | Dbh, Drd3 | Kidney |
| | | Disorder, Digestive System, | | |
| | | Parkinson's Disease, Heart | | |
| | | Failure, Hyperactivity, | | |
| | | Hyperactivity Disorder, | | |
| | | Restless Legs, RLS, | | |
| | | Schizophrenia, Shock, | | |
| | | Sodium, Restless Legs | | |
| | | Syndrome, Tremor | | |
| Dronabinol | 4 | | Cnr1, Cnr2 | Adr, Mus, WFAT |
| Droperidol | 1.4 | | Adra1a | BFAT, Heart, Kidney, |
| | | | | Lung, Mus, WFAT |
| Drotrecogin alfa | 5.5 | Sepsis | Serpine1, Pf4, Ggcx, | Adr, Aorta, BFAT, BS, |
| | | | Procr, Cp, Thbd, F8, | Cere, Heart, Hypo, |
| | | | F5, Serpina5 | Kidney, Liver, Lung, |
| | | | | Mus, WFAT |
| Droxidopa | 2 | | Adra1d, Adrb2, Adrb3, | Adr, Aorta, BFAT, |
| | | | Adra1a, Adra2b, | Heart, Kidney, Liver, |
| | | | Adra1b, Pah, Adrb1, | Lung, Mus, WFAT |
| | | | Adra2a | |
| Dydrogesterone | 5 | Dysfunctional Uterine | Pgr | Aorta |
| | | Bleeding, Endometriosis, | | |
| | | Hormone Replacement | | |
| | | Therapy, HRT, Infertility, | | |
| | | Menopause, Premenstrual | | |
| | | Syndrome | | |
| Dyphylline | 2 | | Pde4a, Adora1, Pde4b, | Aorta, BFAT, BS, Cere, |
| | | | Pde7a, Pde7b, | Heart, Hypo, Kidney, |
| | | | Adora2a, Pde4c | Liver, Lung, Mus, |
| | | | | WFAT |
| Eletriptan | 4 | Headache, Migraine | Htr1b, Htr7, Htr1d | Adr, BFAT, BS, Lung |
| Enalapril | 2 | Blood Pressure, Heart | Ace | Heart, Lung |
| | | Failure, Hypertension | | |
| Encainide | 1 | | Scn5a | Heart |
| Enoxacin | 3 | Cancer, Cystitis, Gonorrhea, | Top2a | Hypo |
| | | Insomnia, Sexually | | |
| | | Transmitted Diseases, | | |
| | | Urinary Tract Infection | | |
| Enoxaparin | 4.5 | Sodium | Serpinc1 | Liver |
| Enoximone | 4 | Heart Failure, Liver | Pde3a | BFAT, Heart, Kidney, |
| | | | | Mus |
| Enprofylline | 1.9 | Adenosine, Chronic | Pde4a, Adora1, Pde4b, | Aorta, BFAT, Cere, |
| | | Obstructive Lung Disease | Adora2a, Adora2b | Heart, Hypo, Liver, |
| | | | | Lung, Mus, WFAT |
| Enzalutamide | 1 | Breast, Breast Cancer, | Ar | Aorta, BFAT, BS, |
| | | Cancer, Prostate Cancer, | | Kidney |
| | | Prostate Specific Antigen, | | |
| | | PSA | | |
| Ephedrine | 3 | Hypotension | Adra1a, Ache | BFAT, Heart, Kidney, |
| | | | | Lung, Mus, WFAT |
| Epinephrine | 0.033 | Hypertension, Stress | Adrb2, Adra1d, Adrb3, | Adr, Aorta, BFAT, |
| | | | Adra1a, Adra2b, | Heart, Kidney, Liver, |
| | | | Adra1b, Pah, Adrb1, | Lung, Mus, WFAT |
| | | | Adra2a | |
| Epirubicin | 3 | Breast, Breast Cancer, | Top2a | Hypo |
| | | Cancer, Chemotherapy, | | |
| | | Gastric Cancer, Lung | | |
| | | Cancer, Lymphomas, | | |
| | | Ovarian Cancer | | |
| Eplerenone | 4 | Heart Failure, Myocardial | Nr3c2 | Heart, Lung, Mus |
| | | Infarction, Potassium | | |
| Eprosartan | 5 | Blood Pressure, | Agtr1a | Adr, Heart, Kidney, |
| | | Hypertension | | Liver, Mus |
| Eptifibatide | 2.5 | | Itgb3 | Lung |
| Ergoloid mesylate | 3.5 | | Adra1d, Slco2b1, | Adr, Aorta, BFAT, BS, |
| | | | Htr1b, Gabra3, | Cere, Heart, Hypo, |
| | | | Gabrg3, Gabrq, | Kidney, Liver, Lung, |
| | | | Adra1a, Htr7, Gabrb1, | Mus, WFAT |
| | | | Htr2a, Gabra2, Htr2c, | |
| | | | Gabra5, Gabrb2, | |
| | | | Gabrb3, Adra2b, | |
| | | | Adra1b, Gabra6, | |
| | | | Htr1d, Htr6, Gabrp, | |
| | | | Adra2a | |
| Ergonovine | 1 | | Adra1a | BFAT, Heart, Kidney, |
| | | | | Lung, Mus, WFAT |
| Ergotamine | 2 | Migraine | Htr1b, Adra1d, | Adr, BFAT, BS, Cere, |
| | | | Adra1a, Htr2a, Htr2c, | Heart, Kidney, Liver, |
| | | | Adra2b, Adra1b, | Lung, Mus, WFAT |
| | | | Htr1d, Adra2a | |
| Esmolol | 2 | | Adrb1 | Lung |
| Esomeprazole | 1 | Dyspepsia, Gastroesophageal | Atp4a | Liver |
| | | Reflux Disease, Liver, Peptic | | |
| | | Ulcer, Reflux, Ulcer | | |
| Ethinamate | 2.5 | Insomnia | Car2 | BS, Kidney, Liver, Lung |
| Ethionamide | 2 | Extensively Drug-Resistant | Inha | BS, Cere, Heart |
| | | Tuberculosis, TB, Drug- | | |
| | | Resistant Tuberculosis | | |
| Ethopropazine | 1 | | Grin3a, Chrm2 | Adr, BS, Heart |
| Ethotoin | 3 | Epilepsy | Scn5a | Heart |
| Ethoxzolamide | 2.5 | Epilepsy, Glaucoma, Peptic | Car4, Car2 | Adr, BS, Heart, Kidney, |
| | | Ulcer, Ulcer | | Liver, Lung |
| Etidronic acid | 1 | | Ptprs, Atp6v1a | Cere, Hypo, Liver, |
| | | | | Lung, Mus, WFAT |
| Etodolac | 1 | Liver, NSAID | Rxra, Ptgs2, Ptgs1 | Adr, Aorta, BFAT, |
| | | | | Heart, Kidney, Liver, |
| | | | | Lung |
| Etomidate | 1.25 | Emergency Medicine, | Gabra3, Gabrg3, | Adr, Aorta, BFAT, BS, |
| | | Intubation, Liver | Gabrq, Gabrb1, | Cere, Hypo, Kidney, |
| | | | Gabra2, Gabra5, | Liver, Lung |
| | | | Gabrb2, Gabrb3, | |
| | | | Adra2b, Gabra6, | |
| | | | Gabrp | |
| Etoposide | 4 | Liver | Top2a, Top2b | Hypo, Kidney, Mus |
| Fenoldopam | 5 | Liver, Sodium | Adra1d, Adra1a, | Adr, BFAT, Heart, |
| | | | Adra2b, Adra1b, | Kidney, Liver, Lung, |
| | | | Adra2a | Mus, WFAT |
| Fenoprofen | 3 | Rheumatoid Arthritis, Pain | Ptgs2, Ptgs1 | Aorta, Heart, Kidney, |
| | | | | Lung |
| Filgrastim | 3.5 | E. Coli, Escherichia Coli | Csf3r, Elane | Lung, WFAT |
| Finasteride | 4.5 | Baldness, Benign Prostatic | Srd5a1, Akr1d1, | BFAT, Cere, Kidney, |
| | | Hyperplasia, Birth Defects, | Srd5a2 | Liver |
| | | BPH, Liver | | |
| Flucytosine | 2.4 | | Dnmt1 | Adr, Lung |
| Fludrocortisone | 3.5 | | Ar, Nr3c1, Nr3c2 | Aorta, BFAT, BS, Cere, |
| | | | | Heart, Kidney, Lung, |
| | | | | Mus |
| Flumazenil | 4 | Hypersomnia | Gabra3, Gabrg3, | Adr, Aorta, BFAT, BS, |
| | | | Gabrq, Gabrb1, | Cere, Hypo, Kidney, |
| | | | Gabra2, Gabra5, | Liver, Lung |
| | | | Gabrb2, Gabrb3, | |
| | | | Gabra6, Gabrp | |
| Flunisolide | 1.8 | Allergic Rhinitis, Inhalation, | Nr3c1 | BFAT, Cere, Mus |
| | | Rhinitis | | |
| Fluocinolone | 1.3 | Liver, Skin Inflammation | Nr3c1 | BFAT, Cere, Mus |
| Acetonide | | | | |
| Fluorouracil | 0.166 | Cancer, Infusion, Liver | Tyms | Aorta |
| Flurazepam | 2.3 | Anxiety, Dental, Insomnia, | Gabra3, Gabrg3, | Adr, Aorta, BFAT, BS, |
| | | Liver, Pregnancy | Gabrq, Gabrb1, | Cere, Heart, Hypo, |
| | | | Gabra2, Gabrr2, | Kidney, Liver, Lung, |
| | | | Gabra5, Gabrb2, | Mus, WFAT |
| | | | Gabrb3, Gabra6, | |
| | | | Gabrp, Gabrr1 | |
| Flurbiprofen | 4.7 | Cancer, Clinical Trials, | Ptgs2, Ptgs1 | Aorta, Heart, Kidney, |
| | | Liver, Pain | | Lung |
| Fluvastatin | 3 | Hepatitis, Hepatitis C, | Hmgcr | Liver |
| | | Hypercholesterolemia | | |
| Fosphenytoin | 0.25 | Liver, Sodium | Scn5a | Heart |
| Furosemide | 2 | Edema, Heart Failure, | Car2 | BS, Kidney, Liver, Lung |
| | | LASIK | | |
| Fusidic Acid | 5 | Sodium | Cat | Adr, Liver, Lung, Mus |
| Gabapentin | 5 | Anxiety, Anxiety Disorder, | Grin3a, Adora1, | Adr, Aorta, BFAT, BS, |
| | | Bipolar Disorder, Diabetic | Cacna2d1, Grin2b, | Cere, Heart, Hypo, |
| | | Neuropathy, Epilepsy, | Grin2c, Cacna1b, | Kidney, Liver, Lung, |
| | | Insomnia, Neuropathic Pain, | Grin3b, Grin2d | WFAT |
| | | Neuropathy, Pain, Restless | | |
| | | Leg Syndrome | | |
| Gallium nitrate | 1 | | Atp6v1b2, Rrm2, Il1b | Heart, Kidney, Liver, |
| | | | | Lung |
| Galsulfase | 0.15 | | Plin3 | BS, Cere, Heart, Liver |
| Gamma | 0.5 | Cataplexy, Depression, | Gabrb1 | BFAT, BS, Cere |
| Hydroxybutyric Acid | | Excessive Daytime | | |
| | | Sleepiness, Insomnia, | | |
| | | Narcolepsy, Potassium, | | |
| | | Rape, Sleepiness, Sodium | | |
| Ganaxolone | 1.3 | | Gabra3, Gabra2, | Adr, Aorta, BFAT, BS, |
| | | | Gabra5, Gabra6 | Cere, Hypo, Kidney, |
| | | | | Liver |
| Gemcitabine | 0.7 | Chemotherapy | Tyms, Rrm1, Cmpk1 | Aorta, Kidney, WFAT |
| Gemfibrozil | 1.5 | | Ppara | Adr, BFAT, Heart, |
| | | | | Kidney, Liver, Lung, |
| | | | | WFAT |
| Gentamicin | 3 | E. Coli, Escherichia Coli | Lrp2 | Kidney, Lung |
| Glimepiride | 5 | Type 2 Diabetes | Kcnj11, Abcc8, Kcnj1 | BFAT, Cere, Hypo, |
| | | | | Kidney |
| Glipizide | 2 | Liver, Potassium | Abcc8, Pparg | Hypo, Kidney, Mus |
| Glyburide | 1.4 | Liver | Abcc9, Abca1, Kcnj8, | Adr, Aorta, BFAT, BS, |
| | | | Cpt1a, Kcnj11, Kcnj5, | Cere, Heart, Hypo, |
| | | | Abcc8, Kcnj1, Cftr, | Kidney, Liver, Lung, |
| | | | Abcb11 | Mus, WFAT |
| Glycodiazine | 4 | | Abcc8, Kcnj1 | Hypo, Kidney |
| Glycopyrrolate | 0.6 | | Chrm3, Chrm2 | Adr, BS, Heart, Kidney, |
| | | | | Liver, Lung |
| Gonadorelin | 0.033 | | Gnrhr | Adr, BFAT, Lung |
| Goserelin | 4 | Breast, Cancer, Prostate | Gnrhr | Adr, BFAT, Lung |
| | | Cancer | | |
| Heparin | 1.5 | Dialysis, Liver, Sodium | Selp, Serpinc1 | BFAT, Liver |
| Heroin | 0.166 | Hepatitis, Inhalation, | Oprm1, Oprd1 | BS, Lung |
| | | Pregnancy, Smoking | | |
| Hexylcaine | 0.166 | Convulsion, Headache, | Scn5a | Heart |
| | | Sodium, Tinnitus | | |
| Hydralazine | 3 | Blood Pressure, | P4ha1, Aoc3 | Adr, Aorta, BFAT, BS, |
| | | Hypertension | | Cere, Heart, Hypo, |
| | | | | Kidney, Lung, Mus, |
| | | | | WFAT |
| Hydrochlorothiazide | 5.6 | Blood Pressure, | Car4, Car2, Car12, | Adr, BS, Cere, Heart, |
| | | Hypertension, Pregnancy | Car9 | Kidney, Liver, Lung |
| Hydrocodone | 1.25 | Cough | Oprm1, Oprd1 | BS, Lung |
| Hydroflumethiazide | 2 | | Car4, Atp1a1, Car2, | Adr, BFAT, BS, Cere, |
| | | | Car12, Car9 | Heart, Kidney, Liver, |
| | | | | Lung |
| Hydromorphone | 2.6 | Swallowing | Oprm1, Oprd1 | BS, Lung |
| Hydroxyurea | 3 | | Rrm1 | Kidney |
| Hyoscyamine | 2 | | Chrm3, Chrm2, Chrm4 | Adr, BS, Heart, Kidney, |
| | | | | Liver, Lung |
| Ibritumomab | 0.8 | | C1qa, C1qc, C1qb, | Adr, Heart, Kidney, |
| | | | Ms4a1, Fcgr2b, Fcgr3, | Liver, Mus, WFAT |
| | | | Fcgr4, C1rb | |
| Ibuprofen | 2 | CP, Febrile Seizures, | Bcl2, Ptgs2, Thbd, | Adr, Aorta, BFAT, BS, |
| | | NSAID, Nursing, Pain, | Ptgs1, Plat, Pparg, | Cere, Heart, Hypo, |
| | | Pediatrics | Cftr | Kidney, Lung, Mus, |
| | | | | WFAT |
| Icatibant | 1.4 | Angioedema | Anpep | BS, Liver |
| Idursulfase | 0.733 | | Plin3 | BS, Cere, Heart, Liver |
| Iloprost | 0.333 | Blood Pressure, | Pde4a, Pde4b, Ptger1, | Aorta, BFAT, Cere, |
| | | Hypertension, Inhalation, | Plat, Pde4c | Heart, Hypo, Liver, |
| | | Pulmonary Hypertension, | | Lung, Mus, WFAT |
| | | Raynaud's Phenomenon | | |
| Indomethacin | 4.5 | | Glo1, Ppara, Ptgs2, | Adr, Aorta, BFAT, |
| | | | Ptgs1, Pparg | Heart, Kidney, Liver, |
| | | | | Lung, Mus, WFAT |
| Insulin Detemir | 5 | Hemoglobin, Hypoglycemia, | Insr | Liver, Lung |
| | | Type 2 Diabetes | | |
| Insulin Glulisine | 0.7 | Hyperglycemia | Insr | Liver, Lung |
| Insulin Lispro | 1 | | Igflr, Insr | Kidney, Liver, Lung |
| Interferon Alfa-2a, | 2 | | Ifnar2 | Adr, Cere, Liver, Mus |
| Recombinant | | | | |
| Interferon Alfa-2b, | 2 | | Ifnar2 | Adr, Cere, Liver, Mus |
| Recombinant | | | | |
| Interferon alfacon-1 | 1.3 | | Ifnar2 | Adr, Cere, Liver, Mus |
| Interferon alfa-n1 | 1.2 | | Ifnar2 | Adr, Cere, Liver, Mus |
| Ipratropium bromide | 2 | | Chrm3, Chrm2 | Adr, BS, Heart, Kidney, |
| | | | | Liver, Lung |
| Iron Dextran | 5 | | Hba-a1, Fth1 | Adr, Kidney, Liver |
| Isoniazid | 0.5 | Liver, Prevention | Inha | BS, Cere, Heart |
| Isosorbide Dinitrate | 1 | | Npr1 | Adr, Aorta |
| Isosorbide | 5 | Blood Pressure | Gucy1a2 | Adr, Kidney, Lung, Mus |
| Mononitrate | | | | |
| Ketamine | 2.5 | Allergies, Complex Regional | Grin3a, Chrm3, Htr1b, | Adr, BS, Cere, Heart, |
| | | Pain Syndrome, Emergency | Tacr1, Oprm1, Chrm2, | Kidney, Liver, Lung |
| | | Medicine, Liver, Pain, | Htr2a, Htr2c, Oprd1, | |
| | | Respiration | Chrm4, Htr1d | |
| Ketobemidone | 2.42 | Cancer, Pain | Grin3a, Oprm1, | Adr, BS, Cere, Kidney, |
| | | | Grin2b, Grin2c, | Lung |
| | | | Grin3b, Oprd1, Grin2d | |
| Ketoconazole | 2 | Dandruff, Dermatitis, Liver | Cyp19a1, Ar | Adr, Aorta, BFAT, BS, |
| | | | | Kidney |
| Ketoprofen | 1.1 | NSAID | Ptgs2, Ptgs1 | Aorta, Heart, Kidney, |
| | | | | Lung |
| Ketorolac | 2.5 | Allergies, Allergy, Liver, | Ptgs2, Ptgs1 | Aorta, Heart, Kidney, |
| | | NSAID, Pain | | Lung |
| Lansoprazole | 1.5 | Heartburn, Intubation, Liver | Atp4a | Liver |
| Latanoprost | 0.283 | Glaucoma, Hypertension | Ptgfr | Heart, Lung, Mus |
| L-DOPA | 0.833 | | Drd4, Drd3 | Adr, Hypo |
| Lenalidomide | 3 | Multiple Myeloma, | Cdh5, Ptgs2 | Aorta, BFAT, Heart, |
| | | Myeloma | | Hypo, Liver, Lung, Mus |
| Leptin | 0.415 | Obese, Obesity | Lepr | Lung |
| Leuprolide | 3 | | Gnrhr | Adr, BFAT, Lung |
| Levallorphan | 1 | Depression | Oprm1 | BS |
| Levamisole | 4.4 | Agranulocytosis, Cancer, | Chrna3 | BS, Hypo |
| | | Chemotherapy, Colon | | |
| | | Cancer, Head and Neck | | |
| | | Cancer, Liver, Melanoma, | | |
| | | Neck Cancer, Prevention | | |
| Levosimendan | 1 | Heart Failure | Kcnj8, Kcnj11, Pde3a, | Adr, BFAT, Cere, Heart, |
| | | | Tnnc1 | Kidney, Liver, Mus |
| Lidocaine | 1.8166 | Dental, Liver, Pain | Scn5a, Egfr | Heart, Lung |
| Lornoxicam | 3 | NSAID, Pain | Ptgs2, Ptgs1 | Aorta, Heart, Kidney, |
| | | | | Lung |
| Losartan | 1 | Blood Pressure, | Agtr1a | Adr, Heart, Kidney, |
| | | Hypertension, Myocardial | | Liver, Mus |
| | | Infarction, Nursing, | | |
| | | Potassium, Prevention, Renal | | |
| | | Disease, Type 2 Diabetes | | |
| Lovastatin | 5.3 | Hypercholesterolemia, | Itga1, Hdac2, Hmgcr | Adr, Kidney, Liver, |
| | | Hyperlipidemia | | Lung |
| Loxapine | 4 | Inhalation, Liver, | Htr1b, Chrm3, Drd4, | Adr, BFAT, BS, Cere, |
| | | Schizophrenia | Slc6a4, Htr7, Adra1a, | Heart, Hypo, Kidney, |
| | | | Htr2a, Chrm2, Htr2c, | Liver, Lung, Mus, |
| | | | Drd3, Adra2b, Adra1b, | WFAT |
| | | | Htr1d, Htr6, Adrb1, | |
| | | | Chrm4, Adra2a | |
| Lubiprostone | 0.9 | Constipation, Irritable Bowel | Clcn2 | Adr, BS, Cere, Heart, |
| | | Syndrome | | Kidney, WFAT |
| Lumiracoxib | 4 | Liver, NSAID | Ptgs2, Ptgs1 | Aorta, Heart, Kidney, |
| | | | | Lung |
| Mebendazole | 2.5 | AS | Tuba1a | BFAT, Heart, Kidney, |
| | | | | WFAT |
| Mecasermin | 2 | | Igfbp3, Igf2r, Igf1r, | Adr, Aorta, BFAT, |
| | | | Insr | Hypo, Kidney, Liver, |
| | | | | Lung |
| Mefenamic acid | 2 | Headache, Liver, | Ptgs2, Ptgs1 | Aorta, Heart, Kidney, |
| | | Menstruation, Migraine, | | Lung |
| | | Migraine Headache, NSAID, | | |
| | | Pain, Prevention | | |
| Melatonin | 0.5833 | Circadian Rhythm, Clinical | Calr, Esr1, Rorb, | Adr, Aorta, BFAT, BS, |
| | | Trials, Sleep Disorders, | Nqo2, Mtnr1a | Cere, Heart, Hypo, |
| | | Insomnia, Liver, Rhythm, | | Kidney, Liver, Lung, |
| | | Sleep, TIPS | | Mus, WFAT |
| Mesalazine | 5 | Colitis, Ulcerative Colitis, | Ptgs2, Ptgs1, Pparg, | Aorta, Heart, Kidney, |
| | | Crohn's Disease, Liver | Chuk, Ikbkb | Liver, Lung, Mus |
| Methamphetamine | 4 | Addiction, Attention Deficit | Slc6a4, Maoa, Slc18a1, | Adr, Kidney, Liver, |
| | | Hyperactivity Disorder, | Adra2b, Maob, Adra2a | Lung, Mus, WFAT |
| | | Drug Addiction, | | |
| | | Hyperactivity, Hyperactivity | | |
| | | Disorder, Inhalation, | | |
| | | Obesity, Psychosis, | | |
| | | Rhabdomyolysis, Substance | | |
| | | Abuse | | |
| Methimazole | 5 | Agranulocytosis, Graves' | Tpo | Liver |
| | | Disease, Hyperthyroidism, | | |
| | | Liver | | |
| Methsuximide | 1.4 | | Cacna1g | Aorta, Lung, WFAT |
| Methyldopa | 1.75 | Hypertension, Liver, PIH, | Ddc, Adra2a | Kidney, Liver, WFAT |
| | | Pregnancy | | |
| Methylphenidate | 3 | Attention Deficit | Slc6a4 | Adr, Kidney |
| | | Hyperactivity Disorder, | | |
| | | Hyperactivity, Hyperactivity | | |
| | | Disorder, Liver, Narcolepsy, | | |
| | | Postural Orthostatic | | |
| | | Tachycardia Syndrome, | | |
| | | Recall, Tachycardia | | |
| Methylprednisolone | 1 | Infusion, Liver | Nr3c1 | BFAT, Cere, Mus |
| Metocurine Iodide | 3 | | Chrna2 | BFAT |
| Metoprolol | 3 | Hypertension, Liver | Adrb2, Adrb1 | Adr, Kidney, Lung, Mus |
| Metyrosine | 3.4 | | Th | BFAT |
| Midazolam | 1.8 | Epilepsy, Insomnia, Liver, | Gabra3, Gabrg3, | Adr, Aorta, BFAT, BS, |
| | | Seizures in Children | Gabrq, Gabrb1, | Cere, Heart, Hypo, |
| | | | Gabra2, Gabrr2, | Kidney, Liver, Lung, |
| | | | Gabra5, Gabrb3, | Mus, WFAT |
| | | | Gabrb2, Gabra6, | |
| | | | Gabrp, Gabrr1 | |
| Miglitol | 2 | Diabetes Mellitus, | Gaa | Aorta, Kidney |
| | | Hyperglycemia | | |
| Milrinone | 2.3 | Arrhythmia, CHF, Heart | Pde3a | BFAT, Heart, Kidney, |
| | | Failure, Infusion | | Mus |
| Minoxidil | 4.2 | Baldness, Hair Loss, | Ptgs1, Kcnj1 | Heart, Kidney, Lung |
| | | Prevention | | |
| Misoprostol | 0.33 | Gastric Ulcer, Peptic Ulcer, | Ptger3, Ptger2, Ptger4 | Aorta, BFAT, Heart, |
| | | Ulcer | | Kidney, Lung, WFAT |
| Mivacurium | 1.7 | Chloride | Chrm3, Bche, Chrna2, | Adr, BFAT, BS, Heart, |
| | | | Chrm2 | Kidney, Liver, Lung |
| Moclobemide | 1 | Anxiety, Blood Pressure, | Maoa | Adr, Kidney, WFAT |
| | | Depression, LP | | |
| Moexipril | 1 | Heart Failure, Hypertension | Ace, Ace2 | Heart, Lung |
| Mometasone | 5.8 | | Nr3c1 | BFAT, Cere, Mus |
| Montelukast | 2.7 | Allergies, Allergy, Asthma | Cysltr1 | Heart |
| | | Medications, Liver | | |
| Moricizine | 2 | | Scn5a | Heart |
| Morphine | 2 | Addiction, Chronic Pain, | Oprm1, Oprd1 | BS, Lung |
| | | Depression, Inhalation, Pain, | | |
| | | Sleep, Smoking | | |
| Muromonab | 0.8 | | C1qa, C1qc, C1qb, | Adr, Heart, Kidney, |
| | | | Fcgr2b, Fcgr3, Cd3e, | Liver, Lung, Mus, |
| | | | Cd3d, Cd3g, Fcgr4, | WFAT |
| | | | C1rb | |
| Nabilone | 2 | Antiemetics, Chemotherapy, | Cnr1, Cnr2 | Adr, Mus, WFAT |
| | | Chronic Pain, Chronic Pain | | |
| | | Management, Liver, | | |
| | | Multiple Sclerosis, Nausea | | |
| | | and Vomiting, Neuropathic | | |
| | | Pain, Pain, Pain | | |
| | | Management, Vomiting | | |
| Nafarelin | 3 | Endometriosis, Fibroids, | Gnrhr | Adr, BFAT, Lung |
| | | IVF, Puberty, Uterine | | |
| | | Fibroids | | |
| Nalbuphine | 5 | | Oprm1, Oprd1 | BS, Lung |
| Naloxone | 0.5 | Addiction, Depression, | Creb1, Esr1, Oprm1, | Adr, Aorta, BFAT, BS, |
| | | Hypotension, Liver, Pain | Tlr4, Oprd1 | Cere, Heart, Hypo, |
| | | | | Kidney, Liver, Lung, |
| | | | | Mus, WFAT |
| Naltrexone | 4 | Alcohol Dependence, | Oprm1, Oprd1 | BS, Lung |
| | | Constipation | | |
| Naratriptan | 5 | Liver, Migraine | Htr1b, Htr1d | Adr, BS, Lung |
| Nateglinide | 1.5 | Type 2 Diabetes | Abcc8, Pparg | Hypo, Kidney, Mus |
| Nedocromil | 3.3 | Breathing, Inhalation, | Hsp90aa1, Cysltr2, | Adr, Aorta, BFAT, BS, |
| | | Sodium | Cysltr1 | Cere, Heart, Hypo, |
| | | | | Kidney, Liver, Lung, |
| | | | | Mus, WFAT |
| Nefazodone | 2 | Liver, Liver Transplant | Slc6a4, Adra1a, Htr2a, | Adr, BFAT, BS, Cere, |
| | | | Htr2c, Adra1b, Adra2a | Heart, Kidney, Liver, |
| | | | | Lung, Mus, WFAT |
| Nesiritide | 0.3 | Heart Failure | Npr1, Npr2, Npr3 | Adr, Aorta, Cere, Heart, |
| | | | | Kidney, Lung |
| Niacin | 0.33 | Anemia, Atherosclerosis, | Niacr1 | Adrenal |
| | | Crystals, Necropsy, | | |
| | | Tiredness | | |
| Nifedipine | 2 | Cancer, Hypertension, | Cacna1c, Cacna1h, | Adr, Aorta, BFAT, |
| | | Pulmonary Hypertension, | Kcna1, Cacna2d1, | Cere, Kidney, Lung |
| | | Raynaud's Phenomenon, | Cacna1s, Cacna1d | |
| | | Tetanus | | |
| Niflumic Acid | 2.5 | | Ptgs2, Pla2g4a, Ptgs1 | Aorta, Heart, Hypo, |
| | | | | Kidney, Lung |
| Nimesulide | 1.8 | NSAID, Pain | Ptgs2, Ltf | Aorta, Lung |
| Nimodipine | 1.7 | Blood Pressure, | Cacna1c, Ahr, | Adr, Aorta, BFAT, |
| | | Hypertension | Cacna1s, Cacnb1, | Cere, Heart, Kidney, |
| | | | Cacna1d, Cacnb3, | Lung, Mus, WFAT |
| | | | Cacnb4, Nr3c2 | |
| Nitazoxanide | 3.5 | | Por | Adr, Aorta, BFAT, BS, |
| | | | | Cere, Heart, Hypo, |
| | | | | Kidney, Liver, Lung, |
| | | | | WFAT |
| Nitroglycerin | 0.05 | Cancer, Heart Failure, | Npr1 | Adr, Aorta |
| | | Prostate Cancer | | |
| Nitroprusside | 0.033 | Sodium | Npr1 | Adr, Aorta |
| Norfloxacin | 3 | Chemotherapy, Cystitis, | Top2a | Hypo |
| | | Liver, Neuropathy, | | |
| | | Peripheral Neuropathy, | | |
| | | Prostatitis, Sexually | | |
| | | Transmitted Diseases | | |
| Olopatadine | 3 | Allergies, Allergy, | S100a1, S100a13, | Adr, Aorta, Liver, |
| | | Conjunctivitis | S100b | WFAT |
| Olsalazine | 0.9 | Colitis, Ulcerative Colitis | Tpmt | Kidney |
| Omeprazole | 0.5 | Dyspepsia, Gastroesophageal | Atp4a | Liver |
| | | Reflux Disease, Liver, Peptic | | |
| | | Ulcer, Reflux, Ulcer | | |
| Ondansetron | 5.7 | Cancer, Chemotherapy, | Htr1b, Oprm1 | Adr, BS |
| | | Liver, Motion Sickness, | | |
| | | Nausea and Vomiting, | | |
| | | Radiation Therapy, | | |
| | | Vomiting | | |
| Orlistat | 1 | Blood Pressure, Obese, | Fasn | Hypo, Kidney, Liver, |
| | | Obesity, Overweight, Pill, | | Mus, WFAT |
| | | Type 2 Diabetes | | |
| Oseltamivir | 1 | Chemotherapy, Clinical | Neu1, Neu2, Ces1d | Aorta, BFAT, Cere, |
| | | Trials, EA, Swine Flu, | | Heart, Kidney, Liver, |
| | | Influenza, Liver, MS, | | Lung |
| | | Prevention, Vomiting | | |
| OspA lipoprotein | 1.2 | | Tlr2 | Kidney |
| Oxandrolone | 0.55 | | Ar | Aorta, BFAT, BS, |
| | | | | Kidney |
| Oxcarbazepine | 2 | Anxiety, Anxiety Disorder, | Scn5a | Heart |
| | | Epilepsy, Tics | | |
| Oxprenolol | 1 | Blood Pressure, | Adrb2, Adrb1 | Adr, Kidney, Lung, Mus |
| | | Hypertension, Liver, | | |
| | | Mammary Gland | | |
| Oxtriphylline | 3 | | Pde4a, Adora1, Pde3a, | Aorta, BFAT, Cere, |
| | | | Adora2a, Hdac2 | Heart, Hypo, Kidney, |
| | | | | Liver, Lung, Mus, |
| | | | | WFAT |
| Oxycodone | 4.5 | NSAID, Pain | Oprm1, Oprd1 | BS, Lung |
| Oxymorphone | 1.3 | Liver | Oprm1, Oprd1 | BS, Lung |
| Oxytocin | 0.0166 | Anxiety, Intimacy, Nipple, | Oxt | Kidney |
| | | WS | | |
| Pancuronium | 1.5 | | Chrm3, Chrna2, | Adr, BFAT, BS, Heart, |
| | | | Chrm2 | Kidney, Liver, Lung |
| Pantoprazole | 1 | Liver | Atp4a | Liver |
| Papaverine | 0.5 | Erectile Dysfunction | Pde4b, Pde10a | BFAT, Cere, Heart, |
| | | | | Mus, WFAT |
| Paricalcitol | 4 | | Vdr | Adr, Aorta, BFAT |
| PCK3145 | 0.35 | | Rpsa | Liver, Lung |
| Pegaptanib | 3 | Age-Related Macular | Nrp1 | BFAT, Heart, Kidney, |
| | | Degeneration, Macular | | Liver, Lung, Mus |
| | | Degeneration, Sodium | | |
| Pemetrexed | 3.5 | Cancer, Chemotherapy, | Tyms, Gart, Atic | Aorta, Kidney, Liver, |
| | | Lung Cancer, Mesothelioma | | Lung, WFAT |
| Pentagastrin | 0.166 | Carcinoid Syndrome | Cckbr | BS |
| Pentazocine | 2 | Liver, Pain | Sigmar1, Oprm1 | Adr, Aorta, BS, Kidney, |
| | | | | Liver, Lung, WFAT |
| Pentobarbital | 5 | Liver, Sodium | Gabra3, Gria2, | Adr, Aorta, BFAT, BS, |
| | | | Gabrg3, Grin3a, | Cere, Hypo, Kidney, |
| | | | Gabrq, Gabrb1, | Liver, Lung |
| | | | Gabra2, Grin2b, | |
| | | | Gabra5, Chrna4, | |
| | | | Grin2c, Gabrb2, | |
| | | | Gabrb3, Grin3b, | |
| | | | Gabra6, Gabrp, | |
| | | | Grin2d | |
| Pentosan Polysulfate | 4.8 | | Fgf4, Fgf1, Fgf2 | Adr, Aorta, BFAT, BS, |
| | | | | Kidney, Liver, Lung, |
| | | | | WFAT |
| Pentostatin | 5.7 | Chronic Lymphocytic | Ada | Hypo |
| | | Leukemia, Leukemia, Liver | | |
| Pentoxifylline | 0.4 | CP | Pde5a, Nt5e, Adora1, | Adr, Aorta, BFAT, |
| | | | Pde4a, Pde4b, | Cere, Heart, Hypo, |
| | | | Adora2a | Kidney, Liver, Lung, |
| | | | | Mus, WFAT |
| Perhexiline | 2 | Consumption | Cpt1a, Cpt2 | Adr, Aorta, BFAT, |
| | | | | Cere, Heart, Hypo, |
| | | | | Kidney, Liver, Lung, |
| | | | | Mus |
| Perindopril | 1.2 | Blood Pressure, Coronary | Ace | Heart, Lung |
| | | Artery Disease, Heart Failure | | |
| Pethidine | 1 | | Chrm3, Slc6a4, | Adr, BS, Cere, Heart, |
| | | | Grin2b, Chrm2, | Kidney, Liver, Lung |
| | | | Oprm1, Grin2c, | |
| | | | Grin2d, Chrm4 | |
| Phenelzine | 1.2 | Liver | Maoa, Abat, Gpt, Gpt2, | Adr, Aorta, BFAT, BS, |
| | | | Aoc3, Maob | Cere, Hypo, Kidney, |
| | | | | Liver, Lung, Mus, |
| | | | | WFAT |
| Phenindione | 5 | Breast | Vkorc1 | Adr, BFAT |
| Phentolamine | 0.3166 | | Adra1a, Adra2a | BFAT, Heart, Kidney, |
| | | | | Lung, Mus, WFAT |
| Phenylephrine | 2.1 | Blood Pressure, Liver | Adra1d, Adra1a, | Adr, BFAT, Heart, |
| | | | Adra1b | Kidney, Liver, Lung, |
| | | | | Mus, WFAT |
| Phenylpropanolamine | 2.1 | COLD, Cold, Cough, | Adrb2, Adra1a, Adrb1, | Adr, BFAT, Heart, |
| | | Urinary Incontinence | Adra2a | Kidney, Lung, Mus, |
| | | | | WFAT |
| Pilocarpine | 0.76 | Cancer, Dry Mouth, | Chrm3, Chrm2 | Adr, BS, Heart, Kidney, |
| | | Glaucoma, Head and Neck | | Liver, Lung |
| | | Cancer, Neck Cancer, Oral | | |
| | | Surgery, Radiotherapy, | | |
| | | Xerostomia | | |
| Pindolol | 3 | Liver | Adrb2, Htr1b, Adrb1 | Adr, BS, Kidney, Lung, |
| | | | | Mus |
| Pioglitazone | 3 | Liver | Pparg | Kidney, Mus |
| Pirfenidone | 2 | | Furin | BFAT, Kidney |
| Plerixafor | 4.4 | Cancer, Stem Cells | Cxcr4 | BFAT, Heart, Mus, |
| | | | | WFAT |
| Podofilox | 1 | | Tuba4a, Top2a, Tubb5 | BFAT, Cere, Hypo, |
| | | | | Kidney, Liver, WFAT |
| Pralidoxime | 1.233 | Chloride | Bche, Ache | Adr, BFAT, Kidney |
| Pramlintide | 0.8 | BMS, Diabetes Mellitus | Ramp1, Ramp3 | BFAT, Lung |
| Prazosin | 2 | Anxiety, Blood Pressure, | Adra1d, Adra1a, | Adr, BFAT, Heart, |
| | | Panic Disorder, PTSD | Kcnh2, Adra2b, | Kidney, Liver, Lung, |
| | | | Adra1b, Kcnh6, Kcnh7, | Mus, WFAT |
| | | | Adra2a | |
| Prednisolone | 2 | Hepatitis | Nr3c1 | BFAT, Cere, Mus |
| Prednisone | 2 | Cancer, Fatty Liver, Liver | Nr3c1, Hsd11b1 | BFAT, Cere, Heart, |
| | | | | Liver, Lung, Mus |
| Preotact | 1.5 | Escherichia Coli, Menopause | Pth1r | Liver |
| Primaquine | 3.7 | Liver | Krt7, Nqo2 | Aorta, Hypo, Kidney, |
| | | | | Liver, Lung, WFAT |
| Primidone | 3 | Anemia, Bipolar Disorder, | Gabra3, Gria2, | Adr, Aorta, BFAT, BS, |
| | | Birth Defects, Cerebral | Gabrg3, Gabrq, | Cere, Hypo, Kidney, |
| | | Palsy, Depression, | Gabrb1, Gabra2, | Liver, Lung |
| | | Depressive Disorder, | Gabra5, Chrna4, | |
| | | Essential Tremor, Liver, | Gabrb2, Gabrb3, | |
| | | Migraine, Neuropathic Pain, | Gabra6, Gabrp | |
| | | Pain, Seizure, Sodium, | | |
| | | Tonic-Clonic Seizure, | | |
| | | Tremor, Trigeminal | | |
| | | Neuralgia | | |
| Procainamide | 2.5 | Arrhythmia, Liver | Dnmt1, Scn5a | Adr, Heart, Lung |
| Procaine | 0.1283 | Dental, Pain, Sodium | Grin3a, Kcnmb2, | Adr, BFAT, BS, Heart, |
| | | | Kcnn1, Maoa, Chrna2, | Kidney, Liver, Lung, |
| | | | Kcnn3, Kcnmb1, | Mus, WFAT |
| | | | Kcnn4, Maob | |
| Progabide | 4 | Epilepsy | Gabbr1 | Kidney |
| Propafenone | 2 | | Scn5a, Kcnh2 | Heart, Lung, WFAT |
| Propofol | 1 | Emergency Medicine, Liver, | Gabra3, Gabrg3, | Adr, Aorta, BFAT, BS, |
| | | Pain, Sodium | Gabrq, Scn4a, Gabrb1, | Cere, Heart, Hypo, |
| | | | Gabra2, Gabra5, | Kidney, Liver, Lung |
| | | | Gabrb2, Gabrb3, | |
| | | | Scn2a1, Gabra6, | |
| | | | Gabrp | |
| Propranolol | 4 | Anxiety, Hypertension | Adrb2, Adrb3, Htr1b, | Adr, Aorta, BFAT, BS, |
| | | | Adrb1 | Kidney, Lung, Mus |
| Propylthiouracil | 2 | Agranulocytosis, Anemia, | Tpo | Liver |
| | | Graves' Disease, | | |
| | | Hyperthyroidism, Liver | | |
| Pyridostigmine | 3 | | Bche, Ache | Adr, BFAT, Kidney |
| Quinapril | 2 | Heart Failure, Hypertension | Ace | Heart, Lung |
| Rabeprazole | 1 | Liver, Sodium | Atp4a | Liver |
| Ramelteon | 1 | Liver, Sleep | Mtnr1a | Liver |
| Ramipril | 2 | Blood Pressure, Heart | Ace | Heart, Lung |
| | | Failure, Hypertension, Liver | | |
| Rasagiline | 3 | Parkinson's Disease, Liver, | Bcl2, Maob | Adr, Aorta, BFAT, |
| | | RAS | | Heart, Kidney, Liver, |
| | | | | Lung, Mus |
| Regadenoson | 0.033 | Adenosine, Stress | Adora2a | Heart, WFAT |
| Remifentanil | 0.016 | Pain | Oprm1, Oprd1 | BS, Lung |
| Remoxipride | 4 | Anemia, Mania, MI, MS, | Drd4, Sigmar1, Htr2a, | Adr, Aorta, BS, Heart, |
| | | Schizophrenia | Drd3 | Hypo, Kidney, Liver, |
| | | | | Lung, WFAT |
| Repaglinide | 1 | Liver | Abcc8, Pparg | Hypo, Kidney, Mus |
| Riboflavin | 1.1 | Crystals, Liver | Blvrb | Adr, BFAT, Kidney |
| Risedronate | 1.5 | | Fdps | Adr, Aorta, BFAT, |
| | | | | Kidney, Liver |
| Ritodrine | 1.7 | | Adrb2 | Adr, Kidney, Lung, Mus |
| Rituximab | 0.8 | Infusion, Leukemia | C1qa, C1qc, C1qb, | Adr, Heart, Kidney, |
| | | | Ms4a1, Fcgr2b, Fcgr3, | Liver, Mus, WFAT |
| | | | Fcgr4, C1rb | |
| Rivastigmine | 1.5 | Dementia, Alzheimer's | Bche, Ache | Adr, BFAT, Kidney |
| | | Disease, Parkinson's | | |
| | | Disease, Liver, Nausea and | | |
| | | Vomiting, Vomiting | | |
| Rizatriptan | 2 | Headache, Migraine | Htr1b, Htr1d | Adr, BS, Lung |
| Rocuronium | 1 | | Chrna2, Chrm2 | BFAT, BS, Heart |
| Rosiglitazone | 3 | Diabetes Mellitus, Heart | Acsl4, Pparg | Adr, Kidney, Liver, |
| | | Attack, Liver, Myocardial | | Lung, Mus |
| | | Infarction, Type 2 Diabetes | | |
| Rotigotine | 5 | Depression, Depressive | Drd4, Drd3, Adra2b | Adr, Hypo, Kidney |
| | | Disorder, Parkinson's | | |
| | | Disease, Restless Legs, | | |
| | | Liver, RLS, Restless Legs | | |
| | | Syndrome | | |
| Salbutamol | 1.6 | Infusion, Liver | Adrb2, Adrb1 | Adr, Kidney, Lung, Mus |
| Salmeterol | 5.5 | COPD, Inhalation | Adrb2 | Adr, Kidney, Lung, Mus |
| Salsalate | 1 | | Ptgs2, Ptgs1 | Aorta, Heart, Kidney, |
| | | | | Lung |
| Saxagliptin | 2.5 | BMS, Heart Failure, Type 2 | Dpp4 | Kidney |
| | | Diabetes | | |
| Scopolamine | 4.5 | CP, Liver, Motion Sickness, | Chrm3, Chrm2, Chrm4 | Adr, BS, Heart, Kidney, |
| | | Nausea and Vomiting, | | Liver, Lung |
| | | Vomiting | | |
| Selegiline | 1.2 | Dementia, Depression, | Maoa, Maob | Adr, Kidney, Liver, |
| | | Depressive Disorder, | | Lung, Mus, WFAT |
| | | Parkinson's Disease, Liver | | |
| SGS742 | 4 | | Gabbr1, Gabbr2 | Kidney, Liver |
| Sibutramine | 1.1 | Liver, Obesity | Slc6a4 | Adr, Kidney |
| Sildenafil | 4 | Erectile Dysfunction, | Pde5a, Pde6g, Pde6h | Adr, BFAT, Hypo, |
| | | Hypertension, Liver, | | Kidney |
| | | Pulmonary Hypertension | | |
| Simvastatin | 3 | Breastfeeding, | Hmgcr | Liver |
| | | Hypercholesterolemia, Liver, | | |
| | | Pregnancy, Prevention | | |
| Spironolactone | 0.166 | Heart Failure, Hypertension, | Cacna1c, Cacna1h, Ar, | Adr, Aorta, BFAT, BS, |
| | | Liver, Potassium | Pgr, Cacna1g, Nr3c1, | Cere, Heart, Hypo, |
| | | | Srd5a1, Cacng1, | Kidney, Liver, Lung, |
| | | | Cacna2d1, Cacna1s, | Mus, WFAT |
| | | | Cacnb1, Cacna1i, | |
| | | | Cacna1d, Nr3c2, | |
| | | | Cacnb3, Cacnb4, | |
| | | | Cacna1b, Cacna1a, | |
| | | | Srd5a2 | |
| Stannsoporfin | 3.8 | | Hmox1, Hmox2 | Adr, Aorta, Heart, |
| | | | | Kidney |
| Streptozocin | 0.0833 | | Slc2a2 | Kidney, Liver, Mus |
| Sufentanil | 4.416 | Clinical Trials | Oprm1, Oprd1 | BS, Lung |
| Sulfasalazine | 5 | Rheumatoid Arthritis, | Acat1, Ptgs2, Slc7a11, | Adr, Aorta, BS, Heart, |
| | | Pregnancy | Ptgs1, Pparg, Tbxas1, | Hypo, Kidney, Liver, |
| | | | Chuk, Ikbkb | Lung, Mus, WFAT |
| Sulfinpyrazone | 4 | Adenosine, Gout | Abcc1, Abcc2 | BFAT, Heart, Hypo, |
| | | | | Kidney, Liver, Lung |
| Sumatriptan | 2.5 | Migraine | Htr1b, Htr1d | Adr, BS, Lung |
| Tacrine | 2 | Alzheimer's Disease | Bche, Ache | Adr, BFAT, Kidney |
| Talampanel | 3 | | Gria2, Gria4, Gria1, | Adr, Cere, Kidney, Lung |
| | | | Gria3 | |
| Tamoxifen | 5 | Breast, Breast Cancer, | Prkcd, Prkci, Esr1, | Adr, Aorta, BFAT, BS, |
| | | Cancer, Liver, Menopause | Prkce, Prkca, Prkcb, | Cere, Heart, Kidney, |
| | | | Prkcq, Esr2 | Liver, Lung, Mus, |
| | | | | WFAT |
| Tamsulosin | 5 | Benign Prostatic | Adra1d, Adra1a, | Adr, BFAT, Heart, |
| | | Hyperplasia, BPH, Enlarged | Adra1b | Kidney, Liver, Lung, |
| | | Prostate | | Mus, WFAT |
| Tapentadol | 4 | Chronic Pain, CP, Pain | Slc6a4, Oprm1, Oprd1 | Adr, BS, Kidney, Lung |
| Tenecteplase | 1.9 | Blood Clot, Liver | Serpine1, Anxa2, Calr, | Adr, Aorta, BFAT, BS, |
| | | | Canx, Lrp1, Clec3b, | Cere, Heart, Hypo, |
| | | | Plaur, Krt8, Fga | Kidney, Liver, Lung, |
| | | | | Mus, WFAT |
| Teniposide | 5 | Acute Lymphocytic | Top2a | Hypo |
| | | Leukemia, ALL, Cancer, | | |
| | | Chemotherapy, Leukemia, | | |
| | | Liver | | |
| Terbutaline | 5.5 | Inhalation, Liver, Parenting, | Adrb2 | Adr, Kidney, Lung, Mus |
| | | Pregnancy, Prevention | | |
| Terfenadine | 3.5 | Allergy, Arrhythmia, Liver, | Chrm3, Chrm2, Kcnh2, | Adr, BS, Heart, Kidney, |
| | | Potassium, Rhythm, | Chrm4 | Liver, Lung, WFAT |
| | | Tachycardia | | |
| Testosterone | 0.166 | Consumption, Infusion, | Ar | Aorta, BFAT, BS, |
| | | Liver, Prevention | | Kidney |
| Thalidomide | 5 | Anxiety, Blindness, Cancer, | Ptgs2, Fgfr2, Nfkb1 | Aorta, BFAT, BS, Cere, |
| | | Deafness, Gastritis, | | Kidney, Liver, Lung, |
| | | Immunotherapy, Insomnia, | | Mus |
| | | MS, Multiple Myeloma, | | |
| | | Myeloma, Strabismus | | |
| Thiopental | 3 | Sodium | Gabra3, Gria2, | Adr, Aorta, BFAT, BS, |
| | | | Gabra2, Gabra5, | Cere, Hypo, Kidney, |
| | | | Chrna4, Faah, Gabra6 | Liver |
| Thyrotropin Alfa | 5 | | Tshr | Adr, Aorta, BFAT, |
| | | | | Kidney, Mus |
| Tiaprofenic acid | 1.5 | Cystitis, Liver, NSAID, | Ptgs2, Ptgs1 | Aorta, Heart, Kidney, |
| | | Pain, Plastic Surgery, Renal | | Lung |
| | | Disease | | |
| Timolol | 2.5 | Glaucoma, Hypertension, | Adrb2, Adrb1 | Adr, Kidney, Lung, Mus |
| | | Liver, Myocardial Infarction | | |
| Tinzaparin | 1.366 | | Cxcl12, Itga4, Serpinc1 | Adr, Aorta, BFAT, BS, |
| | | | | Kidney, Liver, Lung, |
| | | | | Mus, WFAT |
| Tirofiban | 2 | | Itgb3 | Lung |
| Tizanidine | 2.5 | ALS, Back Pain, Clinical | Adra2b, Adra2a | Kidney, WFAT |
| | | Trials, Liver Function, | | |
| | | Headache, Hypotension, | | |
| | | Orthostatic Hypotension, | | |
| | | Liver, Migraine, Multiple | | |
| | | Sclerosis, Pain, Sleep | | |
| Tofacitinib | 3 | Rheumatoid Arthritis, | Jak2, Jak1 | Cere |
| | | Clinical Trials, CP, | | |
| | | Prevention, Psoriasis | | |
| Tolmetin | 2 | Rheumatoid Arthritis, Pain | Ptgs2, Ptgs1 | Aorta, Heart, Kidney, |
| | | | | Lung |
| Tolterodine | 1.9 | Urinary Incontinence | Chrm3, Chrm2, Chrm4 | Adr, BS, Heart, Kidney, |
| | | | | Liver, Lung |
| Topotecan | 2 | Cancer, Infusion, Liver, | Top1mt, Top1 | BFAT, BS, Hypo, |
| | | Lung Cancer, Ovarian | | Kidney, Liver, Lung, |
| | | Cancer | | WFAT |
| Tositumomab | 0.8 | Cancer, Chemotherapy | C1qa, C1qc, C1qb, | Adr, Heart, Kidney, |
| | | | Ms4a1, Fcgr2b, Fcgr3, | Liver, Mus, WFAT |
| | | | Fcgr4, C1rb | |
| Tranylcypromine | 1.5 | Anxiety, Anxiety Disorder, | Maoa, Maob | Adr, Kidney, Liver, |
| | | Depression, Liver | | Lung, Mus, WFAT |
| Travoprost | 0.75 | Glaucoma, Hypertension | Ptgfr | Heart, Lung, Mus |
| Trazodone | 1 | Depression, Sleep | Slc6a4, Adra1a, Htr2a, | Adr, BFAT, BS, Cere, |
| | | | Htr2c, Adra2a | Heart, Kidney, Lung, |
| | | | | Mus, WFAT |
| Treprostinil | 2 | Hypertension, Infusion, | Ppard | Adr, Kidney, Liver |
| | | Inhalation, Liver | | |
| Tretinoin | 0.5 | Acne, Leukemia | Rxrb, Aldh1a1, | Aorta, BFAT, BS, |
| | | | Aldh1a2, Rxrg, Rarg, | Heart, Kidney, Liver, |
| | | | Gprc5a | Lung, Mus |
| Triamcinolone | 1.466 | Inhalation, Liver | Nr3c1 | BFAT, Cere, Mus |
| Triamterene | 4.25 | Edema, Hypertension, | Scnn1a, Scnn1g, | Adr, Aorta, BFAT, |
| | | Potassium | Scnn1b | Kidney, Liver, Lung |
| Triazolam | 1.5 | Insomnia, Liver | Gabra3, Gabrg3, | Adr, Aorta, BFAT, BS, |
| | | | Gabrq, Gabrb1, Tspo, | Cere, Heart, Hypo, |
| | | | Gabra2, Gabrr2, | Kidney, Liver, Lung, |
| | | | Gabra5, Gabrb2, | Mus, WFAT |
| | | | Gabrb3, Gabra6, | |
| | | | Gabrp, Gabrr1 | |
| Trifluridine | 0.2 | Cancer, CF, Herpes, herpes | Tyms | Aorta |
| | | simplex virus, Keratitis | | |
| Trihexyphenidyl | 3.3 | | Chrm3, Chrm2, Chrm4 | Adr, BS, Heart, Kidney, |
| | | | | Liver, Lung |
| Tubocurarine | 1 | Chloride | Chrna2, Ache | BFAT, Kidney |
| Urokinase | 0.2 | Cancer, Nursing | Serpine1, Nid1, Plat, | Adr, BFAT, Heart, |
| | | | Plaur, Plau, Lrp2, | Hypo, Kidney, Liver, |
| | | | Serpina5, St14 | Lung, Mus, WFAT |
| Valsartan | 1 | Blood Pressure, CHF, Heart | Agtr1a | Adr, Heart, Kidney, |
| | | Failure, Hypertension, MI, | | Liver, Mus |
| | | Myocardial Infarction | | |
| Vapreotide | 0.5 | | Tacr1 | BS |
| Vardenafil | 4 | Erectile Dysfunction | Pde5a, Pde6g, Pde6h | Adr, BFAT, Hypo, |
| | | | | Kidney |
| Vasopressin | 0.166 | Blood Pressure, Stress, WS | Avpr1a, Avpr2 | BFAT, Kidney, Liver, |
| | | | | Lung |
| Vecuronium | 0.85 | | Chrna2 | BFAT |
| Velaglucerase alfa | 0.1833 | Gaucher Disease, Infusion | Gba | Lung |
| Venlafaxine | 5 | Anxiety, Anxiety Disorder, | Slc6a4 | Adr, Kidney |
| | | Blood Pressure, | | |
| | | Constipation, CP, | | |
| | | Depression, Depressive | | |
| | | Disorder, Dry Mouth, EA, | | |
| | | GAD, Headache, Insomnia, | | |
| | | Liver, Panic Disorder | | |
| Verapamil | 2.8 | Arrhythmia, Cluster | Cacna1c, Slc6a4, | Adr, Aorta, BFAT, BS, |
| | | Headaches, Headache, | Cacna1g, Cacna1s, | Cere, Heart, Hypo, |
| | | Hypertension, Liver, | Cacnb1, Kcnj11, | Kidney, Lung, WFAT |
| | | Migraine | Cacna1i, Cacna1d, | |
| | | | Cacnb3, Cacnb4, | |
| | | | Kcnh2, Scn5a, | |
| | | | Cacna1b, Cacna1a | |
| Vildagliptin | 1.5 | Diabetes Mellitus, | Dpp4 | Kidney |
| | | Hyperglycemia, | | |
| | | Hypoglycemia, Type 2 | | |
| | | Diabetes | | |
| Vincristine | 5 | Cancer, Chemotherapy, | Tuba4a, Tubb5 | BFAT, Cere, Hypo, |
| | | Liver, PFS | | Kidney, Liver, WFAT |
| Vitamin A | 1.9 | | Dhrs3, Retsat, | Adr, Aorta, BFAT, BS, |
| | | | Aldh1a3, Rdh13, | Cere, Heart, Hypo, |
| | | | Aldh1a1, Rbp1, | Kidney, Liver, Lung, |
| | | | Aldh1a2, Rdh5, Lrat, | Mus, WFAT |
| | | | Rdh11, Rdh14, Rdh8, | |
| | | | Dhrs4 | |
| Vorinostat | 2 | Liver | Hdac1, Hdac3, Hdac8, | Adr, Aorta, BFAT, BS, |
| | | | Hdac6, Hdac2 | Heart, Hypo, Kidney, |
| | | | | Liver, Lung |
| Warfarin | 1 | Blood Clots, CP, Prevention | Vkorc1 | Adr, BFAT |
| Yohimbine | 0.6 | Type 2 Diabetes | Htr1b, Kcnj8, Kcnj11, | Adr, BFAT, BS, Cere, |
| | | | Kcnj12, Htr2a, Htr2c, | Heart, Hypo, Kidney, |
| | | | Drd3, Adra2b, Kcnj1, | Lung, Mus, WFAT |
| | | | Htr1d, Adra2a | |
| Zaleplon | 1 | Insomnia, Sleep | Tspo | BFAT, Lung |
| Zanamivir | 2.5 | Influenza, Inhalation, | Neu2 | BFAT, Kidney, Liver, |
| | | Prevention | | Lung |
| Zidovudine | 1.1 | HIV, Liver | Tert | Lung |
| Zolmitriptan | 3 | Headache, Liver, Migraine | Htr1b, Htr1d | Adr, BS, Lung |
| Zolpidem | 2.6 | Cancer, Insomnia, Liver, | Gabra3, Gabra2 | Adr, Aorta, BFAT, BS, |
| | | Prevention, Seizure, Sleep | | Cere, Hypo, Kidney |
| Zopiclone | 5 | Addiction, Depression, | Gabra3, Tspo, Gabra2, | Adr, Aorta, BFAT, BS, |
| | | Insomnia, Liver, Liver | Gabra5 | Cere, Hypo, Kidney, |
| | | Enzymes, Pill, Sleep | | Lung |

Data regarding circadian oscillations, including coding and non-coding genes, are available via the World Wide Web (www) bioinf.itmat.upenn.edu/circa, a subset of which is summarized in Table 2, infra.

TABLE 2
Circadian Oscillations in Transcript Expression Data
(numbers represent circadian time in hours)

| | | | Brown | Brain- | Cere- | | Hypo- | | | | | White |
|---|---|---|---|---|---|---|---|---|---|---|---|---|
| Target Gene | Adrenal | Aorta | Fat | stem | bellum | Heart | thalamus | Kidney | Liver | Lung | Muscle | Fat |
| AAAS | | | | | | | 6 | | | | | |
| AACS | | | | | | | | | | 8 | | |
| AADAC | | | | | | 21 | | 22 | | | | |
| AAED1 | | | | | | | | 9.5 | | | | |
| AAGAB | | | | | | | | | | 6 | 8 | |
| AAK1 | | 17 | | | | | | | | | | |
| AAMP | | | | | | | | | 0 | | | |
| AASDH | | | | | | | | | 21 | | | |
| AASDHPPT | | | 4.5 | | | | | | | | | |
| AASS | | | | | | | | 21.5 | 21 | | | |
| AB041803 | | | | | | | | | 6.5 | | | |
| ABAT | | | 21 | | | | | | | | 11.5 | |
| ABCA1 | | | | | | | | | | 1.5 | | |
| ABCA12 | | | | | | | | | 22 | | | |
| ABCA13 | | | | | | | 7 | | | | | |
| ABCA17 | | | 11.5 | | | | | | | | | |
| ABCA2 | | | | | | | | 22 | | | | |
| ABCA3 | | | | | | | | | 16 | 19 | | |
| ABCA4 | | | 21 | | | | | | | | | |
| ABCA5 | | | 23 | | | | | | | | | |
| ABCA6 | | | | | | | | 22 | | | | |
| ABCA7 | | | | | | | | | 2 | | | |
| ABCA8 | | | | | | | | | | | 6 | |
| ABCA8A | | | | 21 | | | | | | 22 | | |
| ABCA9 | | | 23 | | | | | | | | | |
| ABCB1 | | | | | | 5 | | | | | | |
| ABCB10 | | | | | | | | | | | 8 | |
| ABCB11 | | | | | | | | | 0 | 12 | | |
| ABCB4 | | | | | | | | | 21.5 | | | |
| ABCB6 | | | | | | | | 9 | | | | |
| ABCB7 | | 9.5 | 10 | | | | | | | | | |
| ABCB8 | | | | | | | | 12 | | | | |
| ABCC1 | 19 | | | | | | | | 21 | 22 | | |
| ABCC10 | | 8 | | | | | | | | | | |
| ABCC2 | | | | | | | | | 23 | | | |
| ABCC4 | | 7 | 7 | | | | | | | | 8 | 6 |
| ABCC5 | | | | | | | | 11 | | | | 8.5 |
| ABCC9 | | | | | | | | | 2 | | | |
| ABCD1 | | 8 | | | | | | | | | | |
| ABCD2 | | | | | | | | | | | 5 | |
| ABCD4 | | | | | | | | 23 | | | | |
| ABCE1 | | | | | | | | | 0 | | | |
| ABCF1 | | | | | | | | | 6 | | | |
| ABCF2 | | 10 | 11 | | | | | | | | | 11 |
| ABCF3 | | | | | | | | 20 | | | | |
| ABCG1 | 18 | | | | | | | | | | | |
| ABCG2 | | 20.5 | | | | | | | 19.5 | | | |
| ABCG4 | | | | | | | | 21 | 19 | | | |
| ABCG5 | | | | | | | | 20 | | | | |
| ABCG8 | | | | | | 1.5 | | | | | | |
| ABHD11 | 19 | | 22 | | 0.5 | | | | | | | |
| ABHD14A | | | | | | | | 22 | 20.5 | | | |
| ABHD14B | | 5.5 | 5 | 5 | | | | | | 7 | | |
| ABHD15 | | | | | | | | | 0.5 | | | |
| ABHD16A | | | | | | | | | | 20 | | |
| ABHD2 | | | | | | | | 23 | | | 7 | |
| ABHD3 | | | | | | | | | 7 | | | 7 |
| ABHD4 | | 14.5 | | | | | | 12 | | 13 | | |
| ABHD6 | | | 23 | | | | | 0 | | 23 | 23 | |
| ABHD8 | | | | | | | | | 18.5 | 18 | | |
| ABI1 | | | | | | | | 23 | 23 | | | |
| ABI2 | | | | | | 13 | | | | | 8 | |
| ABI3 | | | | | | | | 23 | | | | |
| ABL1 | | | | | | | | 23 | | | | |
| ABLIM1 | | | | | | | | 22 | | | | |
| ABLIM2 | | | | | | | | | 21 | | | |
| ABLIM3 | | | | | | | | | 21 | | | |
| ABP1 | | | | | | | | 23 | | 4 | | |
| ABR | | | | | | | | 20.5 | | | | |
| ABRA | | | 6 | | | | | | | | | |
| ABRACL | | | | | | | | 3 | | | | |
| ABTB1 | | | | | | 5 | | | | | | |
| ABTB2 | | | | | | | | | | 9 | | |
| AC027184.1 | | | | | | | | | 21 | | | |
| AC083948.1 | | | | | | 21 | | 21 | 18 | | | |
| AC091683.2 | | | | | | | | | 21 | | | |
| AC101527.1 | | | | | | | | 21 | | | | |
| AC109305.2 | | | | | | | | | 20 | | | |
| AC122012.1 | 20 | | | | | | | | | | | |
| AC122260.2 | | | | | | | | | | 14 | | |
| AC122872.1 | | | | | | | | 2 | | | | |
| AC130208.1 | | | | | | | | | | 15 | | |
| AC132253.3 | | | | | | | | 23.5 | | 12.5 | | |
| AC132457.1 | | | | | | | | 19 | | | | |
| AC133509.2 | | | | | | | | 19 | | | | |
| AC139157.1 | | | | 21 | | 18 | | | | | | |
| AC141881.34 | | | | | 23 | 9.5 | | | | | 10 | |
| AC150897.1 | | | | | | | | | 22 | | | |
| AC153928.2 | | | | | | | 19 | | 1 | | | |
| AC158295.1 | | | | 20 | | | | | | | | |
| AC159129.1 | | | | | | 15.5 | | | | | | |
| AC168120.1 | | | | | | | | | | | 6 | |
| AC174597.1 | | | | | | | | 1 | | | | |
| AC225448.1 | | | | 21 | | | | 18.5 | | 17 | | |
| ACAA1 | | | | | | | | 21.5 | | | | |
| ACAA2 | | | | | | | | | 21.5 | | | |
| ACACA | | | | | | | | | | | 2 | |
| ACACB | | | | | | 18 | | | | | 19.5 | |
| ACAD10 | 20 | | | | | | | 23 | 22.5 | 0 | | |
| ACAD11 | | | | | | | | 8 | | 2.5 | | |
| ACAD8 | | | | | 19 | | | | | | | |
| ACADL | | | | | | | | 9 | | | | |
| ACADM | 1.5 | | | | | | | | | | | |
| ACADVL | 18.5 | 21 | | 22 | | | | 23 | 20.5 | | 8 | |
| ACAN | 21 | | | | | | | | 22 | | | |
| ACAP2 | | | | | | | | | 22 | 16 | | |
| ACAP3 | | 22 | | | | | | 22 | | | 2 | |
| ACAT2 | | | | | | | | | 0 | | | |
| ACAT3 | | | | | | | | | 21 | | | |
| ACBD4 | | | | | | | | | 22 | | | |
| ACBD5 | | | 6 | | | | | 23 | 22.5 | | | |
| ACCS | 22.5 | | | | 16 | | | 5 | | | | 6 |
| ACE | | | | | 22.5 | | | | | | | |
| ACER2 | | | | 10 | | | | | | | | |
| ACHE | | | | | | | | | 5.5 | | | |
| ACIN1 | | 16.5 | 18 | | | 19.5 | | | 18 | 16 | 1 | |
| ACLY | | | | 19 | | | | | | | | |
| ACMSD | | | | | | | | | 13 | | | |
| ACN9 | | 10 | | | | | | | | | | |
| ACO2 | | | | | | | | 1 | 3 | | | |
| ACOT1 | | | 2 | | | | | | | 13 | | |
| ACOT11 | | | | | | | 4 | | | | | |
| ACOT12 | | | | | | | | 20 | | | | |
| ACOT2 | | 6 | | | | | | | 20.5 | | | |
| ACOT4 | 5.5 | | | | | | | | 19.5 | | | 1 |
| ACOT9 | | | | | | | | 22.5 | 23 | | | |
| ACOX1 | | | | | | | | 23 | | | | |
| ACOX2 | | | 5 | | | | | | | | | |
| ACOX3 | | | | | | | | 23 | | | | |
| ACOXL | | | 22 | | | | | | | 15 | | |
| ACP2 | | | | | | | | | 21 | | | |
| ACP6 | | | | | | | | | 22 | | | |
| ACPP | | | | | | | | 5.5 | | | | |
| ACSF3 | | | | | | | | | 20.5 | | | |
| ACSL1 | | | | | | | | | | 14 | | 13 |
| ACSL3 | | | | | 20 | | | | 21 | | | |
| ACSL4 | | | | | | | | | 21 | | | |
| ACSL5 | | | 0 | | | | | | | | | 23.5 |
| ACSM5 | | | | | | | | 22 | 23 | 17 | | |
| ACSS2 | | | | | | | | | | 5.5 | | |
| ACSS3 | | | | | | | | | | 17 | | |
| ACT1 | | | | | | | 8 | | | | | |
| ACT2 | | | | | | | | | 23 | | | |
| ACTA2 | | | 8 | | | | | | | | | |
| ACTB | | | | | | | | | | 12 | | |
| ACTN1 | | | | | | | | 22 | | | | |
| ACTN4 | | | | | | | | 2 | | | | |
| ACTR1B | | | | | | | | 22 | | | | |
| ACTR3 | | 11.5 | | | | | | | | | | |
| ACTR3B | | 23 | 22 | | | 19 | | 20 | 21 | | | |
| ACVR1 | 7.5 | | | | 10 | | | 11.5 | | | | |
| ACVR1B | | | | | | | | 19 | | | | |
| ACVR1C | | | | | | 15 | | | | | 8 | |
| ACVR2A | | | | | | | | 22 | | | | |
| ACVR2B | | | | | | | | | 23 | | | |
| ACY1 | 22 | 23 | 21 | | | | | | | | | |
| ACY3 | 15.5 | | 17 | | | 18 | | | | 15 | | |
| ACYP1 | | | | | | | | 22.5 | | | | |
| ADAL | | | | | | | | | 22 | | | |
| ADAM10 | | | | | | | | | 22.5 | | | |
| ADAM12 | | | | | | | | | 22 | | | |
| ADAM17 | | | | | 21 | | | 21 | | | | |
| ADAM19 | 18 | | | 21 | | | | | 22 | | | |
| ADAM22 | | | 0.5 | | | | | 22.5 | | | | |
| ADAM23 | | | | | | | | 22 | | | | |
| ADAM28 | | | | | | | | | 20 | | | |
| ADAM29 | | | | | | | | | 8 | | | |
| ADAM32 | | | | | | | | 7 | | | | |
| ADAM6B | | | 0 | 5 | | | | 0 | | | 0 | 0 |
| ADAM9 | | | | | | | | | | 8 | | |
| ADAMDEC1 | | | | | | | 12 | | | 12 | | |
| ADAMTS1 | | | | | | 18.5 | | | | | | |
| ADAMTS10 | | | | | | 21 | | | | | | |
| ADAMTS12 | | | | 10 | | | 9 | | | | | |
| ADAMTS15 | | | | | | | | | | 1.5 | | |
| ADAMTS16 | | 12 | | | | | | | 8 | | | |
| ADAMTS17 | | | | | | | | 0 | | | | |
| ADAMTS2 | | | | | | | | 0 | | | | |
| ADAMTS3 | | | | | | | | | | 10.5 | | |
| ADAMTS4 | | | | | | | | 6 | | | | |
| ADAMTS5 | | | | 2.5 | | | | | | | | |
| ADAMTS6 | | | | | | | | 21 | 21 | | | |
| ADAMTS8 | | | | | | 10 | | | 9.5 | | | |
| ADAMTS9 | | | | | | | | 22 | | | | |
| ADAMTSL1 | | | | 20 | | | 18 | | | | | |
| ADAMTSL3 | | | | | | | | | 4 | | | |
| ADAMTSL4 | | | 22 | | | | | | | | | |
| ADAMTSL5 | | | | | 16.5 | | | | | 13 | | |
| ADAP1 | | | | | | | | | | 17 | | |
| ADAP2 | | | | | | | | | | 9 | | |
| ADAR | | | | | | | | | | 2 | | 5 |
| ADARB1 | | | | | | | | 20.5 | | | | |
| ADAT1 | | | | | | | | 22 | 22.5 | | | 23 |
| ADAT2 | | | | | | | | | 0.5 | | | |
| ADC | | | | | | | | 10 | | 11 | | |
| ADCK2 | | | | | | | | 18.5 | | | | |
| ADCK3 | | | | | | | | | | | 11 | |
| ADCK4 | | | | | 21 | | | | 22 | | | 9 |
| ADCK5 | | | | | | | | | 1 | 4 | | |
| ADCY3 | | | | | | | | | | 13 | | |
| ADCY4 | | | | | 20.5 | | | | | | | |
| ADCY5 | | | | | | | | 21 | | | | |
| ADCY6 | | | | | | | | | | 12 | | |
| ADCY7 | | | | | | | | | 0.5 | | | |
| ADCY8 | | | | | | | | | | 12 | | |
| ADCY9 | | 9 | | | | | | | | | | |
| ADCYAP1R1 | | | 8.5 | | | | | | | | | |
| ADD3 | | | | | | | 18 | | | | | |
| ADH1C | | | 21 | | | | | | | 13 | | |
| ADH4 | | | 11.5 | | | | | | | | | |
| ADH6-PS1 | | | | 22 | | | | | 22 | | | |
| ADH7 | | | | 9 | | | | | 2 | | | |
| ADHFE1 | | | | | | 12.5 | | 10 | | | | |
| ADI1 | | | | | | | | 10 | | | | |
| ADIPOQ | | | | 14 | 13 | 14.5 | 13 | | | | 18 | |
| ADIPOR2 | | | | | | | | | 5 | | | |
| ADM | | | | | | | | | 21 | | | |
| ADNP2 | | | 22.5 | | | | | 2 | | | | 20.5 |
| ADO | | | | | | | | | 6 | 11 | | 10 |
| ADORA1 | | | | | | | | 12 | | | | |
| ADORA2A | | | 7 | | | | | | | | | |
| ADORA2B | | | | | | | | | 19 | | | |
| ADPGK | | | | | | 18 | | 17 | 17 | | | |
| ADPRHL1 | | | | 20.5 | | | | 19 | 22 | 16 | | |
| ADPRHL2 | | | | | | | | 9 | | 9 | | |
| ADRA1A | | 8 | 10 | | | | | 6 | 5 | | | |
| ADRA1B | | 22 | 22 | | | 21 | | 22 | 22 | | 21.5 | |
| ADRA1D | | | | | | | | 22 | | | | |
| ADRA2B | | | | | | | | | | 9 | | |
| ADRB1 | | | | | | | | 19 | | | | |
| ADRB2 | | | | | | | 8 | | | | | |
| ADRB3 | | | | | | | | | | 12 | | |
| ADRBK2 | | | | | | | | 0 | 3 | | | |
| ADRM1 | | | | | | | | | | 16 | | |
| ADSS | | | | | | | | 20 | 21 | | | |
| ADTRP | | | 22 | | | 22 | | | | | | |
| AEBP1 | | | | | | | | 12 | | | | |
| AFAP1 | | | | | 9 | | | | | | | |
| AFAP1L2 | | | | 1.5 | 3 | 7 | | 14 | | | 2 | |
| AFF1 | | | | | | | | 9 | 9 | | | |
| AFF2 | | | | | | 7 | | | | | | |
| AFF3 | | | | | | | 5.5 | | | | | |
| AFF4 | | | | | | | | | 23 | | | |
| AFG3L2 | | | | | | | | 0 | | | | |
| AFMID | | | 1 | | | | | | | | | |
| AFTPH | | | | | | | | | 4 | | | |
| AGA | | | | | | | 21 | 19.5 | | | | |
| AGAP1 | | | | | | | | 19 | 16 | | | |
| AGAP2 | 5 | | 8 | 7.5 | | 10 | | 6 | | | | |
| AGAP3 | | | | | | | | | 19 | | | |
| AGBL5 | | | 19 | | | | | | 22.5 | | | |
| AGFG1 | | 13 | 15 | | 13 | 16 | | | 14 | 12.5 | | |
| AGFG2 | | | | 20 | | 0 | | 17 | | | | |
| AGL | | | | | | | | | 3 | | | |
| AGMAT | | 6 | | | | | | | | | | |
| AGMO | | | | | | | 22 | | 8.5 | | | |
| AGPAT1 | | | | | | | | | | 4 | | |
| AGPAT2 | | | | | | | | | 15 | | | |
| AGPAT3 | | | | | | | | | 2 | | | |
| AGPAT4 | | | | | | | | | | 12 | | |
| AGPAT5 | | | | | | | | 22.5 | | | | |
| AGPAT6 | | | | | | | 6 | | | | | |
| AGPAT9 | | | | | | 0.5 | | 23 | | 2 | 23 | |
| AGPHD1 | | | | | | | | | 23 | | | 8 |
| AGPS | | 21.5 | | | | | | | | | | |
| AGTPBP1 | | | | | | | | | 23 | | | |
| AGTR1 | | | | 3 | | | | | | | | |
| AGXT | | | | | | | | | 17 | | | |
| AGXT2L1 | | | 21 | | | | | | | | | |
| AHCTF1 | | | | | | | | | 5 | | | |
| AHCYL2 | | | | | | 12 | | 13 | | | | |
| AHDC1 | | | | | | 17 | | | | | | |
| AHK | 19 | | 1 | | | | | | | | | |
| AHK2 | | | | | | 13.5 | | | | | | |
| AHSA1 | | 7 | 7 | | | | | 8.5 | | | | |
| AHSA2 | | | | | | | | 6.5 | | | | |
| AI182371 | | | | | | 14 | | | 8 | | 13 | |
| AI317395 | | | | | | | | | 22 | | | |
| AI606181 | | | | | | | | | 22 | | | |
| AI607873 | | | 5 | | | | | | | | | |
| AIF1L | | | | | | | | 23 | | | | |
| AIFM1 | | | | | 6 | | | | | 15 | | |
| AIFM2 | | | | | | | | | | 16 | | |
| AIG1 | | | | | | | | | | 16 | | |
| AIMP1 | | | | | | | | | | 22 | | |
| AIRN | | | | | | | | | | 8 | | |
| AK1 | | | | | | | 17 | | | | | |
| AK2 | | | | | | 17 | | | | | | |
| AK3 | 14 | 14 | 15 | | 17 | 14 | | 13 | 12.5 | 13 | 13 | 14 |
| AK4 | 21.5 | | | | | | | | 2.5 | | | |
| AK5 | | | | | | | | 2 | 6 | | | |
| AK8 | | | | | | | | | | 7 | | |
| AKAP1 | | 21.5 | | | | | | | | | | |
| AKAP11 | | | | | | | | | 23 | | | |
| AKAP12 | | | | | 18 | | | | 21 | | | |
| AKAP13 | | | | | | | | | 22.5 | | | |
| AKAP17B | | | | | | | | | 22 | | | |
| AKAP5 | | | | | | 18 | | 14.5 | | 15 | | |
| AKAP9 | | | | | | | | | 1 | 10.5 | | 9 |
| AKIP1 | 20 | | | | | | | 21 | | | | |
| AKIRIN2 | | | | | | | | | 21 | | | |
| AKR1B10 | | | | | | | | 21 | | | | 22 |
| AKR1B7 | | | | | | | | | | 5.5 | | |
| AKR1C1 | | | | | | 23 | | | | | 22 | 6 |
| AKR1C14 | | | | | | | 1 | | | | | |
| AKR1C19 | | 6.5 | 7 | | 7 | | | 5 | | | | |
| AKR1C20 | | | | | | | 10 | | | | | |
| AKR1D1 | | | | | | | | 4 | 2 | | | |
| AKR1E2 | | | | | | | | | | 14.5 | | |
| AKT1 | | | | | | | | | 22.5 | | | |
| AKT2 | 5 | | 9 | | | | | 9.5 | 9 | 2 | | |
| AKT3 | | | | | | | | 23 | | | | |
| AKTIP | | | | | | | | | 21 | | | |
| AL731554.1 | | | | | | | | | 15 | | | |
| AL807771.1 | 19 | | | | | | | | 23 | | | |
| ALAD | | | | | | | | | 2 | | | |
| ALAD2 | | | | | | | | 7.5 | | | | |
| ALAS1 | | | | | | | | | | 6 | | |
| ALCAM | | | | | | | | | 23.5 | | | |
| ALDH18A1 | | | | | | | | 20 | | | | |
| ALDH1A1 | 22 | | | | 6 | 23 | 6 | | | | | |
| ALDH1A2 | | | | | | | | | | | 22 | |
| ALDH1A7 | | | | | | | | | | 13 | | |
| ALDH3A1 | 4 | 5 | | | | | 8 | 6 | 3 | 5 | | |
| ALDH3A2 | | | | | | | | | 10 | | | |
| ALDH3B1 | | | | | | | | | 21 | | | |
| ALDH3B2 | | | | | | | | | 0 | | | |
| ALDH7A1 | 7 | | | | | | | | 22 | | | |
| ALDH8A1 | | | | | | | | | | 8 | | |
| ALDH9A1 | | | | | | | | | | | | 5 |
| ALDOA | | | | | | | | 20 | | 16 | | |
| ALDOB | | | | | | | | 12 | | | | |
| ALDOC | | | | 19.5 | | | | | | | | |
| ALG11 | | 15 | | | | | | | | | | |
| ALG12 | | | | | | | 8 | | | | | |
| ALG14 | | 2.5 | | | | 23 | | | | | | |
| ALG3 | | | | | | | | 11 | | | 8.5 | |
| ALG5 | | 23 | | | | | | | | | | |
| ALG6 | | | | | 2 | | | 23 | | | | |
| ALG8 | | | | 1 | 1 | 1 | 23 | 2.5 | | | | |
| ALKBH3 | | | | | | | | | | 16 | | |
| ALKBH6 | | | | | | | | 16.5 | | | | |
| ALKBH7 | | | 1 | | | | | | | | | |
| ALKBH8 | | | | | | | | | 0.5 | | | |
| ALMS1 | | | 6 | | | | | 8.5 | 6 | 7 | | |
| ALOXE3 | | | | | | | | 23 | 22 | | | |
| ALPK1 | | | | | | | | | 6 | | | |
| ALPK2 | | | | | | | | | 20 | | | |
| ALPL | | | 7 | | | | | | | | | |
| ALS2 | | | | | | | 6 | | | | | |
| ALS2CL | | | | | | | | | | 15 | | |
| ALS2CR12 | 16 | | 20.5 | | 17 | | 20 | 16 | 14.5 | 15 | | 18 |
| ALYREF | | | | | | | 5.5 | | 9 | | | |
| ALYREF2 | | | | | | | | | | 13 | | |
| AMACR | | | 20 | | | | | | | | | |
| AMDHD1 | 5 | | | | | | | | | 6 | | |
| AMDHD2 | | | | | | | | 23 | | 14 | | |
| AMFR | | | | | | | | | | | 4.5 | |
| AMIGO2 | | | | | | | | | | | 4.5 | |
| AMMECR1 | | | | | | | | | | | 7 | |
| AMOT | | | | | | | | | | | 5.167 | |
| AMOTL1 | | | | | | | | | | | 5.833 | |
| AMOTL2 | | | | 3.167 | | | | | | | | |
| AMPH | | | | | | | | 13 | | | | |
| AMT | | | | | | | | | | | 3.167 | |
| ANGEL1 | | | | | | | | | | | 4.833 | |
| ANGEL2 | | | | | | | | | | | | 6 |
| ANGPT1 | 5 | | | | | | | | | | | |
| ANGPT2 | | | | | 14.5 | | | | | 0.5 | 2.833 | |
| ANGPTL1 | | | | 12 | | | | | | | | |
| ANGPTL2 | | | | | | | | | | 10 | | |
| ANGPTL4 | | | | | | 1 | | | | | | |
| ANGPTL7 | | | | | | | | | | 23 | | 23 |
| ANK1 | | 8 | 9 | | | 21 | | | 13.5 | | | |
| ANK2 | | 3 | 3 | | | | | | | | | |
| ANK3 | | | 23 | | | | | | | | | |
| ANKH | | | | | | | | | 21 | | | |
| ANKLE2 | | | | | 15 | | | | | | | |
| ANKMY2 | 12 | | | | | 15 | | 20 | 13 | 0 | | |
| ANKRA2 | | | | | | | | | | 22 | | |
| ANKRD11 | | | | | | | | | | 5.5 | | |
| ANKRD12 | | 10.5 | 10 | | 3 | | | | | | | |
| ANKRD13A | | | | | 6 | | | | | | | |
| ANKRD13C | | | | | | | | | | 6 | | |
| ANKRD16 | | | | | | | | | | 8 | | |
| ANKRD17 | | | | | | | 8 | | 22 | | | |
| ANKRD23 | | | 22.5 | | | | | 20 | | 17 | | 23 |
| ANKRD28 | | | | 6.5 | | | 7 | | | | | |
| ANKRD33B | | | | | | | | | | 12 | | |
| ANKRD34C | | | 0.833 | | | | | | | | | |
| ANKRD40 | | | | | | | | 21 | | | | |
| ANKRD44 | | | | | | | | 22.5 | | | | |
| ANKRD46 | | | | | | | | | | 14 | | |
| ANKRD49 | | | | | | | | 11 | | 11 | | |
| ANKRD5 | | | 20 | | | | | | 17 | | | 21 |
| ANKRD50 | | | 22 | | | | | 2 | | | | 2 |
| ANKRD52 | | | 11 | | | | | 22 | | | | |
| ANKRD9 | | | | | | 0.5 | | | | | 2 | |
| ANKS1A | | | | | | | | 20 | 19 | | | |
| ANKUB1 | | | 20 | 22 | 3 | | | | | | | |
| ANLN | | | | | | | | 22 | | | | |
| ANO3 | | | | | | | | | | 17 | | |
| ANO4 | | | | | | | | 8 | | | | |
| ANO6 | | | | | | | 16.5 | | | | | |
| ANO8 | | | 5 | | | | | | 1 | | | |
| ANP32A | | | | | | 12 | | | 21 | 13 | 11.5 | |
| ANP32-PS | | | | 15 | | | 14 | | | | | |
| ANPEP | | | | | | 18 | | 19.5 | 17 | | | |
| ANTXR1 | | | 0 | 21 | | | | 22.5 | | | | |
| ANTXR2 | | | | | | | | | 22 | | | |
| ANXA11 | | 6.5 | 7 | | | | | | | 9 | | |
| ANXA2 | | | | | | | | | | | | 6.5 |
| ANXA3 | | | | 21.5 | | | | 19 | | | | |
| ANXA5 | | | 3.5 | | | | | 23 | | | | 0 |
| ANXA7 | | | | 20 | | | | 19 | | | | |
| ANXA8 | | | | | | 7 | | | | | | |
| ANXA9 | | | | | | | | 21 | | | | 22 |
| AOC3 | | | | | | | 6 | | 9 | | | |
| AP1AR | | | | 22 | | 20 | | | 23 | 20 | | |
| AP1G1 | 11 | 12.5 | 13 | 12 | 12 | 12 | 12 | 12 | 12 | 12 | 11 | 14 |
| AP1M1 | | | | | | | | | 5 | | | |
| AP1S1 | | | 4.167 | | | | | 21.5 | | | | |
| AP1S2 | | | | | | | | 21 | | | | |
| AP2A1 | | | | | | | | | | 12 | | |
| AP2A2 | | | | | | | | 17 | | | | |
| AP2S1 | | | | | | | | 21.5 | | | | |
| AP3D1 | | | | | | | | 19 | | | | |
| AP3M1 | | | | | | | | 11 | | | | |
| AP5S1 | | | | | | | | | 9 | | | |
| APAF1 | | 8 | | | | | | 10 | | | | |
| APBA3 | | | | | | | | | 23.5 | 21 | | |
| APBB1IP | | | | | | | | | | 11 | | |
| APBB3 | | | | | | | | | 22 | | | |
| APC | | | | | | | | | | 9.5 | | |
| APC1 | | | | | | 7.5 | | | | | | |
| APC10 | | | | | | | | | | 10 | | |
| APC11 | | | | | 12 | | | | | | | |
| APC13 | | | | | | | | | 18 | | | |
| APC16 | | | | | | | | | 23 | | | |
| APC7 | | | | | | | | 9.5 | 11 | | | |
| APCDD1 | | | | | | | | | | 6 | | |
| APEX1 | | | | | | 5.5 | | | 17 | 13 | | |
| APEX2 | | | | | | | | | 10 | | | |
| APH1B | | | | | | | | | | | 8 | |
| APH1C | | | | | | | | | | 8 | | |
| API5 | | | | | | | | 22 | | | | |
| APIP | | | | 4 | | | | | | | | |
| APLN | | | 8 | | | | | | | | | |
| APLNR | | | | | | | | | 21 | | | |
| APOA1BP | 15 | | | | | | | | | | | |
| APOA5 | | | | | 19.5 | | | | | | | |
| APOBEC1 | | | | | | 22 | | | | | | |
| APOBEC2 | | | | | | | | | 5.5 | | | |
| APOC1 | | | | | | | | | | 14.5 | | |
| APOC2 | | | | | | | | | 7 | | | |
| APOC3 | | | 22 | 20 | | | | 20 | | | | |
| APOD | | | | | | | | 12 | | | | 14 |
| APOE | | | | | 1 | | | | | | | |
| APOL6 | | | 9 | | | | | | | | | |
| APOLD1 | 19 | | | | | | | | | | | |
| APON | | | 21.5 | 21.5 | | | | 19.5 | 5.5 | | | |
| APPL1 | | | | | | | | 22 | | | | 0 |
| APPL2 | | 8 | 8 | | | 6.5 | | | | | | |
| APRT | | | | 12 | | 10.5 | | | | 9 | | |
| AQP1 | | | 6.5 | | | | | | | | | |
| AQP11 | | | 9.5 | | | | | | | | | |
| AQP3 | | | | | | | | | 5.5 | | | |
| AQP4 | | 5 | 7 | | | | | | 20 | | | |
| AQP6 | | | | | | | | 0 | | | | |
| AQP7 | 2.5 | | | | | | | | | | | |
| AQP8 | | | | | | | | 18.5 | 18 | | | |
| AQP9 | 18 | | 23.5 | 21 | | | | 18 | 19 | | | |
| AQR | | | | | 0 | | | | 19 | 17 | | |
| AR | | 7 | 7 | | | | | | 3 | | | |
| ARAF | | | | | | | | | | | 5 | |
| ARAP1 | | 8.5 | 6 | | | | | | 6 | | | |
| ARAP2 | | 7 | | | | | | | | 9 | | |
| ARAP3 | 15 | 15 | 15 | | | 17 | | | 14 | 14 | | 14.5 |
| ARF1 | | | | | | | | | 18.5 | | | |
| ARF3 | | | 22 | | | | | 20 | 23 | | | |
| ARF5 | | | | | | | | | 23 | | | |
| ARFGAP1 | | | | | 23 | | | 21 | | | | |
| ARFGEF1 | | | | | 21 | | | | | 17 | | |
| ARFGEF2 | | | | | | | | 11 | | | | |
| ARFIP1 | | | | | | | | 3.5 | | | | |
| ARFIP2 | | | | | | | | | 23.5 | | | |
| ARGLU1 | | | 10 | | 11 | | | | | 10 | | |
| ARHGAP1 | | | | | | 23 | | | | | | |
| ARHGAP10 | | | 8.5 | | | 8 | | 9 | | | | 9 |
| ARHGAP12 | | 4 | | | | | | | | | | |
| ARHGAP17 | | | | | | | | | 0 | | | |
| ARHGAP18 | | | | | | | | | 23 | | | |
| ARHGAP20 | | | | | | | 18 | | 23.5 | | | |
| ARHGAP21 | | | | | | | | | 16 | | | |
| ARHGAP23 | 4 | | | | | | | | | | | |
| ARHGAP24 | | | | | | | | 2.5 | 4 | | | |
| ARHGAP25 | | | 23 | | | 21 | | | | | 0 | |
| ARHGAP26 | | | | 21.5 | | | | | | | | |
| ARHGAP27 | | | | | | | | | | 18 | | 11 |
| ARHGAP29 | | | | | | | | | | | | 0 |
| ARHGAP30 | | | | 21 | | 3.5 | | 20.5 | 18.5 | | | 0 |
| ARHGAP31 | | | | | | | | 20 | | | | |
| ARHGAP35 | | | | | | | | 22 | | | | |
| ARHGAP39 | | | | | | | | | | 8 | | |
| ARHGAP5 | 9 | 9 | | | | | | 14 | | | | 11 |
| ARHGDIB | | | | | | | | 8 | | | 5.167 | |
| ARHGEF1 | | | 11 | | | | | | | | | |
| ARHGEF10L | | | | | | | | | | 5 | | |
| ARHGEF12 | | | | | | | | | 0 | | | |
| ARHGEF15 | | | 9 | | | | | | | | | |
| ARHGEF17 | | | | | | | | | 11 | | | |
| ARHGEF18 | | | | | | | | 8.5 | | | 5.167 | |
| ARHGEF19 | | | | | | | | | 1 | | | 22.5 |
| ARHGEF26 | | 5.5 | 7 | | | | | | 17 | | | |
| ARHGEF3 | | | | | | | 6 | | | | | |
| ARHGEF37 | | | | | | | | | 0.5 | | | |
| ARHGEF5 | 8 | | 9 | | | | | 4 | | | | 9.5 |
| ARHGEF7 | | | | | | | | 22 | | | | |
| ARHGEF9 | | | | 12 | | | | | | | | |
| ARID1A | 20 | | | | | | | | | | | |
| ARID1B | | | | | | | | | 16 | | | |
| ARID5B | 19 | | | | | | | | | | | |
| ARIH1 | | | | | | | | | | 15 | | |
| ARIH2 | | | | | | | | | 22 | | | |
| ARL10 | | | | | | | | | | 17 | | 22 |
| ARL15 | | | | | | | | 16 | | | | |
| ARL2BP | | | | | | | | | 19 | | | |
| ARL3 | | | | | | 18 | | | | | | |
| ARL4A | | | | | | | | 12 | | | | |
| ARL4D | | | 22 | | | | | 23 | 13.5 | | | |
| ARL5B | | | 23 | | | | | | | | | |
| ARL6IP1 | | | | | | | | 15 | | | | |
| ARL6IP5 | | | 15 | | | | | 13.5 | | 14 | | 13 |
| ARL8A | 11 | | | | | 15 | | | 12 | 12 | | |
| ARL8B | | | | | | | | | 4 | | | |
| ARMC2 | 23 | | | | | | | | | 21.5 | | |
| ARMC9 | | | | | | | | 22 | | | | |
| ARMCX1 | | | | | 22 | | | 20 | 19 | 18.5 | | |
| ARMCX3 | | | | | | | | | 2 | | | |
| ARMCX4 | | | | | | | 8 | | | | | |
| ARNT | | | | | | | | | 18 | | | |
| ARNTL | | | | | | | | | 19 | | | |
| ARPC1A | | | | | | | | | 1 | | | |
| ARPP19 | | | 21 | 19 | | | | 16 | | 15.5 | | 17 |
| ARRB1 | 20 | | | | | | | | | | | |
| ARRDC2 | | | | | | | 9 | | | | | |
| ARRDC3 | | | | | | | | 23 | | | | |
| ARRDC4 | | | 23.5 | | | | | 22 | 22 | | | 23 |
| ARSA | | | | | | | | 17 | 23 | | | |
| ARSB | | | | | | 21 | | | | | | |
| ARSG | | | 23 | | 6 | | | 21 | 22 | | | |
| ARSJ | | | 23 | | | | | 20 | | | | 23 |
| ARSK | 20 | | | | | | | | | | | |
| ART1 | | | | | | | 9 | | | | | |
| ART3 | | | | | | | | | 14.5 | | | |
| ART4 | | | | | | | | | 15 | | | |
| ARVCF | | | | | | 12 | | | | | | |
| AS3MT | | | | | | | | | | 16 | | |
| ASAH1 | | | | | 21.5 | | | | | | | |
| ASAP1 | | | | | | | | | 22 | | | |
| ASAP2 | | | 0 | | | | | | 21 | | | |
| ASAP3 | | | 23 | | | | | | | 16 | | |
| ASB10 | | | | | | | | | 21 | | | |
| ASB12 | | | 21 | | | | | | | | | |
| ASB13 | | | | | | | | | | | | 10.5 |
| ASB18 | | 3.5 | | | | | | 7 | 15.5 | | | |
| ASB2 | | | | | | | | 14 | | | | 14 |
| ASB4 | | | | | | | 23 | | | | | |
| ASB9 | | | | | | | | 11 | | | | |
| ASCC3 | | | 7 | | | | | | 6 | | | 4.5 |
| ASF1A | | | 12 | | | | | 9 | | | | |
| ASIC5 | | | 21 | | | | | | | | | |
| ASL | | | | | | | | 0 | | | 6 | |
| ASNS | 18.5 | | | | | | | | | | | |
| ASPA | | | 4 | 6 | 9 | | | | | | | |
| ASPDH | | | | | | | | 23 | | | | |
| ASPG | | | | 20 | | | 20 | 22 | | | | |
| ASPH | | | | | | | | 2 | | | | |
| ASPSCR1 | | 7.5 | | | | | | | | | | |
| ASTE1 | | | | | | | 8 | 12 | 22 | | | |
| ASTN2 | | | | | | | | | 23 | | | |
| ASXL3 | | | | | 20 | | | | | | | |
| ATAD1 | | | | | | | | | | | 2 | |
| ATAD2 | | | | | 9 | | | | | | | |
| ATAD2B | | | | | | | | | | 7 | | |
| ATAD3A | | 1.5 | | | | | | | 23 | 21 | 3 | |
| ATAD5 | | | | | | | | | 22 | | | |
| ATAT1 | | | | | | | | | | | 6 | |
| ATF2 | | | | | | | | | | 17 | | |
| ATF5 | 11 | 12 | 12.5 | 11 | 9 | 11.5 | 10 | 13 | 15 | 12 | 10.5 | 14.5 |
| ATF6 | | | | | | | | | 22.5 | | | |
| ATF7IP | | | | | | | | | | 17 | | |
| ATG16L1 | 0 | | | | | 0 | | | 0 | 1 | 0 | |
| ATG16L2 | | | | | | | | | | 11 | | |
| ATG2A | | | | | | | | 21.5 | 23 | | | |
| ATG3 | | | | 19 | | | | | | | | |
| ATG7 | | | | | | | | | | 19 | | |
| ATHL1 | | | | | | | | 2 | | | | |
| ATIC | | | | | | | | | | 15 | | |
| ATL1 | 9 | 15 | 15.5 | | | | | 12 | 14 | 12 | | 15.5 |
| ATMIN | | | | | | | | | | 13 | | |
| ATOX1 | 2 | 0 | 0 | | | | | 16.5 | | | 0 | |
| ATP10A | | | | 22 | | | | | | | | |
| ATP10B | 14.5 | 14.5 | 16 | 15 | 14 | 14 | 14 | 15 | 15 | 13 | 16.5 | 16 |
| ATP10D | | | | | | | | 17 | | | | 14 |
| ATP11A | | | | | | | | | | 10 | | |
| ATP11C | | | 9 | | | | | 10 | 9.5 | | | |
| ATP13A3 | | | 6 | | | | | 6 | | | | |
| ATP13A4 | | | | | | | | 20.5 | | | | 10 |
| ATP1A1 | | 19.5 | 21 | | | | | | | | | 23 |
| ATP1A2 | | | | | | 12 | | | | | 8.5 | |
| ATP1B1 | | | | | | 5.5 | | | | | | |
| ATP1B2 | | | | | | 1 | | | | | | |
| ATP1B3 | | | | 6 | | | | | | | | |
| ATP2A3 | | | | | | | | | 21 | | | |
| ATP2B1 | | | | | | | | | | | 7 | |
| ATP2B2 | | | | 21 | | | 22 | | | | | |
| ATP2B4 | 19 | | | | | | | | | | | |
| ATP2C1 | 14 | 13 | 15 | | | | | | | 15 | | |
| ATP4A | | | | 23 | | | | | | | | |
| ATP5A1 | | | | | | | | | | 22 | | |
| ATP5D | | | | | | 9 | | 10 | 18.5 | 4 | | |
| ATP5I | 19 | 22 | | | | | | | | | | |
| ATP5J | | | | | | | | | 18 | | | |
| ATP5J2-PTCD1 | | | | | | | | | | | 23 | |
| ATP5O | | | | | | | | | | | 6.5 | |
| ATP5S | | 8 | | | | | | | | | | |
| ATP5SL | | | | | | | | 22.5 | | | | |
| ATP6AP1 | | | | | | | | 22 | | | | |
| ATP6V0A1 | | | | | | | | 10.5 | | | | |
| ATP6V0A2 | 16 | | | | | | | | | | | |
| ATP6V0A4 | | | | | | | | | 19.5 | 12 | | |
| ATP6V0B | | | | | | | | | | 3 | | |
| ATP6V1B1 | | | | | | | | | | 22 | | |
| ATP6V1B2 | | | | | | | | | | 2 | | |
| ATP6V1C1 | | | | | | | | | 12 | 11 | | |
| ATP6V1C2 | | | | | | | | 3 | | | | |
| ATP6V1D | | 2 | 2 | | | | | 23.5 | 22 | | | |
| ATP6V1F | | | | | | | | | | 17 | | |
| ATP6V1G3 | | | | | | | | | | | | 7.5 |
| ATP6V1H | | | | | | | | | 23 | | | |
| ATP7B | | | | | | | | | 2 | | | |
| ATP8A1 | 1.5 | | | | | | 5.5 | 1.5 | | | | 23 |
| ATP8A2 | | | | | | | | | 6.5 | | | |
| ATP8B1 | | | 8.5 | | | 12 | 12 | | | 13 | | |
| ATP9A | | | | | | | | 8 | | | | |
| ATPBD4 | | | | | | 11.5 | | | | | | |
| ATPIF1 | | | | | | | | 6 | | | | |
| ATRNL1 | | | | | | | | | 22 | | | |
| ATXN1 | | | | | | | | | 23 | | | |
| ATXN2 | | | | | | | | | 10 | 12.5 | | |
| ATXN2L | | | | | 8 | | | 11 | | | | |
| ATXN3 | | | | | | | | | 22 | | | |
| ATXN7 | | | | | | | 7 | | | | | |
| ATXN7L1 | | | 21 | | | | | | | | | |
| ATXN7L3B | | | | | | | | 21 | 19 | | | |
| AUH | | | | | | | | 11 | 13 | 15 | | |
| AUTS2 | | | | | | | | 13 | 12 | | | |
| AVEN | 6 | | | | | | | | 2.5 | | | |
| AVL9 | | | | | | | | | 17 | | 23.5 | |
| AVPI1 | | | | | | | | | 19 | | | |
| AVPR1A | | 7 | 9 | | | | | | 15 | 10 | | |
| AVPR2 | | | | 20 | 19 | | | 19 | 21 | | | |
| AW551984 | | | | | | | | | 21.5 | | | |
| AXIN2 | | | | | | | | | 16 | 11 | | 0 |
| AXL | | | 21 | | | | | | | | 23 | |
| AZIN1 | | | | | | 1.5 | | | | | | |
| B230307C23RIK | | | 21 | | | 21 | | | | | | 22 |
| B230314M03RIK | | | | | | | | | | | 19 | |
| B330016D10RIK | | | | | | 17 | | | | 16 | | |
| B3GALNT2 | | | | | 22 | | | | 21 | | | |
| B3GALT1 | | | | | | | | 11 | | | | |
| B3GALT2 | | | | | | | | | 23 | | | |
| B3GALTL | | | | | | 3 | | | | | | |
| B3GAT3 | | | | | | | | | 22 | | | 23.5 |
| B3GNT8 | | | | | | 16.5 | | | | 16 | | |
| B3GNT9 | | | | | | | | | 17 | | | |
| B430212C06RIK | | | 17 | | | | | | 17 | | | 19 |
| B430219N15RIK | | | | 11.5 | | 11 | | 2 | | | | |
| B4GALNT1 | | 23.5 | | | 5 | 1 | | | | | 3 | 1.5 |
| B4GALNT3 | | | | | | | | 13 | | | | |
| B4GALT1 | | | | 23 | | | | | 0 | | | |
| B4GALT2 | | | | | | | | | 0 | | | |
| B4GALT5 | 21 | | | | | | | | | 14 | | |
| B4GALT6 | | | | | | | | | 8.5 | | | |
| B4GALT7 | | | | | | | | 4.5 | 21 | | | |
| B530045E10RIK | | | 9 | | | | | 11 | | | | |
| B9D2 | | | | | | | | | 14 | | | |
| BAAT | 6 | 7 | 6 | | | 11 | | | 5 | 10 | | |
| BABAM1 | | | | | | | | | 4 | | | |
| BACE1 | | | | | | | | 11 | 15 | | | |
| BACH1 | | | | | | | | | 23 | | | |
| BACH2 | | | | | | | | 0 | | | | |
| BAG3 | | | | | | | | | 0 | | | |
| BAG4 | | | | | | | 18 | | 15 | 14 | | |
| BAG6 | | | 9 | | | | | | | | | |
| BAI2 | | | | | | | | 13 | | | | |
| BAIAP2 | 21 | | | | | | | 22.5 | | | | |
| BAIAP2L1 | | | 2.167 | | | | | | | | | |
| BANF1 | | 10.5 | 11.5 | | | | | 12 | | 10 | 11 | 11 |
| BANK1 | 11 | | | 12 | | | | 14 | | 11 | | |
| BANP | | | | | 11 | | | | 15 | | | |
| BARD1 | | | | | | | | | | | 6 | |
| BASP1 | | | | 23 | | | | | | | | |
| BATF2 | | | | | | | | | | | | 10 |
| BAZ1A | | | | | | | | | 6 | | | |
| BAZ1B | | | | 6 | | | 9 | | | | | |
| BAZ2A | | | | | | | | 13 | 17 | | | |
| BBOX1 | | | | | | | | 13 | 13.5 | 12 | | |
| BBS12 | | | | | | | | 8 | 22 | | | |
| BBS2 | | | | | | | | | 23 | | | |
| BBS4 | | | | | | 21 | | | | | 7 | |
| BBS7 | | | | | | | | 7 | | | | |
| BBS9 | | 15 | | | | | | | | | | |
| BC005561 | | 17 | 18 | 13 | | 17 | | 13.5 | | 18 | | |
| BC018242 | | | | | | 9.5 | | | | | | 19 |
| BC021614 | | | | | | | | | 21 | | | |
| BC021785 | | | | | | | | 11.5 | | | | |
| BC023105 | | | | | | | 7 | | | | | |
| BC025920 | | | | | | | | | 9 | 0 | 3 | |
| BC026585 | | | | | | 0.5 | | | | | | |
| BC029722 | | | | | | | | | 2.5 | | | |
| BC030307 | | | | | | | | 11 | | | | |
| BC030500 | | | | | | | | | | | 23 | |
| BC048679 | 0 | | | | | 2 | | 22 | 22 | | 2.5 | |
| BC051142 | | | | | | 1.5 | | | | | | |
| BC067074 | | | | | | | | | | 5.5 | | |
| BCAN | | | | | | | | 22 | | | | |
| BCAP29 | | | | | | 11 | | | | | | |
| BCAR1 | | | | | | | | | 9.5 | | | |
| BCAR3 | | 14.5 | | | | 14.5 | | 17 | | 13 | | 13 |
| BCAS3 | | | | 3 | | | | | | | | |
| BCAT1 | | | | | | | | | | 21 | | |
| BCHE | | | | | | | | | | | 2.5 | |
| BCKDHA | | | | | | | | 22.5 | | | | |
| BCKDHB | | | | 5 | 3 | | 6 | | | 13 | | |
| BCKDK | | | | | | | | | 22 | | | |
| BCL2 | | | | | | | | 3 | | | | |
| BCL2L1 | | | | | | | | | 22 | | | |
| BCL2L10 | | | | | | | | 10.5 | | 8 | | |
| BCL6B | | | | | | | | 9 | | | | |
| BCL7A | | | | | | | | | 23 | | | |
| BCL7C | | | | | | | | | 23 | | | |
| BCL9L | 6 | 8 | | 10 | 8.5 | 7 | | 5 | | 9 | 9 | 9 |
| BCO2 | | | | | | | | | 19.5 | | | |
| BCORL1 | | | | | | | | | 21.5 | | | |
| BCR | 23 | | | | | | | | | | | |
| BCS1L | | | | | | | 18 | | | | | |
| BDH1 | | | | | | | | | 23 | | | |
| BDKRB1 | | 22 | 23 | | | | | 0 | | | | |
| BDNF | | | | | 4.5 | | | | | | | |
| BECN1 | | | 12.5 | | | | | 10 | | 10 | | |
| BEGAIN | 0 | | | | 0.5 | | | | | | | |
| BEND5 | 12 | 13 | 13 | 13 | 15 | 14 | | 14 | 14 | 13 | 15 | 14 |
| BEND6 | | | 5 | | | | | 10 | | | | |
| BET1 | 9 | 7 | | | | | | | | 10 | | 8 |
| BET1L | | | | 20.5 | | | | | | | | |
| BEX1 | | | | 22 | | | | 16 | | | | |
| BEX6 | | | | | | 12 | | 11 | | | | |
| BFAR | | | | | | | | 20 | | | | |
| BFSP1 | | | | | 0.5 | | 23 | | | | | |
| BHLHB9 | | | | | | | | | 13 | | | |
| BHLHE40 | | | 20 | | 2 | | | 19 | | | | |
| BHLHE41 | | | | | | 0 | | | | | | |
| BHMT2 | | | | | | | | 15.5 | | | | |
| BICC1 | | | 19.5 | | | | | | | | | 21 |
| BICD2 | | | 13 | | | | | | 18 | | | |
| BIK | 15 | | | | | | | | | | | |
| BIRC3 | 19.5 | 19.5 | 20.5 | 21 | | | 21.5 | 21 | 19 | 18 | | |
| BLCAP | | | | | | | | 23.5 | | | | |
| BLOC1S3 | 21 | | | | | | | | | | | |
| BLVRB | | | | | | | | 10 | | | | |
| BMF | | | | | | | | | | | | 8 |
| BMP1 | | | | | | | | | 22.5 | | | |
| BMP2 | | | | | | | | 11 | | | | |
| BMP2K | | | | | | | | | | | 4.167 | |
| BMP3 | 7 | | 11 | | | | | | | | | |
| BMP4 | | 14 | | | | | | | | | | |
| BMP5 | | | | | | | | 8.5 | | | | |
| BMP6 | | | | | | | | | 1 | | | |
| BMPR1A | | | 4 | | | | | | 22.5 | | | 7 |
| BMPR1B | | | | | | | | 15 | | 13 | | |
| BMYC | | | | | | | | | | 7 | | |
| BNC2 | | | | | | | | | 23 | | | |
| BNIP3 | | | | | | | | 16 | | | | |
| BOK | | | | | | | | | | 11.5 | | |
| BOLA1 | | 9 | 10 | | | | | | | 12 | | |
| BPHL | | | | | | | | 15 | | 15 | | |
| BPIFB5 | | | | | | | | | | 11.5 | | |
| BPNT1 | | | | | | 7 | | | | | | |
| BPTF | | | | | | | | | 2 | | | |
| BRAF | | | | | | | | | | 10 | | |
| BRAP | | | | | | | | 21 | 23 | | | |
| BRCA2 | | | | | | | | 14.5 | 16 | 15 | | |
| BRD1 | | | | | | 11 | | | | | | |
| BRD2 | | | | | | 22 | 8.5 | | | | | |
| BRD7 | | | | | | | | 6 | 3 | | | |
| BRF2 | | | 23 | | | | | | | | | |
| BRI3 | | | | | | | | | 0 | | | |
| BRI3BP | | | 11 | | | | | | 0 | | | |
| BRIP1 | | | | | | | | 12 | | | | |
| BRK1 | | | | | | | | | 22 | | | |
| BRMS1L | | | 20.5 | | | | | | | | 22 | |
| BRP44L | | | | | | | | | 23 | | 22.5 | |
| BRSK2 | | | | | | | | | 23 | | | |
| BRWD3 | | | | | | | | 8 | | | | |
| BSCL2 | | | | 8 | | | | | | | 23 | |
| BSDC1 | | | | | | 22 | | 23 | | 21 | | |
| BSPRY | | | | | | 22 | | | | | | |
| BST2 | | | | | | | | | 23 | | | |
| BTBD11 | | | | | | | | 23.5 | | | | |
| BTBD3 | | 8 | 11 | | | 10 | | | | | | |
| BTBD6 | | | | | | | | | 22 | | | |
| BTBD9 | 1 | | | | | | | | | | | |
| BTD | | | | | | | | 12 | 21 | 10.5 | | |
| BTF3 | | | | | | | | | 21 | | | |
| BTF3L4 | | | | | | | | | 21.5 | | | |
| BTG1 | | | | | | | | | 22 | | | |
| BTNL9 | | | | | 10.5 | | | | | | | |
| BZW1 | | | | | | | | 5 | 1 | | | |
| BZW2 | | | 8 | 7 | | | | 7.5 | | | | |
| C10ORF107 | 4 | | 6 | | | | | | | | 2 | |
| C10ORF11 | | | | | | | | 22 | 21 | | | |
| C10ORF140 | | | | 2.5 | | | | | | | | |
| C10ORF2 | 16 | | | | | | | | | | | |
| C10ORF27 | | | | | | 20 | | | | | | |
| C10ORF46 | | | | | | 19 | | | | | | |
| C10ORF47 | | | | | | | | 8 | | | | |
| C10ORF71 | | | | | | | | | | | 0 | |
| C10ORF90 | | | | | | | | | 14.5 | | | |
| C11ORF1 | 9 | 9.5 | 10 | | | | | | | | | |
| C11ORF24 | | | | | | | | | | 11 | | |
| C11ORF31 | | | 6.5 | | | | | | | | | |
| C11ORF41 | | | 9 | | | | | | | | | |
| C11ORF46 | | | 8 | | | | | | | | | |
| C11ORF51 | | | | | | | | | 22 | | | |
| C11ORF52 | | | | | | | | 22 | | 1 | | |
| C11ORF53 | | | | | | | | | 21 | | | |
| C11ORF54 | | | | | | | | 4 | 10 | 17 | 5 | |
| C11ORF65 | | | | | | | | 8.5 | | | | |
| C11ORF71 | | | | | | | | | 22 | | | |
| C11ORF73 | | | 23 | 22.5 | | | | | | | | |
| C11ORF83 | | | 20.5 | | | | | | | 2 | | |
| C11ORF86 | | 6 | 6 | | | | | | | | | |
| C11ORF87 | | | | | | | | | | 13 | | |
| C11ORF96 | | | | | | | | 2 | | | | |
| C12ORF10 | | | | | | | | | 12 | | | |
| C12ORF23 | | | | | | | | | 0 | | | |
| C12ORF29 | | 7 | | | | | | | | | 7 | |
| C12ORF34 | | | | | | | 21 | | | | | |
| C12ORF35 | | | | | | | | | | | | 21.5 |
| C12ORF4 | | | | | | | | | | 7 | | |
| C12ORF44 | | | 19 | | | 22 | | | 13 | 13.5 | | |
| C12ORF45 | | | | | | | | | | 11 | | |
| C12ORF49 | | | | | | | | | | | 5.167 | |
| C12ORF5 | | | | | | | | | 13 | | | |
| C12ORF56 | | | | | | | | 11.5 | | | | |
| C12ORF65 | | | | 19.5 | | | | | 22 | | | |
| C12ORF68 | | | | | | | | | 3 | | | |
| C12ORF69 | | | | | | 0 | | | 3 | 6 | | |
| C14ORF101 | | | | | | | | 9 | | | | |
| C14ORF118 | 21 | | | | | | | | | | | |
| C14ORF126 | | | | | | | | 1 | | | | |
| C14ORF129 | | | | | | | | 10 | | | | |
| C14ORF135 | | | 0 | | | | | 8 | 23.5 | | | |
| C14ORF149 | | | | | | | | 8 | | 10 | | |
| C14ORF159 | | | | | | | | | | 10 | | |
| C14ORF45 | | | | | | | | 23 | | | | |
| C14ORF49 | | | | | | | | | | 18 | 16 | |
| C14ORF79 | | | | | | | | 7 | 1 | | | |
| C14ORF93 | | | | | | | | | | 13 | 21.5 | |
| C15ORF24 | | | | | | | | | 23.5 | | | |
| C15ORF39 | | | | 10 | 10 | | | 10 | | 17 | | |
| C15ORF40 | | | | | | | | | | | | 8 |
| C15ORF41 | | | | | | | | 16 | 17 | | | |
| C15ORF52 | | | | | | | | 8 | 22 | 22.5 | | |
| C15ORF58 | | | | | | | | 23 | 22 | | | |
| C15ORF61 | | | | | | | | | 21 | | | |
| C16ORF5 | | | | | | | | | 22 | | | |
| C16ORF62 | 12 | | | | | | | | | | 8 | |
| C16ORF70 | | | | | | | 7 | | | | | |
| C16ORF72 | | | 13 | | | | | | | | | |
| C16ORF73 | | 16 | 13 | 13 | 13 | 14 | | | 15.5 | 15 | | |
| C16ORF80 | | | | | 8 | | | | | 10.5 | 2 | |
| C16ORF87 | | | | | | | 22 | | 21 | | | |
| C16ORF88 | | | | | | | | | 13 | | | |
| C16ORF89 | | | | | | | | 22 | | | | |
| C16ORF96 | | | | | | | | | | | | 19 |
| C17ORF101 | | | | | | | | | | | 21 | |
| C17ORF103 | | | | | | | | 1.5 | | | | |
| C17ORF109 | 22 | | 10 | | | 16 | | 17 | | 14.5 | | 9 |
| C17ORF39 | | | | | | | | | | 22 | | |
| C17ORF53 | | | | | | | | | | | 2 | |
| C17ORF63 | | 5 | | | | | | | | 8 | | |
| C17ORF70 | | | | | | | | | | 6 | | |
| C17ORF78 | | | | | | | 20 | 21 | | | | |
| C18ORF1 | | | | | | | | | 23 | | | |
| C18ORF32 | | | | | | | | | 23 | | 3.5 | |
| C18ORF8 | | | | | | | | | | 4.5 | | |
| C19ORF12 | | | | | | 13 | | 17 | 16 | | 20 | 19 |
| C19ORF40 | | | | | | | | | | | 7 | |
| C19ORF42 | | 7 | | | | | | | 6 | | | |
| C19ORF43 | 2 | 3 | 4.5 | | | 2 | | 3 | 0 | 3.5 | 4 | 5 |
| C19ORF44 | | | | | | | | | 23 | | | |
| C19ORF46 | | | | | | | | | 22 | | | |
| C19ORF6 | | | | | | | | | 19 | | | |
| C19ORF60 | | | | | | 10.5 | | | | | | |
| C19ORF66 | | | | | | | | | | | | 19.5 |
| C19ORF69 | | | | | | 18 | | | | | | |
| C1GALT1 | | | | | | 11 | | | | | | |
| C1GALT1C1 | 3 | | | | | | | 5 | | | | 5 |
| C1ORF100 | | | | | | 18 | 20.5 | 19 | | | | 23 |
| C1ORF106 | | | | | | | | 20 | | | | |
| C1ORF110 | | | | | | | | 21 | | | | |
| C1ORF115 | | | 19 | | | | | | | | | |
| C1ORF116 | | 10.5 | | | | | | | 0 | | | |
| C1ORF123 | | | | | | 17 | | 0 | 22 | | | |
| C1ORF127 | | | | | | | | 12 | 15 | | | |
| C1ORF131 | | | | | | | | | | | 23 | |
| C1ORF168 | | | | 11.5 | | 13 | | | | 11 | | |
| C1ORF172 | | | | | | 17 | | 6 | 11 | | | |
| C1ORF192 | | | | | | | | | 9.5 | | | |
| C1ORF198 | 9.5 | | | | | 15 | | | | | | |
| C1ORF21 | | | | 23 | | | | | | 1.5 | | |
| C1ORF210 | | | | | | | 6.5 | | | 12.5 | | |
| C1ORF228 | | | | | | | | | | | 4.5 | |
| C1ORF31 | 14 | | | | | | | 20.5 | 17 | 5.5 | | |
| C1ORF38 | | | | | | | 8.5 | | | | | |
| C1ORF50 | | 8 | | | | | | | | | | |
| C1ORF51 | | | | | | | | 22.5 | 0 | | | |
| C1ORF86 | | | | | | | | 23 | | | | |
| C1ORF87 | | | | | | | | 19.5 | | | | |
| C1QL3 | | 13 | | | | | | 15 | | 14 | | |
| C1QTNF1 | | | | | | | 9 | | | | | |
| C1QTNF2 | | | | | | 16 | | | | | | |
| C1QTNF3 | | | | | | | | | | 11.5 | 14.5 | |
| C1QTNF9 | | | | | | | | 7 | | | | |
| C20ORF173 | | | | | | | | 22 | 22 | | | |
| C20ORF194 | | | | | | | | 7 | | | | |
| C20ORF24 | | | | | | | 1 | | | | | |
| C20ORF3 | 6 | 9 | 10 | | | | | | | 14 | | |
| C20ORF43 | 21 | | 13 | | | | | | 17 | | | |
| C20ORF72 | | | | | | | | 22 | 23 | | | |
| C20ORF94 | | | | | | | | | 0 | | | |
| C20ORF96 | | | | | | | | | | 9.5 | | |
| C21ORF7 | | | | | | | 14 | | | 9 | | |
| C21ORF91 | | | | | 5 | | | | 22 | | | |
| C22ORF13 | | | | | | | | | | 15 | | |
| C22ORF25 | | | | 3.167 | | | | | | | | |
| C22ORF39 | | | | | | | | | | 18 | | |
| C22ORF40 | | | | | | | | 22.5 | | | | |
| C230004F18RIK | 9 | 9.5 | 9 | | 9.5 | 9.5 | | 9 | 8 | 10 | 8.5 | 9 |
| C2CD2 | | | | | 4 | | | | | | | |
| C2CD2L | 18.5 | | | | 23.5 | | | 19 | 22 | | | |
| C2CD3 | 11 | 12 | 12 | 11 | 13 | 13 | | 11 | 12 | 12 | 10.5 | 12 |
| C2ORF28 | | | | | | | | | 22 | | | |
| C2ORF43 | | | | | | | | | 22 | | | |
| C2ORF44 | | 7 | 9 | | | | | | | | | |
| C2ORF54 | | | | | | | | | | 12 | 0 | 23 |
| C2ORF68 | | 18.5 | 20 | | | 22 | | 18.5 | | | | |
| C2ORF74 | 5.5 | | | | 22 | | | | 21 | | | |
| C2ORF80 | | | | 7 | | | | | | | | |
| C3 | | | | | | | | 0 | | | | |
| C330021F23RIK | | | | | | | | | | 20 | | |
| C3ORF14 | | | | | 19 | | | | 11 | | | |
| C3ORF18 | | | | | | | | | 11 | | | |
| C3ORF23 | | | | | | | | | | 5 | | |
| C3ORF33 | | | | | | | | | | 6 | | |
| C3ORF37 | | | | | | | | | 21.5 | | | |
| C3ORF38 | | | | | | 5 | | | 3.5 | | | |
| C3ORF58 | | | | | | | | | 16 | | | 19 |
| C3ORF67 | | | | | | 4.5 | | | | | | |
| C3ORF80 | | | 13.5 | | | | | | | 5.5 | | |
| C4B | | | | | | | | | 19 | | | |
| C4ORF21 | | | | | | | | 19 | | | | |
| C4ORF32 | 6 | 15 | | | | | | | | | | |
| C4ORF33 | | | | | 22 | | | | 22 | | | |
| C4ORF34 | | | | | | | | | 13 | 8 | | |
| C5 | | | | | | | | | | | | 0 |
| C530005A16RIK | | | | | | | | 9 | | 13 | | |
| C5AR1 | | | | | | | 9 | | | | | |
| C5ORF15 | | | | | | | | 22 | | 9 | | |
| C5ORF34 | | | | | | | | 0 | | | | |
| C5ORF44 | 21 | | | | | | | | | | | 5 |
| C5ORF45 | | | | | | 10 | | | | 10 | 12 | |
| C5ORF51 | | | | | | | | 1 | | | | |
| C5ORF62 | | | | | | 16 | | | | | | |
| C5ORF63 | | | | | | | | 9 | | | | |
| C5ORF65 | | | 11 | | | | | | | | | |
| C6 | | | | | | | | | 21 | | | |
| C6ORF106 | | | | | | | | | 17 | | | |
| C6ORF108 | | | 20.5 | | | | | 23 | | | 0 | 20 |
| C6ORF118 | 20 | | | | | | | | 23 | | | |
| C6ORF170 | | | 5 | | | | 7.5 | | | | 7 | |
| C6ORF211 | | | | | | | | | 3.5 | | | |
| C6ORF222 | 10 | | | | | | | | | 9.5 | | |
| C6ORF47 | | | | | | | | | | 7.5 | | |
| C6ORF62 | | | | | | | | 21 | | | | |
| C6ORF70 | | | | | | 23 | | | | | | |
| C6ORF89 | | | | | | | | | 23 | | | |
| C7ORF26 | | | | | | 19 | | | | | | |
| C7ORF31 | | | 0 | | | | | | | | | |
| C7ORF45 | | | | | | | | | | 11.5 | | |
| C7ORF49 | | | | | | | | | 17 | 11 | | |
| C7ORF53 | | | | | | | 9 | | 12 | | | |
| C7ORF57 | | | | | | | | | 22 | | | |
| C7ORF58 | | | | | | | | 7 | | | | |
| C7ORF59 | | 8 | 9 | | | | | | 7 | | | |
| C7ORF60 | | | 6.5 | | | | | | | | | |
| C7ORF63 | | | | | | | | 7 | 11 | | | |
| C7ORF70 | | | | | | | | | 6 | | | 8 |
| C8A | | | | | | | | 23 | 20 | | | |
| C8G | | | 8.5 | | | | | | | | | |
| C8ORF34 | | | | | | | | | | | 10 | |
| C8ORF4 | | 9 | | | | | | | 8 | 12 | | |
| C8ORF40 | | | | | | | | | | 10.5 | 19 | |
| C8ORF42 | | | | | | | | | 2 | | | |
| C8ORF83 | | | | | | | 8 | | | | | |
| C8ORF84 | | | | | | | | | 22.5 | | | |
| C8ORF85 | | | | | | | | 1 | | 13 | | |
| C9ORF102 | | | 8 | | | | | | | | | |
| C9ORF114 | | | | | | | | | | 10 | | |
| C9ORF123 | | | | | | | | | | | 5.167 | |
| C9ORF142 | | | | | | | | | 23 | | | |
| C9ORF16 | | | | | | | | 22 | 23 | | | |
| C9ORF167 | | | | | | | 22 | 20 | 23 | | | |
| C9ORF174 | | | 22 | | | | | | | | | |
| C9ORF40 | | | | | | | | | | | | 8.5 |
| C9ORF46 | | | | | | | | | | | | 18.5 |
| C9ORF85 | 6 | | | | | | | | | 6 | | |
| C9ORF86 | | | 23 | | | | | | | | 0 | |
| C9ORF89 | | | | | | | | | | 10 | | |
| C9ORF93 | | | 23.5 | | | | | | | | | 23.5 |
| CA11 | | | | | | | 8.5 | 14 | | | | 21 |
| CA12 | | | | | | | | | 7 | | | |
| CA13 | | | | | | | | 14 | | | | |
| CA14 | 23 | | 12 | | | | | | | | | |
| CA2 | 12 | | | | | | | | | | | |
| CA3 | | | | | | | | 2 | | | | |
| CA4 | | | 17 | | | | | | 22 | | | |
| CA5B | | | | | | | | | 22 | | | |
| CA8 | | | | | | | | | | 11 | | |
| CAAA01083517.1 | | 11 | | | | | | | | | | |
| CAAA01083517.2 | | | | | | | | 23 | | | | |
| CAB39 | | | | | | | | | 2 | | | |
| CABIN1 | | | | | | 11 | | | | | | |
| CABLES1 | | | | | | | | | | | 10 | |
| CAC1C | | | | | | | | | 23.5 | | | |
| CAC1D | | | | | | | | | 22 | | | |
| CAC1H | | | | | | 18 | | | | | | |
| CACHD1 | | | | 23 | | | | | | | | |
| CACNB2 | | | | | | | | | 23 | | | |
| CACNB3 | 10 | | | | | 16 | | | 22 | 10 | | 10 |
| CACNB4 | | | | | | | | | 18 | | | |
| CACNG4 | | | 3 | | | | | | | | | |
| CACNG5 | | | | | | | | 22 | 1.5 | | | |
| CACNG8 | | | 3.833 | | | | | | | | | |
| CACYBP | | | | | | | | 13 | | | | |
| CAD | 11 | | | | | | | 14 | 21 | 16 | | |
| CAD | | | | | | | | | 22 | 12 | | |
| CADM4 | | | | | | | | | 22 | 8 | | |
| CALB1 | | | | | | | | | 22 | | | |
| CALB2 | | | 9 | | | | | | | | | |
| CALCOCO1 | | 20 | | | | | | | | 12 | | |
| CALCRL | 22 | | | 4 | 5 | | | 23 | | 22.5 | 0.5 | 1 |
| CALD1 | | | | | | | | | 23 | | | |
| CALM1 | | | | 23 | | | | | | | | |
| CALM2 | | | | | | | | | | 21 | | |
| CALML5 | | 22 | | | | | | | | | | |
| CALN1 | 20.5 | | | | | | | 20 | | 11 | | |
| CALR | | 11.5 | | | 7 | | | | | | | 5.5 |
| CALU | | | | | | | | 23 | 22 | | | |
| CAMK1D | | | | | | | | | 7.5 | | | |
| CAMK2B | | | 12 | | | | | | | | | |
| CAMK2D | | | | | | | | 14 | | | | |
| CAMK2G | | 17 | 19 | | | | | | | | | 19.5 |
| CAMK2N1 | | | | | | | | | | 13 | | |
| CAMKK1 | 10 | 13.5 | | | | | | | | | | |
| CAMKK2 | | | 13 | | | 11.5 | | | 11 | | 8 | |
| CAMKMT | | | | | | | | | | | | 10.5 |
| CAMSAP1 | | | 23.5 | | | | | | | | | |
| CAMSAP3 | | | 17 | | | | | | | | | |
| CAMTA1 | | | | | | | | | | 4 | | |
| CAMTA2 | | | | | | 13 | | | 13 | | | |
| CAND1 | | | | | | | | 20 | | | | |
| CAND2 | | | | | | | | | 19 | | | |
| CANX | | | | | | | | | | | | 16 |
| CAP1 | | | 18.5 | | | | | 3 | | | | |
| CAPN2 | | 11 | 11 | | | | | | | 14.5 | | |
| CAPN5 | | | | | | | | | 3 | | | |
| CAPNS1 | 8 | | | | | | | | | 12 | | |
| CAPRIN1 | | | | | | | | 12 | | | | |
| CAPRIN2 | | | | | | | | 12 | 13 | | | |
| CAPZA2 | | | | | | | | | 22 | | | |
| CAPZB | | 6 | | | | | | | | 8 | | |
| CARD10 | | | | | | | | 22.5 | | | | |
| CARHSP1 | | | | | | | | | 23 | 19 | | |
| CARHSP1 | | | | | | | | 8 | 6 | | | |
| CARNS1 | | | | | | | | 0 | 0 | | | |
| CARS | | | 9 | | | | | | | | | |
| CARS2 | | | | | | | | 1 | 7.5 | | | |
| CASC4 | | 15 | | 13 | 13 | | | 14 | | 13 | | |
| CASD1 | | | | | | | | 5.5 | | | | |
| CASP2 | 17 | | 20 | | | 14 | | 18 | | | | |
| CASP6 | | | | | | | 7 | | | | | |
| CASP8 | | | | | | | | | 21 | | | |
| CASQ2 | | | | | | | | | 11 | | | |
| CASS4 | | 19 | 21 | | | | | | | | | |
| CAT | 22 | | | | | 13.5 | | 2.5 | | | | 21 |
| CATSPER2 | | | | | | | | 0 | | | | |
| CATSPER3 | 18 | | 22 | | 22.5 | | 20.5 | 16 | | 17 | 15 | |
| CAV1 | | 6.5 | 9 | | | | | 10 | 6 | | | |
| CAV2 | | | | | | | | | | 12 | | |
| CBFA2T3 | | | | | | | | | 22 | | | |
| CBL | | | 6 | | | | | | | | | |
| CBLB | | | | | | | | | 23 | | | |
| CBLC | | | | | | | | 4 | | 13 | | |
| CBR1 | | | | | 22 | | | 22.5 | 19.5 | | | |
| CBR2 | | | | | | | | | | | 4.5 | |
| CBS | | | | | | | | 21 | 19.5 | | | |
| CBX1 | | | | | | | | 6 | | | | |
| CBX4 | | | | | 22 | | | | | | | |
| CBX5 | | | | | | | | | | | | 9 |
| CBX7 | | | | | | | 19 | 19 | | | | |
| CC1 | | | | | | 2 | | 21 | | | | |
| CC2D1B | | | | | | | | | | 10 | | |
| CC2D2A | | | | | | | | 0 | | | | |
| CCAR1 | | | | | | | 18 | | | | | |
| CCBE1 | | | | | | | | 19 | | | | |
| CCBL1 | | | | | | | | 13 | 22 | | | |
| CCBP2 | | 22 | | | | 21 | | | | | | |
| CCDC102A | | | | | | | | | 21.5 | | | |
| CCDC108 | | | | | | | | 20 | 4 | | | |
| CCDC112 | | | | | | | | | | 9 | | |
| CCDC126 | | | | | | | | | | 10.5 | | |
| CCDC129 | | | | | | | | 21 | 23.5 | | | |
| CCDC134 | | | | | | | | 21 | | | | |
| CCDC135 | | | | | | | | 18 | | | | |
| CCDC138 | | | | | | | | | | 8 | | |
| CCDC141 | 13 | 13 | 14 | | 16 | 14 | | 12 | 12 | 13 | 11.5 | 13 |
| CCDC153 | | | | | 8 | | | | | | | |
| CCDC159 | | | | | | | | 12 | | | | |
| CCDC162 | | | | 15 | 15 | | | | | | | |
| CCDC163 | | | | | | | 6 | | | | | |
| CCDC166 | | | | | | 2 | | | 3.5 | | | |
| CCDC17 | | | | | | 9 | | | | 6 | | |
| CCDC28A | | | | | | | | | 19 | | | |
| CCDC3 | | | 8 | | | | | | | | | |
| CCDC30 | | | | 4 | | | | | | | | |
| CCDC39 | 18 | 19 | 21 | 22 | | | 23 | 18 | 19 | 17 | | 20.5 |
| CCDC41 | | | | | | | | | 13 | | | |
| CCDC50 | | | 20 | | | 23.5 | | | | | 0 | |
| CCDC57 | | 15 | 19 | 14 | | | | | 23 | 11 | | |
| CCDC6 | | | | | | | | 17 | | | | |
| CCDC60 | | | | | | 12 | | | | | | |
| CCDC64 | | | | | | 10.5 | | | | 11 | | |
| CCDC66 | | | | | | | | | 10.5 | 13 | | |
| CCDC69 | | | | | | | | | 0 | | | |
| CCDC74A | | | | | | | | | 22 | | 4 | |
| CCDC75 | | | | | | | | | | 5 | | |
| CCDC77 | | | | | | | | | 1 | | | |
| CCDC8 | | | | | | | | | 20 | | | |
| CCDC80 | | | | | | | | 10.5 | 12.5 | 15 | | |
| CCDC84 | | | | | | | | | 10 | | | |
| CCDC85A | | | | | | | | | 23 | | | |
| CCDC88C | | | | | | | | 23 | 22 | | | |
| CCDC91 | | | | | | | | | | | | 10 |
| CCDC92 | | | | | | 5 | | | | | | |
| CCKAR | | | | | | 9 | | | 1 | | | |
| CCKBR | | | | | | | | | 4.5 | | | |
| CCL11 | | | | | | | | | 3 | | | |
| CCL17 | | | | | | | | 22.5 | | | | |
| CCL20 | | | | | | | | 9.5 | | 8 | | |
| CCL25 | | | | | | | | 12 | | | | |
| CCNC | | | | | | | | 22 | | | | |
| CCND1 | | | | | | 22 | | 9 | | | | |
| CCND3 | | | | | | | | | 22 | | | |
| CCNE1 | | | | | 2 | | | | | | | 6.5 |
| CCNF | | 4 | 4 | | 7 | | | 5 | 4 | 3 | | 2 |
| CCNG1 | 0 | | 13 | | | | | | | 16 | | |
| CCNG2 | | | 2.5 | | | | | | 5 | | | |
| CCNH | | | | | | 23.5 | | 0.5 | 23 | | | 19 |
| CCNJL | | | 8.5 | | | | | | | | | |
| CCNL2 | | | | | | | | | 13 | | | 22 |
| CCNT2 | 8 | 11 | 13 | | | | 17 | 19 | | | | 10 |
| CCNY | | | | | | | | | | 19 | | |
| CCR5 | | | | | | | | | 23 | | 23 | |
| CCR7 | | | | | | | | 21 | | 18 | 7.5 | |
| CCRL1 | | | | | | | | | | 14 | | |
| CCRL2 | | | | | | | | | | 10 | | |
| CCRN4L | | | | | | | | 20 | | 21 | | |
| CCS | | | | | | | | 13 | | 12 | | |
| CCT2 | | | | | | | | | 11.5 | | | |
| CCT3 | | | | | | | | 7 | | | | |
| CCT5 | | | 14 | | | | | | | | | |
| CCT7 | | | | | | | | | | 17 | | |
| CD14 | | | | | | 17 | | | | | | |
| CD151 | | | | | | | 9 | | | | | |
| CD163 | 19.5 | | | | | | | | | | | |
| CD164 | | | | | | | | 23.5 | 23.5 | | | |
| CD164L2 | | | | | | 3.5 | | 22 | | | | |
| CD180 | | | | | | | | | 18.5 | | | |
| CD19 | | | | | | | | | | 6.5 | | |
| CD200 | | | | | | | | 23 | | | | |
| CD200R1 | | | | | | | | | 19.5 | | | |
| CD200R1L | | | | 21.5 | | 22 | | | | | | |
| CD209D | | | | | | | | | 5 | | | |
| CD244 | 10.5 | | 8 | | | | | | | | | |
| CD274 | | | | | | | | | 23 | | | |
| CD28 | | | | | 21 | | | 22.5 | 21.5 | | | |
| CD2AP | | | 17 | | | | | | | | | |
| CD300A | | | | | | | 22.5 | | 19 | | | |
| CD300LG | | | | | | | | 17 | | | | |
| CD302 | | | | | | | | | 19.5 | | | |
| CD33 | | 15 | 17 | | | 10 | | 16 | 15 | | 9.5 | 17 |
| CD34 | | | | | | | | 19 | 20 | | 0 | |
| CD36 | | | | | | | | | 23.5 | | | |
| CD37 | | | 1.5 | | | | | 0 | | | | |
| CD38 | | | | 23 | | 23 | | 1 | | | | 23 |
| CD4 | | | | | | 21 | | | | | | |
| CD40 | | | | | | | | | 21.5 | | | |
| CD44 | | | | 20 | | | 19 | | | | | |
| CD47 | | | | | | | | | 23 | 7 | 0 | |
| CD52 | | 16 | | | | | | 19 | 14 | | | |
| CD59 | | | | | | | | | 3 | | | |
| CD59A | | | | | | | | 8 | | | | |
| CD68 | 19 | | | | | | | | | | | |
| CD69 | | 9 | | | 12 | | | | 2 | 13 | | |
| CD74 | | 21 | 22.5 | | 5 | | | 23 | | | | |
| CD79A | | | | | 1.5 | | | | | | | |
| CD79B | | | | | | | | | 22 | | | |
| CD81 | | | | | | | 22 | | | | | |
| CD82 | | | | | | | | 8 | | | | |
| CD8A | | | | | | | | | | 13 | | |
| CD8B | | | | | | 5.5 | | | | | | |
| CD93 | | | | | | | | 20 | | | | |
| CD97 | | | | | | 22 | | 23 | 22 | | | |
| CDA | | | | | | | | | | 13 | | 11 |
| CDADC1 | | | | | | | | | 22 | | | |
| CDAN1 | | | | | | | | | 22 | | | |
| CDC14A | | | | | | | | | 20.5 | | | |
| CDC14B | | | | | | | | | | 8.5 | | |
| CDC25A | | | | | | | | 0 | | 0 | | |
| CDC25B | | | | | | | | | | | 1 | |
| CDC27 | | | | | | | | | | 22 | | |
| CDC34 | | | | | | | | 3 | | | | |
| CDC40 | 20 | | | 21 | | | | | | | | |
| CDC42 | | | | | | | 9 | | | | | |
| CDC42BPA | | | | 22 | | | | | 23 | | | |
| CDC42BPB | | | | | | | | 0 | | | | |
| CDC42EP3 | | | | | | | | | | 19 | | |
| CDC42EP4 | | | | | | | | 23 | | | | |
| CDC42EP5 | | | 23.5 | | | 0 | | | 1 | 1 | | 1 |
| CDC42SE1 | | | | | | | | | | 5.5 | | |
| CDC5L | | | | | | 16.5 | | | | 13 | | |
| CDC73 | | | | | | | | | 21.5 | 5 | | |
| CDCA7L | | | | | | | | | | 5 | | |
| CDCP1 | | | | 22 | | | | 17 | | | | |
| CDH1 | | | | | | | | 10 | | 9 | | |
| CDH11 | | | | | | | | | 10 | | | |
| CDH13 | | | | | | | 20 | 23 | | | | |
| CDH19 | | | 1.167 | | | | | | | | | |
| CDH2 | | | | | | | | | | | 22 | |
| CDH20 | | | | | | | | | 23 | | | |
| CDH22 | | | | | | | | 10 | | 9 | | |
| CDH3 | | | 7 | | | | | | 4 | | | |
| CDH4 | | | | | | | | | 22 | | | |
| CDH5 | | | | | | | | | | | 22.5 | |
| CDH8 | | | | | | | | | 12 | | | |
| CDHR3 | | | | 2 | | | | | | | | |
| CDHR5 | | | | | | | | 22 | | | | |
| CDIPT | | | 23 | | | | | 19 | 21 | 19 | | |
| CDK14 | | | | | | 23 | 21 | | | | | |
| CDK17 | | | | | | | | | 17 | | | |
| CDK18 | | | | | | 1.5 | | | | | | |
| CDK19 | | | | | | | | 21 | | | | |
| CDK2 | | | | | | | 6 | | | | | |
| CDK20 | | | | | | | | | 2 | | | |
| CDK2AP2 | | | | | | | | 3 | | | | |
| CDK4 | | | | | | | | | | 7 | | |
| CDK5RAP2 | | | | | | | | | 0.5 | | | |
| CDK5RAP3 | | | | | | | | | | | 6.167 | |
| CDK6 | | | | | 8 | | | | | | | |
| CDK7 | | | | | 21 | | | 18 | | | | |
| CDK8 | 22 | | | | | | | | | | | |
| CDK9 | 2 | | | | | | | | | | | |
| CDKAL1 | | | | | | | | | 22 | | | |
| CDKL1 | | | | | | | | 4.5 | | | | |
| CDKL2 | | | | | | | | 23 | | | | |
| CDKL5 | | | | | 20.5 | | | | | | | |
| CDKN1A | 19 | | 22 | | 20 | | | | 21.5 | 19 | | |
| CDKN1C | | | | | | | | | | | 0 | |
| CDKN2AIP | | | | | | | | | 23.5 | | | |
| CDKN2B | | | 21.5 | | | | | | 22 | | | |
| CDKN2C | | | | | | | | | | | | 2.167 |
| CDO1 | 4 | 3 | 3 | | | | | | | | | |
| CDON | | | | | | 1 | | | | | | |
| CDR2 | | | | | | 23 | | | | | | |
| CDS2 | | | 10.5 | | | 13.5 | | | | | | |
| CDSN | | | | | | 9.5 | | 9 | | | | |
| CDX4 | | | | | | | | | 23 | | | |
| CEACAM1 | | | | | 5.5 | | | | | | | |
| CEBPA | | | 11 | | | | | | | 15 | | |
| CEBPB | | | | | | | | | 5.5 | | | |
| CEBPG | | | 23 | | | | | | | | | 22 |
| CECR2 | | | | | | | | 14 | 15 | 12 | | |
| CECR6 | | | | | | | | | | | 0 | |
| CELF1 | | | | | | | | | | 10.5 | | |
| CELF2 | | | | | 7 | | | | 4 | 12.5 | | |
| CELF4 | 21 | | | | | | | | 23 | | | |
| CELSR1 | 9 | | | | | | 20.5 | 19 | 7 | 13 | | |
| CELSR2 | | | | 23 | | | | | | | | |
| CEND1 | | 0 | | | | | | | | | | |
| CENPA | | | | | | | | | 20 | 16 | | 20 |
| CENPB | | | | | | | | | | 15 | | |
| CENPC1 | | | | | | 19 | | | | | | |
| CENPL | | | | | | 12.5 | | | | 13 | | |
| CENPP | | | | | | | | | | 22 | | |
| CENPQ | | | | | | | | | | 4 | | |
| CEP120 | | | | | | | | | 17 | 10 | | |
| CEP128 | | | | | | | | | | 23 | | |
| CEP135 | 19.5 | | | | | | | 19 | | | | |
| CEP19 | 7 | | 6 | | | | | | 22.5 | | | 8 |
| CEP290 | | | 20.5 | | | | | | 22.5 | | | |
| CEP350 | | | | | | | | | | 21 | | |
| CEP41 | | | | | | | 5 | | | 22 | | |
| CEP44 | | | | | | | | | 21.5 | | | |
| CEP57 | | | | | | | | 23.5 | 22 | | | |
| CEP63 | | | | | | | 21 | | | | | |
| CEP68 | | | | | | | | | | 20 | | |
| CEP76 | | 5 | 6.5 | | | | | | | | | |
| CEP78 | | 22 | | | | | | | | 22 | | |
| CEP85 | | | | | | | | | | 8 | | |
| CEP85L | | | | | | | | 22 | | | | |
| CEP89 | 19 | | 7 | | | | | | | 16 | | |
| CEP95 | | | | | | | | | 22 | | | |
| CEP97 | | 13 | | | | | | 13 | | 11 | | 13 |
| CEPT1 | | | | | | | 21.5 | | 20.5 | | | |
| CERK | | | | | | | | | | | | 7 |
| CERKL | | | 15 | | | | | | | 11 | | 18.5 |
| CERS2 | | | 21 | | | | | | 23 | | | |
| CERS4 | | | | | | | | | | | | 19 |
| CERS6 | | | | | | | | | 23.5 | | | |
| CES1 | | | 1 | | | | | | 22 | | | |
| CES1D | | | | | | | | | | 11 | | |
| CES1F | | | 23 | | | | | | | | | |
| CES2G | | | | | | | | | 21 | | | |
| CETN3 | | | | | | | | | | 7 | | |
| CETN4 | | | | | | | | 23 | | | | |
| CFC1B | | | | | | | | | | | 9 | |
| CFD | | | | | | | | 21 | | | | |
| CFL1 | | | | | | | | 9 | | | | |
| CFL2 | | | 10 | | 18 | 12 | | 12 | 16.5 | 10 | 11 | |
| CFLAR | | | | | | | | | 9.5 | | | |
| CGN | | | | | | | | | 13.5 | 6 | | |
| CGNL1 | | | 10.5 | | | | | | | | | |
| CGRRF1 | | 7 | | | | | | | | | | |
| CHAC1 | | | 8 | | | | | | | | | |
| CHAMP1 | | | | | | | | | 11 | | | |
| CHCHD10 | | | | | | | | | 10.5 | | | |
| CHCHD3 | | | | | | | | | 11 | | | |
| CHCHD5 | | | | | | | | | 11 | | | |
| CHCHD7 | | | | | | | | | | 6 | 9 | |
| CHD2 | | | 13 | | | | | | | | | |
| CHD3 | 1 | | | | | | | | | | | |
| CHD4 | | 6.5 | | | | | | | 11 | | | |
| CHD6 | | 7 | | | | | | | 12 | | | |
| CHD7 | | 7.5 | | | | | | | | | | |
| CHD9 | | | | | | | | | 10.5 | | | |
| CHEK2 | | | | | | | 20 | | | | | |
| CHI3L7 | | | | | | | | | 12 | | | |
| CHIC1 | | | 7 | | | | | | | | | |
| CHID1 | | | | | | | | | 12 | | | |
| CHKA | | | | | | | | | | 11.5 | | |
| CHKB | | | | | | | | | 22 | | | |
| CHMP2A | | | | | | | | 19 | 16 | | | |
| CHMP2B | | | 23 | | | 11 | | | | | | |
| CHMP5 | 8 | | | | | | | | | | | |
| CHN1 | | 4 | | | 5 | 4 | | | | | | |
| CHN2 | | | | | | 18 | | | 18 | | | |
| CHORDC1 | | | | | | | | | 22 | | | |
| CHP | | | | | | | | | | 17 | | |
| CHPF2 | | 20 | 21 | | | | | | | 13 | | |
| CHR4 | | | | | | | | | | 8 | | |
| CHR6 | 10 | | | | | | | | | | | |
| CHRAC1 | | 14 | | | | | | 10 | | 10 | 11.5 | |
| CHRM2 | 6 | | | | 3 | | | | | | | |
| CHRNB1 | | | | | 21.5 | | | | 0 | | | |
| CHST1 | | | 19 | | | | | | | | | |
| CHST11 | | | | | | | | 6.5 | | | | |
| CHST13 | | | | | | | | | | 5.5 | | |
| CHST15 | | | | | | | | | | | 5 | |
| CHST2 | | | | | 9.5 | | | | 10 | | | |
| CHST3 | | | | | | | | | 2 | | | |
| CHST8 | | | | | | 19 | | | | | | |
| CIAPIN1 | | | | | 22 | 17 | 21 | | | | | |
| CIB4 | | | | | | 18 | | | | | | |
| CIDEC | | | | | | | | | 19 | | | |
| CIITA | | | | | | | | 18 | | | | |
| CIRBP | | | | | | | 0 | 22 | | | | |
| CISD1 | | | | | | | | | 0 | | | |
| CIT | | | | | 7.5 | | | | | | | |
| CITED2 | | 10 | | | | | | | | 11.5 | | |
| CKAP4 | | | | | | | | | 0 | | | |
| CKAP5 | | | | | | | | | 2 | | | |
| CKB | | | 6 | | | | | | | | | |
| CLASP1 | | | | | | 22 | | | 22.5 | | | |
| CLASP2 | | | 23 | | | | | | 23 | | | |
| CLCA4 | | | | | | | | 23 | | | | |
| CLCC1 | | | | | | | | 22.5 | 21 | | | |
| CLCN2 | 16 | 18 | 18 | | | 15 | | | 14.5 | | | 16 |
| CLCN3 | | | | | | 22 | | | 2 | | | |
| CLCN5 | | | | | | | | 19 | | | | |
| CLCN6 | | 7.5 | | | | | | 10 | 1 | | | |
| CLCN7 | | | | | | | 9 | | 9.5 | | | |
| CLDN1 | | | 9 | | | 15.5 | | | | | | |
| CLDN10 | | | | | 4 | 22 | | | | | 23 | |
| CLDN11 | | | | | | | | | | 10 | | |
| CLDN12 | | | | | | | | | 23 | | | |
| CLDN15 | | 5 | | | | | | | | | | |
| CLDN2 | | | | | | | | | | 12 | | |
| CLDN5 | | | 21 | | | | | | | | | |
| CLDN7 | | | | | | | | | 14 | | | |
| CLDN8 | | | 18 | | | | | | | | | 15 |
| CLEC2H | | | 19 | | | 17 | | | | | | |
| CLEC3B | | | | | | | | 8 | | | | |
| CLEC4M | 20 | | | 23 | | | | | | | | |
| CLEC5A | | 6 | | | | | | | | | | |
| CLIC4 | | | | | | | | | | 4 | | |
| CLIC5 | | | | | | | | | | 4 | | |
| CLINT1 | | | | | | 14 | | | | | | |
| CLIP1 | | | | | | 18.5 | | 15 | | 17 | | |
| CLIP2 | | | | | | | | 16 | 22 | | | |
| CLK2 | | | | | | | | | 23 | 12 | | |
| CLK3 | | | | | | | | | 8 | | | |
| CLMN | | | | | | | | | 10.5 | | | |
| CLMP | | | | | | | | | 22 | | 23 | |
| CLN3 | | | 9.5 | | | | | 16 | | | | |
| CLN5 | | | | | | | | 13.5 | 14 | | | |
| CLN6 | | | | | | | | 8 | | | | |
| CLNS1A | | | 22.5 | 11.5 | | | | | | | | |
| CLOCK | | | | | | | | | 23 | | | 7 |
| CLPB | | | | 20 | | | | 20.5 | | | 3 | |
| CLPP | | | | | | | | 9 | | | | |
| CLPTM1 | | 10 | 12.5 | | | | | 23.5 | | 3.5 | 14 | |
| CLPX | | | | | | 22 | | | | 13.5 | | |
| CLRN1 | | | | 12 | 10 | | 11 | 11 | | | | |
| CLSTN1 | 8 | | | 15 | | 10.5 | | | 8 | 12 | 15.5 | |
| CLSTN3 | | | | 16 | | | | | | 6 | | |
| CLTB | | 12 | | | | | 6 | | | 20 | | |
| CLTC | 6.5 | | | | | | | 9 | 13.5 | | | |
| CLYBL | | | | 13 | | | | | | 14.5 | | |
| CMA1 | | 10 | 11 | 21 | | 12.5 | | | | 10 | | 10.5 |
| CMAH | 18 | | | | | | | 9 | | | | |
| CMBL | | | | | | | | 12 | | | | |
| CMC2 | | | | | | | | 7 | | | | |
| CMIP | | | | | | | | 22 | | | | |
| CML1 | | | | | | | | 1 | | | | |
| CML2 | 6 | | | | | | | 19 | | 12 | | |
| CML5 | | | | | | | | | | 13.5 | | |
| CMPK2 | | | | | | | | | | | | 17 |
| CMTM3 | | | | | | | | 11 | | | | |
| CMTM4 | 9 | 10 | 10 | | | | | 9 | 10 | | | 12 |
| CMTM6 | | | 21.5 | 21 | | | | | 17 | | | |
| CMTM7 | | | | | | | | 5.5 | | | | |
| CMTM8 | | | 8 | | | | | | 18 | | | |
| CNBD1 | | | | | 10 | | | 12 | | | | |
| CNBP | | 10 | 12.5 | | | | | | | 11 | | |
| CNDP1 | | | | | | | 19 | | | | | |
| CNDP2 | | 9 | 11 | | | | | | 4.5 | | | 9 |
| CNEP1R1 | | | | | | | | | | 8 | | |
| CNIH | | | | | | | | | | | | 10 |
| CNIH4 | | | | 12 | | | | | 21 | | 5 | |
| CNKSR2 | | 16 | 17 | 18 | | 16 | | 14 | 16 | 13 | 16.5 | 15.5 |
| CNKSR3 | | | | | | | | | 21 | | | |
| CNN1 | | | | | | | 9 | | | | | |
| CNN3 | | | | | 6 | | | | | | | |
| CNNM3 | | | | | | | | 11.5 | | 8 | | |
| CNNM4 | | | | | | | | | | 12 | | |
| CNOT2 | | | | | | 12 | | 10 | | | | |
| CNOT6 | | | | | | | | | 0 | | | |
| CNOT7 | | 15 | 15 | 12 | | 14 | | 15 | | 15 | 15.5 | |
| CNP | | 5 | | | | | | | | | | |
| CNPPD1 | 7 | 7 | | | | | | | | 11 | 6 | |
| CNPY2 | | | | | | 2 | | | 12 | | 4 | |
| CNPY4 | | | | | | | | | 23 | | | |
| CNR1 | | 7 | | | | | | 9 | | 9 | | |
| CNTFR | 4 | | 7 | | | | | | | | | |
| CNTN1 | | | | | | | | 23 | | | | |
| CNTN5 | | | | | | | | | 23 | | | |
| CNTP5C | | | | | | | | | 1 | | | |
| COASY | | | | | | | | | 22 | 14 | | |
| COBL | | | | | | | 5.5 | | | | | |
| COBLL1 | | | | | | 17 | | | | | | |
| COBRA1 | | | | | | | | | 23 | | | |
| COG5 | 18 | | 20 | | 20.5 | 20.5 | | 22.5 | 17 | | | |
| COL12A1 | | | | | | 6 | | | | | | |
| COL13A1 | | | | | | | | | | | | 10 |
| COL15A1 | | | | | | | | | | | | 9 |
| COL18A1 | | | | | | | | | | | 0 | |
| COL1A1 | | | | 8 | 7 | | 7 | 9 | 9 | | | |
| COL27A1 | | | | 16 | | | 17 | | 10 | | 19.5 | |
| COL3A1 | | | | | | | | | | 4 | | |
| COL4A1 | | | | | | | | 13 | 14 | | | |
| COL4A2 | 21 | | | | | | | | | 18 | | |
| COL4A3 | | | | | | 10 | | | | | | |
| COL4A4 | | | | | | | | 15 | | | 23 | |
| COL5A1 | | | | | | | | | 22 | 10 | | |
| COL5A2 | | | | | | | | | 11.5 | | | |
| COL5A3 | | | | | | | | | 3 | | | |
| COL6A2 | | | | | | | | 6 | | | | |
| COL6A3 | | | | | | | | | 5 | | | |
| COL6A6 | | | | | | | | | 5.5 | | | |
| COL8A1 | | | | | | | | 4 | | | | |
| COLEC12 | | | | | | | | 10 | | | | |
| COLQ | | 8 | | | 9.5 | | | | | | | |
| COMMD10 | | | | | | | | | 20 | | | |
| COMMD4 | | | | | | | | | 18.5 | | | |
| COMMD5 | | 7 | 7 | | | | | | | | | |
| COMMD6 | | | | | | | | 19 | 17 | | | |
| COMMD7 | | | | | | | | | | 14 | | |
| COMT | 20 | | | | | | | 22 | | | | |
| COPE | | | | | | | | 0.5 | 22 | | | |
| COPG1 | | | | | | 18 | | 19 | | | | |
| COPG2 | | | | | | | | | 22.5 | | | |
| COPS4 | | | | | | | | | | 12.5 | | |
| COPS6 | | | | | | | | | 22 | | | |
| COPS7A | 0 | | | | | | | 11 | | | | |
| COPS8 | | | | | | | | | 23 | | | |
| COPZ1 | | | | 5 | | 10 | | | | | 9 | 8 |
| COPZ2 | 6 | | | | | | | 11 | | | | |
| COQ10A | | 10.5 | | | | | | | | | | |
| COQ10B | | | | | | | | 10 | | | | |
| COQ2 | | | | | | | | | | | | 14.5 |
| COQ4 | | | | | | | | 6.5 | 11 | | | |
| COQ5 | | | | | | | | 7 | | 17 | | |
| CORIN | | | 22 | | | | | | | | | |
| CORO1A | | | | 13.5 | 14.5 | 11 | | | 19 | 19 | | |
| CORO1B | | | | | | | | 3 | | | | |
| CORO2A | | | 12.5 | 8 | | 21 | | 16.5 | 21 | 18 | | |
| CORO6 | | | | | | | | | | | 1 | |
| COX10 | | | | | | | | 10 | | | | |
| COX14 | | | | | | | | | | 7 | | |
| COX18 | | 8 | 8.5 | | | | | | | | | |
| COX19 | | 10 | 11 | | | | | | | | | 12 |
| COX20 | | | | | | | | | | 23 | | |
| COX4I1 | | | | | | | | | 21 | | | |
| COX4I2 | | | | | | | | | | 23 | 10 | |
| COX6A1 | | | | 12 | | 8 | | | | 10 | | |
| COX6B1 | | | | | | | | | | 11 | | |
| COX8A | | 9 | 10 | | | | | | | | | |
| CP | | | | | | | | 3 | | | | |
| CPA1 | | | | | | | | | 8.5 | | | |
| CPA3 | 21 | | | | | | | | 7.5 | | | |
| CPE | | | | | | | | | 19 | | | |
| CPEB1 | | | 9 | | | | | 12 | | | | |
| CPEB2 | | | | | 21.5 | | | | | | | |
| CPEB3 | | | | | | | | | 21 | | | |
| CPEB4 | | 17 | | | | 18 | | | 19 | 18 | | |
| CPLX2 | | | | | | | | | 14 | | | |
| CPLX4 | | | | | | 22.5 | | 7 | | | 21 | |
| CPM | | | 0.167 | | 21 | 18 | | 15 | 21 | 20.5 | | |
| CPN2 | | 0 | | | | | | | | | | |
| CPNE1 | | 12 | 11 | | 12 | 10.5 | | 12.5 | | 12 | | 13 |
| CPNE2 | | 5.167 | | | | | | | 12 | | | |
| CPOX | | | | | | | | | | | 14 | |
| CPQ | | | | | | | | | 9.5 | | | |
| CPSF1 | | | | 20.5 | | | | | | | | |
| CPSF3 | 8 | | | | | | | 4.5 | | | | |
| CPSF3L | | | | | | | | 15 | | | | |
| CPSF4 | 7 | 10 | 11 | | | | | | 5 | | 8 | |
| CPT1A | | | | | | | | 0 | | | | |
| CPT2 | | | | | | | | 15 | | | | |
| CPXM1 | | | | | | | | | | | | 19 |
| CRADD | | | | | 11 | | 7.5 | | | | | |
| CRAT | | | | | | | | 12 | | | | |
| CRCT1 | | | | | | | | 23 | | | | |
| CREB1 | | | | | | | | 8.5 | | | | |
| CREB3L1 | | | | | | | 9 | 10 | | 8 | | |
| CREB3L2 | | | | | | | | | | | 23 | |
| CREBBP | 5.5 | 5.5 | 4.5 | | 1 | | | | 5 | | | |
| CREBL2 | | 13 | | 16 | 15 | | | | | | | |
| CREBRF | | | | | | | | | | 4 | | |
| CREG1 | | | | 10 | | | 10 | | 6 | 11.5 | | |
| CRELD1 | | | | | | | | 12 | | 7 | | |
| CRELD2 | | | | | | | | 13 | | | | |
| CREM | | | | | | | | 9 | | | | |
| CRIM1 | | 8.5 | 10 | | | | | 11 | | | | |
| CRIP2 | | 11.5 | | | | | | 11 | | | | |
| CRISPLD1 | | | | | | 3.5 | | | | 3 | | |
| CRLF3 | | | | | | | | | 12 | | | |
| CRLS1 | | | | | | | | | | 16 | | |
| CRMP1 | | | 12 | | | | | | | | 7.167 | |
| CROT | | | | | 9 | | | | | | | |
| CRP | | | | | | | | | 23 | | | |
| CRTAC1 | | | | | | | | | | | 4.5 | |
| CRTC2 | 4 | | | 6 | | | | 6 | 3.5 | | | |
| CRTC3 | | | | 22 | | | | 17 | | | | |
| CRY1 | | 11 | | 12 | | 13 | | 13 | 18.5 | 11.5 | | 13 |
| CRY2 | | | | | | | 5 | | 21 | | | |
| CRYAB | 10 | 11 | 14 | | | | | | | 0 | | |
| CRYBG3 | | | | | | | | 12 | 12.5 | | | |
| CRYL1 | | | | | | 3 | | | | | | |
| CRYM | | | | 22 | 23 | | | 21 | | | | |
| CRYZ | | | | | | | | | 1.5 | 13.5 | | |
| CS | | | 0 | | | | | | 12 | | | |
| CSAD | 21 | 21 | 21.5 | | 21.5 | | | 20 | 21.5 | | | |
| CSDA | | | | | | | | | 18.5 | | | |
| CSDC2 | | | | | | | | | 20 | | | |
| CSE1L | | | | | 20.5 | | | 9 | | | | |
| CSF2RB | | | | | | | | 6.5 | 1.5 | | | |
| CSF3R | | | | | | | | | 21 | | | |
| CSGALCT1 | | | 20 | | | | | 14 | | | 21 | |
| CSL | | | 18 | | | | | 0 | 13.5 | 12 | | |
| CSMD2 | | | 20.5 | | | | | 19.5 | 21.5 | | | |
| CSMD3 | | | | | | | | | 16.5 | | | |
| CSNK1E | | | | | 8 | | | | | | | |
| CSNK1G1 | 2.5 | | | | 9 | | | | | | | |
| CSNK1G3 | | | | | | | | 17 | | | | |
| CSNK2A2 | | | | | | | | 17 | | | | |
| CSPG4 | | | 20 | 12 | 12 | | 15 | | 14 | 12 | | |
| CSPG5 | | | | | | | | 9 | | | | |
| CSRNP1 | | | | | | 18 | | 23 | | 23 | 1.5 | |
| CSRP1 | | 1 | 2 | 5 | 4 | 18 | | 23 | 22 | 10 | | 21 |
| CSRP3 | | | | | 20 | | | | | | | |
| CST3 | 2 | | | | | | | | 3 | | | |
| CST8 | | | | | | | | | | 12 | | |
| CSTAD | | | | | | | | | | 17 | | |
| CSTB | | | | 12 | | | | 16 | | | | |
| CSTF2 | | | | | | | | 13 | 21.5 | | | |
| CSTF3 | | | | 3.833 | | | | | | | 3.833 | |
| CT025673.2 | | | | | | | | 9 | | | | |
| CT573086.1 | 8 | 7 | 10 | | | | | 9.5 | | | | |
| CTBP1 | | | | | | 13.5 | | | 12 | 11 | | |
| CTC1 | | 10.5 | | | | | | | 17 | 20 | | |
| CTDP1 | | | 7.167 | | | | | 20 | 20 | | | |
| CTDSPL | 10.5 | | | | | | | | | | | |
| CTF1 | | | 15.5 | | | | | | | 9 | | |
| CTGF | | | | | | | | 22 | 18 | | | |
| CTH | | | 22 | | | | | 13 | | 5.5 | | |
| CTHRC1 | | | | | | | | | 16 | | | |
| CTIF | | | | | | | 21 | | | | | |
| CTLA2A | | | | | | | | 11 | 21.5 | | | |
| CTNNB1 | | | | | | | | 19 | 19 | | | |
| CTNND1 | | | | | | | | | 20.5 | | | |
| CTNND2 | | | | | | | | | | 14 | | |
| CTNS | | | | | | | | | | 10 | | |
| CTPS2 | | | 21 | | | | | | | | | |
| CTSA | | | | | | | | | 21 | | | |
| CTSC | 6 | 7 | 9 | | | | | 9 | 8.5 | | | |
| CTSF | | | | | | | | | | 12.5 | | |
| CTSH | | | 22.5 | | | 21 | | 2 | | | | |
| CTSL2 | | | | | | | | | | 22 | | |
| CTSZ | | | | | | | | | 9.5 | 10 | | |
| CTTNBP2 | | | | | | | | 22 | | 12 | | 22 |
| CUEDC1 | | | | | | | | | | | 23.5 | |
| CUEDC2 | | | | | | | | | | 10 | | |
| CUL1 | | | | | | | | | 22.5 | | | |
| CUL2 | 6 | 4 | 4 | | | | | | | | 0 | |
| CUL7 | | | | | | | | 13 | | 12 | | |
| CUL9 | | | | | | | | | 23 | | | 23 |
| CUTA | | | | | | | | | | 3.5 | 22 | |
| CUTC | | | 20 | | | | | | | | | |
| CUX1 | | | | | | | | | | 10 | | |
| CUX2 | | 11 | 12 | | | | | 9 | 14.5 | 9 | | 16 |
| CWC22 | | | | | | | | | | 5 | 21.5 | |
| CX3CR1 | | | | | | 11.5 | | | | | | |
| CXADR | | | | | | | | | 0 | 12.5 | | |
| CXCL12 | | | 4.5 | | | | | | | | | |
| CXCL13 | | | | | | | | | | 12 | | |
| CXCL14 | | | | | | | | | 23 | | | |
| CXCL15 | | | | | | | | 0 | 21 | | | |
| CXCL16 | | | | | | | | | 22.5 | | | |
| CXCL6 | | | | | | 21 | | | | | | |
| CXCL9 | | | | | | | 9 | | | | | |
| CXCR4 | | | | | | | | | | | 22 | |
| CXCR7 | | | | | | | | | | | 7.167 | |
| CXORF26 | | | | | 22.5 | | | | | | | |
| CXORF38 | | | | 23.5 | | | | | | | | |
| CXXC5 | | 8 | 10 | | | | | | | | | |
| CYB561 | | | | | | | | | | | | 9 |
| CYB5A | | 6.5 | | | | | | 8.5 | | 8.5 | | |
| CYB5B | | | | | | | | 0 | 21 | 8 | | |
| CYB5D2 | | | | | | | | 23 | | 9 | | |
| CYB5R2 | | | | | | 11 | | | | | | |
| CYB5R4 | | | 23 | | | | | | | | 2 | 1 |
| CYBASC3 | 0 | 0 | 2 | | | | | | 0 | | | 0 |
| CYBRD1 | | | | | | | | | | 2 | 0 | |
| CYC1 | 22.5 | | | | | | | 14 | | | | |
| CYFIP2 | | | | | | | | | | 13 | | |
| CYGB | | | 22.5 | | | | | 21 | | | | |
| CYLD | | | | | | 5 | | | 21 | 0 | 3 | |
| CYP17A1 | | | | | | | | | 22 | | | |
| CYP1A1 | | | | 23 | | | | 22 | | | | |
| CYP1B1 | | | | | | | | 22 | | | | |
| CYP21A2 | | | | | | | | 9 | 20.5 | 12 | | |
| CYP24A1 | | | | | | | | 11 | 12 | 12 | | |
| CYP26B1 | | | | | 8.5 | | | | | | | |
| CYP2B6 | | | | | 23 | | | | | | | |
| CYP2B9 | | 7 | 7 | | | | | | | | | |
| CYP2C67 | | | | | | | | | | 12 | | |
| CYP2C68 | | | | | | | | | | 10 | | |
| CYP2D22 | | | | | 18 | | | | | 14 | | |
| CYP2D37-PS | | | | | | | | 8 | 20.5 | | | |
| CYP2D6 | | 9 | 9.5 | | | | | | 22 | | | |
| CYP2E1 | | | | | | 8 | | | | | | |
| CYP2G1 | | | | | | | | | 4 | | | |
| CYP2J9 | | | | | | | | 23 | | | | |
| CYP2R1 | | | 16 | 17 | | | | | | | | 11 |
| CYP2U1 | | 7 | 8 | | | | | 11 | | | | |
| CYP39A1 | | | | | | | | | 22.5 | | | |
| CYP3A13 | | | | | | | | | | 8 | | |
| CYP4A28-PS | | | | 12 | | | 12 | 16 | | | | |
| CYP4B1 | | | 23 | | 0 | | | | | | | |
| CYP4F12 | | | 23.5 | | | | | | | 15 | | |
| CYP4F22 | | | | | | | | | 20 | | | |
| CYP4F3 | | | | | | | | | 19 | | 23.5 | |
| CYP4V2 | | 1 | | | | | | | | | | |
| CYP8B1 | | | | | | | | 13 | 2.5 | | | |
| CYR61 | | | | | | | | 8.5 | 3.5 | | | |
| CYS1 | | | | 16 | | | | | | | | |
| CYSLTR2 | | 18 | | | | | | | | | | |
| CYSTM1 | | | | 18 | 18.5 | | 18 | | | | | |
| CYTH1 | | | | | | | | | | 21.5 | | |
| CYTIP | | | | | | 0 | | | 3 | | | |
| CYTL1 | | 7.5 | | | | 11 | | 7.5 | | | | |
| CYYR1 | | | | | | | | | 23 | | | |
| D030046N08RIK | | | 11 | | | | | | 3.5 | | | |
| D130007C19RIK | | | | | 12 | | | | | | | |
| D2 | | | | | | | | | 18 | | | 21 |
| D2HGDH | | | | | | | | | | 11 | | |
| D630013G24RIK | | | | | | | | | 8 | 9.5 | | |
| D630029K05RIK | | | | | | | | 23 | | | | |
| D730003I15RIK | | | 22 | 22 | 20 | | | | | 20 | | |
| D730039F16RIK | | | | | | | | | | 12 | | |
| D930048N14RIK | | | 22 | | | 17 | | | | | | |
| DAAM1 | 19 | | 20 | | | | | 7 | | | | |
| DAB2 | | | | | | | | | | 11.5 | | |
| DAB2IP | | 16 | | | | | | | | | | |
| DACT1 | 20 | | 22 | | | | | | | | | 22.5 |
| DAF2 | | | | | | | | | 3 | | | |
| DAG1 | | | | | 11 | | | | | | | |
| DAK | | | | | | | | | 19 | | | |
| DALRD3 | 9 | | | | | 9 | | 10 | 8.5 | 10 | 9 | 10 |
| DAO | | | | | | | | 22 | 3 | | 23 | |
| DAP | | | | | | | | 20 | | | | |
| DAPK1 | | | 10 | | | | | | | | | |
| DAPK2 | | | | | 3 | | | | 3 | | | |
| DAPK3 | | | | | | | | | | 8 | | |
| DARS2 | | | | | | | | | 13 | | | |
| DAZAP2 | | | | | | | | | 18 | | | |
| DBI | | | | | | | | | 16.5 | | | |
| DBF | | | | | | | | | | | 20 | |
| DBT | | | | | | | | 17 | | 3 | | |
| DCAF12 | | 23 | | | | | | | | | | |
| DCAF15 | 17 | 18 | 22.5 | | | | | 20 | 18 | | | |
| DCAF4 | 1 | | | 6 | | | | 6 | 4 | 11 | | |
| DCAF6 | | 15 | | 16 | | 14.5 | | | 18 | 16 | | |
| DCAF7 | 2.5 | | | | | | | | 3 | | | |
| DCAF8 | 0 | | | | | | | 3 | | 23 | | |
| DCBLD1 | | | | | | | 20 | | | | | |
| DCDC2 | | | | | | | | | | 13 | | |
| DCDC5 | | | | | | | | | 23 | | | |
| DCHS1 | | | | | | | | | | 13 | | |
| DCLK1 | | | 11 | | | | | | | | | |
| DCLK2 | | | | | | | | 23.5 | | | | |
| DCLK3 | | 9 | | | | | | | | | | |
| DCLRE1A | 20 | | | | | | | 21.5 | | | | |
| DCLRE1B | | | | | | | | | | 12 | | |
| DCN | | | 22 | 2 | 2.5 | 1.5 | | | | | 0 | 23 |
| DCTD | | | 13 | | | | | | | | | |
| DCTN2 | | | | 4.5 | | | | 22 | | | | |
| DCTN3 | | | | | 21 | | | | | 14 | 4 | |
| DCTN5 | | | | | | | | 7 | | | | |
| DCTN6 | | | | | | 22 | | | | | | |
| DCTPP1 | | 7.5 | 8 | | | | | | | | | 11 |
| DCUN1D3 | | | | 5 | | | | 4 | 23 | | 1 | |
| DCUN1D4 | | | | | | | | | | 8.5 | | |
| DCUN1D5 | | | | | | | | | | 11 | | |
| DCXR | 6 | | | | | | | | | | | |
| DDAH1 | | | | | | | | 9 | 7 | | | |
| DDAH2 | | | | | | | | | | 7.5 | | 9.5 |
| DDB1 | | | | | | 5 | | | | 4 | | |
| DDC | | | | | | | | 10 | | 13 | | |
| DDHD1 | | | | | | | | | | 13 | | |
| DDHD2 | | | | | | | | | 9 | | | |
| DDIT4 | 8 | | | | | | | 23 | | | | |
| DDIT4L | 23 | | | 22 | | | | 18 | | | | |
| DDO | | | | | | | | 17 | | | | |
| DDR1 | | | | | | | | 10.5 | | 20 | | |
| DDR2 | | | | | | | | 20 | | | | |
| DDRGK1 | | | | | | | | | | 15 | | |
| DDX1 | | | | | | | | 17 | 15.5 | | | |
| DDX17 | | | 8 | | | | | 17 | 15 | | | |
| DDX28 | | | | | | | | 16 | | | | |
| DDX39B | | | 8.5 | | | | | | | | | 9 |
| DDX3Y | | | 7 | | | | | | 14.5 | | | |
| DDX46 | | | | | | | | 16 | | | | |
| DDX49 | | | | | | | | | 15 | | | |
| DDX6 | | | 3 | | | | | 19 | | | | |
| DDX60 | | | | | | | | | | | | 6 |
| DECR2 | | | | | | | | | | | 23 | |
| DEDD | | | | | | | | | 19 | 14 | | |
| DEDD2 | | | | | 10 | | | | 22.5 | 15 | | |
| DEF8 | | | | | | | | 17 | 17 | | | |
| DEFB1 | | | | | | | | 16 | | | | |
| DEFB13 | | | | | | | | | 23 | | | |
| DEGS1 | | | | | | | | | 2 | | | |
| DEGS2 | | | | | | | | | | | 6 | |
| DENND1A | | | | | | | | | | 13 | | |
| DENND1C | | | | | | | | | | 18 | | |
| DENND2D | | | | | | | | 14.5 | | | | |
| DENND4A | | | | | | | | 11 | 16.5 | | | |
| DENND4B | | | | 21 | | | 21 | 19 | | 20.5 | | |
| DENND4C | | | 5 | | | | | | | | | |
| DENND5B | | | | 20 | | | 21 | | 23 | | | |
| DEPDC1B | | | | 14 | | | 17 | 3 | | | 23 | |
| DERL1 | | | | | | | | 10 | 0 | | | |
| DES | | | | | | | | | 22 | | | |
| DEXI | | | | | | | | 7.5 | | | | |
| DFNB31 | 2.5 | | | | | | | | | | | |
| DGAT1 | | 1 | | | | | | | | | | |
| DGAT2 | | | 21 | | | | | | 22 | | | |
| DGCR14 | | | | | | | | | | 11 | | |
| DGCR8 | | | 8 | | | | | 0 | 4 | | | |
| DGKA | | | | | | | | | | | 11.5 | |
| DGKB | | | | | | | | 21 | | | | |
| DGKD | | | | | | | | 12 | | | | |
| DGKG | | | | | | | | 17 | 23 | 14 | | |
| DGKH | | | | | | | | | 0 | | | |
| DGKI | | | 23 | | | | | | | 7 | | |
| DGKQ | | | | | | | | | | 7.5 | | |
| DGKZ | | | | | | | | | 11.5 | 4 | | |
| DGUOK | 5.5 | | | | | 14.5 | | | | | | |
| DH3 | | | 20 | | | | | | | | | |
| DH6 | 13 | 13 | | | | | | | | | | |
| DH7 | | | | | | | | | 0 | | | |
| DH9 | | | | | | | | | | | 5 | |
| DHC12 | | | | | | | | 2 | | | 7 | |
| DHCR7 | 15.5 | | | | | | | | | | | |
| DHRS1 | | 22 | | | | | | | | | | |
| DHRS11 | 18 | | | | | | | | | | | |
| DHRS2 | | | | | | | | | | 12 | | |
| DHRS3 | | | | | | 8 | | | | | | |
| DHRS7B | | | | | | | | 4 | | | | |
| DHRS9 | 20 | 21 | 21 | | | | | 19 | | 17 | | |
| DHTKD1 | | | | | | | | | | 22 | 0 | |
| DHX29 | 10 | | | | | | | | 11 | | | |
| DHX32 | | | | | | | | | | 11 | | |
| DHX33 | | | | | | | | 4 | 2 | | | |
| DHX35 | | | 11 | | | | | | 4 | | | |
| DHX36 | | | | | | | | | 21 | | | |
| DHX37 | | | | | | | | 6 | 3 | | | |
| DHX40 | | | | | | | | | 9 | | | |
| DHX58 | | | | | | | | | | | | 8 |
| DHX9 | | | | | | | | | | 11 | | |
| DIABLO | | | | | | | | 8.5 | | | | |
| DIAPH2 | | | | | | | | | 23 | | | |
| DIAPH3 | | | | | | 17 | | | 13 | | | |
| DICER1 | | | | | | | | | 4 | | | |
| DIDO1 | | | | | | | 9 | | | 8 | | |
| DIMT1 | | | | | | | | | | 13.5 | | |
| DIO2 | | | | | 23.5 | | | | 7 | | | |
| DIP2A | | | | 0 | | 1 | 1.5 | 2 | | | 1.5 | 23 |
| DIP2B | | | | | | | | | 3 | | | |
| DIP2C | | | | | | | | | 17 | | | 20.5 |
| DIRC2 | | | | | | | | | | | 5 | |
| DIS3L | | | | | | | | | | 19 | | |
| DIS3L2 | | | | | | | | | 15 | | | |
| DISP1 | | | | | | | | 0 | | | | |
| DISP2 | | | | | | | | | | 16 | | |
| DIXDC1 | 16 | | | | | | | | | | | |
| DJA1 | | | | | | 21 | | | | | | |
| DJA2 | | | | | 22 | | | | | | | |
| DJA4 | | | | | | | | | 4.5 | | | |
| DJB11 | | | | | | | 0 | 21.5 | 22.5 | | | |
| DJB14 | | | | 22 | | | | | | | | |
| DJB2 | | | 10 | | | | | | 18 | | | |
| DJB4 | | | | | | | | | | 0 | | |
| DJB9 | | | | | | 1 | | 1 | | 5 | | |
| DJC1 | 8.5 | | | | | | | | 21.5 | | | 8.5 |
| DJC10 | | | | | | | | | | 5 | | |
| DJC12 | | | | | | | | | | | | 11 |
| DJC13 | | | | | | | | | | 3 | | |
| DJC14 | | | | | | | | | | 4.5 | | |
| DJC18 | | | 0.5 | 4.5 | 4 | | 4 | | | | | 22.5 |
| DJC22 | 23 | | 2 | | 4 | | | | 1 | | | |
| DJC24 | | | | | | | | | 22 | | | |
| DJC28 | | | | 22 | | | | | | | | |
| DJC3 | | | 22 | | | | | 19 | | 17 | | |
| DJC30 | | | | | | 0.5 | | | 23 | 1.5 | | |
| DJC4 | | 8 | | | | | | | | | | |
| DJC5 | | | | | | | | | 9 | | | |
| DJC5G | | | | | | | | 10 | | | | |
| DJC6 | | | | | | | | | 15 | | | |
| DK | | | | | | | | | 0 | | | |
| DKC1 | | | | | | 12.5 | | | | | | |
| DKK2 | | | | | | | | 22 | 23 | | | |
| DLC1 | | | | | | | | | 18 | 11.5 | | |
| DLEU7 | | | | | | | | | | 23.5 | | |
| DLG1 | | | 19 | | | | | | | | | |
| DLG2 | 11 | | | | | | | | 11 | | | |
| DLGAP1 | | | | | | | | 7 | | | | |
| DLL1 | | | | | | | | 8.5 | | | | |
| DLL4 | | | | | | | | | | | | 9 |
| DMC1 | | | | | | 19 | | 22 | | | | |
| DMD | | | | | | | | | 20.5 | 0 | | |
| DMP1 | | | | | | | | | | | 0 | |
| DMTF1 | | | | | | | | 19 | | | | |
| DNM1 | | | | | 14 | | | 14 | | | | |
| DNM1L | | | | | | | 6 | | | | | |
| DNM2 | | | | | | | | | | 6.5 | | |
| DNM3 | | | 5 | | | | | | | | | |
| DNMT1 | 21 | | | | | | | | | | | |
| DNMT3A | | | | | | | | | 11 | | | |
| DNMT3B | | 12.5 | | | | | | | 18 | 12 | | 13 |
| DNPEP | | | | | | | | | 8 | | | |
| DNTTIP1 | | | | | | | | | 11 | | | |
| DOC2B | | | | | | | 5.5 | | | | | |
| DOCK1 | | | | | 20 | | | | | | | |
| DOCK10 | | | | | | | | 21 | 2 | | | |
| DOCK11 | | 9 | 11 | | | 9.5 | | | | 7 | 12 | |
| DOCK2 | | | | | | | | | 1 | | | |
| DOCK4 | | | | | | | | 21 | | | | |
| DOCK5 | | 9 | | | | | | | | 7.5 | | |
| DOCK6 | | | 10 | 12.5 | | | | | | | | |
| DOCK7 | | | | | | | | | 21 | | | |
| DOCK8 | 12 | | | | | | | | 16 | | | |
| DOCK9 | | | | | | | | | 15 | | | |
| DOK6 | | | 8 | | | 19 | | | | | | |
| DOK7 | | | | | | | | | 4 | | 2 | |
| DOLPP1 | | 22.5 | | | | 3 | | | | 21 | | 23 |
| DOPEY2 | | | | 13.5 | | | | 14.5 | | 15 | | |
| DOT1L | | | | | 23 | | | 23 | | 23.5 | | |
| DPCR1 | | | | | | | | | | | 5.5 | |
| DPEP1 | | | | | | | | 12 | | | | |
| DPM3 | | | | | | | | | | 11.5 | | |
| DPP10 | | | | | | | | | | 12.5 | | |
| DPP8 | | | | | | | | | | 12 | | |
| DPP9 | | | 0 | | | | | 1 | | | | 2 |
| DPT | | | | 6 | | | 6 | | | | | |
| DPY19L1 | | | | | | | | | | 7 | | |
| DPY19L3 | | 8 | | | | | | | | | | |
| DPYD | | | | | | | 6 | | | | | |
| DPYSL2 | | | | | | | | | | 7 | | |
| DQX1 | | | | | | 10 | | | | | | |
| DRAM1 | | | | | | | | | 22 | | | |
| DRAM2 | | | 23 | | | | 22 | 21 | 20 | 16.5 | | |
| DRD4 | | | | | | | | | | | 9 | |
| DRP2 | | | | | | | | 22.5 | | | | 23 |
| DSC2 | | | | | | | | | 22 | | | |
| DSCR3 | | | | | | 11.5 | | | | | | |
| DSCR6 | | | | | | | | | 21 | | | |
| DSE1 | | | | | | | | 23 | | | | |
| DSE1L1 | | | | | | | | 22 | | | | |
| DSE2 | | 8 | | | | | | | | | | |
| DSE2B | | | | | | | | | 17 | | | |
| DSG2 | | | | 21 | | | | | | | | |
| DSN1 | | | | | | | | | | | | 11.5 |
| DST | | | | | | | | | | | | 1 |
| DSTN | | | | | | | | | | 20 | | |
| DSYN1 | | | | | | | | | 22 | | | |
| DT | | | | | | | | 22 | | | | |
| DTNBP1 | | | | | | | | 22.5 | | | | 20 |
| DTX1 | | | | | | | | 7 | | | | |
| DTX2 | | | | | | | | | | 12 | | |
| DTX3L | | | | | 7 | | | | | | | |
| DTX4 | | | | | | | | | | | 6.167 | |
| DTYMK | 18 | 19 | 19 | | 20 | 20 | | 17 | 18 | 17 | | 18 |
| DUS2L | | | | | | | | | | 14 | | |
| DUS4L | | | | | | 0 | | | | | | |
| DUSP1 | | | | | | | | | 22 | | | |
| DUSP10 | | | | 12 | | | | | 22.5 | | | |
| DUSP11 | | | 4.833 | | | | 8 | | 0 | | | |
| DUSP12 | | | | | | | | | 0 | | | |
| DUSP14 | 19 | | | | | | | | | 11.5 | | |
| DUSP15 | | | | 22 | | | 22 | | 21 | | | |
| DUSP16 | | | | | | | | | | 10.5 | | |
| DUSP19 | 18.5 | | 23 | | | | 21.5 | 21 | | | | |
| DUSP6 | 20 | | | | | | | | | | | |
| DUSP7 | | 0.5 | | | | | | | | | | |
| DUSP9 | | 5 | | | | | | | | | | |
| DVL3 | | | | | | | | | | 9 | | |
| DYM | | | | | | | | | | 9 | | |
| DYNC1H1 | | | 5 | | | | | | 5 | | | |
| DYNC1I2 | | | | | | | | | 7.5 | 16 | | |
| DYNC1LI1 | | | | | | 18 | | | | | | |
| DYNC2H1 | | | | 22 | | | | | | | | |
| DYNC2LI1 | | | | | | | | 10 | | | | |
| DYNLL1 | | | | | | | | | | 2 | | |
| DYNLL2 | | | | | | | | | | 11 | | |
| DYNLRB2 | | | | 23 | | | | | | | | |
| DYRK1B | | | | | | | | 8 | | 4 | 23.5 | |
| DYRK2 | | | | | | | 10 | | | | | |
| DYSF | | | | | | 7 | | | | | | |
| DZIP3 | | | 10 | | | | | | | | | |
| E030019B06RIK | 7.5 | | | | | | | | 11 | | | 8 |
| E230001N04RIK | | | | | | | | 0 | | | | |
| E2F2 | | | | | | | | 12.5 | | 15 | | |
| E2F5 | | | | | | | | 11 | | | | |
| E2F6 | | | | | | | | | | 16 | | |
| E2F8 | | | | | | | | 8 | 2 | | | |
| EAF1 | | | 7 | | | | | 2 | | | | |
| EAPP | | | 18 | | | | | | | | 7 | |
| EARS2 | | | | | | | | | 2.5 | | | |
| EBF1 | | | 7 | | | | | | | | | |
| EBF3 | | 15 | | 21 | | | | | 11 | 11.5 | 20 | |
| ECE1 | | | | | | | | | 22 | | | |
| ECHDC1 | 1 | | | | | 2 | | 23 | 0 | 0 | 22 | |
| ECHDC2 | 23 | | 22.5 | | | | | | 1 | | | |
| ECHDC3 | | | | | | | | | | 20 | | |
| ECI1 | | | | | | | | | 19 | | | |
| ECI2 | | 1 | 2 | | | | | 22 | 1 | 22 | | 23 |
| ECM2 | | | | | | | | 22 | | | 22 | |
| EDA | | | 7 | | | | | | | | | |
| EDC3 | 6 | | | | | | | | | | | 9 |
| EDC4 | | | | | | | | | | | | 22.5 |
| EDEM1 | 20 | | | | | | | | | | | |
| EDEM3 | | | | | | | | | 20 | | | |
| EDN1 | | | | | | | | | 22.5 | | | |
| EDN3 | | | | | | | | | 22 | | | |
| EDNRA | 18 | | | | | | 23 | | | | | |
| EDNRB | | | | | | | | | 23 | | | |
| EEF1A2 | | | | | | | | | 6 | | | |
| EEF1E1 | | | | | | | | | 14 | | | |
| EEF1G | | | | | | | | | | 9 | | |
| EEF2K | 8 | | | | | | | | 8.5 | | | |
| EEPD1 | | | | | | | | 20.5 | | | | |
| EF2 | 14 | | 14.5 | | | | | 14 | 14 | | | 14.5 |
| EFCAB1 | | | | | | | | 20.5 | | | | |
| EFCAB2 | | | | | | | | | 2 | | | |
| EFCAB4A | | | | | | | | | 21 | | | |
| EFCAB4B | | 23 | | | | | | | 23 | | | |
| EFEMP1 | | | | | | 19.5 | | | | 16 | | |
| EFEMP2 | | | | | | | | | | 18 | | |
| EFHD1 | | | | | | | | | 3.5 | | | |
| EFHD2 | | | 0 | | | | | | 23 | | | |
| EFNB1 | | | 6 | | | | | | | | | |
| EFNB2 | | 23 | 1 | | | | | | 0 | 20 | | |
| EFNB3 | | | | 0 | | | | | | | | 16 |
| EFR3A | 0 | 0 | | | 0.5 | 1 | | 0 | | 23 | 1 | |
| EFR3B | | | | | | | | | 0 | | | |
| EGFL6 | | | | | | | | | | | | 4 |
| EGFL7 | | | | | | | | | | | 23 | |
| EGFLAM | | | 15.5 | | | 18 | | | | | | |
| EGFR | | 13 | 15 | | | 13 | | 21 | 14 | 11.5 | 11 | |
| EGLN1 | | | | | | | | 20 | | | | |
| EGLN2 | | | | | | | | | 1 | | | |
| EGLN3 | | | | | | 18 | | | | | | 23 |
| EH | | | | | | 22 | | | | | | |
| EHBP1 | | | | | | | | | | 9 | | 10.5 |
| EHBP1L1 | | | | | | | | | 4 | | | |
| EHD1 | | | 12 | | | | | 12.5 | 14 | 9 | | |
| EHD2 | | | | | | | | | | 15.5 | | |
| EHD3 | | 14.5 | | | | | | | | | | 15 |
| EHD4 | | | | | 22 | | | | | | | |
| EHHADH | | | | | | | | | | 11 | | |
| EHMT2 | | | 1 | | | | | | | 7 | | |
| EI24 | | | 4 | | | | | | | | 23 | |
| EID1 | | | | | 14 | | | | 9 | | | |
| EID2B | | | | | | | | | | 15 | | |
| EIF1AX | | | | | | | | 8 | | 0 | | |
| EIF1AY | | | | | | 10 | | | | | | |
| EIF2A | | | | | | | | | 19 | | | |
| EIF2AK1 | | | | | | | | 11 | 21 | | 0 | |
| EIF2B1 | 11 | | | | | | | 13 | | 12 | | |
| EIF2B2 | | | | | | | | 21 | | | | |
| EIF2C2 | | | | | | | | | 22.5 | | | |
| EIF2C3 | | | | | | 5 | | | | | | |
| EIF2C4 | | | | | | | | | 3 | | | |
| EIF2D | 19 | | | | | | | | | | | |
| EIF3B | 22 | | | | | | | | | 1 | | |
| EIF3D | | 4 | | | | | | | | | 2 | |
| EIF3E | | | | | | | | | 21 | 22 | | |
| EIF3F | | | | | | | | | | 11.5 | | |
| EIF4A2 | | | | | | | | | 22.5 | | | |
| EIF4B | | | | | | | | | 1 | 17 | | |
| EIF4E2 | | | 3 | | | | | | 2 | | | |
| EIF4E3 | | | 6.5 | | | | | 8.5 | 10 | | | 10.5 |
| EIF4EBP1 | | | | 21.5 | | | | 16 | | | 22.5 | |
| EIF4EBP2 | 13 | 13 | 14 | | | 15 | | | 12 | 13 | | 13 |
| EIF4EBP3 | | | 12.5 | | | | | | | 6 | | |
| EIF4ENIF1 | | | | | | | | | | 2.5 | | |
| EIF4G1 | | | 23.5 | | | | | 17 | | | 0 | |
| EIF4G2 | | | | | | | | | | 21 | | |
| EIF4G3 | | | | 21 | | | | | | 20 | | |
| EIF5 | | 8 | | | | | | 11 | | 9 | | 11 |
| EIF5A2 | | | | | | | 6 | | 2.5 | | | |
| ELAC1 | | | | | | | | | 22 | | | |
| ELAC2 | | | | | | | | 3 | | | | |
| ELAVL1 | | | | | | | | | | | 2 | |
| ELAVL3 | | | | | | | | 6 | | | | |
| ELAVL4 | | | | | | | | | 2 | | | |
| ELF1 | | | | | | 20.5 | | | | | 22 | 22 |
| ELF2 | | | | | 6 | | | | | | | |
| ELK3 | | | | | | | | | 20 | | | |
| ELL | | | | | | | | | 0 | | | |
| ELL2 | | | 22 | | | 21 | | | | | | |
| ELMO1 | | | | | | | | | 22 | | | |
| ELMO2 | | | | | 5 | | | | | 14.5 | | |
| ELMOD1 | | | | | | | | 23.5 | 3.5 | 5.5 | | |
| ELMOD2 | | | | | | | | | 12 | | | |
| ELMOD3 | | | | | 4 | | 5.5 | | | 18.5 | | |
| ELN | | | | | | 20 | | | 12 | | | |
| ELOVL1 | | | | | | | | | 0 | | | |
| ELOVL2 | | | | | | | | 23 | | 10 | | |
| ELOVL3 | | | | | | 8 | | | | 11.5 | | |
| ELOVL5 | | | | | | | | 20 | 23 | | | |
| ELOVL6 | | | | | | | | 19 | | | | |
| ELOVL7 | 7 | | 9 | | | | 6 | | 8 | | 8 | |
| ELP2 | | | 8 | | | | | 10 | | | | |
| ELP4 | | | | | | | | | 21.5 | | | |
| ELTD1 | 5.5 | | | | | | | | | | | |
| EMB | | | | | | | | 7 | | | | |
| EMCN | | | | 19 | 16 | 19 | | | | 13 | | |
| EMG1 | | | 21 | | | 0 | | | 16 | | | |
| EML1 | | | | | | | | | | | 21 | |
| EML2 | | | | | | | | | 1 | | | |
| EML3 | | | | | | | | | | 5 | | |
| EML4 | 8 | | | | | | | | | | | |
| EML5 | | | 21 | 11 | | | | | | | | |
| EMP1 | | | | | | | | | | 10 | | |
| EMP2 | | | | | | 10 | | | 23 | | | |
| EMR4 | | | | | | | | | | 12 | | |
| ENDOD1 | | | | | | | | | 5 | | | |
| ENDOG | | | 7 | | | | | | | | | |
| ENG | | | | | | | | | | 22 | | 19 |
| ENGASE | 21 | | | | | | | 23 | 22 | | | 20 |
| ENO3 | | | | | | | | | | | 5.833 | |
| ENOX1 | | | | | | | 20 | | | | | |
| ENOX2 | | | | | | | | 0 | | | | |
| ENPEP | | | | | | 17 | | 21 | | | | |
| ENPP1 | | 6 | | | | | | 8 | 7 | | | |
| ENPP2 | | 20.5 | 21 | 22 | | | 23 | 22 | | | | |
| ENPP3 | | | | | | | | 19 | | | | |
| ENPP5 | | | | | | | | | 16 | | | |
| ENPP7 | | | | | | | | | 2 | | | |
| ENTPD1 | | | | | | | | | 21 | | | |
| ENTPD2 | | | | 19 | | | 19 | | 5 | | | |
| ENTPD3 | 19 | | | | | | | 23.5 | 19 | | | |
| ENTPD5 | | | | | | | | | | 17 | | |
| ENTPD6 | | | | | 22 | 10 | | 9 | | | | |
| ENTPD8 | | | 12 | | | | | | | | | |
| ENY2 | | | | | | | | 12 | | | | |
| EOGT | | | | | | 11 | | 12 | 21 | 17 | | |
| EP300 | | | | | | | | | 23.5 | | | |
| EP400 | | | | | | 1 | | | | | | |
| EPAS1 | | 0 | | | | | | | 8 | | | |
| EPB41 | | | 10 | | | | | | | | | |
| EPB41L2 | | | | | | | | | | | | 16 |
| EPB41L3 | | | | 10 | | | | | 22 | | | |
| EPB41L4B | 22.5 | 22 | | | | 21 | | 0 | | 21 | | 0 |
| EPB41L5 | | | | | | 18 | | | | | | |
| EPC2 | | | | | | | | 9 | | 9 | 7.5 | |
| EPDR1 | | | | | | | | | | 3 | | |
| EPG5 | | | | 23 | 23 | 1 | | | 21 | 2 | | |
| EPHA1 | | | | | | | | 0 | | | | |
| EPHA3 | | | | | 4.5 | | | | | | | |
| EPHA4 | | 12 | | | 12 | | | | | 11 | | |
| EPHA5 | | | | | | | | | | | | 9 |
| EPHA6 | | | 13 | | | 13.5 | | | | 13.5 | | |
| EPHA7 | | | | | | | | 22 | 10 | | | |
| EPHA8 | | | 22 | | | | | | | | 0 | |
| EPHB1 | | | | | | 16.5 | | | | | | |
| EPHB4 | | | | 20 | | 18 | | | 19 | | | |
| EPHX1 | | | | | | | | | | | 2 | |
| EPHX3 | | | 14 | 20 | | 14 | | | | 12.5 | | |
| EPM2A | | | | | | | | 12.5 | | 11 | | |
| EPM2AIP1 | | | | | | | | | 13 | 8 | | |
| EPN2 | | | | | | | | 23.5 | | | | 1 |
| EPRS | | | | | | | | | 17 | | | |
| EPS8 | | | | | | | | 10 | | 12.5 | | |
| EPS8L2 | 10 | | | | | 8 | | | | | | |
| EPT1 | | | | | | | | 0 | | | | |
| ERAL1 | | | | | | | | | | 14.5 | | 5.5 |
| ERBB2 | | | | | | | | | 9.5 | | | 8 |
| ERBB2IP | | | | | | | | 8 | | | | |
| ERBB3 | | | | | | 22 | | | 2 | | | |
| ERBB4 | 9 | 11 | 11 | | 15 | | 13 | | | 13 | | |
| ERC1 | | | 21.5 | | | | | | | | | |
| ERC2 | | | | | | | | 23 | | | 23 | |
| ERCC1 | | 20.5 | 18.5 | | | | | | 21.5 | | | |
| ERCC5 | | | 22 | 21 | | 21 | | | | | 0.5 | |
| ERF | | | | | | 18 | | | | 13 | | |
| ERG | | | 22 | | | | | | | | | |
| ERGIC1 | | | | | | | | 0 | | | | |
| ERGIC2 | | | | | | | | 22 | | | | 22.5 |
| ERI1 | | | | | | | | 5 | 23 | | | |
| ERI2 | | | | | | | | | | | | 7.5 |
| ERLIN1 | | | 0 | | | | | | | | | |
| ERLIN2 | | | | | | | 8 | | | | | |
| ERMN | | 5 | | | | | | | | | | |
| ERMP1 | | | | | | | | | 22 | | | |
| ERO1L | | | | | | | | | 22 | | | |
| ERP29 | | | | | | | | | 23 | | | |
| ERP44 | | | | | | | 21.5 | 18 | 19 | | | |
| ESM1 | | | | | | | | | 4 | | | 6 |
| ESR1 | | | | | | | | | 1 | | | |
| ESR2 | | | 2 | | | | | | | | | |
| ESRP2 | | | | | | 8 | | | | | | 8 |
| ESRRA | 15 | | | | | | | | | | | |
| ESRRG | | | | | | | | | | 9 | | |
| ESYT1 | | | | | 22 | | | | | | | |
| ESYT2 | | | | | | | | | 23 | | | |
| ETFB | 19.5 | | | | | | | | 23 | | | |
| ETFDH | | | | | | | | 23 | | | | |
| ETHE1 | | | | | | | | 11.5 | 19 | | | |
| ETNK1 | | | | | | 20 | | | | | 2 | |
| ETNK2 | | | | | | | | | | 17 | | |
| ETS1 | 19 | | | | | | | | | | | |
| ETS2 | | | | | | | | | 13.5 | | | |
| ETV1 | | | | | | | | | 22 | | | |
| ETV5 | | | 21 | | | | | | | | | 21.5 |
| ETV6 | | | | | | | | | 3.5 | | | |
| EVI5 | | | | | | | | 19 | | | | |
| EVI5L | | | | | | | | | 14 | | | |
| EXD1 | | | | | | | | 9 | 12 | 11 | | |
| EXD2 | | | | | | | | | | | | 9 |
| EXOC1 | | | 10.5 | | | | | 5 | | 14.5 | | |
| EXOC2 | | | 7 | | | | | | | | | 9.5 |
| EXOC3 | | | | | | | | | | 16 | | |
| EXOC4 | | | | | | | | 6 | | | | |
| EXOC5 | | | | | | | | | | 10 | | |
| EXOC6 | | | | | | | 19.5 | | | | | |
| EXOC6B | | | | | | | | | | 9 | | |
| EXOC8 | | 9 | 10 | 9 | 7 | | 7 | 9.5 | | 10 | | |
| EXOG | 23 | | | 7 | 10.5 | | | | | | | |
| EXOSC1 | | | 21.5 | | | | | | 0 | | | |
| EXOSC2 | | | | | 2 | | | | | | | |
| EXOSC3 | | | 21 | | | 20.5 | | | | | 22 | |
| EXOSC7 | | | | | | | | | | 22.5 | | |
| EXOSC8 | | | | | | | 21 | 19 | | | | |
| EXOSC9 | | | | | | | | | 0.5 | | | |
| EXPH5 | | | | | | | | | 13 | | | |
| EXT2 | | | | | | | | | 4 | | | |
| EXTL1 | | | | | | | | | | | 9 | |
| EXTL3 | | | | | | | | | 2 | | | |
| EYA1 | | | | | | | | 4 | | | 0 | |
| EYA2 | | | | | | | | | 2 | | | |
| EZH1 | | 5 | | | | | | | | | | |
| EZH2 | | | | | | 19 | | | | | | |
| F11R | | | | | | | | | | 13 | | |
| F2R | | | | | | 22 | | | | | | |
| F2RL1 | | | | | | | | 7.5 | | | | |
| F3 | | | | | | | | | 19 | | | 20 |
| F730043M19RIK | | | 20.5 | | | 4 | | | | | | |
| F830001A07RIK | | | | | | | | | 1 | | | |
| FAAH | | | | | | | | 10 | | 11 | 11 | |
| FABP1 | | | | | | | | 6 | | | | |
| FABP2 | | | 21 | | | 20 | | | | | | |
| FABP7 | 0 | | | | | | | | | | | |
| FADS1 | | | | | | | | | 17 | | | |
| FADS2 | | | | | | | | | 23 | | | |
| FADS3 | | | 11 | | | | | | | | | |
| FADS6 | | | | | | | | | | | 23 | |
| FAF2 | 3 | 1 | 1 | | | 2 | | 4 | 0 | 7 | | |
| FAH | | | | | | | | | 21 | 13 | | |
| FAIM | | | | | | | | 23 | | | | |
| FAM100A | | 6 | 7 | | | | | | | | | |
| FAM101B | | | 23.5 | 4 | | | | | 22 | | | |
| FAM102A | | 7 | | | 9 | | | 9 | | 7.5 | | |
| FAM107A | | | | | | | | | 17 | | | |
| FAM108A1 | | | | | | | | | 23 | | | |
| FAM108B1 | | | | | 22 | | | | 1 | | | |
| FAM108C1 | 2 | | | | | | | | | | | |
| FAM110B | | | 16 | | | | | | 23 | | | |
| FAM111A | | | | | 20 | | | | | | | |
| FAM114A1 | | 22 | | | | | | | | | | |
| FAM115A | | | | | | | | 19 | | | | |
| FAM116B | | | | | 8 | | | | | | | |
| FAM117A | | | | | 11 | | | | | | | |
| FAM117B | | | | | | | | | | 7 | | |
| FAM120A | 2 | | | | 22 | | | 0 | 3 | 3 | | |
| FAM120B | | | | | | | | | 22 | | | |
| FAM120C | | | 6 | | | | | | | | | |
| FAM123B | | | | | | | | | 5 | 8 | | |
| FAM123C | | | | 3 | 5 | | | | | | | |
| FAM124A | 16.5 | 19 | 19.5 | | | | | 17 | | 6 | | |
| FAM124B | | | | | | | | | 18 | | | |
| FAM125A | | | | | | | | | 23 | | | |
| FAM125B | | | | | | | | | | 2 | | |
| FAM126B | | | | | | 0 | | | | | 1 | |
| FAM129A | | | | | | | | | | 11 | | |
| FAM132A | | | | | 21 | | | 2.5 | | 5 | 22 | |
| FAM134A | | | | | | | | | | 12 | | |
| FAM134B | 21 | | | | | | | | | | | |
| FAM13A | | | | | | 19 | | | | | | |
| FAM13B | | | | | | | | 16 | | 19 | 22 | 20 |
| FAM149B1 | | | | 22.5 | | | | | | | | |
| FAM150B | | | | | | 15 | | | | | | |
| FAM155A | | | | | | | | | | 22.5 | | |
| FAM160A1 | | | | | | | | | | 6.167 | | |
| FAM160A2 | | | | | | | | | | 9 | | |
| FAM161B | | | | | | | | | 20 | | | |
| FAM162B | 15 | | | | | | | | | | | |
| FAM163A | | | | | | 14 | | 5 | | 13 | | |
| FAM169B | | | | | | | | 14.5 | | | | |
| FAM171A1 | | | | | | 12 | | 14 | | 12 | | |
| FAM171B | | | | | | | | 3 | | | | |
| FAM172A | | | | | | 15 | | | | 12 | 13.5 | |
| FAM173B | | | | | | | | 20 | | | | |
| FAM174B | | 21 | 22 | | | | | | | 3 | 0 | 21 |
| FAM175B | | | | | | 17 | | | | | | |
| FAM178A | | 20 | | | | | | 20 | | | | 18 |
| FAM184A | | | | 13 | | | | | | | | |
| FAM188A | | | | | | | | | | 22 | | |
| FAM188B | | | | | | | | 23.5 | 23 | | | |
| FAM189A2 | | | 10 | | | | | | | | | |
| FAM190B | | | | 17 | | | | | | | | |
| FAM195A | | | | | | 2.5 | | | 6 | | | |
| FAM195B | | | | | | | | | 4 | | | |
| FAM198A | | | | | | | | | 22.5 | | | |
| FAM198B | | | | | | | | | | 17 | | |
| FAM19A1 | | | | | | | 7 | | | | | |
| FAM204A | | | 23.5 | | | | | | | | | |
| FAM206A | | | | | | | | | 22 | | | |
| FAM208B | 7 | | | | 9 | | | | | 7 | | |
| FAM20A | | | | | | | | | 0.5 | | | |
| FAM20C | | | | | | | | | | | | 21 |
| FAM210A | | | | | | | | 18 | | | | |
| FAM210B | | | 6 | | | | | | | 7.5 | | |
| FAM213A | | | | | | | | 21 | | | | |
| FAM214A | | | | | | | | 22 | 23 | | | 22 |
| FAM216A | | | | | | | 6 | | | 7 | | |
| FAM219A | | | | | | | | 19 | | | | |
| FAM219B | | | 22.5 | | | | | 21 | | | | |
| FAM21A | | | | | | 10 | | 22 | | | | |
| FAM26E | | | | | | | | 21 | | | | |
| FAM3C | | | | | | | | | 8 | 11.5 | | |
| FAM40B | | | | | | | | | | | 9 | |
| FAM45A | 7 | | | | | | | | | | | |
| FAM46A | | | | | | | | | 11.5 | | | |
| FAM47E | | | | | | | | | | 8.5 | | |
| FAM48A | 23 | | | | | | | | | | | |
| FAM49A | | | | | | | | 14 | | | | |
| FAM49B | | | | | | | 9 | | | | | |
| FAM50A | | 7 | | | | | | | | | | |
| FAM53B | 21 | | | | | | | | | | | |
| FAM54B | | | | | | | | 0 | 4 | | | |
| FAM55B | | | | | | | | 22 | | | | |
| FAM55C | | | | | | 1.833 | | | | | | |
| FAM55D | | | | | | | | | 23 | | | |
| FAM57A | | | | | | | | 13.5 | | | | |
| FAM5C | 18 | | | | | | | | | | | |
| FAM63B | 15 | | | | | | | 21 | | | | |
| FAM65A | | 7 | 8 | | | | | | | 7 | | |
| FAM65B | | | | | | | | | | 12 | | |
| FAM69A | | | | | | | | 5 | | | | |
| FAM73A | | | | | | | | | | | | 9 |
| FAM73B | | | | | | | | 9 | | | | |
| FAM76A | | | | | | | | 11 | | 9.5 | | |
| FAM78B | 0 | | | | | | | | | | | |
| FAM82A1 | | | | | | | | 0 | | | | |
| FAM82A2 | | | | | | | | | | | 2.833 | |
| FAM83A | | | 8 | | | | | | | | | |
| FAM83D | | | | | | | | | | 16 | | |
| FAM83F | | | | | | 7 | | | | | | |
| FAM83H | | | | 21 | | | | | | | | |
| FAM84A | | | | | | 0 | | | | | | |
| FAM84B | | | 23 | | | | | | | | | |
| FAM89A | | | | | | | | | 13 | | | |
| FAM96A | | | | | | | | | | 13 | | |
| FAM96B | | | | | | | | 17 | | | | |
| FANCB | | | | | | | | 12 | | | | |
| FAP | 23 | 23 | | | | 22 | | | 0 | 0.5 | 23 | 0 |
| FAR1 | | | | | | | | | | 17 | | |
| FARP1 | | | | | | | | 21 | | | | |
| FARP2 | | | | | | | | | 21 | | | |
| FARSB | | 7 | | | | | | | | | | |
| FAS | | | | | | | | 23.5 | | | | |
| FASN | | | | | | | | | | | | 10 |
| FASTK | | | | | | | | | 6 | | | |
| FASTKD2 | | | 21 | | | 0 | | | | | | |
| FAT1 | | | | | | | | 2 | | | | |
| FAT3 | | | | | | | | 7 | | | | |
| FBLIM1 | | | | | | | | 23 | | | | |
| FBLN2 | | | | | 5.5 | | | 0 | | | | |
| FBLN5 | | | | | | | | | 22 | | | |
| FBN1 | | 15 | 15.5 | | 14.5 | 16 | | | | 12 | 15 | 17 |
| FBN2 | 18 | | 21 | | | | | 15 | 19 | | | |
| FBRSL1 | | | | | | 4 | | | | | | |
| FBXL13 | | | 17 | | | | | | | | | |
| FBXL18 | | | | | | | | 20.5 | 17 | | | |
| FBXL2 | | | | | | 17 | | | | | | |
| FBXL20 | | | | | | | | 12 | | | | |
| FBXL21 | 16 | | | | | 8 | | | 0 | | | |
| FBXL3 | | | | | | | | | | | 1 | |
| FBXL4 | | | | | | | | | | 18 | | |
| FBXO21 | | | | | 2 | 11 | | | | | | |
| FBXO22 | | | | | | | | 4 | 5 | | | |
| FBXO25 | | | | | | | | | 23 | 8 | | |
| FBXO3 | | | 22 | | | 7 | 15.5 | | 11 | | | |
| FBXO30 | | | | | | | | | | 11 | | |
| FBXO31 | | | | | 4 | | | | | | | |
| FBXO32 | | | 0 | | | | | | 21 | | | |
| FBXO33 | | | | | 7 | | | | | | | |
| FBXO34 | | | | | | | | | | 10 | | |
| FBXO36 | | 11 | 13 | | | | | | | | | 13 |
| FBXO40 | | | 20.5 | | | | | | | | | |
| FBXO44 | | | | | | | | | | | 22 | |
| FBXO45 | | | | | | | | | | 4 | 22 | |
| FBXO6 | | | | | | | | | | 15.5 | 2.5 | 22 |
| FBXO8 | 16 | | 6 | | | | | 17 | | | | |
| FBXW2 | | | | | | | | 19 | 20 | | | |
| FBXW8 | 19 | | | | | | | | | 15 | | |
| FBXW9 | | | | | | 2.5 | | | 4.5 | | | |
| FCAMR | | | | | | | | 13 | | | | |
| FCER1G | | | | 21 | | | | | 12 | | | |
| FCGR2B | | | | | | | | | 22 | | | |
| FCGRT | | | | 5 | | 6 | 6 | | | | | |
| FCHSD1 | | | | | | | | 10 | 14 | 12 | | |
| FCHSD2 | | | 5 | | | | | | | | | |
| FCRL1 | | | 6 | | | | | | 22.5 | | | |
| FDFT1 | | | | | | | | | | | 10 | |
| FDPS | | | | | | | | | 23 | 9 | | |
| FDX1 | | | | | | | | | 20 | | | |
| FDXACB1 | | | | 12 | | | | | | | | |
| FEM1A | 14 | | 12 | | | 12.5 | | 16 | | | | |
| FEM1C | | | | | | | | | | 3 | | |
| FER | | | | | | 16 | | | 22 | | | |
| FERMT1 | | | | | | | | | 15 | | | 22 |
| FERMT2 | | | | | | | | | 23 | | | |
| FES | | | | | | | | | | 12.5 | | |
| FFAR2 | | | | | 16 | | | | | 14 | | |
| FGA | 7 | | | | | | | | | 10 | | |
| FGB | 20 | | | | | | | | | | | |
| FGD4 | | | | | | | | | 5.5 | | | |
| FGF1 | | | | | | | | | | 2 | | |
| FGF10 | | | | | | | | 5.5 | | | | |
| FGF11 | | | | | | | | | | | 1 | |
| FGF13 | | 20 | 20 | | | | | 20 | 22.5 | | | |
| FGF16 | | | | | | 1 | | | | | | |
| FGF18 | | | | | | | | 14 | 21 | | | |
| FGF9 | | | | | | | | | 0 | | | |
| FGFBP1 | | | | | | | | | 21 | | | |
| FGFR1 | | | | | | | | | 21.5 | | | |
| FGFR1OP | | | | | | | | | | 11.5 | | |
| FGFR2 | | | | | | | | | 22 | | | |
| FGFR3 | | | | | | | | 22.5 | | | | |
| FGFR4 | | | | | | 21 | | | | | | |
| FGFRL1 | | | | | | | | | 22 | | | |
| FGG | | | | | | | | | | 14 | | |
| FGGY | | | | | | | | 17 | 21 | | | |
| FH | | | | | | | | 22 | 3.5 | | | |
| FHAD1 | | | | | | | | 6 | 0 | 7 | | |
| FHDC1 | | | | | 23 | | | | | | | 18 |
| FHIT | | | | 21 | | | | | | | | |
| FHL1 | | | | | | | | | 7 | | | |
| FHL3 | | | | | | | | | | 14 | | |
| FHOD3 | | | | | | | | 0 | | | | |
| FIBIN | | | | | | | | | | | 0 | |
| FIBP | | | | | | | | | 22 | | 2 | |
| FIGF | | | | | | 12 | | | | | | |
| FILIP1 | | | | | | | | | 5 | | | |
| FILIP1L | | | | | | | | 23 | | | | |
| FIP1L1 | | | | | | | 22 | 22 | 23.5 | | | |
| FIS1 | | | | | | | | | 2 | | | |
| FITM1 | | | | | | | | | 9 | | 23 | |
| FITM2 | | | 23 | | | | | | | | | 21 |
| FKBP10 | 20 | | | | | | | | 3.5 | | | |
| FKBP1A | | | | | | | | | 3 | | | |
| FKBP1B | | | | | | | | | | 21 | | 19 |
| FKBP3 | | | | | | 20 | | | 22.5 | 23 | 20.5 | |
| FKBP4 | | | | | | 18 | | | 18 | | | |
| FKBP5 | | | | | | 14 | | | | 14 | | |
| FKBP7 | | | | | | | | | | 5 | | |
| FKBP8 | | | | | | | | | | | 0 | |
| FKTN | | | | 20.5 | | | | 19 | | | | |
| FLCN | | | | | | | | | | 8 | | |
| FLNB | | | | | | | | | 21 | | | |
| FLOT1 | 10 | | | | | | | | | | | |
| FLOT2 | | | | | | | | | | 0.5 | | |
| FLRT1 | | | | | | | | | | 6 | | |
| FLRT3 | 20 | | | | | | | | | | | |
| FLT1 | | | | | 21 | | | | 22.5 | | | |
| FLT4 | | | | | | | | 23 | | | | |
| FLVCR1 | | 6 | | | | | | | | 7 | | |
| FLVCR2 | | | | | | | | 18 | | | | |
| FLYWCH1 | | | | | | | | | | 11 | | |
| FMN2 | | | | | | | | 22 | 3 | | | |
| FMNL1 | | | | | | | | 23 | | | | |
| FMNL2 | | 6 | | | | | | | 0 | | | |
| FMNL3 | | | | | | | | | 9 | | | |
| FMO1 | 10 | | 5.5 | | | 12 | | | 15 | 12 | 11 | |
| FMO2 | | | | | | 13 | | | | | | |
| FMO3 | | | 15.5 | | | 20 | | | | | | |
| FMO4 | | | | | 23 | | 22.5 | | | | | |
| FMO5 | | | | | | | | | 19 | | | 6 |
| FMOD | | | | | | | | 23 | | | | |
| FMR1 | | | | | | | 6 | | 16 | | | |
| FN1 | | | | | | | | 21 | | | | |
| FN3K | | | 6 | | | 23 | | | | | 3 | |
| FN3KRP | | | 2 | | | 23.5 | | 3 | 4 | | | |
| FNBP1 | | | | | | 20 | | | | | | |
| FNDC3A | | 17 | | | | | | | | | | |
| FNDC3B | | | | | | | | | 23.5 | | | |
| FNDC4 | | | | | | 10 | | | 12.5 | 12 | 8.5 | |
| FNIP1 | | | 6 | | | | | | 8 | | | |
| FNIP2 | 7 | | 8 | | | | | | | 3.5 | | |
| FOLH1 | | | | | | | | 16 | | | 0 | |
| FOLR1 | | | | | | | | 23 | 22 | | | |
| FOLR2 | 8 | | 8 | | | 16 | | 9 | 11.5 | 18 | | 10 |
| FOPNL | | | | | | | | | 10 | | | |
| FOSL2 | | | 16 | | | | | | 0.5 | | | |
| FOXA2 | | | 8 | | | | | | 11.5 | | | |
| FOXA3 | | | 22 | | | 1.5 | 7 | | | 22 | | |
| FOXC1 | 21 | | | | | | | 21 | 23 | | | |
| FOXJ2 | | | | | | | | 19.5 | 21.5 | | | |
| FOXK1 | | 9 | | | | | | 7 | | | | |
| FOXK2 | | | | | | | | | 3.5 | | | |
| FOXN2 | | | | | | | | 7 | | | | |
| FOXN3 | | | | | | | | | | | 20.5 | |
| FOXO1 | | | | | | | | 1 | 0 | | | |
| FOXO3 | | | 11 | | | 3.5 | | | | | | |
| FOXP1 | 11 | 11 | 11 | 13 | 11 | 12 | | 12 | 13 | 10 | | 11.5 |
| FOXP2 | | | | 21 | | | | | | | | |
| FOXRED2 | | | | | | | | | | 4 | | 20 |
| FOXS1 | | | | | | | | | 3 | | | |
| FPGS | | | | | | | | | 22 | | | |
| FPGT | 1 | | | | | | | | | 17 | | |
| FRA10AC1 | 0 | | | | | | | | | | | |
| FREM1 | | | | | | 18 | | | 17 | | | |
| FRG2 | | | 3 | | | | | | 23.5 | 20 | | |
| FRMD4A | | | | | | | | | 21 | | | |
| FRMD4B | | | | | | 19 | | | | | | |
| FRMD5 | 20 | | 1 | | | | | | 22 | 22 | | |
| FRMPD1 | | | | | | | | 12 | | | | |
| FRRS1 | | | | | | | | | 20 | | | |
| FRY | | | 20 | | | | | | | | | |
| FRYL | | | | | | | 9 | 11.5 | 23.5 | | | |
| FRZB | | | | | | | | | 16.5 | | | |
| FSCN1 | | 6 | | | | 7 | | | | | | |
| FSIP1 | 12 | | | | | | | | 21 | | | |
| FST | | | | | | | | | 17 | | | |
| FSTL3 | | | | | | | | | | 12 | | |
| FTH1 | | | | | | | | 19 | 19 | | | |
| FTSJ1 | | | | | | | 8 | | | | | |
| FTSJD1 | | | | | | | | | 0 | | | |
| FUBP1 | | | | | | | | 22 | | | | |
| FUBP3 | | | | | | | | | | 11 | | |
| FUCA2 | | | | | | | | | 22 | | | |
| FUK | | | | | | | | | | 0 | | |
| FUNDC1 | | | 10 | | | | | | | | | |
| FURIN | | 15 | 18.5 | 13 | | 16 | | | | 11 | | |
| FUS | | | | | | | | 22 | 23 | | | |
| FUT2 | 23 | 22 | 1.5 | | | | | 22 | 23 | 23 | | |
| FUT8 | | | | | | | | 23 | 22 | | | 21.5 |
| FV1 | 8.5 | | 10.5 | | | | | | | | | |
| FXR1 | | 7 | | | 17 | | | 9 | 10 | 9 | | 9.5 |
| FXYD1 | | | | 22.5 | | | | 23 | 18.5 | | | |
| FXYD4 | | | | | | | | 18 | | | 21 | 22 |
| FXYD5 | | | | | | | | | 9 | | | |
| FYB | | | | | | | | | | | 9.5 | |
| FYCO1 | | | | | | | | 22 | 23 | 14.5 | | |
| FYN | 15 | | | | | | | | | | | |
| FZD1 | | | | | | | | | | 22 | | |
| FZD2 | | | 9 | | | | | | | | | |
| FZD3 | | | | | | | | | 23 | 8 | | |
| FZD4 | | 1 | | | | | | 0.5 | 22.5 | | | |
| FZD7 | 20 | | | | | | | 22 | | | | |
| FZD9 | | | 23 | | | | | 20 | 22 | 15 | | 22 |
| G0S2 | | | | | 4 | 12 | | | | | | |
| G12 | | 7 | 8 | | | | | | | 11 | | |
| G13 | | | | | | | | | 23 | | | |
| G3BP2 | | | 12 | | | | | | | | | 13 |
| G630090E17RIK | | | | | | | | | 21 | | | |
| G6PC | | | | | | | | | 22 | | | |
| G6PD | | | | 6 | | 1 | | | | 4 | | 23 |
| G6PD2 | | | | | | | | | | 17 | | |
| GAA | | | | 0 | | | | | | | | |
| GAB1 | | | | | | | | | | 9 | | |
| GAB2 | | | | | | | | 11 | | | | |
| GABARAPL1 | | | | | | 8.5 | | 15 | | 10 | | |
| GABBR1 | | | | | | | | | | | 2 | |
| GABPA | | | | 0 | | | | | | 9 | | |
| GABPB1 | | | | | | | | | 23 | 12 | | |
| GABPB2 | | | | | | 23 | | | | | | 0 |
| GABRA3 | | | 13 | | | | | | | | | |
| GABRB1 | | | | | | 16 | | 16 | | | | |
| GABRB2 | | | | | | | | | 2 | | | |
| GABRB3 | | | | | | | | | 23.5 | | | |
| GABRE | | | | | | | | | 19 | | | |
| GABRQ | 19.5 | | | | | 10 | | 0 | | | | |
| GABRR2 | | | | | | | | | 19 | 17 | | |
| GADD45G | | | | | | | | | 10 | 5 | | |
| GAK | | | | 7 | 7 | | 7 | | | 22 | | |
| GALE | | | | | | | | | | 9 | | 8 |
| GALM | | 9 | 10 | | | 22 | | | | | | |
| GAINS | 9 | | | 14.5 | | | 15 | 14 | | | | |
| GALNT10 | | | | | | 18 | | | 23.5 | | | |
| GALNT11 | | | | | | | | 14 | 1 | | | |
| GALNT14 | | | | | | | | 17 | | | 0 | |
| GALNT3 | | | | | | | 7 | | | | | |
| GALNT7 | | 9 | 12 | | | | | | | 9 | | |
| GALNTL1 | | | | | | | | | | 10 | | |
| GALNTL4 | | | | | | | | | | | | 10 |
| GALT | | | | | | | | | 8.5 | | | |
| GAMT | | | 11.5 | | | | | | | | | 10.5 |
| GAP43 | | | | | | | | | | | | 8 |
| GARNL3 | 2 | 0.5 | 1 | | | 23 | | | | | 0 | |
| GARS | | | | 12 | | | | | | 13 | | |
| GART | | | | | | | | | 14.5 | | | |
| GAS2 | | | | 0 | | | | | | 7 | 9 | |
| GAS2L3 | | | | | | | | | 14 | 14 | | |
| GAS6 | | | | | | | | 21 | 22 | | | |
| GAS7 | | | | | | | | 8 | | | | |
| GATA4 | | 8 | | | | | | | 10 | | | |
| GATA5 | | | | | | | 6 | | | | | |
| GATA6 | | | | | | | | | | 12 | | |
| GATAD1 | | | | | | | | 22 | 23.5 | | | |
| GATAD2A | | | 22 | | | | | | | | | |
| GATAD2B | | | | | | | | | | | | 12 |
| GATC | | | | | | | | | 12 | | | |
| GATM | | 18 | 18.5 | 17 | 18 | | 17 | | | | | 5.5 |
| GATSL2 | | | | 22 | | | | | | | | |
| GATSL3 | 15 | | | | | | | | | | 0 | |
| GBA2 | | | | | 20 | 16 | | | | 15 | | 18 |
| GBAS | | 6 | 19 | | | | | | 15 | 5 | | |
| GBE1 | | | | | 11.5 | | 9 | | | 13 | | |
| GBF1 | | | 11 | | | | | | | 3 | | |
| GBP11 | | | | | | | | | | 10 | | |
| GBP4 | | | | | | | | 2 | | | | |
| GBP5 | | | | | | | 1 | | | | | |
| GBP8 | | | | | | | | | | | | 5 |
| GCA | 8 | | | | | | | | | 4.5 | | |
| GCDH | | | | | | | | | | 6 | | |
| GCGR | | | 7 | | | | | | | | | |
| GCH1 | | | | | | | | 3 | | | | |
| GCK | | | | | | | | 11 | | | | |
| GCKR | | | 5 | | | | | | 18 | | | 22.5 |
| GCLC | | | | | | | | | 0.5 | | | |
| GCLM | | | | | | | | | 9 | | | |
| GCNT1 | | | | 15 | | | | | | | | |
| GCNT2 | | | 22.5 | | | 6.5 | | | | | | |
| GCSH | | | | | | | | | | 14 | | |
| GDA | | | | 23 | | | | 20.5 | | | | |
| GDE1 | | | | | | | | 23 | | | | |
| GDF10 | | 7 | | | | | | | | | | |
| GDI2 | | | | | | | 8 | | | | | |
| GDNF | | | | | | | | | | 11 | | |
| GDPD1 | | | | | | | | | 17 | | | |
| GDPD2 | | | | | | | | | | | 0.5 | |
| GDPD5 | | | | 6 | | | | | | | | |
| GEMIN6 | | 14 | | | | | | | | 11 | | |
| GEMIN7 | 21 | | | | | | | 22 | | | | |
| GFM1 | 11 | 13.5 | | | | 16 | | 13 | | 12 | | |
| GFM2 | | 9 | 10 | | | | | | | 8 | | |
| GFOD1 | | | | | | | | | 11 | | | |
| GFOD2 | | | | 21.5 | | | | 19 | | | | |
| GFPT1 | | | | | | | | | 22 | | | |
| GFRA1 | | | | | | 10 | | | | | | |
| GFRA2 | | | | | | | | | | 12 | | |
| GGA2 | | | | | | | | 13 | | | | |
| GGCT | | | | | | 23 | | 3 | | | | |
| GGCX | | | | | | | | | 5 | | | |
| GGPS1 | | | | | | | | | 21 | | | |
| GGT6 | | | | | | | | | | | 23 | |
| GH | | | | | | | | 9 | | | | |
| GHDC | 7.5 | | | | | | | | | 11 | | |
| GHR | 9 | 8 | 8 | | | | | | 11 | 10 | | 8 |
| GI1 | | | 6 | | | | | | | | 22.5 | 21 |
| GI3 | | | | | | | | | 22 | | | |
| GIMAP1- | | | | | | | | 8 | | | | |
| GIMAP4 | 10 | | | | | | | | 19 | | | |
| GIMAP5 | | | | | | | | | | | | 10.5 |
| GIN1 | | 17 | | | | 15 | | 20.5 | 14 | 17 | 19 | |
| GIPC1 | | 12 | | | | 12 | | | | 11 | | 13 |
| GIPC3 | | | | | | | | 5 | | | 3 | |
| GIT1 | | | | | | | | | 2 | | | |
| GIT2 | | | | | | | | 11 | 21 | 3.5 | | |
| GJA1 | 7 | | | | | | | | 7 | 0 | | |
| GJA5 | | | | | | | | 22 | 22.5 | | | |
| GJB1 | | | | | | | | | 20 | | | |
| GJB2 | | | | | | | | | 23 | | | |
| GJB6 | | | | | | | | | 22 | | | |
| GJC1 | | | | | | | | | | 3 | | |
| GJC2 | | | | | | | | 8 | 23 | | | 23 |
| GJC3 | | | | | | | | | 1 | | | |
| GK | | | | | | | | | 0 | | | |
| GK5 | 19 | 23 | | | | | | 21 | 22.5 | | | |
| GKAP1 | | | 0 | | | | | | 0 | | | |
| GKN3 | | | | | | | | | 21 | | | |
| GLB1 | 20 | | | | | | | | | | | |
| GLB1L2 | 21 | | | | | | | 22 | | 6 | | |
| GLB1L3 | 7 | 6.5 | 8 | 6 | | | | | | 5.5 | | |
| GLDC | | 18 | 19 | | | | | | 23 | | | |
| GLI2 | | | | | | | | | | | | 4.167 |
| GLIPR2 | | | | | | | | 19.5 | | | | |
| GLIS2 | | | | | | | 19 | 18.5 | | | | |
| GLIS3 | | | | | | | | 8 | | | | |
| GLRX | | | | | | | | | 16 | 12 | | |
| GLRX2 | | | 2 | | | | | 22 | 23 | | | |
| GLRX5 | | | 19.5 | | | 20 | | | | | | |
| GLT1D1 | | | | | | 2 | | | | | | |
| GLT25D1 | | | | | 21 | | 0 | | | | | |
| GLT28D2 | | | | | | 16 | | | | | | |
| GLT8D2 | | | | | | | | | | | 6 | |
| GLTPD2 | | | 22 | | | | | | | | | |
| GLTSCR2 | | | | 21 | | | 19 | | | | | |
| GLUL | 10 | | | | | | | | | | | |
| GLYCTK | | | | | | 19 | | | | | | |
| GM10025 | | | | | 19 | | | 11.5 | | 11 | | |
| GM10032 | | | | | | | | | | 9.5 | | |
| GM10038 | 20 | | | | | | | 7 | | | | |
| GM10167 | | | | | | | | | | 11.5 | | |
| GM10250 | | | | | | | | 17 | | | | 16 |
| GM10287 | | | 20 | | | | | | | | | |
| GM10305 | | | 11 | | 9 | | | | | | | |
| GM10311 | | | | 2 | | | 5.5 | | | | | |
| GM10319 | 9 | | | | | | | 10 | | | | |
| GM10357 | | | | 19 | | | 17 | | | | | |
| GM10565 | 17 | | 21.5 | | | | | | | | | 22 |
| GM10617 | | | | | 22.5 | | | | 19 | | | |
| GM10629 | | 14 | | | | | | | | | | |
| GM10644 | | | 2.5 | | | | | 7 | 7 | | | 8 |
| GM10647 | | | | | | | | | | 16 | | |
| GM10664 | | | | | | 13.5 | | | | | | |
| GM10722 | | | | | | | | | | | | 8 |
| GM10737 | 20 | | | | | | | | | | | |
| GM10762 | 7 | | | | | | | | | | | |
| GM10766 | | | | | | | | | | 10.5 | | |
| GM10768 | | 6 | | | | | | | 0 | | | |
| GM10782 | | 15 | | | | | | | | | | |
| GM10787 | | | | | | 17 | | | 18 | | | |
| GM10800 | | | | | | | | | | 14 | | |
| GM10801 | | | | | | | | | | 7.5 | | |
| GM10804 | 18 | | | | | | | | | | | |
| GM10845 | | | | | 19 | | | | 14 | 12.5 | | |
| GM10863 | | | 0 | | | | | | 12 | | | |
| GM11428 | | | | | | | | | 0 | | | |
| GM11451 | | | | 18 | 16 | | | | | | | |
| GM11567 | | | | | | | | | 19 | | | |
| GM11709 | 15 | | | | | | | | | | | |
| GM11942 | | | | | | | | | 22 | | | |
| GM11971 | | | | | | | | 23.5 | 10 | | | |
| GM12026 | | | | | | | | | 0 | | | |
| GM12060 | 23 | 0.5 | 2 | | | | | 21 | | | 23 | 23 |
| GM12181 | | | | | | | | | | 8.5 | | |
| GM12201 | | | | | | | | | 17 | | | |
| GM12202 | | | 22.5 | | | | | | | | | |
| GM12222 | 8 | 8 | 7.5 | | | 10 | | 11 | 11.5 | 10 | 4.5 | 8.5 |
| GM12247 | 10 | | | | | 21.5 | | | | | | |
| GM12258 | | | | | | 9 | | | 18 | | 10 | |
| GM12642 | | | | | | | | | | | | 10 |
| GM12689 | | 8 | | | | | | 9 | | | | |
| GM12696 | 22 | | | | | | | | | | | |
| GM12699 | | 6.5 | 7 | | | | | | 21 | | | |
| GM12794 | 6 | | 7 | | | | 6 | | | | | |
| GM12902 | 8.5 | | | | | | | 11.5 | | | | |
| GM13152 | | 8.5 | 8 | | | | | | | | | |
| GM13359 | | | | | | | 6 | | | | | |
| GM13375 | | | | | | | | 9 | | | | |
| GM13397 | | | | | | | | | 14 | | | |
| GM13436 | | | | 21.5 | | 23 | 19.5 | | 20 | | | |
| GM13440 | | | | | | | | | 23.5 | | | 21 |
| GM13487 | | 8 | 8 | | | | | | | | | |
| GM14006 | 11 | 12 | 13 | 12 | 13 | 12 | 12 | 11 | 12 | 11 | 10.5 | 12 |
| GM14150 | 14 | 14 | 13 | 13 | 15 | 13 | 12.5 | 14 | 15 | 13 | 11 | 14 |
| GM14296 | 10 | 11 | 11 | 11 | 12 | 10 | 12 | 11 | 11 | 11 | 10 | |
| GM14403 | | 21.5 | 21 | | 22 | | | | 19 | | | |
| GM14639 | | | | | | | | | | 7 | | |
| GM14830 | | | 19 | | | | | 20 | | | | |
| GM14964 | | | | | | | | | 0 | | | |
| GM15024 | | | | | | | | 11 | | | | |
| GM15401 | | 4 | | 23 | | | | 4 | 21 | | | 6 |
| GM15440 | 21 | 23 | 22 | | | | | | | | | 22.5 |
| GM15441 | | | | | 1 | 21 | 1 | 2 | | 5 | 23 | |
| GM15470 | | | | | | | | | | 10 | | |
| GM15514 | | | | | | 23 | | | 23 | | | |
| GM15542 | | | | | | | | 22 | | | | |
| GM15770 | 11 | | | | | | | | | 2.5 | | |
| GM15998 | | | | | | | | 16 | | | | |
| GM16042 | | 1 | | 23 | | 16 | | | | | | |
| GM16223 | | | | | | | | 19 | 20 | | | |
| GM16314 | | | | | | | | 12.5 | 17 | | | |
| GM16373 | | 15 | 16 | | | 16 | | 13 | 16 | 14 | 13 | |
| GM16425 | | | 23 | | | | | 22 | | | | |
| GM16432 | | | | | | | | 21 | 1.5 | | | |
| GM16493 | 7 | | | | | | | | | | | |
| GM16516 | | | | | | | | | | 10.5 | | |
| GM17252 | | 9 | 9.5 | | | 8.5 | | | | | | |
| GM17383 | | | 23 | | | 9.5 | | | | 17 | 10 | |
| GM17484 | | | | | | | | | | | 22 | |
| GM17535 | 21 | | | | | | | | | 11 | | |
| GM19840 | | | | | | | | | 21 | | | |
| GM20396 | 5 | 4 | 4 | | | 5 | | | 22.5 | 6 | | |
| GM20695 | | | | | | | | 9 | | | | 12 |
| GM266 | | 23 | | 22 | | 23 | 21 | 20.5 | | 21 | | |
| GM2A | | | | | | | | 18.5 | | | | |
| GM3571 | | | | 21 | | 1 | 21 | 20 | | | 23 | |
| GM4759 | | | | 21 | | 1 | 21 | 22 | | | | |
| GM4788 | | | | | | | | | 11 | | | |
| GM4875 | 0 | | | | | | | | | | | |
| GM4876 | | | | | | | | | | 4 | | |
| GM4952 | | | | | | | | 0.5 | | 21 | | |
| GM4956 | 23 | 22 | | | | | 18 | | | | | |
| GM498 | | 5.5 | 21 | | | | | | 23 | | | |
| GM4989 | | | | | | | | | | 4.5 | | |
| GM5393 | | | 23 | | | | | | | | | |
| GM5405 | | | | | 22 | | | | 22 | | | |
| GM5406 | 21.5 | | | | | | | | | | | |
| GM5535 | | | 10 | | | | | | | | | |
| GM5546 | | | | | 22 | 11.5 | | 4 | | | | |
| GM5548 | | | | | | 18.5 | | | 16.5 | | | |
| GM609 | | | 14 | | | | | | | | | |
| GM6116 | | | | | | | | | | 16.5 | | |
| GM6180 | 21.5 | | | | | | | | | | | |
| GM6181 | | | | 22.5 | | | | | | | | |
| GM6245 | | | | | | | | | | | 22 | |
| GM6471 | | 3 | | | | | | | 5.5 | 9 | 3.5 | |
| GM6640 | 9 | | | | | | | | | | | |
| GM6728 | | | 6 | | | 3 | | | | | 2 | |
| GM6762 | 21 | | | | | | | 2 | | | 2 | |
| GM6829 | | | | | 8 | | | | | | | |
| GM7091 | 13 | | | | | | | 3 | | 14 | | 12.5 |
| GM7108 | | | | | | | | | 23 | | | |
| GM7278 | | | | | | | | | 23 | | | |
| GM7293 | | | 23 | | 23 | 2 | 19 | 23 | | | | 22 |
| GM7820 | | | | | 23 | | | | | | | |
| GM7887 | 5 | | 12 | | | 8.5 | | 7 | 1 | 8 | | 10 |
| GM8098 | 10 | | | | | | | | 9.5 | | | |
| GM826 | | | | | | | | 22 | | | | |
| GM839 | | | | | | | | | | 16 | | |
| GM8682 | | | | | | | | 22 | | | | |
| GM884 | | | | | | | | 16 | 18 | 16 | | |
| GM9434 | | | | | | | | | | 9.5 | | |
| GM9750 | | 14 | 15.5 | | | | | | | 7.5 | | |
| GM9930 | | | | | | | | | 9 | | | |
| GM9945 | 18 | | | | | | | | | | | |
| GM9947 | | | | | | | | | 5.5 | | | |
| GM9949 | | | | 13.5 | 10 | 13 | | | | | | |
| GM9951 | | | | | | | | | 4 | | | 7 |
| GM9956 | | | | | 20 | | | | | | | |
| GM9958 | 6 | | | | | | | 15 | | | | |
| GM9968 | | | | 8 | | | 9 | | | | | 9 |
| GM9974 | 9 | | | | | | | | | 13 | | |
| GM9982 | | | | | 8 | | | | | | | |
| GM9996 | | | | | | 4 | | | | | | |
| GMCL1 | | | | | | | 12 | | | | | |
| GMEB1 | | | | | | | | | | 10.5 | | |
| GMEB2 | | | | | | | | | | | | 6.5 |
| GMFB | | | | | 22 | | | | | | | |
| GMIP | | | | | | 12 | | | | | | 14 |
| GMNC | 22.5 | | | | | 23.5 | | 0 | | | | 22 |
| GMPR | | | | | | 23 | | | | 2 | | 23 |
| GMPR2 | | 17 | | | | | | | | | | |
| GNB1 | | | | | | | | | 2.5 | | | |
| GNB2 | | | | | | | | | | 7 | | |
| GNB2L1 | | | | | | | | | 22 | 21.5 | | |
| GNE | | | | | | | | | 6.5 | | | |
| GNG12 | 8 | 10 | 9 | | | | | | 11 | | | |
| GNG2 | | | | | 10.5 | | | | | | | |
| GNGT2 | | | | | 7 | | | | 23 | | | |
| GNL1 | | | | | | | | | 15.5 | | | |
| GNL2 | 20 | | 21.5 | | | | | | | | | |
| GNPAT | | | | | | 0 | | | 4.5 | | | |
| GNPDA2 | | | | | | | | 7 | 7.5 | | | |
| GNPTAB | | | | | | | | | | 13 | | |
| GNRH1 | | | | | | | | 17 | | | | |
| GNS | | | | | | 8 | | | | 11 | | |
| GO1 | | | | | 2 | | | 22 | | | 3.5 | |
| GOLGA4 | | | 22 | | | | | | | 1.5 | | |
| GOLIM4 | | | 22.5 | | | | | | 0 | | | |
| GOLPH3 | | | | | | 5.5 | | | 1 | | | |
| GORASP1 | | | 17 | | | | | | 20 | | | 19.5 |
| GOT1 | | | | | 6.5 | | | | 4.5 | | | |
| GP49A | | | | | | | | | 23 | | | |
| GPAA1 | | | | | | | | 8.5 | | | | |
| GPAM | 16 | 23 | | | | | | | | | | |
| GPATCH1 | 10 | | | | | | | | | | | |
| GPBP1L1 | | | 23 | | | | 4 | | 22 | | | |
| GPC4 | | | | | | | | | | | | 8.5 |
| GPCPD1 | | | | | | | | | | | 3.5 | |
| GPD1 | | | | | | | | | 23 | | | 19 |
| GPD1L | 21 | | | 19 | 18 | | 17 | | | | | |
| GPD2 | | | | | | 18 | | 16.5 | | 13.5 | | |
| GPHN | | | | 14 | 13 | | 13 | | | | | |
| GPI | | | | | | 19 | | | | 15 | | |
| GPIHBP1 | | | | | | | 8 | 21 | 20 | | | |
| GPM6B | | 16 | 5.5 | | | | | | | 15 | | |
| GPN2 | | | | | | | | | 20 | | | |
| GPR110 | | | | | | | | 17 | | 12.5 | | |
| GPR116 | | 20 | 18 | 19.5 | | | | 22 | | | | |
| GPR123 | | | | | | | | | | 13 | | |
| GPR124 | | | | | | | | | 1 | | | |
| GPR125 | | | | | | 20 | | | | | | |
| GPR133 | 12 | 12 | | | | | | | | | | |
| GPR137 | | | | | | | 19 | 10 | | 18.5 | | |
| GPR137C | | | 21.5 | | | | | | | 8 | | |
| GPR146 | | | | | | 0 | | | 7 | | | |
| GPR155 | | | | | | | | 22 | | | | |
| GPR157 | | | | | | 16 | | | | | | |
| GPR158 | | | | | | | | | | 1 | | |
| GPR160 | | | | | | | | | | | | 19.5 |
| GPR17 | | | | | | | | | | 17 | | |
| GPR182 | | | | | | | | | 23.5 | | | |
| GPR19 | | | | | | | | | 12 | | | |
| GPR22 | | | | | | | | | | | 20 | |
| GPR37 | | | | | | | | | 22 | | | |
| GPR4 | | | | | | | | 11 | | | | |
| GPR55 | | | | | | | | 8 | | | | 10 |
| GPR56 | | | | | | 3 | | | | 14 | | |
| GPR63 | | | | | | 19.5 | | | | | | |
| GPR64 | | 6 | 6.5 | | | | | | | 7 | | |
| GPR75 | | | | | | | | 20 | | | | |
| GPR75-ASB3 | | | | | 7 | | 7 | | | | | |
| GPR88 | | | | | | | | 21 | | | | |
| GPR97 | | | | | | | 4 | | | | | |
| GPRC5B | | | | | | | | | 17 | 14.5 | | |
| GPRC5C | | | | 18.5 | | | | 18 | | 5.5 | | |
| GPRC6A | | | | | | 12 | | | | | | |
| GPRIN3 | | | | | | | | | 23 | | | |
| GPSM1 | | | | | | | | | 10 | 9 | 9 | |
| GPSM2 | | | | | | | | | | 13 | | |
| GPT | | | | 15.5 | | 5.5 | | 14 | 14 | | | |
| GPT2 | | | | | | | | | 6 | | | |
| GPX1 | | | | | | | | | | 21 | | |
| GPX3 | | | 11 | | | | | | 9 | | | |
| GPX4 | | | | | | | | | 19 | | | |
| GPX8 | | | | | | | | | 1 | | | |
| GRAMD1A | | | | | 20.5 | | | | | | | |
| GRAMD1B | | | | | | | | | 21.5 | | | |
| GRAMD2 | | | | | | | | 0 | | 20 | | |
| GRAMD3 | | | | | | | 8 | | | | | |
| GRAMD4 | | | | | | | | | 14.5 | | | |
| GRASP | | | | | | | | | 5 | | | |
| GRB10 | | | | | | | | | | 11 | | |
| GRB14 | | | 20 | | | | | | 20.5 | | 23 | |
| GRB7 | | | 9 | | | | | | | | | |
| GREM2 | | | | | | | | | | 15 | 2 | |
| GRHPR | | | | | | | 8 | 11 | | | | |
| GRI | | | 18.5 | | | | | | | 4 | | |
| GRIA1 | | | | | | | | 22 | | | | |
| GRIA3 | 21 | | | | | | | 22.5 | 17 | | | 2 |
| GRID1 | | 17 | 19 | | | | | 14 | 1 | 14 | | 20 |
| GRIK5 | | | | | | | 0 | 1 | | | | |
| GRIP2 | | | | | | | | 13 | | | | |
| GRK5 | | | | | | | | 22 | | | | |
| GRK6 | | | | 0.5 | | | | | | 7 | | |
| GRN | | | 19 | | | | | 7.5 | | 11 | | |
| GRP | | | 12 | | | | | | 14 | 11 | | |
| GRPEL1 | | | | | 1 | 22.5 | | 7 | | 6 | | |
| GRPEL2 | | | | | | | | | | 12.5 | | |
| GRPR | | | | | | | | 8 | | | | |
| GRTP1 | 9 | | | | | 18 | | | 21 | | | |
| GS | | | | | | | | 22 | | | | |
| GS | | | 11 | | | | | 8 | | | | 9.5 |
| GSK3A | | | | | | | | | 23 | 13 | | |
| GSK3B | 10 | | 9 | | | | | | 20 | | | |
| GSPT1 | | | | | | | | | 22 | | | |
| GSR | | | | | | | | | | | 22 | |
| GSS | | | 6.5 | | | | 8 | | | | | |
| GSTA3 | 19 | | | | | | | | | | | |
| GSTA4 | | | | | | | | | 23 | | | |
| GSTCD | | | | | | | 9 | | | 2 | | |
| GSTK1 | | | | | | | | | | 3.167 | | |
| GSTM1 | | | | | | | | | | | 4.5 | |
| GSTM2 | | | 5.833 | | | | | | | | | |
| GSTM3 | | | | | | | | | 11 | | | |
| GSTM4 | | | | | | | | | 19 | | | |
| GSTM5 | | | | | | | | | | 16.5 | | |
| GSTO2 | | | | | | | | | | | 5.167 | |
| GSTT1 | | | | | | | | | | | 3.5 | |
| GSTT2 | | | 4.5 | | | | | | | | | |
| GTF2A1 | | | | | | | | | | | 2.5 | |
| GTF2B | | | | | | | | | | | 4.833 | |
| GTF2F2 | | | | | | | | | | | 6.167 | |
| GTF2H2 | | | | | | | | | | | 4.5 | |
| GTF2H3 | | | | | | | | | | | 0 | |
| GTF2H4 | | | | | | | | | | | 4.833 | |
| GTF2H5 | | 4.5 | | | | | | | | | | |
| GTF2I | | | | | | | | | | | 1.167 | |
| GTF2IRD1 | | | | | | | | | | | 7.167 | |
| GTF3A | | | | | | | | | | | 1.833 | |
| GTF3C1 | | | | | | | | | | | 5.167 | |
| GTF3C5 | | | | | | | | | | | 7.167 | |
| GTPBP2 | | | 1.833 | | | | | | | | | |
| GTPBP8 | 10.5 | | | | | | | | | | | |
| GUCY1A2 | | | | | 23 | | | | | | | |
| GUCY1A3 | | | 11 | | | | | 14 | | 13 | | |
| GUSB | 3 | | 4.5 | | | | | | | | 1 | 2 |
| GXYLT2 | | | | | 2 | | | | | | | |
| GYG1 | | | | 5 | 6 | | 6 | | 12 | | | |
| GYLTL1B | | 19 | | | | 20.5 | | 19 | 18 | | | |
| GYPC | | | 22 | | | | | | 23 | | | |
| GYS1 | | | 9.5 | | | | | | | | | |
| GYS2 | | | | | | | | | | | 6.167 | |
| H1F0 | | | | | | | | | | 11 | | |
| H2AFV | | | | | | | | | | | 4.167 | |
| H2AFY | | | 9 | | | | | | | | | |
| H2-M9 | | | | | | | | | | | 3.167 | |
| H2-Q10 | | | | | | | | | | | 5.167 | |
| H2-Q4 | | | | | | | | | | | 5.167 | |
| H2-Q5 | | | | | | | | | | | 4.833 | |
| H6PD | | | 9 | | | | | | | | | |
| HACE1 | | | | | | | 15 | | | | | |
| HACL1 | | | | | | | | | | | 3.5 | |
| HADH | | | | | | | | | | | 3.833 | |
| HADHA | | | | | | | | | | | 0.833 | |
| HAGH | | | | | | | | | 11 | | | |
| HAGHL | | | | | | | | | | | 5.5 | |
| HAL | | | | | | | | | | | 6.167 | |
| HAMP | | | | | | | | | | | 7.833 | |
| HAO2 | | | | | | | | | | | 1.833 | |
| HAPLN1 | | | | | | | | | 11 | | | |
| HAT1 | | | | | | | | | | | 4.833 | |
| HAUS4 | | | | | | | | | | | 4.5 | |
| HBA-PS4 | | | | | | | | | | | 4.833 | |
| HBEGF | | | 7 | | | | | | | | | |
| HBP1 | | | | | | | | | | | 6.5 | |
| HBS1L | | | | | | | | | | | 1 | |
| HCAR2 | | | | | | | | | | 22 | | |
| HCFC2 | | | | | | | | | | | 4.833 | |
| HCN1 | | | | | | | | | | | 2.833 | |
| HDAC10 | | | | | | | | | 10 | | | |
| HDAC11 | | | | | | | | | | | 1 | |
| HDAC3 | | | | | | | | | | | 7.833 | |
| HDAC4 | | | | | | | | | | | 1.833 | |
| HDAC5 | | | | | | | | | | | 5.833 | |
| HDAC6 | | | | | | | | | | | 7.833 | |
| HDAC7 | | | | | | | | | | | 4.167 | |
| HDAC8 | | | | | | | | | | | 4.833 | |
| HDAC9 | | | | | | | | 9 | | | | |
| HDC | | | | | | | | 0 | | | | |
| HDGF | | | 23 | | | | | | | | | |
| HDHD3 | | | | | | | | | 21 | | | |
| HEATR1 | 19 | | 23.5 | 20 | | | | | | | | |
| HEATR5A | | | | | | 10 | | 20 | | | | |
| HEATR5B | 7 | 7 | | | | | | | | | | |
| HEBP1 | | | | 22 | | | | | | 13.5 | | |
| HEBP2 | | | 23 | | 7.5 | | | | | | | |
| HECA | | | | | | | | | | | 6.5 | |
| HECTD1 | | | | | 20 | | | | | | | |
| HECTD2 | | | | | | | | | 0.5 | 7 | | |
| HECTD3 | | | | | | | | | | 11 | | |
| HECW1 | | | | | | | | | 18.5 | | | |
| HECW2 | | | | | | | | 4 | 2 | | | |
| HEG1 | | | | | | | | | 23 | | | |
| HELZ | | | | | | | | | 10 | | | |
| HEPH | | | | | | 11.5 | | | 23 | | | |
| HERC3 | | | | | | | | | | 22.5 | | |
| HERC4 | | | | | | | | 19 | | 22 | | |
| HERPUD1 | | | | | | | | | 21.5 | | | |
| HES6 | | 6 | 5 | | | | | | 9 | | | |
| HEXDC | | 9 | 9 | | | | | | | | | |
| HEY1 | | | | | | | | | 3 | 17 | | |
| HEY2 | | | 23 | | | | | | | | | |
| HEYL | | | | | | | | 18 | | | | |
| HFE | | | | | | | | 22 | | | | |
| HFE2 | | | | | | | | | 18 | | | |
| HGF | | | | | | | | | 14 | | | |
| HGS | | | | | | | | | | 22 | | |
| HGST | | | | | | | | | 22.5 | | | |
| HHAT | 10 | | | | | | | | | | | |
| HHATL | | | | | | | | | | | 1 | |
| HHEX | | | | 20 | | | | | 0.5 | | | |
| HHIP | | | | | | | | | 23.5 | | | |
| HHIPL1 | 19 | | | | | 21.5 | | | 20 | | 1 | |
| HIAT1 | | | | | | | | | | | | 5 |
| HIBADH | | | | | | | | | 1 | | | |
| HIF1A | 17 | | | | | | | | | | | |
| HIF3A | 6.5 | | | | | | | 7 | | | | |
| HIGD2A | 17 | | | | | | | | 22 | | | |
| HILPDA | | | | | | | | | | 11 | | |
| HINT3 | | | | | | | | 1 | 2 | | | |
| HIPK3 | | | | | | | | | 22.5 | | | |
| HIST1H1C | | | | | | 10 | | | | | | |
| HIST1H1D | | | | | | | | 22.5 | | | | |
| HIST1H2BD | 5 | | 23 | | | | | | | 20 | 0 | |
| HIST1H2BG | | | | | | | | | 12 | | | |
| HIST1H2BG | | | | | | | | | 22 | 13.5 | | |
| HIST1H2BJ | | | | | | | | 16 | | | | |
| HIST1H4D | | | | | | | | | | | 12 | |
| HIST1H4F | | | | | | 7.167 | | | | | | |
| HIST2H2BF | | | | | | | | 7 | | | | |
| HIST2H3A | | | 1 | | | | | | 22.5 | | | |
| HIST2H4 | 17 | | | | | | | | | | | |
| HIST3H2BB | | | | | | | | | 0.5 | | | |
| HIVEP1 | | | 0 | | | | | | 16.5 | | | 23 |
| HIVEP2 | | | | | | | | | 19 | | | |
| HJURP | | | 22 | | | | | | | | | |
| HK2 | | 3 | 4 | | | | | | | | | |
| HLA-B | | | | | | | | | 21.5 | | | |
| HLA-C | | | | | 7 | | | | | | | |
| HLA-C | | | | | 19 | | | | | | | |
| HLA-DMA | | | | | | | | 3.5 | | | | |
| HLA-DMB | | | | | | | | | 21 | | | |
| HLA-DOA | 18 | | | | | | | 16.5 | | 22 | | |
| HLA-DOB | | | | | | | | | 22 | | | |
| HLA-DQB1 | 1 | | | | | | | 21.5 | 23.5 | 11.5 | | |
| HLA-DRB1 | 12 | | | | | 21 | | | | | | |
| HLA-E | | | | | | | | 13.5 | | 13 | | |
| HLCS | 21 | | | | | 23 | | | | | | |
| HLF | | 1 | | | | | | | | | | |
| HLTF | | | | | | | | | 14 | 11 | | |
| HLX | | | | | | | | 15.5 | | 4.5 | | |
| HM13 | | | | | 7 | | | | | 0 | | |
| HMCN1 | | | | | | | | | 4 | | | |
| HMCN2 | | | | | | 10 | 6.5 | | 0 | | | |
| HMGCS1 | | | | | | | | | 3.5 | | | |
| HMGCS2 | | | | | | | | | 0.5 | | | |
| HMGN5 | | | | | 22 | | | | | | | |
| HMGXB4 | | 22 | | | | | | | | | | |
| HMHA1 | | | | | 20 | | | | | | | |
| HMOX1 | | | | | | 1 | | | | | | |
| HMOX2 | | | | | | 5 | | 22 | | 0 | | |
| HN1 | | | | | | 8.5 | | 19 | 21 | | 13 | |
| HN1L | 11 | 11 | 11.5 | | | | | 12 | 11 | | | 13.5 |
| HNF1A | | | | | | | 23 | | | | | |
| HNF1B | | | 21.5 | | | | | | 14 | | | |
| HNRNPA0 | | | 7 | | | | | | | | | |
| HNRNPC | | | | | | | | | | 4 | | |
| HNRNPD | | 11 | 12 | | | | | | | | | |
| HNRNPH3 | | | | | | 10 | | | | | | |
| HNRNPL | | | | | | | | 12 | | | | |
| HNRNPM | 23 | 22 | 22 | | 1 | 20.5 | | | 20 | 22 | | 22 |
| HNRNPR | | | | | | | | | 12 | | | |
| HNRNPU | | | | | | | | | 10.5 | | | |
| HNRNPUL1 | | | | | | | | | 10 | | | |
| HNRNPUL2 | | | | | | | | | 9 | | | |
| HNRPDL | | | | | | | | | 10.5 | | | |
| HOGA1 | | | | | | | | | 10 | | | |
| HOMER1 | | | | | | | | | 12 | | | |
| HOMER2 | | | | | | | | | 10 | | | |
| HOMEZ | | | | | | | | | | | 7.5 | |
| HOOK1 | | | | | | | | 23.5 | | | | |
| HOOK3 | | | | | | | | | | | | 12 |
| HOPX | | | | | | | 17 | | 11 | | | |
| HOXA10 | | | | | | | | | 20.5 | | | |
| HOXA5 | | | | | | | | 8 | 6 | 11 | | |
| HOXC8 | 9 | 10 | 10 | 12 | 13 | 11 | 12 | 9.5 | 9 | 10 | 9.5 | 10 |
| HOXD8 | 7 | 8 | 7 | 8 | 9.5 | 7 | 8 | 7 | 8 | 8 | 5 | 8 |
| HP | 22.5 | | | | | | | | 4 | | | |
| HPD | | | | | | | 23 | | 0 | | | 22.5 |
| HPGD | | | | | | | | | | 10 | | |
| HPN | | | 15 | 20 | 22 | | | 23.5 | | 12.5 | | |
| HPRT1 | | | | | | | | | | 13 | | |
| HPS1 | | | | | | | | | | 13.5 | | |
| HPS3 | | 16.5 | | | | | | | 13 | 12.5 | | |
| HPS4 | | | 0 | | | | | | | | | |
| HPS5 | | | 8 | | | | | | | | | |
| HPSE2 | | | | | | | | 22 | | | | |
| HRAS | | | | | | | | 9 | | | | |
| HS1BP3 | | | | | | 23 | | | | | | |
| HS3ST1 | 22 | | | | | | | 23 | | | | |
| HS3ST3A1 | | | | | | | | 23 | | | | |
| HS3ST5 | | | | | | | | 2.5 | | | | |
| HS6ST3 | | | | | | | | | | 6 | | |
| HSBP1 | 19 | | | | 22 | | 20 | | | | | |
| HSD11B1 | 10 | | 9 | | | 10 | | | 9 | 22 | 7 | |
| HSD17B10 | | | | 4 | 5 | | | | | | | |
| HSD17B11 | 23 | 0 | 0 | 23 | 23.5 | 0 | | 23 | 0 | 23 | 2 | 0 |
| HSD17B2 | | | | 21 | | | | | | | | |
| HSD17B4 | | | | | | | | 16 | 9 | 13 | | |
| HSD17B7 | | | | | | | | | | 11 | | |
| HSD3B2 | | | 21 | | | 0 | | | | | 23.5 | |
| HSD3B5 | | | | | | | | | | | | 7 |
| HSD3B7 | | | | | | | | | 1 | | | |
| HSDL2 | | | | | | | | | | 11 | | |
| HSF1 | | | | | 9 | | | | | | 11 | |
| HSF4 | | | | | | | | 16 | 19.5 | | | |
| HSP90AB1 | | | | | | | | | 16 | | | |
| HSP90B1 | | | | | | | | 15 | | | | |
| HSPA12A | | | | | | | | 22 | | | | |
| HSPA13 | | | | | | | | | 0 | | | |
| HSPA14 | | | | | 7.5 | | | | | | | |
| HSPA1L | | | | | 23 | | | | | | | |
| HSPA4 | | | | | | | | | | 9 | | |
| HSPA4L | | | | | | | | | | 0.5 | | |
| HSPA5 | | | | | | | | | 17 | | | |
| HSPA8 | | | 22.5 | | | | | | | | | |
| HSPA9 | | | | | | | | 20.5 | | | | |
| HSPB2 | | | 22 | | | | | | 21 | 18 | | |
| HSPB7 | | | | | | | | 5.5 | | | | |
| HSPB8 | | | | | | | | | | | 3 | |
| HSPBAP1 | | | | | | | | 12 | 15 | | | |
| HSPBP1 | | | | | 10 | | | | 0 | | | |
| HSPD1 | | | | | | | | 6 | 4 | | | |
| HSPE1 | | | 19 | | | | | | | | | |
| HSPG2 | | | | | | | | | 21 | | | |
| HSPH1 | | | | 19 | | | | | | | | |
| HTATIP2 | | | | | | | | 9 | | | | |
| HTR1B | | | | | | | | | | | | 20 |
| HTR2A | 8 | | | | | | | | | 6 | | |
| HTR2C | | | | | | 1 | | | | | | |
| HTRA1 | | | | | | | | | | 6.5 | | |
| HTRA3 | | | | 20 | | | | 17 | | | 2 | |
| HTRA4 | | 7 | 10 | | | | | | 23 | | | |
| HUNK | | | 0 | | | | | 5.5 | | | | |
| HUS1 | | | | | | | | | | 13 | | |
| HUWE1 | | 17 | 18 | | | | | 15.5 | | 13 | | 18 |
| HYDIN | | | | | | | | | 3 | | | |
| HYI | 6 | | | | | | | | 23 | | | |
| HYLS1 | | | | | | | | | 0.5 | | | |
| HYOU1 | 21 | | | | | | | | | | | |
| I | | | | 6 | | | 7 | | | | | |
| I830012O16RIK | | 6 | 6 | | | | | 23 | 22 | | | |
| IAPP | | | | | | | | 19 | | | | |
| IARS | | | | | | | | 0 | 22 | 16 | | |
| IBTK | | | | | | | | | 23 | 18 | | |
| ICA1 | | | | | | | | | 22 | | | |
| ICAM2 | | | | | | | | 9 | | | | |
| ICK | | | | | | | | | 13 | 11 | | |
| ICMT | | | | | | | | | 22 | | | |
| ICOSLG | | | | | | | | | 22.5 | 19 | | |
| ID1 | | | | | | | | 21.5 | | | | |
| ID2 | | | | | | | | 13 | | 10 | | |
| IDE | | | | | 22 | | | | | 19 | | |
| IDH1 | | | | | | | | 19 | | | | |
| IDH2 | | | | | | | | | 6.5 | | | |
| IDH3A | | | | | | | | | | 9 | | |
| IDNK | | | | | | 1.167 | | | | | | |
| IDO2 | | | | | | | | | 23.5 | | | |
| IDUA | | | | | | | | | 8 | 11 | | |
| IER3 | | | | | | | | | 22.5 | | | |
| IER5 | | | | | | | | | 10.5 | | | |
| IFI204 | 23.5 | 21 | 21 | | 1 | 21 | | 21 | 21 | 22 | 20 | 21 |
| IFI205 | | 15 | 15 | | | 12 | 13 | | 18 | | | 17 |
| IFI27L2 | | | | | | | | 12 | | | | |
| IFI30 | | | | | | 10 | | 12 | | | | |
| IFI44 | | | 4 | | | | | | | 23 | | |
| IF147 | | 15 | 15 | 16 | | | | 19 | | 16 | 11 | |
| IFIT1B | | | 12 | | | | | | | | | |
| IFIT3 | | 9 | | | | | | | | | | |
| IFITM1 | | | | | | | | | | | 1 | |
| IFITM6 | | | | | | | | 2 | | | | |
| IFLTD1 | | | 6 | | | 0 | | | | | 4.5 | |
| IFNGR2 | | | | | | 21 | | | | | | |
| IFR2 | 2 | | | | | | | 3.5 | | | | |
| IFRD1 | | | | | | | | | | | | 4.5 |
| IFRD2 | | | | 16 | | | | | | | | |
| IFT122 | | | | | | | | | 21.5 | | | |
| IFT140 | | | | | | | | 14 | 19 | 14 | | |
| IFT172 | | | | | 9 | | | 7 | | 15 | | |
| IFT20 | 15 | | | | | | | | | | | |
| IFT27 | 8 | | | 12 | | | 12 | 20 | 22 | | | |
| IFT80 | | | 20 | | | 4.5 | | | | 23.5 | | |
| IFT81 | | | | | 7.5 | | | | | 8 | | |
| IGF1 | | | | | | | | | | | 1 | |
| IGF1R | | | | | | | | | 23 | | | |
| IGF2BP2 | | | | | | | | | 14 | | | |
| IGF2R | | | | | | | | 8 | | | | |
| IGFALS | 22.5 | | | | | | | 23.5 | | | | |
| IGFBP1 | 12 | | | | | | | | | 7 | | |
| IGFBP2 | | | | | | | | 1 | 22 | | | |
| IGFBP3 | 18 | | | | | 0 | | | | | | |
| IGFBP4 | | | | | | | | 2.5 | | | | |
| IGFBP5 | | | | | | | | | 22 | | | |
| IGFBP6 | | | | | | | | | | 19.5 | | |
| IGKV2-112 | | | | 0 | | | | | | | | |
| IGKV8-21 | | | | | | 0 | | | | | | |
| IGSF10 | | | | | | | | | 21 | | | |
| IGSF11 | | | 5.5 | | | | | | | | | |
| IGSF21 | | | | | | | 7 | | | | | |
| IGSF3 | 16.5 | | | | | | | | | | | |
| IGSF6 | | | | | | 6 | | | 1 | | | |
| IGSF8 | 17 | | | | | | | | | 17 | | |
| IGSF9B | | | | | | | | | 4 | | | |
| IIGP1 | | 4 | | | | | | | | | | |
| IKBIP | | | | | | | | | 21.5 | | | |
| IKBKG | | | | | | | | | 21 | 20 | | |
| IKZF2 | | | | | | | | | 22.5 | | | |
| IKZF3 | | | | | | | | | | 6 | | |
| IKZF4 | | | | | | | | | 21.5 | | | |
| IL13RA1 | | | | | | | | | 22 | | | |
| IL15RA | | | | | | | | | 23 | | | |
| IL17D | | | | | | | | 17 | 14.5 | | | 18 |
| IL17RB | | | | | | | | | 23 | | | |
| IL18R1 | | | | | | | | | 11.5 | | | |
| IL1A | | | | | 18 | | | | | | | |
| IL1R1 | 7 | | | | | | | | | | | |
| IL1R2 | | | 8 | | | | | 9.5 | 1 | 6 | | |
| IL1RL2 | | | | | | 15 | | | | | | |
| IL20RB | | | | | | | | 15 | | | 4 | |
| IL23R | | | | | | | | 23 | 2 | | | 9 |
| IL2RA | | | 9 | | | | | | | | | |
| IL33 | | | | | | | | | 11 | | | |
| IL34 | | | | | | 2 | | 10 | 11.5 | | | |
| IL4R | | | | 17 | | | | | | 13 | | |
| IL6R | | | | | | | | | | 11 | | |
| IL6ST | | | | | | | | | 16.5 | | | |
| ILDR1 | | 23 | 23 | | | | | 2 | | 23 | | 0 |
| ILF2 | | | | | | | | | 0.5 | | | |
| ILK | | | | | | | 20 | | 21 | | | |
| IMP4 | 15 | | | | | 17 | | | 19 | 15 | | |
| IMPDH1 | | | | | | | | | 4 | | | |
| INCA1 | | | | | | | | | 13 | | | |
| INF2 | | | | | | 7 | | 22 | | | | |
| ING1 | | | | | | | | 0 | | | | |
| ING4 | | | 1.5 | | | | | 23 | | | | |
| INHA | | | | | | | | 20 | | | | |
| INHBA | | | | | | | | | | | 8.5 | |
| INHBC | | | | | | | | | | 10.5 | | |
| INMT | | | | | | | | 18.5 | | | | |
| INO80 | | | | | | | | 21 | 19 | | | |
| INPP4B | | | | | | | | 23.5 | | | | |
| INPP5A | | | | | | | | 7 | | | | |
| INPP5B | | | | | | | 6 | | | | | |
| INPP5J | | | | | | | | | 2 | 1 | | |
| INPP5K | | | | | | | | 5 | | | | |
| INSC | | | | 12 | | | | | | | | |
| INSIG1 | | | | | | | | 12 | 19 | | | |
| INSIG2 | | 21 | | | | | | 21 | | | | |
| INSL5 | | | | | | | | | 21 | | | |
| INTS10 | | | | | | | | 7 | | | | |
| INTS12 | | | | | | | | | 23.5 | | | |
| INTS2 | | | | | | | | | | | 21.5 | 10 |
| INTS3 | | | | | | | | | 6 | | | |
| INTS7 | | | | | | 18 | | | | | | |
| INTS8 | | | | | | | | 13 | | | | |
| INTS9 | | | | 12 | | | | 22 | | | | |
| INTU | | | | | | | | | | | 18 | |
| INVS | | | | | | | | | | 10.5 | | |
| IP6K3 | | | | | | | | | 20 | | | |
| IPCEF1 | | | 3 | | | | | 23 | | | | |
| IPMK | | | | | | | | 23.5 | | | | |
| IPO11 | 14 | | | | | | | | | | | |
| IPO13 | | | | | | | | 10.5 | | | | |
| IPO4 | | | | | | | | | | 9 | | |
| IPO5 | | | | | | | | | | 5 | | |
| IPO9 | | | | | | 2 | | | 0 | 0 | 4 | |
| IQCJ-SCHIP1 | 21.5 | | | | 3.5 | | | | | 15 | | |
| IQCK | | 11 | | | | | | | | 11.5 | | |
| IQGAP2 | | | | | | | | | 23 | 13 | | |
| IQGAP3 | 0 | | | | | | | | | | | |
| IQSEC1 | 10 | | | | | | | 12 | | | | |
| IRAK1BP1 | | | | | | | | | | 7.5 | 0.5 | |
| IRAK2 | 22.5 | | | | | | | 16 | | 3.5 | 2 | 22 |
| IRAK3 | | | | | | 12 | | | | | 13 | |
| IRAK4 | | | | | | | | 23.5 | | | | |
| IRF2 | | 11 | 14 | | | 13 | | | | | | 14 |
| IRF2BP2 | | | | | 11 | | | | 23 | | | |
| IRF6 | | | 20 | | | | | | | | 10.5 | |
| IRF7 | 6 | | | 10 | | | | | | | | |
| IRF9 | | 21 | | | | | | | | | | |
| IRGM | | | 0.5 | | | | | | | | | |
| IRGM2 | | | | | | | | 9 | | 12.5 | | |
| IRS1 | | | 12 | | | | | | | | | |
| IRS2 | | | 23 | 22 | | | | | | | | |
| IRS3 | | | | | | | | | | | 18 | |
| IRX1 | | | | | | | | | 12 | | | |
| IRX2 | | | | | | | | | 0 | | | |
| IRX3 | | | | | | 1 | | | | | | |
| ISLR | | | 20 | | | | | | | | | |
| ISM1 | 7 | | | | | | | | | | | |
| ISOC1 | | | | | | | | | 16 | | | |
| ISY1-RAB43 | | | | | | | | | | 8 | | |
| ITCH | | | | | | | | | | | 11 | |
| ITFG3 | | | | | | | | | 22 | | | |
| ITGA1 | | | | | 9.5 | | | | | 9 | | |
| ITGA11 | | | | | | | | | | 16 | 9 | |
| ITGA3 | | | | | | | | | | 12 | | |
| ITGA4 | | | | | | | | | | | 13.5 | |
| ITGA5 | | 6.5 | | | | | | | | 7 | | |
| ITGA6 | | | | | | | 6 | | 2.5 | | | |
| ITGA7 | | | | 5 | | | | | 22 | | | |
| ITGA8 | | | 11.83 | | | | | | | | | |
| ITGA9 | | | | | | | | 23 | 0 | | | |
| ITGAL | | | | | | | | | | 17 | | |
| ITGB1 | | | | | | | | 13 | 1 | 7 | | |
| ITGB3 | | | | | | | | | 20 | | | |
| ITGB4 | | | | | | 2 | | | | | 2.5 | |
| ITGB5 | | | 21 | | | 1 | | | | | | |
| ITGB6 | | | 8 | | | | | | | | | |
| ITGB8 | | | | | | | | | | 6.5 | | |
| ITIH1 | | | | | | | | | | 4 | | |
| ITIH2 | | | 9 | | | | | | | | | |
| ITK | | | | | | | | | | | 0.167 | |
| ITM2A | | | | | | 1 | | 2 | | | | |
| ITM2B | | | | | | | | 23.5 | 8 | | | |
| ITPK1 | | | | | 19 | | | | | | | |
| ITPKB | | | | | | | | | 1 | | | |
| ITPR1 | | | 15 | | | | | | 18 | | | |
| ITPR2 | | | | | | 9 | | | | 8 | | |
| ITPR3 | | 12.5 | 13 | | | 2.5 | | 11 | 13 | | | |
| ITPRIP | | | | | | | | | 17 | | | |
| ITPRIPL1 | | | | | | | | 22 | | | | |
| ITPRIPL2 | | | | | | | | 10 | 7 | | | |
| ITSN1 | | | | | | | | | 0 | | 4 | |
| ITSN2 | | | | | | | 5 | | 23 | | | |
| IVD | | | 3 | | | | | | | | | 0.5 |
| IVNS1ABP | | | | | | | | 17 | | | | |
| IWS1 | | | | | | | | | | | 3.833 | |
| IYD | 6.5 | | | | | | | | | 13 | | |
| JAG2 | | | | | | | | | | 12 | | |
| JAM2 | | | | | | | | | | 11.5 | | |
| JAMS | | | 22 | | | | | 22 | 22 | | | 22 |
| JARID2 | 11 | 13 | | | 12 | 11 | 11 | 11.5 | 13 | 11 | | 13 |
| JDP2 | | | | | | | 5.5 | | 1 | | | 21.5 |
| JMJD1C | | | | | | | | | 23 | | | |
| JMJD8 | | | | | | | | 19 | | | | |
| JOSD2 | | | | | | 2 | | | | | | |
| JPH1 | | | | 23 | 19.5 | | | | | | | |
| JTB | | | | | 6.5 | | | | 23 | 14 | | |
| JUN | | | | | | | | | 0.5 | | | |
| JUND | | 20.5 | | | | | 2 | | 22 | | | |
| KALRN | | | | | | 7 | | 3 | 2 | 2 | | |
| KANK1 | | | 6 | | | | | | | | | |
| KANK2 | | | | | | | | | 4.5 | | 5.5 | |
| KANK3 | 5.5 | | 10.5 | 12.5 | 14 | 11 | 8.5 | 6 | | | | |
| KANK4 | | 12 | 11 | | 14 | 14 | 10 | 6 | | | | |
| KANSL1L | | 8 | | | | | | | | | | |
| KANSL3 | | | 21 | | | | | | | | | |
| KAT6A | | | | | | | | | 1.5 | | | |
| KAT7 | | | | | 23 | 5.5 | | | | | | |
| KAT8 | | 20 | 20 | | | 0 | | 21.5 | | | | |
| KATL1 | | | | 22 | | | | | | | | |
| KAZN | | | | | | | | | 3 | | | |
| KBTBD12 | | | | | | | | 23 | | | | |
| KBTBD2 | | | | | | | | | | 18 | | |
| KBTBD7 | | | | | | | 17 | | | 2 | | |
| KC1 | | | | | | 11.5 | | | | 13.5 | | |
| KC2 | | | | | | 6 | | | | 4 | | |
| KCMF1 | | | | | | | | | | | 21.5 | |
| KCNB1 | 17 | | | | | | | | | | | |
| KCNC1 | | | | | | | 0 | | 22 | | | |
| KCNC3 | | | | | | | | | | | | 21 |
| KCND2 | | | | | | | | | 21 | | | |
| KCNE2 | | | 3 | | | | | | | | | |
| KCNF1 | | | | | | | | | 22 | | | |
| KCNG1 | | | | | | | | | 14 | | | |
| KCNG4 | 11 | | | | | | | | | | | |
| KCNH2 | 19 | | | 21 | | | | 20 | | | | 22 |
| KCNIP3 | | | | | | | | | 21 | | | |
| KCNIP4 | | | | | | | | | 21 | | | |
| KCNJ10 | | | | | | | | | | | | 22 |
| KCNJ11 | | | | | | | | | 23 | | | |
| KCNJ12 | | | | | | | | | 23 | | | |
| KCNJ13 | | | | | | | | | | | | 19 |
| KCNJ15 | | | | | | | | 0 | | | | |
| KCNJ3 | | | | | | | | | 22 | | | |
| KCNJ8 | | | | | | | | | 21 | | | |
| KCNK1 | | | | | | | | | 21.5 | | | |
| KCNK10 | | | | | | | | | 23 | | | |
| KCNK2 | | | | | | | | | 20 | | | |
| KCNK3 | | | | | | | | | | 8 | | |
| KCNQ2 | | | | | | | | 8 | | | | |
| KCNQ4 | | | | | | | | | 22 | | | |
| KCNT1 | | | | | | | | | 19 | | | |
| KCTD10 | | | | | 13 | | | | | | | |
| KCTD13 | | | | | | | 6.5 | 11 | 23 | | | |
| KCTD15 | | | | | | | | | 0 | | | |
| KCTD21 | | | | | | | | | 21 | | | |
| KCTD5 | | | | | | 8.5 | | | | | | |
| KCTD7 | | 22 | | | | | | | | | | |
| KCTD9 | | | | | | | | | | | 4.5 | |
| KDELC1 | | | 18 | | | | | | 0 | 0.5 | | |
| KDELR1 | | | 5 | | | | | | | | | |
| KDELR2 | | | | 12.5 | | 19 | | | | 2 | | |
| KDELR3 | | | | 2.5 | | | | | | | 23 | |
| KDM1B | | | | 23 | | | | | | | | |
| KDM3A | 21 | | | | | | | | 21 | | | |
| KDM3B | | | | | | 14 | | | | | | |
| KDM4A | | | 16 | | | | | | | | | |
| KDM4B | | | 13 | 13 | | | | | | | | |
| KDM5A | 0 | | | | | 2 | 0 | 1 | 23 | 1 | | |
| KDM5B | 15 | | 17 | | | 16 | | 15 | 14 | 13 | | 17 |
| KDM5C | | | | | | | | | 21.5 | | | |
| KDM6B | | | 3.5 | | | 23.5 | | | | | | |
| KDR | | | | | 9 | | | | | | | |
| KDSR | | | 5.167 | | | | | | | | | |
| KHDRBS1 | | | 21 | | 20 | | | 1 | 5.5 | 17 | | |
| KHDRBS3 | | | | | | | | 22 | | | | |
| KHNYN | | | | | | | | | | 15 | | |
| KIAA0040 | | | | | | | | 16 | 18 | | | |
| KIAA0146 | | | | | | | | 13 | | 15 | | |
| KIAA0182 | | | 19 | | | | | 17 | | | | |
| KIAA0195 | | | | | | | | | 0 | | | |
| KIAA0196 | | | 23 | | | | | | 19.5 | 12.5 | | |
| KIAA0226 | | | 23 | | | | | | | | | |
| KIAA0226L | 19.5 | | 21 | | | | 21 | 22 | | | | |
| KIAA0232 | | | 12 | | | | | 11 | | | | |
| KIAA0247 | | | | | | | | 10 | 6 | 5.5 | | |
| KIAA0284 | | | | | | | | 22 | 2 | | | |
| KIAA0317 | | 6 | | | | | | | | | | |
| KIAA0408 | | | | | | | | | 22 | | | |
| KIAA0415 | | | | | | | | 19 | 21 | | | |
| KIAA0430 | | | 23 | | | | | | | 20.5 | | 23 |
| KIAA0513 | | | | | | | | | 14 | | | |
| KIAA0528 | | | | 20.5 | | | | | | | | |
| KIAA0556 | | | | | | 16 | | | | | | |
| KIAA0564 | | | | | | | | | | | | 4.833 |
| KIAA0664 | | | | | | | | 12 | | | | |
| KIAA0895 | | | | | | | | | 21 | | | |
| KIAA0907 | 7 | | | | | 11 | | | | | | |
| KIAA0922 | | | | | | | | | 0 | | | |
| KIAA0930 | | | | | | | | 5 | | | | |
| KIAA0947 | | | | | | | | 0 | | 22 | 23.5 | |
| KIAA1033 | | | | | | | | | 21.5 | | | |
| KIAA1109 | | | | | | 18 | | | | | | |
| KIAA1161 | | | | | | | | | 22 | 8 | | |
| KIAA1191 | 4 | | 1 | | | | | | | 2.5 | | 3 |
| KIAA1217 | | | | | 6 | | | | | | | |
| KIAA1244 | | | | 6.5 | 5.5 | | | | | | | |
| KIAA1274 | | | | | | | | | 19.5 | | | |
| KIAA1279 | 2.5 | | 23 | | | 23 | | 0 | 12.5 | 3 | | 0.5 |
| KIAA1324 | | 2.5 | 23 | 1 | 2 | 1 | 1.5 | 4 | | 2 | | 4 |
| KIAA1324L | | | | | | | | | | | 3 | |
| KIAA1328 | | | | | | | | 4.5 | | 3.5 | | |
| KIAA1377 | | | | | | | | | 22 | | | |
| KIAA1383 | | | | | | | | 20.5 | | | | 23 |
| KIAA1429 | | | | | | | | | 18 | 20 | | 21 |
| KIAA1432 | 0.5 | | 10 | | | | | | | | | |
| KIAA1456 | | | 21 | | | | | | | | | |
| KIAA1462 | | | | | | | | 17 | | 20 | | |
| KIAA1467 | | | | | | | | | 23 | | | |
| KIAA1468 | 17 | | | 21 | | | 21 | 19 | 20.5 | 15 | | |
| KIAA1522 | | | | | | | | | 22.5 | | | |
| KIAA1598 | | | | | | | | | | 11 | 23.5 | |
| KIAA1644 | | | | | | | | | 22 | | | |
| KIAA1715 | | | | | | 23.5 | | 19.5 | 23 | | | |
| KIAA1737 | | | | | | | | | 18 | 14 | | |
| KIAA1797 | | | | | | | | | | 11 | | |
| KIAA1841 | | | | | | | | | | 8 | | |
| KIAA1967 | | | | | | | | | | 6.5 | | |
| KIAA2018 | | | | | | 10 | | | | 6 | | |
| KIDINS220 | 19 | | | | 21 | | 21 | | 21 | | | |
| KIF12 | | | | | | | | | 21 | | | |
| KIF13A | | | | | | 11.5 | | | | | | |
| KIF13B | 20.5 | | | | | | | | | | | |
| KIF16B | | | | | | | | | | 13 | | |
| KIF1B | | | | | | | | | 23 | | 10 | |
| KIF21A | | | | | | | | | | 15.5 | | |
| KIF2A | | | | | | | | | 2 | | | |
| KIF3A | | 15 | 16 | | | 17 | | 13.5 | 12.5 | 12 | | |
| KIF5B | | | | | | | | | 15 | 10.5 | | |
| KIFAP3 | | | | | 23 | | | | | | | |
| KIFC2 | | 15 | 16 | | | | | 23.5 | 21 | | | |
| KIFC3 | | | | 20 | | | 18.5 | 20 | | 18 | | |
| KIRREL | | | | | | | | | 19.5 | | | |
| KIRREL3 | | | | | | | | | | 14 | | |
| KIT | | | | | | | | | | 2 | | |
| KITLG | | | | | | | | | | | | 23 |
| KLB | | | 23 | | | | | | | | | |
| KLC1 | | | | | | 13 | | | | 10 | | |
| KLC4 | | | | | | | | 23 | | | | |
| KLF10 | | | | | | | | | 4.5 | | | |
| KLF11 | | | 6 | | | | | | | | | |
| KLF12 | | 0 | | | | | | | | | | |
| KLF13 | | 21 | | | | | | | | | 5.5 | |
| KLF15 | | | 19 | | | | | | | | 7.5 | |
| KLF16 | | | | 2.5 | | | | | | | | |
| KLF3 | | | | | | | | | | | | 8 |
| KLF5 | | | | | | | | 4 | | 5.5 | 0.5 | |
| KLF6 | | 15 | 17 | | | | | 13 | 11 | 14 | | |
| KLF9 | | | | | | | | 2.5 | | | | |
| KLHDC2 | | | 8 | | | | | | | | | |
| KLHDC3 | | | 21 | | 23 | | | 22 | | | 0 | |
| KLHDC8A | | | | | | | | | 23 | | | |
| KLHL11 | | 20 | | | | | | | | | | |
| KLHL13 | | 1 | | | | | | 23 | 21 | 22 | 0.5 | 20.5 |
| KLHL21 | | | | | | | | | | 5 | | |
| KLHL22 | | | 19 | | | 15 | | | | | 14 | |
| KLHL24 | | | | | | | | | 2 | | | |
| KLHL29 | | | | | | | | 22 | 22 | | | |
| KLHL30 | | | | | | 22 | | | | | | |
| KLHL32 | | | 23.5 | | | 23 | | | 21 | | | |
| KLHL38 | | | | | | | | | | 4 | 22.5 | |
| KLHL4 | 12 | | | | | | | | | | | |
| KLHL5 | | | | | | | | | 10 | | | |
| KLHL7 | | | | | 5 | | | | 23 | | | 21.5 |
| KLHL8 | | | | | | | | | | | 18 | |
| KLK3 | | | | | | | | | | 7 | | |
| KLK3 | | 6.5 | 6 | | | 22 | | | 9 | | | |
| KLK5 | | | | 13 | | 16 | | | | | | |
| KLRC4-KLRK1 | | | | | | | | 8 | | | | |
| KLRD1 | | | | | | | | | 16 | | | |
| KMO | | | 21 | 21.5 | | | 20 | | 5 | | | |
| KP1 | | | | | | | | 22 | 22 | | | |
| KP3 | 10 | | | | | | | 10 | | 8 | | |
| KP4 | | 6 | | | | | | | | 10 | | |
| KPNB1 | | | | | | | | 22 | | | | |
| KPTN | | | | | | | | | 1 | | | |
| KRAS | | | 22.5 | | | | | 19.5 | 22 | | | |
| KRBA1 | | | | | | | | 9 | | | | |
| KREMEN1 | | | | | | | | 1 | | | | |
| KRT18 | | | | | | | | | | | | 12.5 |
| KRT23 | | | | | | | | | | 8 | | |
| KRT7 | | | | 18 | 15 | | 17 | | 12 | 5.5 | | |
| KRT8 | | | | | | | | | 21 | | | |
| KRT80 | 4 | | | | | | | 6 | | | | |
| KRTAP12-2 | | | | | | | | | | 10 | | 11 |
| KSR1 | | | | | | | | | 9.5 | | | |
| KTI12 | | | | | | 18 | | | 17 | 13 | | |
| KY | | | | | | | | 19 | | | | |
| KYNU | | | | | 0 | | | | 22 | | | |
| L3MBTL3 | | | | | | | | 18 | 17 | | | |
| LACC1 | | | | 17 | | 15 | | | | 15 | | |
| LACE1 | | | 5.5 | | | | | | | | | |
| LAD1 | | | | | 23 | | | 0 | | 20 | | |
| LAMA2 | 18 | | | | | | | 20 | 21.5 | | | |
| LAMA3 | | | | | | | | | | 16 | | |
| LAMA4 | 6 | | | 9.5 | | 11 | 8 | | | | | |
| LAMA5 | | | | | | 13 | | | 20 | 15 | | |
| LAMB1 | | | | | | 22 | | | | | | |
| LAMB2 | | | 1 | | | | 21 | 21 | 23 | | | 21 |
| LAMB3 | | 9 | | | | | | | | | | |
| LAMC1 | | | | | | | 0 | | 0 | | | |
| LAMTOR1 | | | 12 | | | | | | | | | |
| LANCL2 | | | | | | | | | 23.5 | | | |
| LAP3 | | | | | | | | 21 | | | | |
| LAPTM4B | | | 12.5 | | | | 13 | 13 | | 13.5 | | |
| LARGE | | | | | | | | | | 10 | | 8 |
| LARP1 | | | | | | | | | 21 | | | |
| LARP1B | | | | | | | | | 17 | | | 22 |
| LARP4B | | | | | | | | 0.5 | 21 | | | |
| LARP7 | | | | | | | | 22 | | | | |
| LAS1L | | | | | 16 | | | 0 | 21 | | | 21 |
| LASP1 | | | | | | | | | | 8.5 | | |
| LATS2 | 22 | | | | | | | | | | | |
| LAYN | | | | | | | | | 23 | 8.5 | | |
| LBH | | | | | | | | | | | | 23 |
| LBP | | | | | | | | 21 | | | | |
| LCA5L | | | | | | | | 22 | 23 | | | |
| LCK | | | | | | | | | 23 | | | |
| LCLAT1 | | | | | | | | | 4.5 | | | |
| LCMT1 | | | | | | | | 0 | 23.5 | | | |
| LCMT2 | | | 1 | | | | | | 22 | | | |
| LCN12 | | | | | | 22 | | 23.5 | | | | |
| LCP1 | | | 10.5 | | | 17 | | | | | | |
| LCP2 | | | | | | | | | 5 | 7 | | |
| LDB1 | | | 9.5 | | | | | | 1 | | | |
| LDB2 | | | | | | | | 10 | 14 | 13.5 | | |
| LDHB | | | | | | | | | | | 0 | |
| LDLR | | 23 | 0 | | | | | | 21 | | | |
| LDLRAD3 | | 6 | 8 | | 9 | | 10 | | | | | |
| LDLRAP1 | | | | | | | | | 22 | | | |
| LDOC1L | | | | | | | | | | 11 | | |
| LEAP2 | | | | | | | | | 17 | | | |
| LECT2 | | | | | 7 | | | | | | | |
| LEFTY1 | 21 | | 22.5 | | | | | | | | | |
| LEKR1 | | | | | | | | 20.5 | | | | |
| LEMD2 | | | | | | | | 7 | | | | |
| LENG9 | | | | | | | | | 19 | | | |
| LEO1 | | | | 20.5 | | | | | | | | |
| LEP | 21.5 | | 23 | | | | | | 2 | | | 0 |
| LEPR | | | | | | | | | 2 | | | |
| LEPREL1 | | | | | | | | | | 17 | | |
| LEPROT | | | | | | | | | | | 21.5 | |
| LEPROTL1 | | | 2 | | | | | | | | | |
| LETM1 | | | | | | | | 20 | | | 20 | |
| LETM2 | | 9 | | | | | | | 13 | 13 | | |
| LETMD1 | | | | | | | | | | | 21 | |
| LFNG | | | | | | | | | 3 | 15.5 | | |
| LGALS1 | | | 2.5 | | | | | | 23 | | | 4 |
| LGALS12 | | | | | | | | | | 12 | | |
| LGALS3 | | | | | | | | | 22 | | | |
| LGALS3BP | | | | | | | | 10 | | | | |
| LGALS4 | | | | | | | | 13 | | | | |
| LGALS8 | | | | | | | | 17 | 21 | 5.5 | | |
| LGALS9 | | | | | | 13 | | | | 10.5 | | |
| LGALSL | | | | | | | | | 21 | | | |
| LGI2 | | | | | 20 | 23 | | 22 | | | 23.5 | |
| LGI4 | | | 12 | 3 | | | | | | 22 | | |
| LGMN | 6 | | 1 | 3 | 6 | 2 | 4 | | | 7.5 | 0 | |
| LGR4 | | | | 11 | | | | | | | | |
| LGR6 | | | 21.5 | | | | | | | | | |
| LHFPL2 | | | | | | | | 5 | 6 | | | |
| LHPP | | | | | | | | 3 | | | | |
| LHX5 | | | 20 | | | 16 | | 13 | 14 | | | |
| LHX6 | | | 23 | | | | | | | | | 23 |
| LIFR | 16.5 | | | | | | | | 19.5 | | | |
| LILRB3 | | | | | 22 | | | | | | | |
| LILRB3 | | | | | | | | 21 | 23 | | | |
| LILRB4 | | | | | | | | | | | 3 | |
| LIMA1 | | | | | | | | | | | | 23.5 |
| LIMCH1 | | 12 | | | | 13 | | | | 13 | | 12 |
| LIMD1 | | | | | | | | | 22 | | | |
| LIMD2 | | | | | | 13 | | | | 10 | | |
| LIMK1 | | | | | | 19 | | | 17 | | | |
| LIMK2 | | | 5.5 | | | | | | | | | |
| LIMS1 | | | 20 | | | | | | | | | |
| LIMS2 | | | | | | | | | | | 12 | 9 |
| LIN37 | | | | | | | | | 22 | | 2 | |
| LIN52 | | | | | 12 | | | | | | | |
| LIN54 | | | 21 | | | 19 | | | | | | |
| LIN7A | | | | | | | 9 | | | | | |
| LIN7C | | | 18 | | | | | | 20.5 | | | |
| LIN9 | 15 | 15.5 | 17 | | | 16 | | 15.5 | 15 | 13.5 | | 15.5 |
| LINGO2 | | | | | | | | | | 19 | | |
| LINGO3 | | | | | | | | | | 11 | | |
| LINGO4 | | | | | | | | 12 | | | | |
| LIPA | 0 | | 0 | | | 2 | | 1 | | | 1 | 1 |
| LIPC | 20 | | | | | | | 22 | | | | |
| LIPE | | | 7 | 22 | | | | | | | | |
| LIPG | | 15 | 18 | 22 | | 18 | | 16 | 16 | 14 | | 17 |
| LIPH | | | | | | | | | 21 | | | |
| LITAF | | | | | | | | | | | | 8 |
| LIX1L | | 6.5 | 7 | | | | | 0 | 2 | | | |
| LLGL2 | | | | | | | | 20 | | | | |
| LMAN1 | | | | | | 23.5 | | | | | | |
| LMAN2 | | | | | 6 | | 7 | | | | | |
| LMAN2L | 7.5 | | | | | | | | | | | |
| LMBR1L | | | | 12 | | 21 | | | | | | |
| LMBRD2 | | | | | | | | | 23 | | | |
| LMCD1 | | | | | | | | | 2 | | | |
| LMLN | | | | | | | | | 23 | 8 | | |
| LMO4 | | | 18.5 | | | | | 15 | 17 | | | 18 |
| LMO7 | | 11 | 9 | | | | | 10.5 | | 10 | | 9 |
| LMOD1 | 18 | | | | | 8.5 | | | | | | |
| LMOD2 | | | | | | | | | | | | 6 |
| LMTK2 | | | | | | | 16 | 22 | 0 | | | |
| LMTK3 | | | 23 | | | 2.5 | | | | | | |
| LNX1 | 22 | | 3 | | | | | | 0 | 4 | | 1 |
| LNX2 | | | | | | | | 9 | | | | |
| LOC100129480 | | | | | | | | | | 16 | | |
| LOC100129924 | | | | | | | | | | 13 | | |
| LOC100505478 | | 12.5 | 13 | | | | | | | 12.5 | 13 | 15 |
| LOC100652815 | | | | | | | | 22 | 11 | | | |
| LOC375190 | | | | | | 23 | | | 22.5 | | 23 | |
| LOC388630 | | 7 | 9 | | | 7 | 9 | 6 | 4.5 | 6.5 | | 8.5 |
| LOC441617 | | | 19 | | | | | 10 | 12 | | | |
| LOC646851 | 11 | 12.5 | | | | | | | 14 | 12 | | 14.5 |
| LONP1 | | | | | | | | | 22 | | | |
| LONP2 | | | | | | | | | | 9.5 | | |
| LONRF1 | | | | | | | | | | 11 | | |
| LONRF2 | | | | | | | | 21 | | | | |
| LONRF3 | | | | | | | | 0 | | | | |
| LOX | | | | 18.5 | | 0 | | 18 | | 17 | | |
| LOXL1 | | | 9.5 | | | | | | | | | |
| LOXL4 | | | | | | | | | 1 | | | |
| LPAR2 | | | | | | 0 | | 21 | | | | |
| LPAR3 | | | | | | | | | | 8.5 | | |
| LPAR4 | | | | | | | | | | 9 | | |
| LPAR6 | | | | | | 13 | | 17 | 15 | 16 | | |
| LPCAT1 | | | | | | | 17 | 20 | | | | |
| LPCAT2 | | | | | | | | 5 | | | | 0 |
| LPCAT2B | | | | | | | | 20 | 19.5 | | | |
| LPCAT3 | | | | | | 3 | | 21 | 18 | 19 | | |
| LPCAT4 | | | | | | | | 20 | | | | |
| LPGAT1 | | | | | | 5 | | | | 15 | | |
| LPHN1 | | | | | | | | 6.5 | | | | |
| LPHN2 | 2 | | | | 23 | | | | 2 | | | 1.5 |
| LPHN3 | | | | | | | 3 | | | | | |
| LPIN1 | | | | | 8 | | | | 21.5 | | | |
| LPIN2 | | | | | 6 | | | | | | | |
| LPIN3 | | | | | | | | | | | 6.5 | |
| LPL | | | | | | | | | 9 | 11 | | |
| LPP | | | | | | | | | 10.5 | | | |
| LPPR1 | | 6 | 7 | | | | | 7 | | | | 5 |
| LRAT | | | | | | | | 3 | | | 0 | |
| LRFN3 | 7 | | | | | | | 12 | 0 | 14 | | |
| LRFN5 | | | | | | | | | | 16 | | |
| LRG1 | 20 | | | 23.5 | | | 1 | 23.5 | | 2 | 23 | |
| LRIF1 | 18.5 | | | | | | | | | | | |
| LRIG1 | | | | | | 16 | | | | | | |
| LRIG2 | | | | | | | | | 22.5 | | | |
| LRIT1 | | 16 | 19 | | | 16 | | | | 15 | | |
| LRP1 | | | 18 | | | | | | 15.5 | | | |
| LRP10 | | | | | | | | 19 | | | | |
| LRP11 | | | 20 | | | | | | | | | |
| LRP12 | 23 | | | | | | | 11 | 0 | | | |
| LRP2 | | | | | | | | | 6 | | | |
| LRP3 | | | | | | | | | 23 | | | |
| LRP4 | | | | | | | | | 23 | | | |
| LRP5 | 21 | | | | | | | | | | | |
| LRP6 | | | | | | | | | 23 | | | |
| LRPPRC | | | | | | | 3 | | | | | |
| LRRC1 | | | | | | | | | | | 3.833 | |
| LRRC10 | | | | | | | | | | | 23 | |
| LRRC14B | | | | | | | | | 21.5 | | | |
| LRRC16A | | | | 23 | | | | | | | | |
| LRRC17 | | | | 21 | | | | 21.5 | | 18 | | 23.5 |
| LRRC27 | | | | | | | 16 | | | | | |
| LRRC28 | | | | | | | | 23 | | | | |
| LRRC3 | | | | | | | | | 8 | | | |
| LRRC30 | | | | | | | 20 | | | | | |
| LRRC31 | 2 | | | | | | | | | | | |
| LRRC32 | | | 12 | | | | | | | 5.5 | | |
| LRRC36 | | | | | | | | 2 | | | | |
| LRRC4 | | | | | | | | | | 4.5 | | |
| LRRC41 | | | | | | | | | 22 | | | |
| LRRC49 | 17 | | | | | | | 20 | 21 | 12 | | |
| LRRC52 | | | | | | | | | 21 | | | |
| LRRC55 | | | 23 | | | | | | | 3 | | |
| LRRC57 | | | | | | | | | 17 | | | |
| LRRC58 | | | | | | | | 22 | | | | |
| LRRC61 | | | 22 | 23 | | | | | | | | |
| LRRC8A | | | 4.5 | | | | | | 22 | 11.5 | | |
| LRRC8B | | | 21 | | | | | 10 | | | | |
| LRRC8D | | | | | 18 | | | | | | | |
| LRRC8E | | | | | 17 | | | | | | | |
| LRRFIP1 | 8 | | | | | 11 | | | | | 9 | |
| LRRIQ1 | 21 | | | | | | | | | 5 | | |
| LRRK1 | | | | | | | | | | 10.5 | | |
| LRRK2 | 21 | | | | | | | | | | | |
| LRRN1 | | | | | | | | | 0 | | | 23 |
| LRRN3 | | 14.5 | | | | | | | | 14 | | |
| LRRN4 | | | | | | | | | | 7 | | |
| LRRN4CL | | | | | | 1 | | | 22.5 | | | |
| LRRTM2 | | | | | | | | | | 11 | | |
| LRRTM3 | | | 6 | 4.5 | | 3 | | 0 | 1 | | | 1 |
| LRTM1 | | 6 | | | | | 8 | 8 | | | | |
| LSM10 | | | | | | 17 | | | | | | |
| LSM11 | | | | | 2 | | | | 23 | | | |
| LSM14A | 20 | 19.5 | 20.5 | | | 18 | | 13.5 | 18.5 | | | |
| LSMD1 | | | | | | | | | 23 | 14 | | |
| LSP1 | 18.5 | | | | | | | | | | | |
| LSR | | | | | | | | 16 | | | | |
| LSS | | 22.5 | | | 3.5 | 22 | | | | | | |
| LTA4H | | | | | | | | | 22 | | | |
| LTB | | | | | | | | | | | | 9 |
| LTBP1 | | | 18 | | | | | | | | | 20 |
| LTBP2 | | | | | | | | | | 15.5 | | |
| LTN1 | | | | | | | | | | 10 | | |
| LUC7L | | | | | | 21.5 | | | | | 20 | |
| LUC7L2 | | | | | | | | | 18.5 | | | |
| LUC7L3 | | | | | | | | | 14.5 | | | |
| LUM | | | | | | | 2.5 | | | | | |
| LURAP1L | | | 21 | | | | | | | 11 | | |
| LUZP1 | | | | | | | | | | 14 | | |
| LY6A | | | | | | 20 | | | | | | |
| LY6E | | | | | | 13 | | | | | | |
| LY6G6E | | | | | | 4 | | 23 | | | | |
| LY86 | | | | | | | | | 14 | | | |
| LY96 | | | | | | | | | | | 10 | |
| LYPD6 | 21 | | | | | 21.5 | | | 0 | | | |
| LYPLA2 | | | | | | | | 13.5 | 14 | | | |
| LYRM1 | | 11.5 | 10 | | | | | | | | | |
| LYRM4 | | | | | | | | | | 22.5 | | |
| LYRM5 | | | | | | | | | 16 | | | 12 |
| LYSMD2 | | | | | | | | | 22 | | | |
| LYSMD3 | | | 10 | | | | | | | | | |
| LYST | | | 12 | | | | | | 6 | | | |
| LYVE1 | | | | | | | | | 3 | | | |
| LZIC | | | 0 | | | | | 22 | 23 | | | |
| LZTS2 | | | | | | | | | | 7 | | |
| MAB21L2 | | | | | | | | 1 | | | | |
| MACF1 | | | | | | | | | | 5 | | |
| MAD1L1 | | | | 20.5 | | | | | | | | |
| MAD2L2 | | | | | | | | | 20.5 | | | |
| MAF | | | | | | 11 | | 2 | 18 | 18 | | |
| MAF1 | | | | | | | | 3 | | | | |
| MAFB | | | | | | | | | 5 | 6 | | |
| MAFF | | | | | | | | | 3 | | | |
| MAFK | | | | | | | | 7 | | | | |
| MAG | | | | | | | | | 11.5 | 12 | | |
| MAGED1 | | | 17 | | | 22 | | 12 | 12 | 11 | 0 | |
| MAGEE2 | | | | | | | | | | 16 | | |
| MAGI1 | | | | 22.5 | | 7 | | 5 | 4 | 4 | | |
| MAGI2 | | | | | | | | 13 | | | | |
| MAGI3 | | | | | | | | | 18 | | | |
| MAGOH | | | | | | | | | | 8 | | |
| MAGT1 | 0.5 | | | | | | 4 | | | 22.5 | | |
| MAL2 | | | 2.5 | | | | | | | | | |
| MALT1 | 0 | | | | | | | | | | | |
| MAN1A2 | | | | | | | | | | 3 | | 17 |
| MAN2A1 | | | | | | | | | 20 | | | |
| MAN2B2 | | | 22.5 | | | | | | | | | 23 |
| MAN2C1 | | | | | | | | | | 9 | | |
| MANBA | | | | | | | | 8 | 10 | | | |
| MANBAL | | | | | | | | 0 | 23 | | | |
| MANEA | | | | | | 3 | | 0 | | 3 | | |
| MANF | | 23 | 22 | | | | 19 | | | | | 0 |
| MANSC4 | 18.5 | 18 | 19.5 | | 19 | 19 | | 19 | 19.5 | 18 | 18 | 20 |
| MAOA | | | | 21 | | | | | | | | |
| MAP1A | 14 | 13 | 15 | | | 15.5 | | 13 | 16 | 14 | | 14 |
| MAP1LC3A | | | | | | | | 8 | 4 | | | |
| MAP2K1 | | 6 | | | | | | | | | | |
| MAP2K3 | | | | | 19 | | | | | | | |
| MAP2K6 | | | 19.5 | | | | | | | | | |
| MAP2K7 | 16 | | 20 | | | | | | | | | 19.5 |
| MAP3K1 | | | | | | | | | 22 | | | |
| MAP3K5 | | | | | | | | 23.5 | | | | |
| MAP3K6 | | | | | | | | | | | 7.167 | |
| MAP3K9 | | | | | | | 9 | | | | | |
| MAP4 | | | | | | | | | | | 5 | |
| MAP4K2 | | | | | | | | | 21 | | | |
| MAP4K4 | | 8.5 | 8 | | | 10.5 | | 12 | | | | |
| MAP4K5 | | | | | | | 7 | | | | | |
| MAP7 | | | 23 | 22.5 | | | | | | 23 | | |
| MAP7D3 | | | | | | 10 | | | | | | |
| MAPK10 | | | | | | 15 | | | | | | |
| MAPK12 | | | | | | 10 | | | | 14 | | |
| MAPK14 | | 9.5 | | | | 20 | | | | | | |
| MAPK15 | | | | | | | | | 5.5 | | | |
| MAPK1IP1 | | | | | | | | 0 | | | | |
| MAPK1IP1L | | | | | | | | 22 | | | | |
| MAPK4 | | | | | | | | | | 9.5 | | |
| MAPK6 | | | | | 21 | | 23 | | 20 | | | |
| MAPK8IP1 | | | | | 23.5 | | | | 19 | | | |
| MAPK8IP3 | | | | | | | | 20 | 15 | | | |
| MAPKAP1 | | | | | | | | 21.5 | 23 | | | |
| MAPKAPK2 | | | | | | 9 | | | | | | |
| MAPKBP1 | 1 | 0 | 2 | | 2.5 | 1 | | 0 | 22.5 | 0 | | 3 |
| MAPRE1 | | | | | | | | | | 23 | | |
| MAPRE2 | | | | | | | | | 11 | 20 | | |
| MAPRE3 | 8 | 7 | | | | | | | | 3 | | |
| MAPT | | | | | | | | | 3 | | | |
| MARCKS | | | | | | 11 | | 11.5 | 21 | | | |
| MARK2 | | | | | | | | | 16 | | | |
| MARK4 | | | | | | 3 | | | | | | |
| MARS2 | | | | 23 | | | 2 | | | | | |
| MARVELD1 | | | | | | | | | | 14 | | |
| MASP2 | | | | | | | | | 14 | | | |
| MAST1 | | | | | | | | 15.5 | | | | |
| MAST2 | | | | | | | | | | 14 | | |
| MAST3 | | | | | 20 | | | | | 15 | 7 | |
| MAST4 | 21.5 | | | | | | | | | | | |
| MAT2A | | | | | | | | | | 14 | | |
| MATN2 | | | | | | 9 | | | | | 8 | |
| MATR3 | | | | | | | | | | | 11.83 | |
| MAVS | | | | | | | | | | 13.5 | | |
| MAX | | | | | | | | 9 | | | | |
| MAZ | | | | | | 18 | | | | | | |
| MBD1 | | | 23 | | | | | | | | | |
| MBD4 | | | | | | | | | | 11 | | |
| MBD5 | | | | | | 6 | | | | 3 | | |
| MBL1 | | | | | | 10 | | | | | | |
| MBLAC1 | | | | | | | | | | 3 | | |
| MBLAC2 | | | | | | | 13 | | | | | |
| MBNL1 | | | | | | 16 | | | | | | |
| MBNL2 | | | | | | | | | | 19 | | |
| MBOAT1 | 5 | | | | | | | 7 | | | | |
| MBTD1 | | | 21 | | | | | | | | | |
| MC2R | | | 6 | | 4 | 8 | | | | 6 | | |
| MC5R | | | | | | | | 4 | | | | |
| MCAM | | | | | | | | 11 | | | | |
| MCART1 | | | | | 7 | | 7 | | | | | |
| MCC | | | | | | | | | | 9 | | |
| MCEE | | | | | | 12.5 | | 22 | 22 | | | |
| MCF2L | | | | | | | | | 2 | | | |
| MCFD2 | | | | | | | | | | 19 | | |
| MCHR1 | | | | | | | | 10 | | | | |
| MCM10 | | | | | | | | | 0 | | | |
| MCM4 | 6 | | | | | | | | | | | |
| MCM5 | | | 17 | | | | | | | | | |
| MCM7 | | | 23 | | | | | | | 9 | | |
| MCM8 | | | | 12 | 12 | | 10 | 13.5 | | | | |
| MCM9 | | | | | | | | 9 | | | | |
| MCOLN1 | | | | | | | 20 | | 21 | | | |
| MCOLN3 | | | | | | | | | 22 | | | |
| MCPH1 | | | | | | | | | 0 | | | |
| MCPT4 | | | | | | | | | 5 | | | |
| MCTP1 | | | 22 | | | | | | | | | |
| MCU | | | | | | | | | | 13 | | |
| MDFIC | | 16 | 18 | | | | | 16 | | | | |
| MDGA2 | 19.5 | 23 | 23 | | 23.5 | 0 | | 22 | 22 | 22 | 2 | 23 |
| MDH2 | | | | | | | | 15 | | | | |
| MDM1 | | 5.5 | | | | | | | | | | |
| MDN1 | | | | | | | | 22 | | | | |
| ME1 | 10 | | | | | | | | | | | |
| ME3 | | | | | | | | | 22.5 | | | |
| MECOM | | | | | | | | | 21.5 | | | |
| MECP2 | | | | | | | | 23.5 | | | | |
| MED11 | | 7.5 | | | | | | | | | | |
| MED12 | | | | | | | | | 12 | | | |
| MED12L | | 18 | | | | 0 | | 20 | 16 | | 1 | |
| MED13 | | | | | | | | | | 4 | | |
| MED14 | | | | | | 3 | | | | 6.5 | | |
| MED15 | 8 | 9 | | | | | | 9.5 | | 9 | | 10 |
| MED16 | | | | | | | | | | 4 | | 1 |
| MED20 | | | | | | | | | | | 23 | |
| MED23 | | | | | | | | | 22 | | | |
| MED24 | | | | | | | | | 22.5 | | | |
| MED28 | | | | | | | | 3.5 | | | | |
| MED30 | | | | | | | | | | 21 | | |
| MED31 | | | | | | | | | | | 19.5 | |
| MED6 | | | | 4.833 | | | | | | | | |
| MED7 | | | | | | | | | 0 | 8 | | |
| MED8 | | | 5.5 | | | 7.5 | | | | | | |
| MEF2D | | | | | | 14 | | | | | | |
| MEGF10 | | | | | 18 | | | | | | | |
| MEGF6 | 23.5 | 0 | | | | 22 | | | 23 | | | |
| MEGF9 | | 7 | | | | | | | | | | |
| MEIS1 | | | | | | | | 22 | | | | |
| MEIS2 | | | | | | 19 | | | | | | |
| MEMO1 | | | | | | | | | | 16 | | |
| MEN1 | | | | | | 22.5 | | | 23 | 0 | | |
| MEPCE | | | 1 | | | | | | | | | 0 |
| MERTK | | | | | | | | | 0 | 19 | | |
| MESDC2 | | | | 3 | 2 | | 3 | | | | | |
| MEST | | | | | | | | | 0.5 | | | |
| MET | | | | | | | | | 23 | | | |
| METTL10 | | | 21.5 | | | | | | | | | |
| METTL13 | | | | | | 10 | | | | 11 | | |
| METTL16 | | | | | | | | | | | | 8 |
| METTL17 | | 6 | | | | 0 | | | | | | |
| METTL20 | | | | | | 22 | | 9 | | 7.5 | | |
| METTL22 | | | 22 | | | | | | | | | |
| METTL24 | | | 21 | | | | | | | | | |
| METTL3 | | | | | 19 | | | | | 7 | | |
| METTL4 | | | | | | | | | 20 | | | |
| METTL6 | | | | 22 | | | | | | | | 21 |
| METTL7A | | | | | | | | | 20 | | | |
| METTL7B | 17 | | | 19 | | | | 23 | | 17 | | |
| METTL8 | | | | | | | | 5.5 | 1 | | | |
| METTL9 | | | | | | | | | | | 8 | |
| MFHAS1 | | | | | | | | 20 | | | | |
| MFN1 | | | | | | | | | | | 1.5 | |
| MFNG | | | 3.833 | | | | | | | | | |
| MFSD1 | | | | | | | | | | 15 | | |
| MFSD12 | | | | | | | | 8 | | | | |
| MFSD2A | | | | | | | | 16 | | 13 | | |
| MFSD4 | | | | | | | | | | 3 | | |
| MFSD5 | | | | | | | | | | 7 | | |
| MFSD6 | | | | | | | | | 5 | | | |
| MFSD7 | | | | | | | | 9.5 | | | | |
| MFSD8 | | | | | | | | 19 | 21.5 | | | |
| MFSD9 | 3 | | | | | | | | | | | |
| MGA | | | | | | | | | 21 | | | 0 |
| MGAT1 | | | 20 | | | | | | | | | 20.5 |
| MGAT4B | | | | | | 5.5 | | 22 | | | | |
| MGAT5 | | | | | | | | 20 | | | | |
| MGEA5 | | | | | | | | | 1.5 | | | |
| MGLL | | | | | 7.167 | | | | | | | |
| MGMT | | | | | | | | | | 5.5 | | |
| MGP | | | | | | | | | | | 1.833 | |
| MGRN1 | | | | | | | | 11 | | | | |
| MGST1 | | | | | | | | | 22 | | | |
| MGST2 | 20 | | 22 | | | | | | | | 3.5 | |
| MI | 18.5 | | | | | | | 20 | 22 | 5.5 | | |
| MIA2 | 22.5 | | | | | | | | | | | |
| MIB1 | | | | | | | 23 | | | | | |
| MICAL1 | | | | | | 10 | | | | | | |
| MICAL2 | | | | | | 10 | | | | | | |
| MICAL3 | | | 22 | | | 23 | | | | | 0 | |
| MICALCL | | | | | | | | | | 13 | | 12.5 |
| MICU1 | 4.5 | | | | | | | 3 | 5 | | | |
| MID1IP1 | | | | | | | | | | | | 8 |
| MID2 | 8 | | | | | 17 | | | 7.5 | 12 | | 9.5 |
| MIF4GD | 0 | | 23.5 | | | | | | | 0 | 3 | 2 |
| MIR107 | | | | | 23 | | | | | | | |
| MIR122A | | | | | 6 | 1 | | 23 | 22 | | | |
| MIR125B-1 | | | | | | | | 8.5 | | 11 | | |
| MIR128-1 | | | | | | | | 23.5 | | | | |
| MIR146 | 8 | 9 | 9 | 11 | | 10 | | | 5 | 12 | 9 | |
| MIR181B-2 | | | | | | | | | | 10 | | |
| MIR218-1 | | | | 13 | | | | | | | | |
| MIR218-2 | | | | | | 16.5 | | | | | | |
| MIR219-2 | | | | | | | | | | | | 8 |
| MIR29B-2 | 8 | | 11 | 12 | 12 | | | 11 | 11.5 | 12 | | |
| MIR365-1 | | 14 | 14 | | 12 | 12 | 12 | 11.5 | 12 | | | |
| MIR365-2 | | | | 0 | | | | | | | | |
| MIR499 | 6 | | | | | | | 7.5 | | 6 | | |
| MIR505 | 7 | | | | | | | | 9 | | | |
| MIR687 | | | | | | | | | | 2 | | |
| MIR694 | | | 23.5 | | | | | | | | | |
| MIR701 | 2 | 2 | 2 | | | | | | | | 0 | 23.5 |
| MIR708 | | | 12 | | | | | | 16 | | | |
| MIRLET7A-2 | | | | | | 4 | | | | | | |
| MIS12 | | | | 7.5 | | | 6 | | 10 | | | |
| MITF | | | | | | | | | | | | 5 |
| MKKS | | | | | | | | | 19 | | | |
| MKL2 | | | | | | | | | | 18 | | |
| MKLN1 | 19 | | | | | | | 22.5 | 23.5 | | | |
| MKNK1 | | | 22 | | | | | 22 | | | | |
| MKNK2 | | | | | | | 22.5 | 22 | | | | |
| MKRN1 | | | | | | 0 | | | | | | |
| MKRN2 | | | 19 | | | 21 | | | | | | |
| MKS1 | 11 | | | | | | | | 6 | | | |
| MLC1 | | | | | | | | 0.5 | | | | |
| MLEC | | | | | | | | | 20 | | | |
| MLF1 | | | | | | | | | | 11 | | |
| MLH1 | | | | | | | | 18.5 | | | | |
| MLH3 | 21 | | | 22 | | | 23.5 | 2 | | | | |
| MLIP | | | 23 | | | | | | | | | |
| MLL2 | | | 6 | | | | | 6.5 | 23 | | | |
| MLL4 | | | | | | | | | | 12 | | |
| MLL5 | | | | | | | | | 22 | | | |
| MLLT3 | | 11 | 11.5 | | | 14 | | | 17 | 12 | | |
| MLLT4 | | | 23 | | | | | 22 | | 10 | | |
| MLST8 | 22.5 | | | | | | | | | | | |
| MLXIP | | 0.5 | 1 | 1 | | | | 23.5 | 0 | | | |
| MLXIPL | | | | | | | | | 8 | | | |
| MMAA | | | | | | | | | 1 | | | |
| MMAB | | | | | | | | | 19 | | | |
| MMACHC | 21 | | | | | | | | | | | |
| MMD | | 2 | 1 | | 3 | | | | 1.5 | | | |
| MMD2 | | | | | | | | | | | 2 | |
| MME | | | | | | | | 22 | | | | |
| MMGT1 | | | | | | | | 21 | | | | |
| MMGT2 | 20 | 3.5 | | | | | | 1 | | | | |
| MMP11 | | | | | | 7 | | | 1 | | | |
| MMP12 | | | | | | | | | | 13 | | |
| MMP14 | | | | | | | | | | 3 | | |
| MMP15 | | | | | | | | | 23 | | | |
| MMP19 | | | | | | | | | | 10 | | |
| MMP2 | | | | | | | | | | 15 | | |
| MMP23B | | | 2 | | | 0.5 | | 0 | | 4.5 | | 4 |
| MMP9 | | | | | | | | | 17 | 16 | | |
| MMRN2 | | | 18 | | | | | | | | | |
| MN1 | 21.5 | 0 | 1 | | | | | 0 | 23 | | | 23.5 |
| MNF1 | | | | | | | 19 | | | | | |
| MOB1A | | | 21.5 | | | 23 | | 23 | | | | |
| MOB3B | 9 | 10 | 11 | | | | 6.5 | 5 | 7.5 | | | |
| MOCOS | | | | | | | | | | 7 | | |
| MOCS1 | | | | | | 17 | | | | | | |
| MOCS2 | | 19 | | | | | | | | | | |
| MOGAT2 | | | 6 | | | | | 0 | 0 | | | |
| MOGS | | | | | | | | | | | 7 | |
| MON1A | | | | | | | | 6 | 2 | | | |
| MORC2B | | | | | 22 | | | 2 | | | | |
| MORC3 | | | 20 | | | | | | 17 | 9 | | |
| MORC4 | | | | | 22 | | | 22.5 | | 18.5 | | |
| MORF4L2 | | | | | | | | | 3 | | | |
| MORN2 | | | | 19.5 | | | | | | | 3.5 | |
| MOSPD1 | | | 11 | | | | | | | | | |
| MOSPD2 | | | | | | | | | | 12 | | |
| MOV10 | | | | 13 | | 17 | 13 | | | | | |
| MPDU1 | | | | | | | | | 15 | | | |
| MPDZ | | | | | | | | | | | 19 | |
| MPHOSPH6 | | | | | | | | | | | 23 | |
| MPI | | | | | 6 | | | 4 | | | | |
| MPLKIP | | 7 | 11 | | | | | 6 | | | | |
| MPP1 | | | 3.167 | | | | | | | | | |
| MPP2 | 21.5 | | | | | | | 23 | | | | |
| MPP4 | | | | | | | | | | 12 | | |
| MPP5 | | | | | | | | | 23 | | | |
| MPP6 | 19 | | | 21.5 | | | 21 | 20 | | | | |
| MPP7 | | | | | | | | | 8 | 7 | | |
| MPPE1 | | | | | | | | | | 14 | | |
| MPPED1 | | | | 22.5 | | 1 | | 21 | | | 2 | |
| MPRIP | | | 9 | | | | | | | 11 | | |
| MPST | | | | 18 | | | | | | | | |
| MPT | | | | | | | | | 0 | | | |
| MPZL1 | | | 7 | | | | | | | | | |
| MPZL2 | | | | | | | | | | 14 | | |
| MPZL3 | | | | | | | 12 | | 23 | | | |
| MR1 | | | | | | | | | 1 | | | |
| MRAP | | | | | | | | 0 | 4 | | | |
| MRAP2 | | | | | | | | | 21 | | | |
| MRAS | | | 2 | | | | | | | | | |
| MRC2 | | | | | | 5 | | 18 | | | | |
| MRE11A | | | 11 | | | | | | | | | |
| MREG | | | | | | | | 22 | | | | |
| MRGPRA4 | | | | | | | | | 2 | | | |
| MRGPRF | | | | | | | | | | | 7 | |
| MRGPRH | | | | 4 | | | | | | | | |
| MRP63 | | | | 22 | | | | | | | | |
| MRPL1 | | 7 | | | | | | | | 5.5 | | 8.5 |
| MRPL10 | 7.5 | | | | | | | | | | | |
| MRPL14 | | | | | | | | 23 | | | | |
| MRPL15 | | | | | | | | 7 | | | | |
| MRPL16 | | | | | | | | | | | | 8.5 |
| MRPL2 | | | | 23 | | | | | | | | |
| MRPL24 | | 7 | 9 | | | | | | | | | |
| MRPL34 | | | | | | | | | | 8 | | |
| MRPL35 | | | | | | | | | 11 | | | |
| MRPL36 | 9 | | | | | | | | | | | |
| MRPL4 | | | 10 | | | | | | | | | |
| MRPL49 | | | | | 18 | | | | | | | |
| MRPL50 | | | | | | | | 9 | | 21 | | |
| MRPL51 | | | | | | | | | | 23.5 | | |
| MRPL52 | | | | | | 18 | | | | | | |
| MRPS17 | | | 19.5 | | | | | | | | | |
| MRPS18B | | | | | | | | 18 | | | | |
| MRPS2 | | | | | | | | | | 7.5 | | |
| MRPS21 | | | | | | 12 | | | | 15.5 | | |
| MRPS22 | | | | | | 12 | | | | | | |
| MRPS23 | 20 | | | | | | | | | | | |
| MRPS24 | | | | | | | | 10 | | | | |
| MRPS27 | | | | | | | | | | | 19 | |
| MRPS35 | | | | | | | | | | 20 | | |
| MRPS6 | | | | | 21 | | | | | | | |
| MRPS7 | | | | | | | | | 19 | 5 | | |
| MRRF | | | 22 | | | | | | | | | |
| MRVI1 | | | 0 | | | | | | | | | |
| MS4A1 | | | | | | | | 19.5 | | | | |
| MS4A4C | | | | | | | | | | 22 | | |
| MS4A4D | | 6 | 6 | | | | | | | | | |
| MS4A6C | | | | | | | | 21 | | | | |
| MS4A8B | | | | 21.5 | | | | | | | | |
| MSH6 | | | 22 | | | | | | | | | |
| MSI2 | | | | | | | | 7 | | 11 | | |
| MSL1 | | | | 22 | 22 | | 21 | | | | | |
| MSMO1 | | | | | | | | 22 | | | | |
| MSN | | | | | | | 0 | | | | | |
| MSRB2 | | | | | | | | | | | 23 | |
| MST1R | | 7.5 | | | | | | 9 | | | | |
| MSTO1 | | | | | | | | | | 16 | | |
| MT1E | | | | | | | | | | 17 | | |
| MT1H | 20.5 | | | | | | | 13 | | 12 | | |
| MTA1 | | | 18 | | | | | | | 14 | | |
| MTA2 | | | | 23 | 0.5 | | 23 | | | | | |
| MTA3 | | | 18 | | | 17 | | | | 15 | | |
| MTAP | | | | | 9 | | 6 | | | | | |
| MTBP | 10 | | | | | | | | | | | |
| MTCH1 | | | | | 12 | | | | | | | |
| MTCP1NB | | | | | | | | | 12.5 | | | |
| MTDH | | | | | | 17 | | | 22 | | | |
| MTERFD1 | | | | | | | | | 21.5 | | | 21 |
| MTFMT | 5.5 | | | | | | | 20 | 21 | | 21 | |
| MTHFD1 | | | 12 | | | | | | | 13 | | |
| MTHFD1L | | | 13 | | | | | | | 8.5 | 8.5 | 9.5 |
| MTHFR | | | | | | | | 22 | | | | |
| MTM1 | | | | | | | | 19.5 | | 17 | | |
| MTMR12 | | | | | | | | | | | | 8 |
| MTMR14 | | | | | | | | | | 23.5 | | |
| MTMR2 | | | | | | | | | 5 | | | |
| MTMR4 | | | | | | | | | 13.5 | | | |
| MTMR6 | | | | | | | | | | 16 | | |
| MTMR9 | | | | | | | | | | | 23.5 | |
| MTNR1A | | | | | | | | | 22.5 | | | |
| MTOR | | | | | | | | | | 11 | | |
| MTR | | | 10.5 | | | | | | | | | |
| MTRR | | | | | | | | | | 9 | | |
| MTSS1 | | | | | | 12 | | 8 | | 9 | | |
| MTSS1L | | | | | 16 | | | | 18 | | | |
| MTTP | 21 | | | | | 23 | | | 16 | 2 | 0.5 | 21 |
| MTUS1 | | | 9 | | 5 | 1 | | | | | | |
| MTUS2 | | 2 | 3 | | | | | | 23 | 19 | | |
| MTX2 | | | 22 | | | | | | | | | |
| MTX3 | | | | | 7 | | | | | | | |
| MUC13 | | | | | | | | | | | 22.5 | |
| MUC15 | | | | 13 | | | | 11 | | | | |
| MUC20 | | | | | | | | | | 4 | | |
| MUC5B | | | | 19 | | | | | | | | |
| MUL1 | | | | | | 1 | | 0 | | 2 | | |
| MUM1L1 | | | | | | | | | 22 | | | |
| MURC | | | | | | | | | | 9 | | |
| MUS81 | | | | | | | | | | 4 | | |
| MUSTN1 | | | | | | | | 23.5 | | | | |
| MUT | | | | | | | | | | 4.5 | | |
| MUTED | | | | | | | | 1 | | 6.5 | | |
| MUTYH | 6.5 | | | | | | | 5 | 4 | | | |
| MVD | | | | | | | | | | 22 | | |
| MVK | 0 | | 3 | | | 3 | | 22 | | | | 2.5 |
| MVP | | | | | | | | | 21 | | | |
| MXD1 | | | | | | | | | | | 0 | |
| MXD4 | | | | | | | | | | 11.5 | | |
| MXI1 | | 19 | 19.5 | | | | | | | | | |
| MXRA7 | | | | | 2 | | | 23 | | | | |
| MYADM | 3 | | 11 | | | 21 | | | | | | |
| MYADML2 | | | | | | | | | 12 | | | |
| MYB | | | | | | | | | 21 | | | |
| MYBBP1A | | | 4.333 | | 5.5 | | | | 21 | 4 | | |
| MYCBP | 17 | | | | | | | | | | | |
| MYCBP2 | | | | 12 | | | | | | | | |
| MYD88 | | | | | | | | | 11 | | | 1.833 |
| MYEF2 | | | | | | | | 9 | | | | |
| MYEOV2 | | | | | | 15 | | | | | | |
| MYF6 | | | | | | | | | | 23 | | |
| MYH10 | 7 | | | | | | | | | 10 | 7 | |
| MYH11 | | | | | | 23 | | | | 7 | 22 | |
| MYH14 | 6 | | | | | | | | | | | |
| MYL12A | | | | | | | | | 23 | | | |
| MYL12B | | | | | | | | | 2 | | | |
| MYL4 | 5.5 | | | | | | | | | | | |
| MYL6B | | | | | | | | | 23 | 17 | | |
| MYL9 | | | | | | | | 21 | 22 | | 22 | |
| MYLIP | | | | | | | | | | 8 | | |
| MYLK | | | | | | | | | 21.5 | | | |
| MYLK4 | | 7 | 8 | | | | | | | | | |
| MYO10 | | | | | 23 | | | | | | | |
| MYO18A | | | | | | | | 23 | | 12 | | |
| MYO19 | | | | | | | | | | 9 | | |
| MYO1A | | | | | | | | | | | | 8.5 |
| MYO1B | 15 | | | | 9 | | | | | | | |
| MYO1C | 22 | | | | | | | | | | | |
| MYO1D | | | | | | | | 21 | | 21 | | |
| MYO1E | 20 | | 2 | | | | | 21 | | | | |
| MYO1G | 12 | | | | | | | | 13 | | | |
| MYO1H | | 7 | | | | | | 7 | | | | |
| MYO3B | | | | | | | 9.5 | | | 11 | | |
| MYO5A | 20.5 | | | | | | | | 2 | | | |
| MYO5B | | | | | | 16 | | | | | | |
| MYO5C | | | | | 22.5 | | | 0 | | | | |
| MYO7A | | | | | | | | | | | 16 | |
| MYO9B | | | | | | | | | 20 | 15.5 | | |
| MYOD1 | | | | | | 11 | | | | | | |
| MYOF | | | | | | | | | 23.5 | | | |
| MYOM2 | | | | | | | | 19 | | | | |
| MYOT | | | | | | 2 | | | 23 | | | |
| MZT1 | 20 | | | | | 19.5 | | 19 | 20 | | | |
| N4BP2L1 | 17 | | | | | | | | | | | 21 |
| N4BP2L2 | | | | | | | | 22 | 0 | | | |
| N6AMT1 | 18 | | | | | 22 | | 20 | 20 | 18.5 | | 21 |
| N6AMT2 | | | | | | | 23 | | | | | |
| NBAS | | | | | | | | | 0 | | | |
| NBEAL2 | | | | | | 2 | | 20 | 19 | | 1 | |
| NBL1 | | | | | | | | | 21 | | | |
| NBR1 | | | | | | | | 9 | | 11 | | |
| NCALD | | | | | | 21 | | | | | | |
| NCAM1 | | 22 | | | | | | | | | | |
| NCAPD2 | | | | | | | | | | | | 21.5 |
| NCBP1 | | | 22 | | | 2 | | | | 3 | | |
| NCBP2 | | | | | | | | | 6 | 8 | 7 | |
| NCCRP1 | | 14 | 15 | | 11 | 13 | | 12 | 16 | | 13 | |
| NCDN | | | | | | | | | 17 | | | |
| NCEH1 | | | | 21 | | | | | | | | |
| NCK1 | | | | | 20 | | | | | | | |
| NCK2 | | | | | | | | | 23 | | | |
| NCKAP1 | | | | | | | | | | 15 | | |
| NCKAP5 | | | | | | | | | | 12 | | 12 |
| NCL | | 7.5 | 9.5 | | | | 8 | 9.5 | 3 | | | |
| NCOA2 | | | | | | | | | | 6 | | |
| NCOA3 | | | | | | | | | 20 | | | |
| NCOA5 | | | | | | | | | | 10 | | |
| NCOA6 | | | | | | | | | | 17 | | |
| NCOA7 | | | | | | | | 23 | | | | |
| NCOR2 | | | | | | | | | 20 | | | |
| NCSTN | | | | | | | | 12 | 12 | | | 12 |
| NDE1 | 20 | | | | | | | | 22 | | 23 | |
| NDEL1 | | | | | | | | | | 15 | | |
| NDFIP1 | | 16 | | | | 13.5 | | 18 | | | | |
| NDFIP2 | | | | | | | | | | | 22 | |
| NDRG1 | 20 | | | | | | | | | | | |
| NDRG2 | | | | | 21 | | | | | | | |
| NDRG3 | | | 5.833 | | | | | | | | | |
| NDST1 | | | 20 | | | | | | | | | |
| NDST2 | | | | | | | | | | 0.5 | | |
| NDST3 | | | | | | | | | | | 1.167 | |
| NDUFA5 | | | | | | | | | | 0 | | 4.5 |
| NDUFA6 | | | | | | | | 12 | | | | |
| NDUFAF4 | | 18 | 18 | | | | | | | 17 | | |
| NDUFB11 | 9 | 7 | 9 | | | | | 12 | 9 | | | |
| NDUFB3 | | | | | | | | 22 | | | | |
| NDUFC1 | | | | | | | | | | 23 | | |
| NDUFS2 | | | | | | | | | | 4 | | |
| NDUFS6 | | | | | | | | | | | 2 | |
| NDUFS7 | | | | | | | | | 2 | | | |
| NDUFV1 | | | | | | 10 | | | | | | |
| NDUFV3 | | | | | | 10 | | | 5 | | | |
| NEB | | | | | | | | | | 22 | | |
| NEBL | | | | | | | 8 | | | 13 | | |
| NECAB1 | | | | | | | | | | 2.5 | | |
| NECAB2 | | | | | | | | 8 | | | | |
| NECAP1 | | | | | 6 | | | 12.5 | | | | |
| NEDD4 | 0 | | | | | | | 0 | 3 | | | |
| NEDD4L | | | | | 4 | 2.5 | | | | 2.5 | 4 | |
| NEDD9 | | | | | | | | | | | 21 | |
| NEFM | | | 5.167 | | | | | | | | | |
| NEGR1 | | | | 12 | | | | | | | | |
| NEIL1 | 0 | | 19 | | | | | 0.5 | | | | |
| NEK2 | | | | | | | | 12 | 13 | | | |
| NEK3 | | | | | | | | | | 0.5 | | |
| NEK4 | | | | | | | | | | 11 | | |
| NEK5 | | | | | | | | 6 | | | | |
| NEK7 | | | | | | | | | 17 | | | |
| NEK8 | | 4 | | | | | | | | | | |
| NEK9 | | | | | | 0 | | | | 20 | | |
| NELF | | | | | | | | 6 | | | | |
| NELL1 | | | 20 | | | | | | 15 | 0 | | |
| NENF | | | | | | | | | 23 | | | |
| NEO1 | 19 | | | | 20 | | | 22 | 22 | | | |
| NET1 | | | | | | | | | | | 10 | |
| NETO2 | | 20.5 | | | 23 | | | | 19 | 20 | | |
| NEU1 | | | | 22 | | | | | | | | |
| NEU2 | | | | | | | | | 21 | | | |
| NEU3 | | | | | 21 | | | | 22 | | | |
| NEU4 | 9 | 9 | 10 | | | | | | 15 | 9 | | 12 |
| NEURL2 | | | | | | | | | 11 | 16.5 | | |
| NEURL3 | | | | | | | | | | | 23.5 | |
| NEURL4 | | | | | | | | 6.5 | 5 | | | |
| NF1 | | | | | | | | | | 7 | | |
| NFAT5 | | | | | | | | | | 3 | | |
| NFATC3 | | | | 14 | | | 15 | 16 | 20 | 16.5 | 16.5 | |
| NFATC4 | | 14 | | 14 | 12.5 | | 14.5 | | | 14 | | |
| NFE2L1 | | | | 12 | | | | | | | | |
| NFE2L2 | | | | | | | | | 1 | | | |
| NFE2L3 | 23 | 2 | | | | | | 22.5 | | | | |
| NFIA | | | | | | | | | 22.5 | | | |
| NFIB | | | | | | | | | | 22 | | |
| NFIC | | | | | | 13 | | | | | | |
| NFIL3 | | | | | | | | | | 2.5 | | |
| NFIX | | 0 | | | | | | | 22 | | | |
| NFKB1 | | | | | | 14.5 | | | | | 11 | |
| NFKBIA | | | | | | | 4 | | | | | |
| NFKBIB | | | | | | | | | 21 | | | |
| NFKBID | | | | | | | | | 23 | | | |
| NFRKB | | | | | | | | 20.5 | | | | |
| NFX1 | | | | | 12 | | | | | 9.5 | | |
| NFXL1 | | | | | | | | 23 | | | | |
| NFYA | | | | | | | | 21 | | | | |
| NGEF | 21.5 | | | 3 | | | | | | | 22 | |
| NGLY1 | | | | | 12.5 | | | | | | | |
| NGRN | | | | | | 14.5 | | | | 11 | | |
| NHEJ1 | | | | | | | | | 15.5 | | | |
| NHLRC2 | | 5.5 | | | | | | | | 12 | | |
| NHP2 | | | 7 | | | | | | | 9 | | |
| NHSL1 | | | 4.5 | | | | | | | | | |
| NICN1 | | | | | | | | 20 | | | | |
| NID2 | | | | | | 9 | | 20.5 | 19 | | | |
| NIF3L1 | | | | | | | | | | | 2.833 | |
| NINJ1 | | 14 | | | | | | | | 13 | | 16 |
| NINJ2 | | | | 21.5 | | | | | | | | |
| NINL | | | | 22 | | 0 | | 19 | 17 | | | |
| NIPA1 | | | | | | | | | | | 23 | |
| NIPSP1 | 5 | | | | | | | 9 | | | | |
| NIT2 | | | | | | | | | | 6 | 5.5 | |
| NKAIN1 | | | | | | | | 6 | | | | |
| NKD2 | | | | | | | | | 22 | | | |
| NKIRAS1 | | | | | | | | 22 | | | | |
| NKIRAS2 | 18 | | | | | 13 | | | | | | |
| NKTR | 0 | | 19.5 | | | | | | | 0 | | |
| NKX2-1 | | | 23 | 21.5 | | | | 0 | | | | |
| NLK | | | | | 6 | | | | | | | |
| NLRC5 | | | | | | 9.5 | | | | | | |
| NLRP2 | | | | 21 | | | | | | | | |
| NLRX1 | | | | | | | | | 0 | | | |
| NMBR | | | | 21 | | | 22.5 | 17 | 16 | | | 18 |
| NME1 | | | | | | | | | | 6 | | |
| NME6 | 19 | | | | | | | 21.5 | 5.5 | | | |
| NME7 | 19 | | | | | | 21 | 21 | | | | |
| NMRAL1 | | | | | | | | | 22 | | | |
| NMRK1 | | | | | | | | 3 | 22.5 | 10 | | |
| NMRK2 | | | 21 | | | | | | | 14 | | 5.5 |
| NMT1 | | | | | | | | | | 9 | | |
| NMT1 | | | | | | 17 | | | | | | |
| NMT2 | 15 | | | | | | | | | | | |
| NNT | 18 | 18.5 | 20.5 | | | | | | 15.5 | 17 | | 21 |
| NOA1 | | | | 21 | | 0.5 | 21 | 18 | | 19.5 | | |
| NOD2 | 18.5 | 21 | 22 | | 20 | | | 21 | 19 | 18 | | 21 |
| NOL3 | 20 | | | | | | | 20 | | | | 20.5 |
| NOL4 | | | | | | | | | | 18.5 | | |
| NOL6 | | | | | | | | | | 11 | | |
| NOL7 | | | | | | | | 19 | | | | |
| NOL8 | | | | | 3 | 0.5 | | 21 | | | | |
| NOLC1 | | | | 21 | 21 | | 22 | 21.5 | | 22 | | |
| NOMO2 | 20 | 20.5 | 22 | | | | | 19.5 | 19.5 | 19 | | |
| NOP16 | | | | | | | | | 14 | | | |
| NOP56 | | | | 22 | | 23 | | | | | | |
| NOS1 | | | | | | | 22 | | | | | |
| NOSTRIN | | | | | | | | | 3 | | | |
| NOTCH1 | | | | | | | | | 5 | | | |
| NOTCH2 | | | | | | | | 14.5 | | | | |
| NOTCH4 | | | | | | 7.5 | | | | | | |
| NOX4 | | | | | | | | | | 4 | | |
| NOXRED1 | | | | | | | | | 3 | | | |
| NPAS2 | 7 | 8.5 | 9 | | | 8 | | 12 | | | | |
| NPAS3 | | | | | | | 23 | | 23.5 | | | |
| NPC1 | | | | | | | | | | 17 | | |
| NPEPPS | | | | | | | | | 23 | | | |
| NPHP1 | | | | | 10.5 | | | | | | | |
| NPHS2 | | | | | | | | | | | 7 | |
| NPL | | | | | | | | | | 20.5 | | |
| NPNT | | | | | 2 | | | | | 17 | | |
| NPPB | | | | | | | | | 4 | | | |
| NPR2 | | | | | | | | | 15 | 11 | | |
| NPR3 | | | | | | | | | | 8 | | |
| NPRL2 | | | | | | | | 19 | | | | |
| NPS | | | | | | | | | 1.5 | | | |
| NPTN | | | | | | | | | | | | 0 |
| NPTX1 | | | | | | | | | 5 | | | |
| NPW | | | | | | | | | 23 | | | |
| NPY1R | | | | | | | | 10 | | | | |
| NQO1 | | | 23 | | | | | 23.5 | | 16 | | |
| NQO2 | | | | | | | | | | 11.5 | | |
| NR0B2 | | | | | | | | | | | 22.5 | |
| NR1D1 | | | | | | | | 19 | | | | |
| NR1D2 | | | 19.5 | | | | | | | | | |
| NR1H2 | | | | | | | | | | 16.5 | | |
| NR1H3 | | | | | | | | | 9 | | | |
| NR1I3 | | 7 | | | | 10 | | | | | | |
| NR2F2 | | | | | | | | 21 | | | | |
| NR2F6 | | | | | | | | 21 | 14.5 | | | |
| NR4A1 | | | | | | 0.5 | | | | | | |
| N-R5S162 | | | | | | 15 | 16 | | 13 | | | |
| N-R5S168 | | | | 20 | | 18 | | | | | | |
| N-R5S176 | | | | | | | | 11 | 8.5 | 10 | | |
| N-R5S2 | | | | 9 | | 11 | | 6 | | | | |
| N-R5S205 | 19 | | | 20 | | | | 7 | | | | |
| N-R5S25 | | | | | | | | 7.5 | | | | |
| N-R5S28 | | | | | | | 18 | | | | | |
| N-R5S5 | | | | | | | | 0 | | | | |
| N-R5S54 | 18 | | | 21 | 22 | | 20 | 22 | | | | |
| NRADD | | | | | | | | 3 | 6 | 4 | | |
| NRBP1 | | | | | | | 20.5 | | | | | |
| NRBP2 | | | | | | 9 | | | 9 | | | |
| NRCAM | | | | | | | | | 22 | | | |
| NRD1 | | | | | | | | | | 7 | | |
| NREP | | | | | | | | | 23 | | | |
| NRF1 | | | | | | | | | 20 | | | |
| NRG4 | | | | | | | | 0 | 21.5 | | | |
| NRIP1 | | | | | | | | 23 | | | | |
| NRP1 | 5 | | | | | | | | | | | |
| NRP2 | 8.5 | | | | | | | | | | | |
| NRXN1 | | | | | | | | 7 | | | | |
| NSDHL | | | | | | | | | 0.5 | | | |
| NSF | | | | | | | 20 | | | | | |
| NSFL1C | | | | 3 | 3 | | 1 | | | 4 | | |
| NSMCE2 | | | | 6 | | | | | 2 | | | 0 |
| NSUN3 | | | | | | | | 8 | | | | |
| NSUN6 | | | 4.5 | | | 22.5 | 3 | | | | | |
| NSUN7 | | | 9 | | | | | | | | | |
| NT | | | | | | | | | | | 10 | |
| NT5C | | | | | | | 20 | | | | | |
| NT5C1A | 12 | 12 | 12 | 13 | 13.5 | 12 | | 13 | 14 | 12 | 11 | 12 |
| NT5C2 | | | | | | 9 | | | | | | |
| NT5DC2 | | | | | | | | | 8 | | 23 | |
| NT5DC3 | | | | | | | | | | 6 | | |
| NT5E | 6 | | | | | | | | | 6 | | |
| NT5M | | | | | | | | | | 4 | | |
| NTF3 | | | | | | | | | | | 22.5 | |
| NTN3 | | | | | | | | | | 5 | | |
| NTN4 | | | | | | | 8.5 | | | | | |
| NTNG1 | 7 | | | | | | | 11.5 | | | | |
| NTNG2 | 7 | | | | | | | | | | | |
| NTPCR | 8 | | | | | | | 7 | 20.5 | | | |
| NTRK2 | | | | | 9 | | | | | 11.5 | | |
| NUAK1 | | | | | 21 | | | | | | | |
| NUBP2 | | | | | | | | | | 16 | 14 | 18 |
| NUCKS1 | 5 | | 4 | | | | | | | | | |
| NUDT1 | 0 | | | | | | | | | | | |
| NUDT11 | | | | | | | | | 22 | | | |
| NUDT13 | 18 | | | | | | | 20 | 19 | | | |
| NUDT16 | | | | | | | | | | 11 | | |
| NUDT17 | | | | | | | | | 22 | | | |
| NUDT18 | | | | | | | | | 22 | | | |
| NUDT19 | 20 | | | | 22 | | | 22 | 22 | 20.5 | | |
| NUDT22 | | | | 3 | 1 | | 3 | 2.5 | 18.5 | | 4 | |
| NUDT3 | | 21.5 | | | | 20 | | | 22 | | | |
| NUDT4 | | | | | | | | | 21 | | | |
| NUDT5 | | | | | | | | | 18 | | | |
| NUDT7 | 1.5 | | 1 | 5 | 4.5 | | | 3 | 19 | | | 1 |
| NUDT9 | | 1 | 2 | | | | | | 8 | | | 6.5 |
| NUMA1 | | | | | | | | | | 20 | | |
| NUMBL | | | | | | | | | | 5 | | |
| NUP205 | | | | | | | | | 23 | | | |
| NUP210 | | | | 13 | 13 | | | | | | | |
| NUP210L | | | | | | | | 18 | | | | 18 |
| NUP37 | | | | | | | 22 | | 0 | 19 | | |
| NUP43 | | | | | | | | 19 | | | | |
| NUP62 | | | | | | 13 | | | | | | 12 |
| NUP85 | 15 | | | | | | 20 | | | | | |
| NUP93 | 0.5 | | | | | | | | | | 23 | |
| NUP98 | | | | | | | | | | | 4 | |
| NUPL1 | | | | | | | | 6 | | | | |
| NUPL2 | | | | | | | | | 14 | | | |
| NUPR1 | | 8 | | | | | | | | | | |
| NUS1 | | | | | | | | 0 | | | | 2 |
| NUTF2 | | | | | | | | | 22 | | | |
| NVL | 2 | | 20 | | | | | | 22 | | | 22 |
| NXF1 | | | | | | | 5 | | | 20 | | |
| NXN | | | 13 | | | | | | | 15 | | |
| NXT2 | | | | | | | | | | 12 | | |
| O3FAR1 | | | | | | | | 8 | | | | |
| OAF | 8 | | | | | | | 11 | 16 | 9 | | |
| OASL | 10 | 11 | | | 14 | 16 | | 12 | 12 | 11 | 13 | |
| OAT | | | | | | | 18.5 | | | | | |
| OAT-RS1 | | 7 | 8 | | 23 | | | 8 | | | | |
| OBFC1 | | | 21.5 | | | | | | | | | |
| OBFC2A | | | | | | | | | 0.5 | | | |
| OBFC2B | | | 7.5 | | | | | 5.5 | | | | |
| OCLN | | | 14 | | | | | | | | | |
| ODC1 | | | | 22 | | | | | | | | |
| ODF2 | | | | | | | | | | 11 | | |
| ODF3B | | | | | | | | | 21 | | | |
| ODZ1 | | | | | | | 20 | | | | | |
| ODZ3 | | | | | | | | 4.5 | | | | |
| ODZ4 | | | | | | | | 7 | | | | |
| OGFOD1 | | | | | | | 7 | | | | | |
| OGFR | | | | | | | | | | 15 | | |
| OGN | | | | | | | | | 17 | | | |
| OGT | | | | | | | | 19 | | 0 | | 22 |
| OLA1 | | | | | | | | | 21 | | | |
| OLFM1 | | | | | | | | 17 | 2 | 12 | | |
| OLFM3 | 22 | | | | | | | | | 2 | | |
| OLFM4 | | | | 23 | | 0 | | | | | 6 | |
| OLFR10 | | | | | | | 21 | | | | | |
| OLFR106-PS | | | | | | | | | | | 23 | |
| OLFR1077-PS1 | | 7 | 6.5 | | | | | | 3.5 | | | |
| OLFR1144-PS1 | | | | | | | | | 21 | | | |
| OLFR1167 | 19 | 17 | 19 | 20 | | 14 | | 20 | | 5.5 | 21 | |
| OLFR1178 | | | | | | | | 11 | | | | |
| OLFR121 | | | | | | | | | | 9 | | |
| OLFR1261 | | | | | | | | | 23 | | | |
| OLFR1307 | | | | | 0 | | | | | | | |
| OLFR1314 | | | | | | | | 21 | | | | |
| OLFR1342 | 4 | | | | | | | | | | | |
| OLFR1346 | | | | | | | | | 9 | | | |
| OLFR144 | | | 2 | | | 7 | | | 3 | | 8 | 5 |
| OLFR1449 | | | | 1 | | | | | | | | |
| OLFR1458 | | | | | | | | 7 | | | | |
| OLFR22-PS1 | | | | | | | | 6 | | | | |
| OLFR295 | | | | | | | 20.5 | | | 14 | | |
| OLFR332 | 18.5 | 16 | | | | | | | 17 | | | |
| OLFR363-PS | 20 | | | | | | | 1 | | | | |
| OLFR367-PS | | | | | | | | | | 8 | | |
| OLFR418-PS1 | | | | | | | | | 0.5 | | | |
| OLFR467 | | | | | 9.5 | | 6 | | | | | |
| OLFR597 | | | | | | | | | | 16 | | |
| OLFR702 | | | | | | | 22.5 | | | | | |
| OLFR74 | | | | | 9 | | | 14 | | | | |
| OLFR827 | | | | | | | | | 23 | | | |
| OLFR849 | 20 | | | | | | | | | | | |
| OLFR855 | | 12.5 | 13 | | | | | | 3 | 14 | | |
| OLFR866 | 8 | | | | | | | | | | | |
| OLFR883 | 8 | | | | | | | | | | | |
| OLFR902 | | | | | | | | | 23 | | | |
| OLFR904 | | | 13 | | | | | | | | | |
| OLFR907 | | | | | 23.5 | | | | | 20 | | |
| OLFR913 | | | | | | 17 | | | | 15 | | |
| OLFR920 | | | | | | | | 2.5 | | | | |
| OLFR934 | | | | | | | | | 14 | | | 23 |
| OLFR967 | | | 10 | | | | | 13.5 | 5.5 | | | |
| OMA1 | 23 | 22.5 | 23.5 | | | 17 | | | | | | 22.5 |
| OPA3 | | | | | | | | | | 5 | | |
| OPALIN | | | | | | | | | | 1 | | |
| OPCML | | | | | | 10.5 | | | | | | |
| OPHN1 | | | | | | | | | 17 | | | |
| OPLAH | | | | | | | | | | 3 | | |
| OPN3 | | | | | | | | 22 | | 20.5 | | |
| OPTN | | | | | | | | 23 | | | | |
| OR10H2 | | 8 | | | | | | | 6 | | | 11 |
| OR10S1 | | | | | | | | | 23 | | | |
| OR10X1 | | | | | | | | | 19 | | | |
| OR11H4 | | | | | | | | | 17 | | | |
| OR12D3 | | 1.5 | 3 | 5 | | 21 | | | 23 | | 1.5 | |
| OR13C8 | | | 14.5 | | | | 22 | | | | | 12.5 |
| OR1L6 | | | | | | | | 9 | | | | |
| OR1N1 | | | | | | | | | 23 | | | |
| OR2A5 | | | | | | | | | 21 | | | |
| OR2B6 | | | | | | | | | 22 | | | |
| OR51D1 | | | | | | | | | | | | 6.833 |
| OR51Q1 | | | | | | | | | 8 | | | |
| OR51S1 | 20 | | 23 | | | | | | 1 | | | |
| OR52D1 | 8 | 9.5 | 11 | | | 10 | | 7 | | | | 10 |
| OR52E6 | | | | | | 16 | | | 22 | 16 | | |
| OR52J3 | | | 10 | | | | | | | | | |
| OR56A5 | 3 | | | | | | | | | | | |
| OR5M3 | 4 | | | | | | | 7 | 3 | 4 | | |
| OR6C65 | | | | | | | | | | 6.5 | | |
| OR7A17 | 3 | | | | | | | | 23.5 | 4 | | |
| OR8B8 | 1 | | | | | | | | | | | |
| OR9Q2 | 3 | | | | | | | | 22 | | | |
| ORAI2 | | | | | | | | | 23 | | | |
| ORAI3 | | | | | | | | 17 | | 11 | | |
| ORC3 | | 18 | 21 | | | 17 | | | 20 | 16 | 20 | 20 |
| ORC6 | | | | | | | | | 12 | | | |
| ORM1 | 3 | | | 9.5 | | | | | | | | |
| ORMDL2 | | | | | | | | 18 | | | | |
| ORMDL3 | | | | | | | | | | 12 | | |
| OS9 | | | 11.5 | | | | | | 9 | | | |
| OSBP2 | | | | | | | | | 21 | | | |
| OSBPL10 | | | | | | | | | 23 | | | |
| OSBPL11 | 2.5 | | 4 | | | | | | | | | |
| OSBPL1A | 20 | | | | | | | | | | | |
| OSBPL2 | | | | | | 19 | | | | | | |
| OSBPL3 | | | | | | | | | | 8 | | |
| OSBPL5 | | | | | | | | | | 13.5 | | |
| OSBPL6 | 6 | | | | | | | | 18 | | | |
| OSBPL7 | | | | | | | | | | | 0 | |
| OSBPL8 | | 23 | 0 | | | | | | 0 | 22 | 0.5 | 0 |
| OSBPL9 | | | | | | 22 | | | | | | |
| OSGEP | | | | | | | 9 | | | 9 | | |
| OSGIN1 | | | | | | | | 3 | | | | |
| OSGIN2 | | | | | | | | | | 13.5 | | |
| OSMR | | | | | | | | | | 15 | | |
| OSR1 | | 12 | 13 | | | | | 13 | | | | |
| OSTALPHA | | | | | | | | | 3 | 13 | | 15 |
| OSTM1 | | | | | | | | | 10 | 11 | 21 | |
| OTOP1 | | | | | | | | | | 6 | | |
| OTUB2 | | 22 | 23.5 | | | | | | 3 | 2 | | |
| OTUD1 | | | | | | | | | 19 | | | |
| OTUD5 | | | | | | | | | | | 8 | |
| OTUD6B | | 23 | 23 | | 3 | 1.5 | 3 | 22 | 22 | 1 | 0 | |
| OXA1L | | | | 0 | 1 | | 0 | | | | | |
| OXCT1 | | | | | | | | | | 11 | | |
| OXD1 | | | | 0 | | | | | | 7 | | |
| OXR1 | | 10 | 11.5 | | | | | | | | | |
| OXSM | | | 9 | | | | | 20 | | | | |
| P1L1 | | | | | | | | | | 11 | | |
| P2RX4 | | | | | | | | | | 18 | 1 | |
| P2RX5 | | | | | | | | | | 10 | | |
| P2RX6 | | | | | 15.5 | | 16 | | 14 | | | |
| P2RY1 | | | | | | | | 13 | | 10 | | |
| P2RY14 | | | | | | | | | | 1 | | |
| P2RY2 | | | | | | 15 | | | | | | |
| P2RY4 | | | | | | | | 7 | 7 | 22 | | |
| P2RY6 | | | | | | | | | | 13 | | |
| P4HA1 | | | | | | 2 | | | 7.5 | | | |
| P4HA2 | | | | | | | | | | 12 | | 13 |
| P4HA3 | | | | | | | | | 2 | 4.5 | | |
| P4HB | | | | 21 | | | | | | 23 | | |
| P4HTM | | | | | | | | | 3 | | | |
| PA2G4 | 19 | | | | 22 | | | | | | | |
| PACS1 | | | | | | | | | 9 | 12.5 | | |
| PACS2 | | | | | 0 | | | | | | | |
| PACSIN2 | | | | 5.5 | | | 4.5 | | | | | |
| PACSIN3 | 20 | | | | | | | | | | | |
| PAFAH1B3 | | | | 17 | | | | 16 | | | | |
| PAFAH2 | | | | | 5 | | | | | | | |
| PAG1 | | | | | | 11.5 | | | | | | |
| PAH | | | | | 1 | | | | 0.5 | | | |
| PAICS | | | | 13 | 13 | 12 | | | 19 | | | |
| PAIP2B | | | | 20 | 19 | | | | | | | |
| PAK1IP1 | | | 12.5 | | | | | | | | | |
| PALLD | | | | | 0.5 | | | | | | | |
| PALM | | | | | | 18.5 | | | | | | 9 |
| PALM3 | | | 19 | | | | | | 19 | 13 | | |
| PALMD | 7 | 8 | | | | 9 | | | 8 | 10 | | |
| PAMR1 | | | | | | 13 | | | | 16 | | |
| PANK1 | | | | | | | | | 22.5 | | | |
| PANK3 | | | | | | 4 | | | | 6 | | |
| PANK4 | | | | | | | | | 17 | 18 | | |
| PAPD4 | | | | | | | | | | 12 | | |
| PAPD7 | | | | | 9 | | | | | | | |
| PAPOLA | | 10 | 12.5 | | | 15 | | 19 | | | | |
| PAPPA | | | | | | | | | 5 | | | |
| PAPSS1 | | | | | | | | | | 11 | | |
| PAPSS2 | | | | | | | | 20.5 | | 19 | | 23.5 |
| PAQR3 | 5.5 | | | | | | | 7.5 | | 8.5 | | |
| PAQR4 | | | | | | | | | 15 | | | |
| PAQR5 | | | | | | | | | 5.5 | | | |
| PAQR7 | | | | | | | | | 9 | | | |
| PAQR8 | | | | | | | | | 13 | | | |
| PAQR9 | | | | | | | | | | | | 18 |
| PARD3 | | | 9 | | | 12 | | | 8.5 | | 9 | |
| PARD6G | | | 22 | | | | | | | | | |
| PARK7 | | | 8.5 | | | | | | | | | |
| PARL | | | | | | | | | | | 9 | |
| PARM1 | 8 | | 8 | | | | | | 19 | | 8.5 | |
| PARP10 | | | | | | | | | 22 | | | |
| PARP11 | 4 | | | | | 6 | | | 4 | | | |
| PARP12 | | | | | | 19 | | 17 | | | | |
| PARP16 | | | | | | | | 10 | | | | |
| PARP2 | | | | | | | | | | 5.5 | | |
| PARP3 | | | | | | | | 1 | | | | |
| PARP8 | | | | | | | | 22 | | | | |
| PARVB | | | | | | | | | | 17 | | |
| PATZ1 | | | | | | 19 | | | 11 | | | |
| PAX8 | | | | | | | | 16 | | | | |
| PAXIP1 | | | 19 | | | | | | | | | 21 |
| PBLD | | | | | | | | 6 | | | | |
| PBRM1 | | | 5 | | | | | | | | | 0 |
| PBX1 | | | | | | | | | | | | 10 |
| PBX3 | | 9.5 | | | 9.5 | | 9 | 11 | | 11 | | |
| PCBD1 | | | | | | 22 | | | | | | |
| PCBP1 | | | | | | | | 5.5 | | 3 | | |
| PCBP2 | | | | | | 7 | | | 10.5 | | | |
| PCBP4 | | | | | | | | | 10.5 | | | |
| PCCA | | | | | | | | | 14 | | | |
| PCCB | | 7 | | | | | | 8 | 7 | | | |
| PCDH1 | | | | | | | | | 20 | | | |
| PCDH11X | | | | | | | | | 22 | | | |
| PCDH12 | | 2.5 | 3.5 | | | | | 4 | | 11 | | |
| PCDH18 | | | | | | | | | | 4 | | |
| PCDH7 | | | | | | | | | | 6 | 22.5 | |
| PCDH9 | | | | | | | 20 | | | | | |
| PCDHB11 | | | | | | | | | 22 | | | |
| PCDHB6 | | | | | | | | | 23 | | | |
| PCDHB7 | | | | | | | | 22 | | 18.5 | | |
| PCDHB8 | | | | | | | 7.5 | | | | | |
| PCGF2 | | | | | | | | | 20 | | | |
| PCGF5 | | 21.5 | | | | | | | | | | |
| PCIF1 | | | | 1.5 | | | | | | | | |
| PCK1 | | | | | | | | | 21 | | | |
| PCLO | | | | | | 5 | | | | | | |
| PCMTD2 | | | 16.5 | | | | | 22 | | | | |
| PCOLCE2 | | | | | | | | | | 12 | | |
| PCP4L1 | | | | | | | | | | 20.5 | | |
| PCSK1 | | | | | 21 | | | | | | | |
| PCSK4 | 14.5 | | | | | | | | | | | |
| PCSK6 | | | | | | | | | | | 1 | |
| PCSK7 | | | | 21 | | | | | 1 | | | |
| PCYOX1 | | | | | | 5.167 | | | | | | |
| PCYOX1L | | | | | | | | | 21 | | | |
| PCYT1A | | | | | | | | | 21 | | | |
| PCYT2 | | | | | | | | | 13.5 | | | |
| PDCD2 | | | | | | | 8 | | | | | |
| PDCD4 | | | | | | | | 23 | | | | |
| PDCD6IP | | | | | | | 23 | | | | | |
| PDCL | | | 20.5 | | | | | | | | | |
| PDCL3 | | | | | | | | | 19 | | | |
| PDDC1 | | | | | | | | | 10 | | | |
| PDE1A | | | | | 12 | | | | | | | |
| PDE1B | | | | | | | | | 23 | | | |
| PDE2A | | | | | | | | 8 | 0 | | | |
| PDE3A | | | | | | | | | 23 | | | |
| PDE3B | | | | | | | | 23 | | | 10 | |
| PDE4A | | | | | | | | | | | 3.5 | |
| PDE4B | | | 23 | | | | | 18 | 17 | 18 | | |
| PDE4DIP | | | | | | | | | 20 | | | |
| PDE5A | | | | | | | | | 21 | | | |
| PDE6D | | | | | | | | | | 19 | | |
| PDE6G | | | 15 | | | | | | | | | |
| PDE7A | | | | | | | | | 4 | | | |
| PDE7B | | | | | | | | | | 19 | | |
| PDE8A | 19 | | | | | | | | | | | |
| PDE8B | | | 0.5 | | | | | | | | | |
| PDE9A | 14.5 | | | | | | | | | | | |
| PDGFA | | | | | | | | | 22 | | | |
| PDGFB | | 16.5 | | | | | | | | | | |
| PDGFC | | | 2 | | | | | | | | | |
| PDGFD | | | | | | 15 | | 16 | 20.5 | 15 | | |
| PDGFRA | | | | | | | | | 3 | | | |
| PDGFRB | | 23 | | | | | | | | | | 23 |
| PDGFRL | 23.5 | | | | | | | | 23 | | | |
| PDHB | | | | | | | | 21 | | | | |
| PDIA3 | | | | | | | | | | 5 | | |
| PDIA4 | | 20.5 | | | | | | 22 | 23 | | | |
| PDIA5 | | | | | | 10 | | | 21.5 | | | |
| PDIA6 | | | | 23 | | | | | | | | |
| PDK2 | | | 11 | | | | | 11 | | | | |
| PDK4 | | | | | | | | | | | 5.833 | |
| PDLIM1 | | | | | | 11 | | | | | | |
| PDLIM2 | | | | | | | | | 0 | | | |
| PDLIM3 | | | | 21 | | | | | | | | |
| PDP1 | | | | | | | | 20 | | | | |
| PDP2 | | | | | | | | | | | 7 | 15 |
| PDPN | | | | | | | | | | | 9 | |
| PDPR | | | | | | | | | 21 | | | |
| PDS5A | | | | | 7 | | | | | 13 | | |
| PDS5B | | | | | | | | 22.5 | 23 | | | |
| PDXK | | | | | | 10 | | | | | | |
| PDXK-PS | | | | | | | | | 0 | | | |
| PDZD11 | | | | | | | | | | | 1.167 | |
| PDZD2 | | | | | | | | | 10.5 | | | |
| PDZK1 | | | | | | | | | 1 | | | |
| PDZRN3 | | | | | | | | | 10.5 | | | |
| PDZRN4 | | | | | | | | | 15 | | | |
| PEA15 | | | | | | | | | 7.5 | | | |
| PEAR1 | | | | | | | | | 18.5 | | | 10 |
| PECAM1 | | | | | | | | | | 6 | | |
| PEG10 | | | | | | | | 10 | | | | |
| PEG3 | | | | | | | | | 23 | | | |
| PELI2 | | | | | 9.5 | | | | | | | |
| PELP1 | | | | 10 | 7 | | | | | 5 | | |
| PENK | | | | | | 13 | | | | | | |
| PEPD | | | | | | | | | 21 | | | |
| PEPLD | | | | | | 14.5 | | | 14.5 | 17 | | 17 |
| PER1 | | | | | | | | | 16 | 17 | | |
| PER2 | | 22 | 22 | | | | | | | | | 23 |
| PER3 | | | | | 12 | | | | | | | |
| PES1 | | | | | | | | | 9 | | | |
| PET112 | | | | | 14 | | | | | | | |
| PEX1 | | | | | | | | | 22 | | | |
| PEX11A | | | | | 9 | | | | | | | |
| PEX11B | | | | | | | | | | 7 | | |
| PEX14 | | 20 | 22 | | | | 23 | | | 18.5 | | |
| PEX16 | | | | | | | | | 21 | | | |
| PEX19 | | | 10.5 | | | | 14.5 | | | | | |
| PEX3 | | | 6.5 | | | | | | | | | |
| PEX5 | | | | | | | | 22 | | | | |
| PEX7 | | | | | | | | | 21 | | | |
| PFDN2 | | | | 18 | | | | | | 16.5 | | |
| PFDN5 | 0 | | | | | | | | | | | |
| PFKFB1 | | | | | | | | 22 | | | | |
| PFKFB2 | | | | | | | 15 | | | | | |
| PFKFB3 | | | | | | | | | 23 | | | |
| PFKFB4 | | | | | | | | | | | 2.167 | |
| PFKL | | | 0 | | | | | | | | | |
| PFKM | | | | | | | | 0 | | | | |
| PFKP | | | | | | | | 21 | | | | |
| PFN4 | | | | | | | | 21 | | | | |
| PGAP1 | | 19 | | | | | | | | | | |
| PGD | | | | | | | | | | 6 | | |
| PGF | | | | | | | | | | 7.5 | | |
| PGLS | | | 12 | | | | | | | | | |
| PGLYRP1 | | | 0.5 | | | | | | | | | |
| PGLYRP2 | | | | | | | | 22 | | | | |
| PGM1 | | | | 0 | | | | | | | | |
| PGM2L1 | | | | | | | | 23.5 | | | | |
| PGM3 | | | | | | | | | 7 | | | |
| PGM5 | | | | | | | | | 23.5 | | | |
| PGP | | | | | | | | | 23 | | | |
| PGPEP1 | | | | | | | | 14 | | | | |
| PGR | | | | | | | | | | | 0.5 | |
| PGRMC2 | | | | | | | | | 10 | | | |
| PHACTR4 | | | 10 | | | | | | | | | |
| PHB2 | | 22 | 22 | | | | | | | | | |
| PHC3 | | | | | | 10.5 | | | | | | 21 |
| PHF1 | | | | | | | | | 23 | | | |
| PHF15 | | | 11 | | | | | | | | | |
| PHF16 | | | | | | | | | | | | 21 |
| PHF17 | | | | | | | | | 20 | 19.5 | | |
| PHF19 | | | | | | | | | | | 11.83 | |
| PHF6 | | | | | | | 22 | | | | | |
| PHIP | | | | | | | | | 1.167 | | | |
| PHKA1 | | | | | | | | 11 | | 9.5 | | |
| PHKA2 | | | | | | | | | 0.5 | | | |
| PHKG1 | | | 23 | | | | | | | | | |
| PHKG2 | | | | 21 | | | | | | | | |
| PHLDA1 | | | | 21.5 | | | | | | | | |
| PHLDA3 | | | | | | | | | 21 | | | |
| PHLDB1 | | | | | | | | 7 | 6 | | | |
| PHLDB2 | | | | | | | | | | | 4.5 | |
| PHLPP1 | | | | | | | | 14.5 | | | 16 | |
| PHLPP2 | | | | | | | | | 23 | | | |
| PHOSPHO2 | | | | | | | | 14 | 17 | | | |
| PHOX2B | 10 | 14 | 15 | | | 16 | | 14 | 11.5 | 12 | 16 | 14 |
| PHTF1 | 7 | | | | | | | | 7.5 | | | 12 |
| PHTF2 | | | | | | | | | 22 | | | |
| PHYH | | | | | | | | | | 17 | | |
| PHYHIP | | | | | | 16 | | | 1 | | | |
| PHYHIPL | | | | | | | | | 18 | 10 | | |
| PI15 | | | 6 | | | | | | | | | |
| PI16 | | | | | | | | | 14 | | | |
| PI4K2A | | | | | | | | | 22 | | | |
| PI4K2B | | 22 | 23 | | | | | 11 | 17 | | | 22 |
| PI4KA | | | 22 | | | | | | | | | |
| PIAS2 | | | | | | 15.5 | | 14.5 | | | | |
| PICALM | | 22.5 | | | | | | | 23 | 0 | 0 | |
| PICK1 | | | | | | | | | | | | 13.5 |
| PID1 | | | | | 19 | | | 19 | 22 | | | |
| PIEZO1 | | | | | | | | | | 11 | | |
| PIEZO2 | | | 22 | | | | | | | | 20 | |
| PIGA | | | | | 7 | | 6 | | | | | |
| PIGB | | | | 14 | 12 | | 15 | | | | 13 | |
| PIGC | 10 | | 6 | | | | | | 9 | 16 | | |
| PIGH | | | | | | | | 19.5 | | | | |
| PIGL | | 5.5 | 9 | | | | | 6.5 | | | | 11 |
| PIGN | | | | | 2 | | | | 21 | | | |
| PIGO | | | | | | | 3 | | | | | |
| PIGR | | | | | | | | | | 10.5 | | 5 |
| PIGS | | | | 16.5 | 17 | | | | | 1 | | |
| PIGT | | | | | | | | 8 | | | | |
| PIGU | | | | | | | | | 3 | | | |
| PIGY | | | | | | | | | | 1 | | |
| PIK3AP1 | | 1 | 0 | | | 1.5 | | 16 | | | | |
| PIK3C2G | | | | | | | | | | 15 | | |
| PIK3C3 | | | 6.5 | | | | | | | | | |
| PIK3CA | | | | | | | | | | 10.5 | | |
| PIK3IP1 | | | | | | | | 6 | | | | |
| PIK3R1 | | | | | 21.5 | | | 23 | | | | |
| PIK3R2 | | | | | | | | | | | 21 | |
| PIK3R3 | | | | | | 2 | | | 19 | | 23.5 | |
| PIK3R6 | | | | | | | | | 22 | | | |
| PILRA | | | | | | | 22 | | | | | |
| PIM1 | | | 0 | | | | | 19 | | | | 4 |
| PIM3 | | | 22.5 | | | | | | | | | |
| PINK1 | | | | | | | | | 0 | | | |
| PION | | | | | | 5.167 | | | | | | |
| PIP4K2A | | | | | | | | | 19 | | | |
| PIP4K2B | | | | 21 | 23 | | 21 | | | 18 | | |
| PIP4K2C | 0 | | | | | | | | 22 | 0 | | |
| PIP5K1B | | | | | | | | 2 | 23 | | | |
| PIPOX | | | | | | | | 5.5 | 20 | | | |
| PIR | | | | | | | | | | 2 | 22 | |
| PIRT | | | | | | | | | 14 | | | |
| PISD-PS2 | | | | | | | | 22 | | | | |
| PITP | | | | | | | | | 21.5 | | | |
| PITPNC1 | | | | | | 20.5 | | | 22.5 | | | |
| PITPNM1 | | | | | | | | 22.5 | 23.5 | | | |
| PITPNM2 | | | | | | | | | 22 | | | |
| PJA1 | | | | | | | | | | | | 21 |
| PKD1 | | | 1 | | | | | | | | | |
| PKD2L1 | | | | | | 23 | | | | | | |
| PKIA | | | | | | | | | | 23 | | |
| PKLR | | | | | | 21 | | | 19 | 13 | | |
| PKM | | | 0.5 | | | | | | | 10 | | |
| PKMYT1 | | | | | | | | | | | 1.833 | |
| PKN1 | | | | | 14 | | | | | 7 | | |
| PKN2 | | | | | | | | | 15 | 13 | | |
| PKNOX1 | | | | | | | | | 18 | | | |
| PKNOX2 | | | | | | | | 23 | | | | |
| PKP2 | | | | | | | | 9 | 9 | | | |
| PLA1A | | | | | | | | 3 | | | | |
| PLA2G12A | | | | 19.5 | | | | | | | | |
| PLA2G12B | | | | | | | | | 1.5 | | | |
| PLA2G15 | | | | | | | | | 23.5 | | | |
| PLA2G4D | | | | | | 18 | | | 15 | | | |
| PLA2G5 | | | | | | | | | 6 | | | |
| PLA2G7 | | | | | | | | 9 | | | | |
| PLA2R1 | | | | | | | | | | 16 | | |
| PLAC8 | | | 22.5 | | | | | | 19.5 | 15 | | |
| PLAGL1 | | | | | | | | | | 5 | | |
| PLAT | | | | 11 | | | | | | | | |
| PLAU | 9 | | | | | 11 | | | | | | |
| PLAUR | | | | | | | | | | 2 | | |
| PLBD1 | | | | | | | | | 22 | | | |
| PLBD2 | | | | | | | | 18 | | 12 | | |
| PLCB1 | | | 23 | | | | | | | | | |
| PLCB4 | | | | | | | | | 9 | 9 | | |
| PLCD3 | | | | | | | | 9 | | 11.5 | | 10 |
| PLCD4 | | | 22 | | | | | | 22 | | | 23 |
| PLCE1 | 8 | | | | | | | 0 | 23.5 | | | |
| PLCG1 | | | | | | | | | 21 | | | |
| PLCL1 | | | | | | | | | | | 7 | |
| PLCL2 | | | | | | | | | | 11 | | |
| PLD1 | | | | | | | | | | | | 21 |
| PLD2 | | | 20 | | | | | | | | | |
| PLD4 | | | | | | | | | | 11 | | |
| PLD6 | | | | | | 0 | | | 22 | | | |
| PLEC | 22 | | | | | | | | 2 | | | |
| PLEKHA1 | | | | | | | | | | 11 | | |
| PLEKHA3 | 11 | | | | 9 | | | | 15 | | | |
| PLEKHA6 | | | | | | | | | 0.5 | | | |
| PLEKHA8 | | | | | | | | | 18.5 | | | |
| PLEKHF1 | | | | | | | | | | 5 | | |
| PLEKHG1 | | | | | | | | | | 8 | | |
| PLEKHG2 | | | | | | | | | | 19 | | |
| PLEKHG3 | 9 | | 21 | | | | | 13 | | | | |
| PLEKHG5 | | 6 | | | | | | | 23 | | | |
| PLEKHG6 | | | 2 | | | 1 | | | | | 3 | |
| PLEKHH1 | | | | | | | | | | 23 | | |
| PLEKHH3 | 20 | | | | | | | | | | | |
| PLEKHN1 | | | | | | | | 18.5 | | | | |
| PLIN1 | | | | | | | | 20 | | | | |
| PLIN2 | | | | | | | | 10 | | | | |
| PLIN3 | | | | | | | | | 22.5 | | | |
| PLIN4 | | | | | | | | | | 12 | | |
| PLIN5 | | | | | | 18.5 | | | | | | |
| PLK3 | | | | | | | | | 21 | | | |
| PLLP | | | 8 | | | | | | | | | |
| PLN | | | | | | 16 | | | | | 10.5 | |
| PLOD1 | | | | 13.5 | | | | | | | | 16 |
| PLOD2 | | | | | | | | | | 15 | | |
| PLOD3 | | | | | | | | | | 13 | | |
| PLRG1 | | | | | | | | 1 | | | | |
| PLSCR1 | | | 13 | | | | | | | | | |
| PLSCR4 | | | | 21 | 20 | | | | | | | |
| PLTP | | | | | | | | 20 | | | | |
| PLX1 | | | | | | | | | | | 22 | |
| PLX2 | | | | | | 2 | | 0 | 2 | | | 23.5 |
| PLX4 | | | | | | | | 0 | | | | |
| PLXDC1 | | | | | | | | 6.5 | | | | |
| PLXDC2 | 5 | 8.5 | 9 | | | | | 3 | 5 | | | |
| PLXNB1 | | | | | | | | | | | 7 | 0 |
| PLXNB2 | | 13.5 | 16 | | 21 | | | | | | | |
| PLXNB3 | | | | | | | | 9 | | | | |
| PLXNC1 | | | | | | | | | 22 | | | |
| PLXND1 | | | | | | | | | 21.5 | | 23 | |
| PM20D1 | | | | | | | | 20 | | | | |
| PM20D2 | | | | | | | | 0 | 23 | | | |
| PMEPA1 | | | | | | | 0.5 | 21.5 | 22 | | | |
| PML | | | | | 23 | | | | 0 | 23 | 23 | |
| PMM1 | | | | | | | | | 10 | | | |
| PMP22 | | | | | | | | | | | 18 | |
| PMVK | | | 21.5 | | | | | | | | | |
| PNISR | | | | | | 0.5 | 15 | | | | 7 | |
| PNKD | 11 | 13 | 13 | | | 12.5 | | | 14 | | | |
| PNKP | 19 | | | | | | | 23 | | | | |
| PNLDC1 | | | | 13 | | | | | | | | |
| PNMAL2 | | | | | | | | 4 | | | | |
| PNMT | | | | | | | | | | | 23 | |
| PNP | | | 21 | | | | | | | | | |
| PNPLA1 | | | | | | 2 | | | | 4 | 22 | |
| PNPLA2 | | | | | 22 | | | | | | | |
| PNPLA3 | | | | | | | | 4 | | | | |
| PNPLA6 | | | | | | | | 15 | 22 | | | |
| PNPLA7 | | | | | | | 19 | | | 17 | | |
| PNPO | | | | | | 19 | 19 | | | | | |
| PNPT1 | 12 | | 12.5 | | | | | | 20 | | | |
| PNRC1 | | | | | | 23 | | | | | | |
| PNRC2 | 6 | 7 | 6 | 8 | | 10 | 9 | 7 | 6 | 6 | | |
| PODN | | | 7 | | | | | 9 | | | | |
| PODXL | | | | | | | | 0 | 21 | | | |
| POF1B | | | | | | | | | 20 | | | |
| POGLUT1 | | | | | | | | | 0 | | | |
| POLA2 | | 7 | | 9 | | | | | 4 | | | |
| POLE | | 18.5 | 19 | | 22 | | 21 | 19.5 | | | | |
| POLDIP3 | | | | | | | | 21 | | | | |
| POLE3 | | | | | | | | | 19 | | | |
| POLE4 | | | | | | | 9.6667 | | | | 9.667 | |
| POLG | | | | | | | | | | 16 | | |
| POLI | | 0 | 23 | | | | | 9 | | 18 | | |
| POLK | | | | | | | | | | 3.5 | | |
| POLR2A | 6 | | | | | 4 | | 6 | | | | |
| POLR2B | | | | | | | | | | | 2 | |
| POLR2E | | | | | | | | | | | 2 | |
| POLR2M | | | | | | 14.5 | | | | | | |
| POLR3G | | | | | | | | 21 | 12 | | | |
| POLR3GL | | | | | 23.5 | | | | | | | |
| POLR3K | | | | 21 | | | | | | | | |
| POM121 | | | | | | 10.5 | | | | | | |
| POMP | | | | 23 | | | | | | | | |
| POMT1 | | | | 0 | | | | | | 21 | | |
| POMT2 | | | | | 7 | | | | | 8 | | |
| PON2 | 17 | | | | | | | | | | | |
| PON3 | | | | | | | | 21 | | | | |
| POP1 | | | | | | | | | 23 | | | |
| POP4 | | | | | | 23 | | | | 22 | | |
| POPDC2 | | | | | | | 6 | 9.5 | | | | |
| POPDC3 | | | | | | | | | | | 3.5 | |
| POR | | | | | | | | | 1.5 | | | |
| PORCN | | | | | | 15 | | 12 | 13 | | | |
| POT1 | | | | | | 22 | | | | | | |
| POU2AF1 | | | | | | | | | 5 | | | |
| POU3F3 | | | | | | | | | | | 4.5 | |
| POU5F2 | | | | | | 19 | | | | | | |
| POU6F1 | | | | | | | | | | 11.5 | | |
| PPA1 | | 22 | | | | 0 | | | | | | |
| PPAP2A | | | | | | | | 21 | | | | |
| PPAP2B | | | | | | | | | 13.5 | | | |
| PPAP2C | | | | | | | | | | 19 | | |
| PPAPDC2 | | | 19 | | | | | | | | | 20.5 |
| PPAPDC3 | | | | | | | | | | | | 20 |
| PPARA | | | | | | 18 | | | | | | |
| PPARD | | | | | | | | 8 | | | | |
| PPARG | | 6 | | 18.5 | 19 | | 18 | 0 | | | | |
| PPARGC1A | | | | | | | | 9.5 | 12 | | | |
| PPARGC1B | | 11 | 9 | | | | | 11 | 7 | 13 | 7 | |
| PPAT | | | | | | | | | | 1 | | |
| PPDPF | | | | | | | | 22 | | | | 5 |
| PPEF1 | | | | | | | | 19 | | | | |
| PPFIA1 | | | 2 | 3.5 | | | | | 3 | | 0 | 8 |
| PPFIBP1 | | | | | | | | | 23 | 15 | | |
| PPFIBP2 | | | | 22 | | | 20 | | | | | |
| PPID | | | | 14 | | | | | | | | |
| PPIF | | | | | | | | | | | | 4.5 |
| PPIG | | | | 12 | | | | | | | | |
| PPIL1 | | | | | | | | | 17 | | | |
| PPIL6 | | | | | | | | 2 | | | | |
| PPIP5K1 | 15 | 15 | | | | | | | | 13 | | |
| PPIP5K2 | 17 | 16 | 16 | 13 | 13 | 15 | 14 | | 15 | 16 | 14 | 18 |
| PPL | | 16.5 | 17 | | | 17 | | 14 | 18 | 17 | 17 | 19 |
| PPM1A | | | | | | | | | | 8 | | |
| PPM1F | | | | | | | | | 1 | 14 | | |
| PPM1H | | | | | | | | 12 | | | 23 | |
| PPM1K | 15.5 | | | | | | | | | | | |
| PPM1M | | | | | | | | | 18 | 16 | | |
| PPME1 | 21.5 | | | | | | | | 6 | 15 | | |
| PPOX | | | | | | | | | | 15 | | |
| PPP1CB | | | | | | | | | | 9.5 | | |
| PPP1R11 | 17 | | 12 | | 0 | 16 | | 18.5 | | | | |
| PPP1R12B | | | | | | | | | | 21.5 | | |
| PPP1R14A | | | | | | | | 17 | | | | |
| PPP1R14C | | | | | | | | | | 11 | | |
| PPP1R15B | | | | | | | | 11 | | 11 | | |
| PPP1R16B | | | | | | | | | 13 | | | |
| PPP1R18 | | 8 | 10 | | | | | | 6 | 11 | | |
| PPP1R21 | | | | | | 18 | | | 22 | | | 20 |
| PPP1R27 | | 8.5 | | | | | | | | 11 | | |
| PPP1R36 | | 20 | | | | | | 20 | | | | |
| PPP1R3A | | 13 | | | 13 | 15 | | 15 | 14 | 12 | | 13 |
| PPP1R3B | | | 21 | | | | 23 | 19 | 19 | 17 | | |
| PPP1R3C | | | 0 | | | | | | | | | |
| PPP1R3D | | | | | | | | | | 13.5 | | |
| PPP1R7 | | | | | | | | | 22 | | | |
| PPP1R9A | 7 | | | 11 | | | | | | 9 | | |
| PPP2CA | | | 10 | | | | | | | | | |
| PPP2CB | | | | | | | | 12.5 | | | | |
| PPP2R1A | | | | | | | | | | 9 | | |
| PPP2R1B | | | | | | | | 22 | | | | |
| PPP2R2B | | | | | | | | | | 23 | | |
| PPP2R2D | | | 23 | | | 19 | | | | | | |
| PPP2R3A | 2 | | 19 | | | | | | | | | |
| PPP2R4 | | | | | | 6.5 | | | | 6.5 | | |
| PPP2R5A | | 13 | | 16 | 16 | 13 | | | | 15 | | |
| PPP2R5C | | | | | | | | 5 | | | | |
| PPP2R5D | | | | | | | | | | | 9 | 5 |
| PPP3CA | | | | | | | | 12 | 22 | | | |
| PPP3CB | | | | | | | | 14 | | | | |
| PPP4R1 | | | | 22 | | | | | | | | |
| PPP4R1L-PS | | | | | | | | | | | 5.167 | |
| PPP6R3 | | | | 21 | | | | | | | | |
| PPPDE1 | | | | | | 4 | | | | | | |
| PPPDE2 | | | | | | | | 2 | 2.5 | | | |
| PPT1 | 9 | | | 3.5 | | | | | 8 | | | |
| PPTC7 | 19.5 | | | | | | | | | | | |
| PPWD1 | | | | | | | | | | | | 9 |
| PQBP1 | | | | 3 | 3 | | | | 5 | 6 | | |
| PQLC3 | | | | | | | | 23.5 | | | | |
| PRAF2 | 10 | | 0 | | | | | | 10.5 | 9 | 9 | |
| PRAMEF8 | | | | | | | | | | 13 | | |
| PRCC | 20 | | | | | | | 1 | | | | |
| PRCP | | | | 23 | | | | 14.5 | | | | |
| PRDM1 | | | | | | 7 | | | | | | |
| PRDM10 | | | | | | 11 | | | | | | |
| PRDM2 | | | | | | 17 | | | | 18 | | |
| PRDM5 | | | | 22 | | | | | | | | |
| PRDM6 | | | | | | | | 0 | 18 | | | |
| PRDX2 | | | | | | 22 | | | | | | |
| PRDX3 | | | | | | | | | 3 | | | |
| PRDX5 | | | | | | | | 22 | 23 | | | |
| PRDX6 | | | 21.5 | | | | | | | | | 4.5 |
| PREB | | | | | | | | | | 4.5 | | |
| PRELP | 0 | | | 4 | | | | 23.5 | 1 | 0 | | 1 |
| PREP | | | | | | | | 21 | | | 6.5 | |
| PREPL | | | 1 | | | | | | | | | |
| PREX1 | | | | | | | | 22 | 22 | | | |
| PREX2 | | 7 | | | | | | | | | | |
| PRG4 | | | | | | | | 22 | 0 | | | |
| PRHOXNB | | | | | | | | | | 12 | | |
| PRIC285 | | 23 | 23 | | | | | 22 | | | | |
| PRICKLE3 | | | 14 | | 9.5 | | | 21 | 1 | | | 14 |
| PRIM1 | | | | | | | | | | 5 | 21 | |
| PRKAA1 | | | | | | | | | | | 23.5 | |
| PRKAA2 | | | | | | 11.83 | | | | | | |
| PRKAB1 | 7.5 | 9 | 10 | | | | | | | | | |
| PRKACA | | | | | | | | 2 | | | | |
| PRKACB | | | | | | | | | 22 | | | |
| PRKAG3 | | | | | | | | 7 | | | | |
| PRKAR1A | | | | | | | | | 16.5 | | | |
| PRKAR1B | | | | | | | | | | 13 | | |
| PRKAR2A | | | | | | | | 9 | | 15 | | |
| PRKAR2B | | 22 | 0 | | | | | | 2 | | | 0 |
| PRKCA | | | | | | | | | 1.5 | | | |
| PRKCB | | | | | | | | 22.5 | | | | |
| PRKCD | | | | | | | | | 21 | | | |
| PRKCDBP | | | | | | 21 | | | | | 5 | |
| PRKCE | | | | | | | | | | 6 | | |
| PRKCG | | | | | | | | | 8.5 | | | |
| PRKCH | 21 | | | | | | | | | | | |
| PRKCI | | | 23 | | | | | | | | | |
| PRKCQ | | 11 | 12 | | | | | | 12.5 | | 10 | |
| PRKCZ | | | | | | | | | | 10 | 6 | |
| PRKD1 | | | | | | | | | | 13 | | 14 |
| PRKD2 | | | | | | | | | 23 | | | |
| PRKD3 | | | 21.5 | | | | | | 22 | | | |
| PRKG1 | 8 | 7 | 8 | | | | | 7 | 7 | | | 10 |
| PRKG2 | | | 6.833 | | | | | | | | | |
| PRL8A1 | | | | | | | | 3 | | | | |
| PRLR | 23 | | | | | | | 8 | | | | |
| PRM1 | | | | | | | | 5 | 6 | | | |
| PRMT10 | | | | | | | | 8 | | | | |
| PRMT3 | | | | | | | | | 23 | | | |
| PRMT8 | | | | | | | | | | 7 | | |
| PRND | | 6 | | | | | | | | | | |
| PRNP | | | | | | 0 | | | | | | |
| PRODH | 3 | | 1 | | 5 | 3 | | 3 | | 2 | 3 | 3 |
| PRODH2 | | | | | | | | 21 | | | | |
| PROM1 | | | | | | 19 | | | | | | |
| PRORSD1 | | | | | | | | | | 9 | | |
| PROSAPIP1 | 20 | | | 21.5 | | | 21 | | | | | |
| PROSC | | | | | | | | 2.5 | 22 | | | |
| PROX1 | | | | | | | | | | | | 23 |
| PRPF19 | | | | 8 | | | | 12 | | 10 | | |
| PRPF38B | | | | | | | | 20 | 17 | | 22.5 | |
| PRPF40A | | | | | | | | | 21 | | | |
| PRPF40B | 19.5 | | 22 | | | | | 20.5 | 21 | | | 21 |
| PRPS1 | | | | 23 | | | 0 | | | 16 | | |
| PRPS2 | | | | | | | | 6.5 | | | | |
| PRPSAP1 | 17 | | | | | | | 0 | | | | |
| PRR13 | | | 21 | | | | | | | | | |
| PRR14 | | | | | | | 19 | | | | | 22.5 |
| PRR15 | | | | | 1.167 | | | | | | | |
| PRR15L | | | | | | | | | 21.5 | | | |
| PRR16 | | | | | | | | | 23.5 | | | |
| PRR5 | | | | | 4.5 | 0 | | | | | | |
| PRRC1 | | | | | | | 17 | | | | | |
| PRRC2C | | | | | | | | | 0 | | | |
| PRRG1 | | | | | | | | | 20 | | | |
| PRRG3 | | | | 14 | | | 14 | | | | 4.5 | |
| PRRG4 | | | | | | | | | | | | 13 |
| PRRXL1 | | | | | | | | | 19.5 | 17 | | |
| PRSS23 | 20.5 | | | | | | | 23 | | | | 22 |
| PRSS37 | 18 | | | 23 | | | | | | | | |
| PRSS8 | 10 | 12 | 11.5 | | | | | | | 12 | 12 | 11 |
| PRT1 | | | | | | 21 | | | 17 | 23 | 21.5 | |
| PRTN3 | | | | | 2 | | | | | | | |
| PRUNE | | | | | | | | 3.5 | 6 | 23 | | |
| PRX | | | | | | | | | 22 | 4 | 22.5 | |
| PSAP | | 7 | 8.5 | | | | | | | | | |
| PSAT1 | | | | | | | | | | 13 | | |
| PSD3 | | | | | | | | | | | 7.833 | |
| PSEN1 | | | | | | | | | 19 | | | |
| PSEN2 | | | | | | | | | | 7 | | |
| PSG19 | | | | | | | | 11 | | | | |
| PSIP1 | | | | | | | | | | | 21 | |
| PSMA1 | 8 | | | | | | | 11 | | | | |
| PSMA6 | | | | | | | | | | | 22.5 | |
| PSMA7 | | | | | | | | | | | | 10 |
| PSMB3 | | | 0.5 | | | | | | | | | 21.5 |
| PSMB4 | 20.5 | 22 | 23 | | | 0 | | 23 | | 21 | 23 | 22 |
| PSMB5 | | | | 0 | | | 0 | 22 | 22 | | | |
| PSMB6 | | | | | | | | | 16.5 | | | |
| PSMB7 | | | | | | | | | 13 | | | |
| PSMB9 | | | | | | | | | 23 | | | 23 |
| PSMC2 | | | | | 22.5 | | | 23 | | | | |
| PSMC3 | | | | | | | | 20 | | | | |
| PSMC4 | | | | | | | 18 | | | | | |
| PSMC6 | | | | | | | | 1 | | | | |
| PSMD1 | | | | | | | | | 22 | | | |
| PSMD12 | | | | | | | 9 | | | | | |
| PSMD13 | 11 | | | | | | | | | | | |
| PSMD14 | | | | | | 10 | | | 15 | 8 | | |
| PSMD2 | | | | | | | | | 22 | | | |
| PSMD4 | | | | | | | | | 23 | 22 | | |
| PSMD5 | | | | | | | | | 4 | | | |
| PSMD6 | | | 17 | | | | | 20 | 15.5 | 14 | | |
| PSMD7 | | | | | | | | | 0 | 0 | | |
| PSME1 | | | | | | | | 21 | | | | |
| PSME2 | | | | | | | | 22 | 22 | | | |
| PSME4 | | | | | | | | 5 | 4 | | | 7 |
| PSMF1 | | | | | | | | | | 5 | | |
| PSTK | | | | | | | | 2.5 | | | 3 | |
| PSTPIP2 | | | | | | | | | 22 | 20 | | |
| PTBP1 | | | | | | | | | 3 | | | |
| PTBP2 | | | | | | 20 | | 12.5 | 17 | | | |
| PTCH1 | | | 23.5 | | | | | | 16 | | | 23 |
| PTDSS2 | | | | | | | | 21.5 | | 0 | | |
| PTER | | | | | | | | | | | | 7.5 |
| PTGDR2 | | | | | | | | 22 | | | | |
| PTGDS | | | | | | | | 22 | | 12 | | |
| PTGER3 | | | | | | | | | | 12 | | |
| PTGER4 | | | | | | | | | 23 | | | |
| PTGES | | | | | 1 | 10 | | | | | | |
| PTGES3 | | | | 21 | | | 21 | | | | | |
| PTGFR | | | | | | | | | 5 | | | |
| PTGFRN | | | | | | | | 20 | | | | |
| PTGIS | | | | | | | | | 20 | 5.5 | | |
| PTGR1 | | | | | | | | 16 | 21 | | | |
| PTGS1 | | | | | | | | 21 | | | | |
| PTK2 | | | | | | | | | | 13 | | |
| PTK6 | 18 | | | | | 18 | | | | | | |
| PTK7 | | | | | | | | | | | | 10 |
| PTOV1 | | | 23 | | | 19 | | | 0.5 | | | |
| PTP4A2 | | | | | | | | | 6 | | | |
| PTP4A3 | | | 21 | | | | | | | | | 0.5 |
| PTPLAD1 | | | | | 0 | | | | 22 | | | |
| PTPLB | 21 | | | | | | | 22 | 21 | | | |
| PTPN11 | 9 | | | | | | | | | 11.5 | | |
| PTPN13 | | | 21.5 | | | | | | 4 | | | |
| PTPN14 | 4.5 | | 9 | | | | | | | | | |
| PTPN22 | | | 11 | 11 | | | | 10.5 | | 12 | | |
| PTPN3 | | | 6 | | | | | | | | | |
| PTPN4 | | | | 19 | | | 19.5 | | | 17 | | |
| PTPN6 | | | | | | | | | | 7 | | |
| PTPN9 | | | | | 8 | | | 12 | 19 | | | |
| PTPRB | | 23 | 22.5 | | | 23 | | | | 23 | 22 | 23 |
| PTPRD | | | | | | | | 10 | | | | |
| PTPRE | | | | | | | | | | | | 6.167 |
| PTPRF | | | 22.5 | | | | | | | | 23.5 | 21.5 |
| PTPRG | | 0 | 0 | | | | | | 22 | | | |
| PTPRJ | | | 15 | | | | | | | 14 | | |
| PTPRK | | | | | | | | | | 10 | | |
| PTPRM | | | | | | 12 | 8 | | | 11 | | |
| PTPRS | | | 7 | | | | | | | | | 11 |
| PTPRT | 4.5 | | | | | | 9 | | 23 | | | |
| PTPRU | | | 23 | | | | | | | | | |
| PTPRZ1 | | | | 9 | | | | | 1 | | | |
| PTRF | | | | | | | | | 22.5 | | | |
| PTRH2 | | | | | | | | | | | 1 | |
| PTTG1 | | | 6 | | | 23 | | | 3 | | | |
| PTTG1IP | | | | | | | | | 11.5 | | | |
| PUF60 | | | | | | | | | | 8 | | |
| PUS7 | 14 | 13.5 | | | 16 | 13 | 18 | | | 13 | | |
| PUSL1 | | | 12.5 | | | | | 14 | 13 | 10 | | |
| PVRL1 | | 2 | 1 | | | 23 | | 23.5 | | 0 | 1 | 1.5 |
| PVRL2 | | | | | | | | | | | 6 | |
| PWP1 | | | | | | | | | 1 | | | |
| PXDC1 | | | | | | | | | | | 23 | |
| PXDN | | | | | | | | | 0 | | | |
| PXK | | | | | | | | 8 | | | | |
| PXMP2 | | | | | | 16 | | | 21 | 13 | | 16 |
| PXMP4 | 15 | | | | | | | | 12.5 | | | |
| PYCRL | 15.5 | | | | | | | 23 | | | | |
| PYGM | 2.5 | 6 | 6 | 6 | 5 | 3 | 4 | 3 | | | | |
| PYGO1 | | | | | | | | | | | 5 | |
| PYROXD1 | | | | | | | | | 9 | | | |
| QARS | | | | | | | | 1 | | | | |
| QDPR | | | 15 | | | 14 | | | | 15 | | |
| QKI | | | | | 4 | | | | | | | |
| QPCTL | | | | | | | | | | 11.5 | | |
| QPRT | | | | | | | | 5.5 | | | | |
| QRSL1 | | | | | | | | 2 | | 14 | | |
| QSOX1 | 5 | 3 | 6 | | | | | | | | 1 | |
| QSOX2 | | | | | | | | 3 | | | | |
| QTRT1 | | | 7 | | | | | | 23 | | | 10 |
| R3HCC1 | | | | | | 15 | | | | | | |
| RAB11FIP3 | | | 21 | 12 | | | | | | 22 | | |
| RAB14 | | | 21 | | | | | | | | | |
| RAB15 | | | | | | | 12 | | 20 | | | |
| RAB17 | | | | | | | | 7 | | | 6.833 | |
| RAB20 | | | | | | | | | | 14.5 | | |
| RAB21 | | | | | | | | 3 | | | | |
| RAB22A | | | | | | | | 1 | | | | |
| RAB27B | | | | | | | | | | 2 | | |
| RAB30 | | | 22.5 | | | | | 22 | | | | |
| RAB31 | | | | | | | | | 19 | | | |
| RAB32 | | | | | | | 7 | | | | | |
| RAB33A | 18.5 | | | | | | | | | | | |
| RAB34 | | | | | | | | | 15.5 | | | |
| RAB35 | | | | | | | 21 | 22 | | | | |
| RAB39A | 23 | | | | | | | 3 | | | | 23.5 |
| RAB3A | | | | | | | | 22 | | | | |
| RAB3B | | | | | | | | | 3 | | | |
| RAB3C | | | | | | | 0.5 | | 22 | | | |
| RAB3GAP2 | | | | | | | | 6.5 | | | | |
| RAB3IP | | | | | | | | | 0 | | | |
| RAB40B | | | 21 | 22 | 20 | | | | 23 | 17 | | |
| RAB43 | | | | | | | | | | | | 7 |
| RAB4A | | | | | | | | | | | | 8 |
| RAB6A | | | | | | | | | 0 | | | |
| RAB6B | | | | | | | | 11 | 22 | | | |
| RAB7A | 21.5 | 15 | | 22 | | | | 6 | | | | |
| RAB7L1 | | 16 | | | 18 | | | | | 16 | | |
| RABAC1 | | | | | | 0 | | | | | | 22 |
| RABEP1 | | | | | | 0 | | | | 4 | 0 | |
| RABGAP1 | | | | | | | | | 12 | | | |
| RABGEF1 | | | | 20 | | | | | 21 | | | |
| RABGGTA | | | | | | | | 1 | 0 | | | |
| RABGGTB | | | | | | | | | 4 | | | |
| RABL2A | | | | | | | | 14 | | | | |
| RABL3 | | | | | | | | | | | | 21 |
| RABL5 | | | | | | | | | | | 22 | |
| RAC3 | | | 9 | | | | | | | | | |
| RACGAP1 | | | 9 | | | | | | 15 | | | 14 |
| RAD18 | | | | | | | | | 23 | | | |
| RAD21 | | | | | | | | | | 10 | | |
| RAD23B | | 13 | | | | | | 13 | 18 | | | 12.5 |
| RAD50 | | | | | | | | 22.5 | | | | |
| RAD51C | | | | | | | | 19 | | | | |
| RAD51D | | | | | | | | 20 | 21 | | | |
| RAD9A | | | | | | | | | 14 | | | |
| RAD9B | 0 | | | | | | | 2 | | | | |
| RADIL | | | | 21 | 21.5 | | | | | | | |
| RAE1 | | | 7 | | | | | | | | | |
| RAF1 | | | | | | | | | | 12.5 | | |
| RAI1 | | | | | 2 | | | | | | | |
| RAI14 | | | | | | | 19 | | | | | |
| RALA | | | | | | | | | 14 | | | |
| RALB | | | | | | | | | 22 | | | |
| RALBP1 | | | | | | | | | | 6.5 | | |
| RALGAPA2 | | 9 | | | | | | 11.5 | 7 | 10 | | |
| RALGAPB | | | | | | | | | 6 | | | |
| RALGPS1 | | | | | | | | | 0 | | | |
| RALGPS2 | | | | | | | | 23 | 22.5 | 15 | | |
| RAMP1 | | | | | | | | | 16 | | | |
| RAMP2 | | | | | | | | | 7 | | | |
| RANBP10 | | | | | | | | 5.5 | | | | |
| RANBP17 | | | | | | | | 4 | 9 | | | |
| RANBP3L | | | | | | | | 4 | | | | |
| RANGAP1 | | | | | | | | | 23 | | | |
| RAP1A | | 23 | 0 | | | | | 20 | 23.5 | | | 23.5 |
| RAP1B | | | | | | | | 3 | 1 | | | |
| RAP1GAP | | | | | 4 | | | 21 | | | | |
| RAP2A | | | | | | | | 23 | | | | |
| RAP2C | | | | | | | | | | 13 | | 14 |
| RAPGEF1 | | | | | | | | | 19 | | | |
| RAPGEF3 | | | | | | | | 7 | | | | |
| RAPH1 | | | | | | | | | | 16.5 | | |
| RARA | | 12 | 12 | 10.5 | 9 | 12 | | 12 | | 11 | 11 | |
| RARB | | 7.5 | | | | | | | | | | |
| RARG | | | | 2 | | | | | | | | |
| RARRES2 | | | | | 9 | | | | | | | |
| RASA2 | | | | | | | | 1 | | 4 | | |
| RASAL2 | | | | 22 | | | | | 12 | | | |
| RASD1 | | | | 21 | | | | | | | | |
| RASD2 | | | | | | 1 | | | | | | |
| RASGEF1B | | | | | | | 18 | | | | | |
| RASGRP1 | | | | | | | | 11 | | 8.5 | | |
| RASGRP3 | | | | | | | | | 0 | | | |
| RASGRP4 | | | | | 23 | | | | | | | |
| RASIP1 | | | | | | | | | 4 | | | |
| RASL10B | | | | | | | | | 10 | | | |
| RASL11A | | | | | | | | | 12 | | | |
| RASL11B | | | | | | | | | | 12.5 | | |
| RASL12 | | | 21 | | | 15 | | | | 14.5 | | |
| RASL2-9 | | | 12 | | 19 | | | | 10.5 | | | |
| RASSF3 | | | | | | | | | 13.5 | | | |
| RAVER2 | | | | | | | | | | | 1 | |
| RBBP5 | | | | | | | | | | 13 | | |
| RBBP9 | | | | | | | | | | 16 | | |
| RBCK1 | | 7 | | | | 10 | | 9.5 | | | | |
| RBFA | | | | | | | | 12 | 23 | | | |
| RBFOX1 | | | | | | | | | 21 | | | |
| RBFOX3 | | | | | | | | 10 | | 11 | | |
| RBL1 | | | | 23 | | | 5.5 | | | | | |
| RBL2 | | | | | | | | 10 | | | | |
| RBM12B | | | | | | | | 5.5 | | | | |
| RBM22 | 19 | | | | | | | | | | | |
| RBM25 | | | | | | | | | 4 | | | |
| RBM27 | | | | 8.5 | | | | | 2 | | | |
| RBM28 | | | | | | | | | 12.5 | | | |
| RBM33 | | | | | | | | | 12 | | | |
| RBM38 | | 3 | 23.5 | | | 2.5 | | | 17 | | 0.5 | 0 |
| RBM39 | | | | | | | | | 2 | | | |
| RBM41 | | | | 20 | | | | | | | | |
| RBM42 | 10 | | | | | | | | 18 | | | |
| RBM45 | | | | | | | | | | 9 | | |
| RBM47 | | | | | | 18.5 | | 21 | | | | |
| RBM4B | | | | | | | | | 17 | | | |
| RBMS1 | | | | | | | | | | 19 | | |
| RBMS2 | | | | | | | | | | 5 | | |
| RBMX | 3 | | | | 2 | 8 | 4 | | | | | |
| RBP1 | | | | | | | | | | 12 | | |
| RBP4 | | | | | | | | 8 | | | | |
| RBP7 | | | | | | | | | | | 23 | |
| RBPJL | | 7 | 6 | | | | | 7 | | 7.5 | | |
| RBPMS | | | | | | | | 6 | | | | |
| RC3H1 | | | | 2 | 0 | | | | | | | |
| RCAN1 | | | 23 | | | | | | | | | 19 |
| RCAN2 | | | | | | | | 22 | | 4 | | |
| RCAN3 | | | | | | | | | | 0 | | |
| RCBTB2 | | | | | | | | 21 | 18 | | | |
| RCC1 | | | 0.5 | | | | | 0 | 1 | | | 0 |
| RCE1 | | | | | | | | | 21.5 | | | |
| RCL1 | | | | | 4 | | | 21 | | | | |
| RCN2 | | | | 23 | | | | | 14 | | | |
| RCOR1 | 21 | 23 | 0 | | | | | 21 | 2 | | 22.5 | |
| RCOR3 | | | | | | | | | 23.5 | | | 23 |
| RDH10 | | | | | | | | 23 | | | | |
| RDH11 | | | | | | | | | 20.5 | 6 | | |
| RDH13 | | 22 | | | | 0 | | | | 23 | | |
| RDH5 | | | 10 | | | | | | 20 | | | 12 |
| RDH9 | | | | | 22.5 | | | 23 | | | | 6 |
| RDX | 21 | | | | | | | | | | | |
| RECK | | | | 5 | | 1 | | | 22.5 | 22 | | 2 |
| RECQL | | | | 5.5 | | | | | 21 | 10.5 | | |
| REEP1 | | | | | | | | 19.5 | | | | |
| REEP4 | | 6 | | | | | | | | | | 7 |
| REEP5 | 22.5 | | | | | 23.5 | | 0 | | 1.5 | 0 | |
| REEP6 | | | | | | | | | | | 7 | |
| REL | | | 2 | | | | | | | | 23 | |
| RELA | | | | | 20 | | | | | | | |
| RELL1 | | | | 2.5 | | | | | | | | |
| RELT | | | | | | | | | | | | 20.5 |
| RENBP | | | | | | | | | 2 | 10.5 | | |
| REPIN1 | | | | | | | | | 23 | | | |
| REPS1 | 15 | | | | | | | | | 16 | | |
| RERE | 0 | | 23.5 | | | | 5 | | | | | |
| RERG | 22 | | 23.5 | | | 2 | | 1 | | 3 | | |
| RET | | | | | | | | 23 | | | | |
| RETN | | | 16.5 | | | | | | 12 | | | |
| RETNLB | | | | | | | | | | 15 | | |
| RETSAT | | | 7 | | | | | | 17 | | | |
| REV1 | | 22 | | | | | | | | | | |
| REXO4 | 8 | 9 | | | | | | | | | | |
| RF | | | | | 7.5 | | | 18 | | | | |
| RFC3 | | | | | | | | | 11.5 | | | |
| RFC4 | 8 | 9 | 9 | | | | | | 7 | | | 11 |
| RFESD | | | | | | | | 0 | 6 | | 8 | |
| RFFL | | | | | | | | | 13 | | | |
| RFK | | | 9.5 | | | | | | | | | |
| RFTN1 | | | | | | | 7 | | | | | |
| RFTN2 | | | | | | | | 20 | 20 | | | |
| RFX3 | | | | | | | | 12.5 | | | | |
| RFX4 | 22 | | 2 | | | | | | 2 | | | |
| RFX5 | | | | 19 | | | | | | 14 | | |
| RFXANK | | | | 22.5 | | | | | 14 | | | |
| RG2 | | | 19 | | | | | | | | | |
| RGCC | | | | | | | | | 14 | | | |
| RGL1 | | | | | | | | | | | 9.5 | |
| RGL3 | | | 6 | | | | | | | | | |
| RGMA | | | | | | | | 19 | | | | |
| RGNEF | | | | | | | | 0 | 22 | | | |
| RGS12 | | | | | | | | 23 | 22 | | | |
| RGS16 | | | | | | | | 1 | | | | |
| RGS19 | 3 | | | | | | | | | | | 23.5 |
| RGS2 | | | | | | | | | | | | 9 |
| RGS4 | | 6 | 5 | | | | | | | | | |
| RGS7 | | | | | | | | 20 | 21 | | | |
| RGS7BP | | | | | | | | | 20 | 15 | | |
| RGS9 | | | | 21.5 | | | | | | | 1 | |
| RHBDD2 | | | | | | 16 | | | | | | 20.5 |
| RHBDD3 | | | 0 | | | | | | | | | |
| RHBDF1 | | | | | | | 17 | | | 15 | | |
| RHBDF2 | | | 2 | 21 | | | | | | | | |
| RHBDL3 | | | | | | 21 | | | | | | |
| RHOA | | | | | | | | | | 12 | | 12 |
| RHOB | | | 0.5 | | | | | | 19 | | | 21 |
| RHOBTB1 | | | | | | 1.5 | | | | | | |
| RHOBTB2 | | | | | | | | 20.5 | | | | |
| RHOBTB3 | | 6 | | | | | | 6 | | | | |
| RHOC | 16.5 | | | | | | | | | | | |
| RHOD | | | | | | 1.5 | | | | 8 | | |
| RHOJ | | | | | | | | 22 | | | | 0 |
| RHOQ | | | | 9 | 8 | | | | | | | |
| RHOU | 21 | | | | | | | | | 8 | | |
| RIC8A | | | | | | 18 | | 21 | 22 | | | |
| RIC8B | | | | | | 13 | | | 13 | 10 | | |
| RICTOR | | | | | 4 | 2.5 | | | | | 3 | 5 |
| RILP | | | | | | | | | | 11.5 | | |
| RILPL1 | | | 22 | 14 | | | | | | | 0.5 | |
| RIN2 | | | | | | | | | | 8 | | |
| RIN3 | | | | | | | | | | 8.5 | | |
| RING1 | | | 7 | | | | | | | | | |
| RINT1 | | | | | | 4 | | | | | 5 | |
| RIOK2 | | | | | | | | | | 16.5 | | |
| RIPK1 | 7 | | | | | 0.5 | | | | | | |
| RIPK2 | | 12 | | | 12 | 13 | | | | 9 | | 12 |
| RIPK3 | | | | | | | | | 16 | | | |
| RIPK4 | | | | | | | | 20 | | | | 20 |
| RIPPLY1 | | | | | 19 | | | | | | | |
| RMI1 | | | | | | | | 7 | | | | |
| RMND1 | | | 15 | | | 19.5 | | 20 | | 3.5 | | |
| RMND5B | | | | 13 | | | | | | | | |
| RND1 | | 18 | 20 | 20 | 19.5 | 20 | 22 | 19 | | 18 | | |
| RND2 | 18 | | | | | | | | | | | |
| RND3 | | | 0 | | | | | | 20 | | 23 | |
| RNF11 | | | | | | | | | | 9 | | |
| RNF114 | | | | | | | | | | 13 | | |
| RNF115 | | | 4 | | | | | | | | | |
| RNF122 | | | | | | | | | | 6 | | |
| RNF125 | 8 | | | | | | | 13 | 11 | 8 | | |
| RNF135 | | | 7.5 | | | | | | | | | |
| RNF14 | | | | | | | | | 2 | | | |
| RNF141 | | | | | | | | 11 | | | | |
| RNF144A | | | | | | | | 21 | | | | |
| RNF144B | | | | | | | 6 | | | | | |
| RNF145 | | | 7.167 | | | | | | | | | |
| RNF146 | | 0 | 1 | | | | | | | | 0 | |
| RNF149 | | | | | | | | | 19 | | | |
| RNF150 | | | | | | | | | 21 | | | |
| RNF152 | | | | | | 21 | | | | | | |
| RNF167 | | | | | | | | | 20 | | | |
| RNF168 | | | 21 | 21 | | | | 20 | 23 | | | |
| RNF169 | | | | | | | | | | 17 | | |
| RNF181 | | 0 | 0 | | | | | | | | | |
| RNF183 | | | | | | | | 12 | | | | |
| RNF19B | | | | | | | 5 | | | | | |
| RNF2 | 19 | | | | | | | 23 | | | | |
| RNF207 | | | | | | | | | | 6 | | 7.5 |
| RNF208 | | | | | 3 | 22 | | | 2 | | 23 | |
| RNF213 | | | | | | 11 | | 6 | 4 | | | |
| RNF214 | | | | | | | | | | 7 | | |
| RNF215 | | | | | | | | 16 | | 17 | | 20 |
| RNF220 | | | | | | | | 19 | 18.5 | 18 | | |
| RNF24 | | | | | | | | | | 3 | | |
| RNF32 | 17 | | | | | | | | 16 | 13 | | |
| RNF34 | | | | | | 0 | | | | | | |
| RNF39 | | | | | | | | 20 | | | | |
| RNF4 | | 6 | | | | | | | | | | |
| RNF40 | | | | | | | | | | 17 | | |
| RNF43 | | | 9.5 | | | 20 | | | | | | |
| RNF8 | | | | | | | | 8 | | | | |
| RNFT1 | | | | | | 13 | | 11.5 | | 13 | 15 | |
| RNFT2 | 22 | | | | | | | | | | | |
| RNGTT | 21 | 20.5 | | | | | | 22.5 | 22.5 | 20 | | |
| RNH1 | | | | | | 16 | | | 23 | | | |
| RNPEP | | | | | | | | 21.5 | | | | |
| ROBO1 | | | | | | | | | | | 11.5 | |
| ROBO4 | | | | | | | | 1 | 22 | | | |
| ROGDI | | | | 19 | 19 | | | | | | | |
| ROMO1 | | | | | | | | | 21.5 | | 4.5 | 22 |
| ROPN1L | | | | | | | | 12 | | | | |
| ROR1 | | | | | | | | | 22 | | | |
| RORA | | | | | | | | | 23 | | | |
| RORC | | | | | | | | 13 | 23 | | | |
| RP24-221A14.2 | 23 | 0 | 0 | 2 | 1.5 | 23 | | 23 | 23.5 | 23 | | 23 |
| RP9 | | | | | | | | 23 | | | | |
| RPA1 | | 0 | 0 | | | | | | | | | 23 |
| RPAIN | 21 | | | | | | | | | | | 10 |
| RPE | 6 | | 7 | | | | | | 7.5 | | | |
| RPF1 | | | | | | 16.5 | | | | 13 | | |
| RPGR | | | | | | | | 11 | | | | |
| RPH3AL | 21 | | | | | | | 21 | | | | |
| RPL15-PS1 | | | 22 | | | | | | 15 | | | |
| RPL23 | | | | | | | | | 9.5 | 12 | | 9 |
| RPL24 | | | | | | | | | | 11 | | |
| RPL7L1 | | | | | | | | | | 16 | | |
| RPLP1 | 6 | | | | | | | 6.5 | | | | |
| RPN1 | | | | | | | | | | 11 | | |
| RPN2 | | | | | | | | 10.5 | | | | |
| RPP21 | | | | | | | | 17 | | | | |
| RPP25 | | | 3.5 | | | | | | 23 | | | 1 |
| RPP25L | | | | | | 6.5 | | | | | | |
| RPP30 | | | | | 22.5 | | | | | | | |
| RPP38 | 10 | | | | | | | | | | | |
| RPRD1A | | | | | | | | | 1.5 | | | |
| RPRD1B | | | | | | | | 19 | | | | |
| RPS11 | | | | | | | | | 22.5 | | | |
| RPS14 | | | | | 20.5 | | | 21 | | | | |
| RPS15A | | | 8.5 | | | | | | | | | |
| RPS19 | 23 | 1 | | | | 2.5 | | 2 | | 1 | | |
| RPS3 | | | | | | | | | 18 | | | |
| RPS6KA1 | | | 23 | | | | | | | | | |
| RPS6KA3 | | | | | | | | | 23 | | | |
| RPS6KA4 | | | | | | | | 21.5 | | | | |
| RPS6KB2 | | | | | | | | | 12.5 | | | |
| RPS6KC1 | | | | | | | | | 22 | | | |
| RQCD1 | | | | | | | | | | 17 | | |
| RRAGC | | | | | | | | | | 14 | | |
| RRAGD | | 7 | | | | | | | | | | |
| RRAS | | | | | | | | | 18 | | | |
| RRAS2 | | | | | | 22.5 | | | | | | |
| RRBP1 | | | | | | 16 | | | | | | |
| RREB1 | | | | 22 | | | | | | | | 8 |
| RRM2 | 10.5 | 10.5 | | | | | | 6 | | 14 | | 14.5 |
| RRP12 | | | | | | | | 5.5 | | | | |
| RRP1B | | | 1 | | | | | | 1 | | | |
| RRP8 | | | | | | | | | 20 | 10 | | |
| RRP9 | | 15 | | 21 | | | 20 | | | 12 | | |
| RS | | | | | | 2 | | | 4.5 | | | |
| RS2 | 7 | | | | | | | | | 4 | | |
| RSAD1 | | | | | | | | | | | 23 | 6.5 |
| RSAD2 | | 15 | | | 3 | | | | | | | |
| RSBN1 | | | | 1 | | | | 23 | | | | |
| RSBN1L | | | | | | | | | | | | 13.5 |
| RSE_MRP | | | | | | | | | | | | 7 |
| RSE4 | | | | | | | | | | 13 | | |
| RSEH2B | | | | | | | | | 21.5 | | | |
| RSEP_NUC | | | | | | | | | 5 | | | |
| RSPO3 | 19 | | | | | | | | | | | |
| RSPRY1 | 18.5 | | | 23 | | 19 | | 21 | | | | |
| RTDR1 | | | | 5.5 | 18 | | | | | | | |
| RTEL1 | | | | | | | 22.5 | 19 | 21.5 | | | |
| RTKN | | | | | | | | | | 11 | | |
| RTN4IP1 | 19 | | | | | | 21 | 23 | 1.5 | | | |
| RTP1 | | | | | 14 | | | | | | | |
| RTP3 | 15 | | | | | | | | | | | |
| RTTN | | | | 0 | | | | | | | | |
| RUFY2 | | | | | | | | 8 | 18 | 11 | | |
| RUFY3 | | | | | | | | | 12 | | | |
| RUFY4 | | | | | | | | | 7 | | | |
| RUNDC3B | 20 | | | | | | | | | | | |
| RUNX1 | | | | | | | | 23.5 | 6 | | | |
| RUNX1T1 | | | | | | | | 21.5 | | | | |
| RUSC2 | | 19 | 18 | | | | 20 | | | 0 | 0 | 20.5 |
| RUVBL1 | | | | | | | | 1 | | | | |
| RWDD1 | | | | | | | | | | | | 8.5 |
| RWDD3 | | | | | | | | | 16.5 | | | |
| RWDD4 | | | | | | | | 10.5 | | | | |
| RXFP4 | | | | | | | | | | | 1 | |
| RXRA | | | | | | | | 10 | | | | |
| RXRB | 4 | | | | | | | | | | | |
| RXRG | | | | | | | | | 22 | | | |
| RYK | | | | 19.5 | | | 20 | 21 | | | | |
| RYR3 | | | | 20 | | | 19 | | | | | |
| S100A1 | | | | | | | | | 22 | | | |
| S100A10 | | | | | | | | 0 | | | | |
| S100A16 | | | | | | | | | 19.5 | | | |
| S100A4 | | | | | | | | | | | | 0.167 |
| S100A9 | | | | | | | | 3 | | | | |
| S100B | | | | | | | | 8 | | | | |
| S100G | | | 5 | | | | | | | | | |
| S1PR1 | | | | | | | | | 19 | | | |
| S1PR3 | | | | | | | | 15.5 | | | | |
| S1PR4 | | | | 19 | | | 19 | | | | | |
| S1PR5 | | | | | | | | 17 | 19 | | | |
| SACS | | | | | | | | 17 | 18 | | | |
| SAE1 | | | | 20.5 | | | 21 | 21 | | | | |
| SAFB2 | 5.5 | | 20 | | | | 21 | 18.5 | | 18 | | |
| SAMD12 | | | | | | 13 | | 13 | 11 | | | 17 |
| SAMD4A | | | | | | | 17 | | 15 | | | |
| SAMD5 | | | | | | | | 5.5 | | 8 | | |
| SAMD8 | | | | | | | | | | | | 17 |
| SAMD9L | | | | | | 3 | | | | | | |
| SAMM50 | | | | | | | | | | 7 | | |
| SAP30L | | | | | | | | | 1 | | | |
| SAR1A | | | | | | | | 6 | 2 | 11.5 | | |
| SAR1B | | | | | | 4 | | 23 | | | | |
| SARS | | | | | | | | | | 10 | | |
| SASH1 | | 5 | | | | | | | | | | |
| SAT1 | | | | 18.5 | 17 | | | | | | | |
| SBDS | | | | | | 6 | | | | | | |
| SBF1 | | | | | | | | | | | | 9 |
| SBF2 | | | | | | | | | | | 2 | |
| SBK1 | | | | | | | | | | | 3.5 | |
| SBNO2 | | | | | | | | | 8 | | | |
| SC5DL | | | | | | | | | 0 | | | |
| SCAF1 | | | | | | | | | 12 | | | |
| SCAF11 | | | | | | | | | 0 | | | |
| SCAMP1 | | | 3.5 | | | | | 3 | | | | |
| SCAMP2 | | | | | | | | | 21.5 | | | |
| SCAMP3 | | | | | | | | 0 | | | | |
| SCAP | | | | | | | | | 23.5 | | | |
| SCAPER | | | 9 | | | | | | | | | |
| SCARB1 | 22 | | 22 | | | | | | | | | 22 |
| SCARB2 | | 18.5 | | | | | | 18.5 | | | | |
| SCD | 6 | | | | | | | 11.5 | | | | |
| SCD2 | | 2 | 3 | | | | | | 23 | | | |
| SCD3 | | | 6.5 | | | | | | | | | |
| SCD4 | 0.5 | 23 | 23 | | | 21 | | 22 | 0 | | | |
| SCEL | | | 5.833 | | | | | | | | | |
| SCFD1 | | | | | | 14 | | 2 | 22 | | | |
| SCGB1A1 | 4 | | | | | | | | | | | |
| SCGN | 16 | | | | | | 6 | | 23 | | | 22.5 |
| SCIN | | | | | | | | 2.5 | | | | |
| SCLT1 | | | | | | | | | | 6 | | |
| SCLY | | | | | | | | | | 4.5 | | |
| SCN1B | | | | | 6 | | | | | | | |
| SCN2A | 15 | 16.5 | 18 | | | | | | | | 18 | 16.5 |
| SCN2B | | | | | | | | | 23 | | | |
| SCN3A | | | | | | | | | | 8 | | |
| SCN3B | | 7 | | | | | | | | 10 | | |
| SCN7A | | | | 21 | | | | | | | | |
| SCNM1-PS | | | | | | | 12 | | | 11 | | |
| SCNN1A | | | | | | | | 3.5 | | | | |
| SCNN1B | 9 | 8 | | | | | | | | 8.5 | | |
| SCO1 | | | | | | | | 20.5 | | | | |
| SCP2 | | | | | | | | 10 | | | 23 | |
| SCPEP1 | | | | | | | | | | | | 22 |
| SCRN1 | | 8 | | | | | | | 2 | | | |
| SCRN3 | | | 15 | 8 | 9.5 | 15 | | 12 | 13.5 | 9 | | 20 |
| SCTR | 8 | 9 | | | | | | 13.5 | | | 0.5 | |
| SCXA | | | 4.5 | | | 18.5 | | | | | | |
| SCYL1 | | | | | | | | | 20 | | | |
| SDC1 | | | | | | | | 22 | | | | |
| SDC2 | | 23 | 1 | | | | 4 | | 11 | | | |
| SDC4 | | | | | | | | 3.5 | | | | |
| SDCCAG8 | | | | | 19 | | | | 9 | | | |
| SDF2 | | | | | | | | 22.5 | 3.5 | | | |
| SDF2L1 | | | | | | | | | 21 | | | |
| SDHAF1 | 9 | | 22 | | | | | | | | | 22 |
| SDHD | 8.5 | | | | | | | | 23.5 | | | 12.5 |
| SDK2 | | | | | | | | | | 3.5 | | |
| SDPR | | | | | | | | | 4 | | | |
| SDR16C5 | | | | | | | | 9.5 | | | | |
| SDR42E1 | | | | | | | | 1 | | | | |
| SDR9C7 | | | | | | | | | | | 1.167 | |
| SEC13 | | | | | | | | 21 | 3 | | | |
| SEC14L1 | | | | | | | | | 22 | | | |
| SEC14L2 | | | | | | | | 10 | | | | |
| SEC14L4 | | | | | | | | | 23.5 | | | |
| SEC14L5 | | | 10.5 | | | | | 12 | 11 | | | |
| SEC16B | | | | | | | | | 4 | | | |
| SEC22B | | | | | | | | | 9.5 | | | |
| SEC23A | | | | | | | 9 | | | 10 | | |
| SEC23B | | | | | | | | 6.5 | 9 | | | |
| SEC24A | | | 9.5 | | | | | | | | | |
| SEC24B | | | | | | | | | 23 | | | |
| SEC24C | | | | | | | | | 23.5 | | | |
| SEC31A | | | | | | | | 23 | 0 | | | |
| SEC61A1 | | | | | | | | | 21 | | | |
| SEC62 | | | 10.5 | | | | | | | | | |
| SEC63 | | | | | | | | 22.5 | | | | |
| SECISBP2L | | | | | | | | | | | | 14 |
| SECTM1 | | | | | 23 | | 0 | | 20 | | | |
| SEL1L | | | | | | | | 23 | | | 21 | |
| SEL1L3 | | | | | | | | | 20 | | | |
| SELE | | | | | | | | | 12 | | | |
| SELENBP1 | | 5.5 | 7 | | | | | 22 | 22 | | | 2 |
| SELENBP1 | | | | | | | | 1 | 22 | | | |
| SELL | | 7 | | | | | | | | 9 | | |
| SELM | | | | 10 | 9 | | | 9 | | | | |
| SELO | | | | | | | | | 3 | 19 | | |
| SELRC1 | | | | | | | | 10 | | 8 | | |
| SEMA3B | | | | | | | | 19.5 | | | | |
| SEMA3G | | | | | | | | | 8 | | | |
| SEMA4A | | | | | | | | | 13 | | | |
| SEMA4B | | | | | | | | | 13 | | | |
| SEMA4C | | | | | | 12 | | | | | | |
| SEMA4D | | | | | | | | 21 | 22 | | | |
| SEMA5A | | | | | | | | 12 | | 13 | | |
| SEMA6A | | | | | | | | | 9 | | | |
| SEMA6B | 18 | | | | | | | 19.5 | | | | |
| SEMA6D | | | | | | | | 20 | | 9.5 | | |
| SEMA7A | | | | | | | 11 | | | | | |
| SENP2 | | | | | | | | | 13 | | | |
| SENP3 | | | | | | | | | | 16 | | |
| SENP6 | | | | | 6 | | | | | | | |
| SEPHS2 | | | | | | | | | | 19 | | |
| SEPSECS | | | | | | | | | | 8 | | |
| SEPW1 | | | | | | | 4 | | | | 6.167 | |
| SERAC1 | | | | | | | | 0 | | 0 | | |
| SERBP1 | | | | | | | | | | 13 | | |
| SERF1B | | | | | | | | 9 | 4.5 | | | 11 |
| SERINC2 | | | | | 8 | | | | | | | |
| SERINC3 | | | | | | | | | 6 | | | |
| SERINC5 | | 4 | 3.5 | | 3 | | | | | 3 | | |
| SERP1 | | | | | | | | | 2 | 6.5 | | |
| SERPI3B | | | | | | | | | 8 | | | |
| SERPI3C | | | | | 19.5 | | | | 19.5 | 16 | | |
| SERPI3F | 9 | 10.5 | 11 | 11 | 11 | 10.5 | 10 | 10 | 10 | 10 | 10 | 11 |
| SERPI3M | | | | | | | | | 23 | 2 | | |
| SERPI5 | | | 10 | | | | | 10 | 2 | | | |
| SERPI6 | | | | | | | | | | 16 | | |
| SERPI7 | | | | | | | | | | 8.5 | | |
| SERPINB1 | | | 22 | | | | | | | | | |
| SERPINB12 | 1 | 0 | 23 | | | | | 23 | 22 | 23 | 0 | 23 |
| SERPINB6B | | | | | | | | | 21 | | | |
| SERPINB9 | | | | 6 | | | | | | | | |
| SERPINE1 | 1 | | | | 22 | | | | | | | |
| SERPINE2 | | | | | | | | | 15 | | | |
| SERPINF1 | | | | | | | | 1 | | | | |
| SERPINF2 | | | 19 | | | | | | | | | |
| SERPINH1 | | | | | 12 | | | | 13 | | | |
| SERPINI1 | | | | | | | | | | 13 | | 10 |
| SERTAD2 | | | | | 5 | | | | | | | |
| SERTAD4 | | 22 | | | | 21 | | | | | | |
| SESN1 | | | | | | | | | 21 | | | |
| SESN2 | | | | | | | | | 21 | | | |
| SESN3 | | | 22 | | | | | | | | | |
| SET | | | | | | | | 8 | | 6 | | |
| SETD1B | | | | | | | | 4 | | 2 | | |
| SETD7 | | | | | | | | | 22 | | | |
| SETD8 | | | | | | | | 0 | | | | |
| SEZ6 | 18 | | | | | | | | | | | |
| SF1 | | | | | | | | | | | | 4.167 |
| SF3A2 | | | 10 | | | | | 21 | | 4 | | |
| SF3B1 | | | | | | | | | | 18 | | |
| SF3B3 | | | | | | 6 | | | | | | |
| SFN | | 6.5 | 8 | | | | | | 9 | | | |
| SFPQ | | | | | | | | | | 5.5 | | |
| SFRP4 | | | | 0 | 23 | | | | | | | |
| SFRP5 | 13 | 14 | | | | 15 | | 12.5 | 14 | 14 | 12 | 16 |
| SFSWAP | | | | | | 0 | | | 5 | | | |
| SFT2D2 | | | | | | | | | 1 | | | |
| SFTPB | | | 14 | | | | | | | | | |
| SFXN1 | | | | | | | | 10.5 | | | | |
| SFXN5 | | | | | | | | | 23 | 0 | 1.5 | |
| SGCD | | | | | | | | | 22 | | | |
| SGCE | | | | | | | | | 19 | 5.5 | 18.5 | |
| SGK1 | | | | | | | | | | | 1 | |
| SGK110 | | | | | | | | | | 13 | 5 | |
| SGK196 | | | | | | | | | 18 | 14 | | |
| SGK2 | | | | | | | | | | 18.5 | | |
| SGK3 | | | | | | | | 23.5 | 23.5 | | 22 | |
| SGMS1 | | | | 21 | 20 | | | 6.5 | | | | |
| SGMS2 | | | | | | | | | 9 | | | |
| SGOL2 | | 13 | | | | | | 11 | | | | |
| SGPL1 | | | | 5 | | | | | | | | |
| SGPP1 | | | | | | | | | 11.5 | | | |
| SGPP2 | | | | | | | 11 | | | | | |
| SGSH | | | | | | | | | 23 | | | |
| SGSM3 | | | | | | | | | 0 | | | |
| SGTA | | | | | | | | | | 14 | | |
| SGTB | | 19 | 19 | | | | | | 21 | 14 | 4 | |
| SH2B2 | | | | | | 16 | | | | | | |
| SH2B3 | | | | | | | | 7 | | | | |
| SH2D3C | | | | | | | | | 20 | | | |
| SH2D4A | | | 22 | | | | | | | | | |
| SH3BGRL2 | | | | | | 17 | | | | 18 | | |
| SH3BGRL3 | | | | | | | | | 18 | | | |
| SH3BP5 | 19 | | | | | | | | | | | |
| SH3D19 | | | 12 | | | 11 | | | | 9.5 | 10 | 10.5 |
| SH3D21 | | | | | | | | 20 | | 4 | | |
| SH3GL2 | | | | | | | | | 16 | | | |
| SH3KBP1 | | | | | | | | 11.5 | | 15 | | |
| SH3PXD2A | | | | | | | | | 23 | | | |
| SH3PXD2B | 19 | | | | | | | | | | | |
| SH3RF2 | | | | | | | 6 | | | | | |
| SH3TC2 | | | | | | | | 23 | | | | |
| SHANK2 | | 23 | 23 | | | | | | 0 | 22.5 | | 23 |
| SHANK3 | | 17 | 20 | | | | | | | | 21 | |
| SHARPIN | | 13 | | | | 12 | | | | 11 | | |
| SHB | | | | | | | | | 2 | | 23 | |
| SHC3 | | | | | | | | | 23.5 | | | |
| SHC4 | | | | | | | | | 18.5 | | | |
| SHCBP1 | | | | | | 20 | | 19 | | | | |
| SHISA2 | | | | | | | | | | | | 20 |
| SHISA4 | | | | | | 5.5 | | | | | | |
| SHISA6 | | | | | | | | | | 22 | | |
| SHKBP1 | | | | | | | | 2.5 | 21 | | | |
| SHMT1 | | | | | | | | | | 2 | | |
| SHMT2 | | | | | 13 | | | | | | | |
| SHOC2 | | | | | | | | | | | 22.5 | |
| SHPRH | | | | 20 | | | | 14.5 | | 5 | | 7 |
| SHROOM2 | | | | | | 23 | | | 21 | 9 | | |
| SI3 | | | | | | | | | | 3 | | |
| SIDT1 | | | | | | | | | 23 | | | |
| SIDT2 | | | | | 8 | | | | | | | |
| SIGIRR | | | | | | | | | | 14 | | |
| SIGLEC1 | | | | | | | | | | 13 | | |
| SIGLEC10 | | | | | | | | | 21.5 | | | |
| SIGMAR1 | | | | | | | | 8 | | | | |
| SIK1 | | | 9.5 | | | | | 8 | | | | |
| SIK2 | | | | | | | | | 22.5 | | | |
| SIKE1 | | | | | | | | | 3 | | | |
| SIL1 | | | | | | | | | 22 | | | |
| SIPA1 | | | | | | | | | | 9.5 | | |
| SIPA1L1 | | | | 21 | | | | | | 1 | | |
| SIPA1L2 | 5.5 | 9 | 10 | | | | | | | | | |
| SIPA1L3 | | | | | | | | | 3 | | | |
| SIRPA | | | | | | | | | | 17 | | |
| SIRT1 | | | | | | | | | | 13 | | |
| SIRT3 | | | | | | | | | | | 23 | |
| SIRT4 | 6 | | | | | | | | | | | |
| SIX1 | | | | | | | | | 5 | | | |
| SIX4 | | | | | | | | | | 16 | | |
| SKA2 | | | | | | | | | 11 | 15 | | |
| SKI | | | | | | | | 2 | | | | |
| SKIL | | | | | | | | 0 | | 0 | | |
| SKIV2L | | | | | | | | | | | 7 | |
| SKIV2L2 | | | | | | 22 | | | | | | 7 |
| SKP2 | 14 | | | 21 | | | | | | | | |
| SLAIN2 | | | 20.5 | | | | | | 1.5 | | | |
| SLC10A2 | | | | 14 | 13.5 | | | | | 13 | 11 | |
| SLC10A5 | | | | | | | | | 21.5 | | | |
| SLC10A6 | | | | | | | | | | 12.5 | | |
| SLC11A2 | | | | | | | | | | 9 | | |
| SLC12A2 | | | | | | | | 6 | 1 | | | |
| SLC12A4 | | | | | | | | | | 22 | | |
| SLC12A7 | | | | | | | | | | | | 11.5 |
| SLC12A9 | 21 | | | | | | | | | | | |
| SLC13A1 | | | | | | | 22 | 20 | | | 1 | |
| SLC13A2 | | | | | | | | 22 | | 5.5 | | |
| SLC13A3 | | | | 9.5 | 11 | | | | | | | |
| SLC13A4 | | | | 7 | | | 5 | | | | | |
| SLC15A1 | | | | | | | | | | 17 | | |
| SLC15A3 | | | | | 11 | | | | | | | |
| SLC15A4 | | | | | | | | | 3 | 3.5 | | |
| SLC16A1 | | | | | | | | | | 5 | | |
| SLC16A10 | 8 | | | | | 11.5 | | | | | | |
| SLC16A11 | | | 5.167 | | 7 | | 8 | | | | | |
| SLC16A12 | | | | | | | | | | | | 15 |
| SLC16A13 | | | 6.5 | | | | | | 6 | | | |
| SLC16A14 | | | | | | | | | 14 | | | |
| SLC16A2 | | | | | 19 | | | | | | | |
| SLC16A4 | | | | | | 16 | | | | 14 | | |
| SLC16A5 | | | | 20 | | | | | | | | |
| SLC16A6 | | | | | 7.5 | | | | | | | |
| SLC16A7 | | | | | | | | | 0 | | | |
| SLC16A9 | | | | | | | | 23 | | | | |
| SLC17A3 | | | | | | | | | | 2 | | |
| SLC17A4 | | 4 | | | | | | | | | | |
| SLC17A5 | | | 23 | | | | | | | | | |
| SLC17A8 | 3 | | | | | | | | | | | |
| SLC17A9 | | | | | | | | | | 21 | | |
| SLC18B1 | | 5 | | | | | | | | | | |
| SLC19A1 | | | | | | | | | 0 | 6 | | |
| SLC19A2 | 6 | | | | | | | | | | | |
| SLC19A3 | 0 | | | | | | | | | | | |
| SLC1A1 | 6 | | | | | | 7 | 13 | | | | |
| SLC1A2 | | 23 | 23.5 | | | 5 | | | 19 | | | 23.5 |
| SLC1A3 | | | | 21.5 | 21 | | | | | | | 22 |
| SLC1A4 | 11.5 | 12.5 | 12 | | 13 | 12 | | 13 | | 11 | 10 | |
| SLC1A5 | 17 | 5.5 | 19 | | | 18 | | 18 | 19.5 | 17 | 19 | 19 |
| SLC20A1 | | | | | | | | | 23 | | 2 | |
| SLC20A2 | | | 8 | | | | | | | | | |
| SLC22A10 | | | | | | | | | | | | 13 |
| SLC22A15 | | | | | | | | | 22 | | | |
| SLC22A17 | | | | | | | 20 | 7 | 3 | | | |
| SLC22A2 | | | | 6 | | | | | | | | |
| SLC22A22 | 21 | | | | | | | 22 | | | | |
| SLC22A23 | | | | | | | 14 | | | | | |
| SLC22A3 | | | | | | | | 19 | | 13.5 | | |
| SLC22A4 | | | 12 | | | | | | 23 | | | |
| SLC22A5 | | | | | | | | | 12 | 14.5 | 8.5 | |
| SLC22A6 | | | | | | | | | 23 | | | |
| SLC22A8 | | 21 | | 18 | | 23.5 | 20.5 | 17 | | 19 | | |
| SLC23A1 | | | | | | | | | 22.5 | | | |
| SLC23A2 | | | | | | | | | | 17 | | |
| SLC24A3 | | | | | | | | | | 13 | | |
| SLC24A4 | | | | | | | | | 12 | 5.5 | | |
| SLC24A6 | | | | | | | | | 4.5 | | | |
| SLC25A1 | | | | | | 1.5 | | | | | | |
| SLC25A10 | | 22 | 0 | | | 1 | | 0 | | 1 | 2 | 2 |
| SLC25A11 | 18.5 | | | | | 18.5 | 18.5 | | | | | |
| SLC25A15 | | | | | | | | | | | 7.167 | |
| SLC25A16 | | | 9 | | | | | 12 | 19.5 | | | |
| SLC25A18 | | | | | | | | 5 | | | | |
| SLC25A19 | | | | | | | | 21 | | | | |
| SLC25A20 | | | | | | | | 13 | | | | |
| SLC25A21 | 7 | | | | | 8 | | 9 | 10 | 6.5 | 7 | |
| SLC25A22 | | | | | | | | | | 11 | | |
| SLC25A25 | | | | | | | | 21 | | | | |
| SLC25A26 | | | | | | | | 6 | 23.5 | 23 | | |
| SLC25A27 | 7 | 7 | | | | | | | | | | 8 |
| SLC25A28 | | | | | | | | | | 18 | | |
| SLC25A30 | | | | | | | | | | 12 | | |
| SLC25A32 | | | | | 8 | | | | | | | |
| SLC25A33 | 10 | | | | | | | | | | | |
| SLC25A34 | | | | | | | | | 23 | | | |
| SLC25A35 | | | | | | | | | | 3 | | |
| SLC25A36 | | | 9 | | | | | | | | | |
| SLC25A37 | | | | | | | | 8 | | | | |
| SLC25A38 | | | | | | | | 21 | 23.5 | | | 23 |
| SLC25A39 | | | | | | | | 11 | | | | |
| SLC25A40 | | | 20 | | | | | | | | | |
| SLC25A42 | 10 | 10 | | | | | | | 7.5 | 11 | 1 | 10 |
| SLC25A44 | 20 | | | | | | | | | | | |
| SLC25A46 | | | | | | | | | | 23 | | |
| SLC25A47 | | | | | | | | | | | 7.833 | |
| SLC26A1 | | | 23.5 | | | | | | 1 | 2 | | 3 |
| SLC26A10 | | | | | | | | | 21.5 | | | |
| SLC26A11 | | | | | | | | | 12.5 | | | |
| SLC26A2 | | | | | | | | | 11 | | | |
| SLC26A4 | | | | | | | | | | 17 | | |
| SLC26A6 | | | | | | | | | | 8.5 | | |
| SLC26A9 | | | | | | | | | 23 | 1 | | |
| SLC27A1 | | | | | | | | | 23 | | | |
| SLC27A2 | | | | | | | | | 22.5 | | | |
| SLC27A6 | | | | | | | | | 22.5 | | | |
| SLC28A1 | | | | | | 21 | | | | | | |
| SLC28A2 | | | | | | | | 7 | | | | |
| SLC29A1 | | | | | | | | 11 | | 12 | | |
| SLC29A3 | | 10 | 11 | | | | | | 8 | | | 12 |
| SLC2A1 | | | | | | | | | | 5 | | |
| SLC2A12 | | | | | | 15 | | | | | | |
| SLC2A13 | | | | | | | | | 21 | | | |
| SLC2A2 | | | | | | | | | 23 | | | |
| SLC2A3 | | | | | | | | | 22 | | | |
| SLC2A5 | 10 | 11 | 11.5 | | 11 | 10 | | 11 | 11 | | 9 | 13 |
| SLC2A8 | 9.5 | 9.5 | 11 | | | | | | 6 | | | |
| SLC2A9 | | | | | | | | | 22 | | | |
| SLC30A1 | | | | | | | | | 23 | | | |
| SLC30A10 | | | | | | | | | 21 | | | |
| SLC30A2 | | | | | | | | 12 | | 12 | | |
| SLC30A6 | | | | | | | | | 23.5 | | | |
| SLC31A1 | 20 | | | | | | | 21 | | | | |
| SLC31A2 | | | | | | | | | 3 | | | |
| SLC33A1 | | | | | | | | 18 | 18.5 | | | |
| SLC34A2 | | | | | | | | | | 11 | | |
| SLC35A1 | | | | | | | | 0 | 0.5 | | | 22 |
| SLC35A5 | | | | | | | | | 23.5 | | | |
| SLC35B1 | | | | | | | | | 23 | | | |
| SLC35B2 | | | | | | 12 | | | | | | |
| SLC35B4 | | 9 | 8 | | | | | | | | | |
| SLC35C1 | | 2.5 | | | | | | | 2 | | | |
| SLC35C2 | | | | | | 0 | | | | 5 | | |
| SLC35D1 | | | | 12 | | | | | 21 | | | |
| SLC35D2 | | | | | | | | | | 19 | | |
| SLC35E1 | | | | | | | | | | | 1.5 | |
| SLC35E2B | | | | | | | | | | 2 | | |
| SLC35F3 | | | 0 | | | | | | | | | |
| SLC35F5 | 21 | 2 | 23 | | | | | | | | | 1.5 |
| SLC35G1 | | | | | | | | | | 0 | | |
| SLC36A1 | 22 | | | | | | | | | | | |
| SLC36A2 | | | | | | | | 22 | | 3 | | |
| SLC37A1 | | | | | | | | 23 | | | | |
| SLC37A2 | | | 3 | | | 3 | | 3 | | | 3 | |
| SLC37A4 | | | 1 | | | 2 | | 2 | | | 2.5 | |
| SLC38A1 | | | 23.5 | | | 4 | | | | 2 | | |
| SLC38A2 | | | | | | | | 14 | 15.5 | | | |
| SLC38A3 | | | | | | 6 | | | | 4 | | |
| SLC38A4 | | | | | | | | | 2 | 4 | | |
| SLC38A6 | 4 | | 1 | | | 3.5 | | 6 | | | 3.5 | |
| SLC38A7 | | | | | | | | | | 1 | | |
| SLC38A9 | | 18 | | | | | 20.5 | | | 15 | | |
| SLC39A10 | | | | | | | | 23 | | | | |
| SLC39A11 | | | | | | | | | 22 | | | |
| SLC39A14 | | | | | | 16.5 | | | 9 | 12 | | |
| SLC39A2 | | | | 22 | | | | | 11 | | | |
| SLC39A4 | | | | | | | | 12 | | | | |
| SLC39A8 | | | | 21.5 | | | 22 | | | | | |
| SLC39A9 | | | | | | | | | | | 5.833 | |
| SLC3A2 | | | | | | | | | | 17 | | |
| SLC40A1 | | 8 | 9.5 | | 15 | 16 | | 17 | 5.5 | 14 | | 13 |
| SLC41A1 | 21.5 | | | | | | | | | | | |
| SLC41A2 | | | | | | | | 21.5 | 20 | | | |
| SLC41A3 | 2 | | | | | | | | | | | |
| SLC43A1 | | | 9 | | | | | | | 10 | | |
| SLC43A2 | | | | | | | | | 0.5 | | | |
| SLC43A3 | | | | | | | | | 7 | | | |
| SLC44A1 | | | | | | | | 23.5 | | | | |
| SLC44A2 | | | | | | | | 22.5 | | | | |
| SLC44A3 | | | | | | | | | 19 | | | |
| SLC44A4 | | | | | | | | 5.5 | 23 | | | |
| SLC44A5 | | | | | | | | 17 | | | | |
| SLC45A1 | | | 11 | | | | | | | | | |
| SLC45A3 | | | | | | | | | 0 | | | |
| SLC46A3 | | | 8 | | | | | | | 8 | | |
| SLC47A1 | | | 8 | | | | | | | | | |
| SLC4A11 | | | | | | | | | 22 | | | |
| SLC4A2 | | | | | | | | | 1 | | | |
| SLC4A4 | | 13 | | | | | | | 21 | | | |
| SLC4A7 | | | | | | | | 12 | | | | |
| SLC4A8 | | | | | | | | | 22 | | | |
| SLC50A1 | | | 7.5 | | | | | | | | | |
| SLC52A2 | | | | | | | | | 4 | | | |
| SLC5A12 | | | | | | | | 2 | | | | |
| SLC5A3 | 0 | 0 | 2 | | | 23 | | 0 | 2 | 0 | 0 | 2 |
| SLC5A6 | | | 9.5 | | | | | | | | | |
| SLC5A8 | | | | 12.5 | | 4 | | | | 2.5 | | |
| SLC6A13 | 0 | | | | 2.5 | | | 21 | 0.5 | 22 | | |
| SLC6A14 | 11 | | | | | | | 15 | 19 | 14.5 | | |
| SLC6A15 | | | | | | | | 17 | | | | |
| SLC6A17 | | | | | | | | 14.5 | | | | |
| SLC6A18 | | | | | | | | 23 | 13 | | | |
| SLC6A19 | | | | | | | | | 20 | | | 21 |
| SLC6A20 | | | | | 5 | | | 10 | 2 | | | |
| SLC6A4 | | | 20 | | | | | | 21 | 5.5 | 23 | 23 |
| SLC6A6 | | | 21 | | | | | | | | | |
| SLC6A8 | | | | | | | | | 22 | | | |
| SLC6A9 | | | | | | | | 22 | | | | |
| SLC7A10 | | | | | | 19 | | | | 15 | | |
| SLC7A11 | | | | | | | | | 21 | | | |
| SLC7A2 | | | | | | | | | | 10 | | |
| SLC7A5 | | | | 22 | | | | | | | | |
| SLC7A6 | 23 | 2 | 2.5 | | | 6 | | 23 | 23 | | | 22 |
| SLC7A8 | | | 10 | | | | | | 19.5 | | | |
| SLC7A9 | | | | | | | | | 22 | | | |
| SLC8A1 | | | | | | | | | 21 | | | |
| SLC9A1 | 22 | 0 | 1 | | 2 | 0 | | 22 | 23 | 23 | 1 | 23.5 |
| SLC9A2 | | | | | | | | 23 | | | | |
| SLC9A3 | | | 12 | | | | | | 9 | | | 10 |
| SLC9A3R1 | | | | | | 18 | | 20 | | 16 | 3.167 | |
| SLC9A3R2 | | | 11.5 | | | | | | | | | |
| SLC9A9 | | | 23.5 | | | 2 | | | | 2 | 1.5 | |
| SLCO1A6 | 8.5 | | | | | | | | | | | 10 |
| SLCO1B3 | | | | | | | | | 21.5 | | | |
| SLCO2A1 | | | | | | | | | | | | 11.5 |
| SLCO2B1 | | | | | | | | 23.5 | | | | 23 |
| SLCO3A1 | 14 | | | | | | | | | 13 | | |
| SLCO5A1 | | | | | | | | 13 | | | | |
| SLFN1 | | | | | | | | | | 14.5 | | |
| SLFN13 | | | 3 | | | | | 21 | 22 | | | |
| SLFN5 | | | | | | | | | | | | 21 |
| SLIT1 | 23 | | | | | | | 20.5 | | 22 | | |
| SLIT2 | | | 20 | | | | | | | | | |
| SLIT3 | | | | | | | | 5 | 7 | | | |
| SLMAP | | | | | | | | 2 | | | | |
| SLMO2 | | | | | | | | | | 6 | | |
| SLN | 2 | 1 | 2 | 23.5 | 0 | 0 | 23.5 | | | | 1 | |
| SLPI | | | | | | | | | 1 | | | |
| SLTM | | | | | | 11 | | | | | | |
| SLU7 | | | | | | | | | | 12 | | |
| SMAD1 | 11 | | | | | | | | | | | |
| SMAD3 | | | | | | | | 0.5 | | | | |
| SMAD6 | 2 | 2 | 2 | | | | 15.5 | 0 | 23 | 10 | | |
| SMAD9 | | | | | | | | | | 13 | | |
| SMAGP | | | | | | | | 21.5 | 22 | | | |
| SMAP1 | 21.5 | | | | | | | | 19 | | | |
| SMAP2 | | | | | | | | 18 | | | | |
| SMARCA2 | 7.5 | | | | | | | 9 | | | | 10.5 |
| SMARCA4 | | | | | | | | | | 17 | | |
| SMARCAD1 | | | | | | | | | | | 4.5 | |
| SMARCAL1 | | | | | 20.5 | | | | | | | |
| SMARCB1 | | | 21 | 23 | | 1 | | | | | | |
| SMARCC1 | | | | | | | | 23 | | 2 | | |
| SMARCC2 | | | | | | | | 22 | 22.5 | | | |
| SMARCD1 | | | | | | 0.5 | | | | | | |
| SMARCD2 | | 13 | | | | | | 3 | 23 | | | |
| SMARCD3 | | | | | | | | | | 11 | | |
| SMC5 | | | | | | | | | 6 | | | |
| SMCHD1 | | 6 | 5 | 10 | | 10 | 10 | 6 | 4 | | | |
| SMCP | | | | | | | | | | 5 | | |
| SMCR7 | | 12.5 | 15 | | | | | 17 | | | | |
| SMCR8 | 12 | | 9 | | | | | | | | | |
| SMG1 | | | | | | 18 | | | | | | |
| SMG5 | | | | | | | | | | 11 | | |
| SMO | | 15 | | | | | | | | | | |
| SMOC1 | | 18 | | | | | | | | | | |
| SMOK2A | | | | | | | | | | | 23 | |
| SMPD1 | | | | | | | | | 18.5 | | | |
| SMPD2 | | 6 | 6.5 | | 4.5 | | | | | | | |
| SMPD3 | 9.5 | | | | | | | | | | | 11 |
| SMPD4 | | | | | | | | 10.5 | | | | |
| SMPDL3A | | | | 21 | | | | | | | | |
| SMPDL3B | | | | | | | | 9 | | | | |
| SMT3H2-PS2 | | | | | | | | | | | 6 | |
| SMTN | | | | | 8 | | | 23 | 21.5 | | | |
| SMTNL2 | | 6.5 | 8 | | | | 7.5 | | | | 8.5 | |
| SMURF2 | | | | | | | | 16 | | | | |
| SMYD1 | | | | | | | 21 | 18 | | | | |
| SMYD2 | | | 14 | | | | | | 12 | | | |
| SMYD5 | | | 17 | 19 | | | | | | 14.5 | | |
| SNCA | 20 | | | | | | | 21 | | | | |
| SNCG | | | | | | | | 23 | | | | |
| SND1 | | | | | | | | | 12 | 9 | | |
| SNF8 | | | 8 | | | | | | | | | |
| SNHG11 | 21 | | | | | 5.5 | | 21 | 22 | | | |
| SNORA21 | | | | | | | 6 | | 21.5 | | | |
| SNORA22 | | | | | | | | 22 | 23 | | | |
| SNORA23 | | | | | 21 | | | | | | | |
| SNORA32 | | | | | | | | | 0 | | | |
| SNORA38 | 5.5 | | | | | | | 20.5 | | | | |
| SNORA42 | | | | | | 21 | | | 13 | 9 | | 9 |
| SNORA54 | | | | | | 14 | | 4.5 | | | | |
| SNORA55 | | 3.5 | 4 | | | | | | | | | |
| SNORA61 | | | | | | | | | | 2 | | |
| SNORA70 | 20 | | | | | | | | | | | |
| SNORA71 | | | | | | | | 12 | 14 | | | |
| SNORA72 | | | | | | | 4.5 | | | | | |
| SNORA73 | | | | | | | | 21 | | | | |
| SNORA74A | | | 6 | | | | | | | | | |
| SNORA7A | | | | | | | | | 21.5 | | | |
| SNORA9 | | | | | | | | | | | | 13 |
| SNORD104 | | | | | | 16 | | | | 13 | | 16 |
| SNORD113 | | | 6.5 | | | | | 23 | | | | |
| SNORD15A | | | | | | | | 23 | 21 | | | |
| SNORD35B | | | | | | | | | 13 | | | |
| SNRK | | | 23 | | | | | | | | | |
| SNRNP200 | | | | | | | | | | 7.5 | | |
| SNRNP27 | | | | | | | | 17 | | | 0 | |
| SNRNP40 | | | | | | | | 11 | | | | |
| SNRNP48 | | | | | | | | | | 3 | | |
| SNRNP70 | | | | | | | | | 12 | | | |
| SNRPB | | | | | | | | | | 7 | | |
| SNRPB2 | | | | | | | | | | 11 | | |
| SNRPD2 | | | | | | 16 | | 17 | 18 | 15 | | |
| SNRPD3 | | | | | | | | | | 15.5 | | |
| SNRPG | | | | | | | | 12.5 | 12 | | | |
| SNTA1 | | | | | | 6 | | | | 9 | | |
| SNTB1 | 6.5 | | | | | | | | | | | |
| SNTB2 | | | | | | | | 1 | | | | |
| SNTG2 | | | | | | 23.5 | | 0.5 | | 1 | | |
| SNUPN | | | | 21 | | | 21 | | 1 | 20 | 1 | |
| SNW1 | 11 | | | | | | | | | | | |
| SNX12 | | | | | | | | | | | | 9 |
| SNX14 | | | 0 | | | | | | | | | |
| SNX16 | | | | | | | | | | | 12 | |
| SNX17 | | | | | | | | | | 12 | | |
| SNX18 | | | | | | | | 21 | 21 | | | |
| SNX21 | 10 | | | | | | | | | 14 | | |
| SNX22 | | | | | | 20 | | | | | | |
| SNX27 | | | | | | | 0 | | | | | |
| SNX29 | | | | | | | | 5.5 | | | | |
| SNX3 | | | | | | 21 | | | 22 | | | |
| SNX30 | | | | | | | 0 | | 0 | | | |
| SNX31 | | | | | | | | 16 | | | | |
| SNX32 | | | | | | | | 23 | | | | |
| SNX33 | | | | | | | | | 23 | | | |
| SNX6 | | | | | | | | 0 | | | | |
| SNX7 | | | | | | 9 | | | | | | 5 |
| SNX8 | | | | | | | | | 2.5 | | | |
| SOAT1 | | | | | | | | | | | 8.5 | |
| SOBP | 17 | | | | | | | | | | | |
| SOCS2 | | | | | | | | | 6 | | | |
| SOCS7 | | | | | | | | 22.5 | | | | |
| SOD1 | | | | | | | | | | 9 | | |
| SOD2 | | 5 | 6 | | | | | | | | | |
| SOGA1 | | | 6.833 | | | | | | | | | |
| SOGA3 | 8 | 8 | | | | | | | | 11 | | 11 |
| SORBS1 | | | | | | | | | 19 | | | |
| SORBS2 | | | | 0 | | | | | | | | |
| SORBS3 | | | | | | 10 | | | | | | |
| SORCS2 | | | | | 21 | | | 19 | | | | |
| SORCS3 | | | | | | | | | | | | 5.167 |
| SORD | | | | | | | | | | | 6.5 | |
| SORL1 | | | | | | | | 17 | | | | |
| SORT1 | | | | | | 13 | | | | | 15 | |
| SOS2 | 8 | 6 | | | | | | | 12 | | | |
| SOSTDC1 | | | | | 22.5 | | | | 0.5 | 19 | | |
| SOWAHB | | | | | | | | 0 | | | | |
| SOX12 | | | | | | | | | | | | 0 |
| SOX17 | | | | | | | | 0 | | 14 | | |
| SOX18 | | | | | | | | 16.5 | | | | 22 |
| SOX4 | | | | | 4.5 | | | | | 5 | 2.5 | |
| SOX6 | | | | | | | | 12 | 5.5 | | 22.5 | |
| SOX7 | | | | | | | | 8 | | | | |
| SOX9 | | | | | | | | | | | | 22 |
| SP1 | | | | | | | | | | 5.5 | | |
| SP100 | | | | | | | | | | 18 | | |
| SP23 | 23.5 | 22 | 22 | 19 | | 22 | | 23 | 22 | 22 | | 23 |
| SP25 | | 17 | | | | | | | | 14 | | |
| SP3 | | | | | | | | | 22 | | | |
| SP4 | | | | | | | | 2 | | 7 | | |
| SP47 | | 21.5 | 22 | | 1 | | | 21.5 | 23 | | | |
| SP91 | | 7.5 | 8 | | | | | | | | | |
| SPA17 | | | | | | | | | 9 | | | |
| SPAG1 | | | 8.5 | | | | | | | | | |
| SPAG6 | | | 13 | | | | | 3 | 8 | | | |
| SPAM1 | | 8 | | | | | | 9 | | | | |
| SPARC | | | 23 | | | | | | 2 | | | |
| SPATA13 | | 7 | 8 | | | 8 | | | | | | |
| SPATA17 | | | | | | | | 19 | | | | |
| SPATA22 | | | | | | | | | 22 | | | |
| SPATA24 | | | 6 | | | | | | | | | |
| SPATA5 | | | | | | | | 6.5 | | | | |
| SPATS2 | | | | | | 12 | | 11.5 | 14 | 13 | | |
| SPC24 | | | | | | 2.5 | | 23 | 23.5 | | | |
| SPC3 | | | 22.5 | 21 | | | | | | | | 2 |
| SPCS2 | | | | | | | | | | 12 | | |
| SPCS3 | | | | | | | | | 20 | | | |
| SPECC1 | | | | 11 | | | | | | | | |
| SPEF2 | | | | | 20 | | | | | | | |
| SPEN | | | | | | | | | | 4 | | |
| SPG20 | | | | | 6 | | | | | | | |
| SPG21 | | 0 | | | | | | | 9 | | | |
| SPHK1 | | | | | | | 6 | | | | | |
| SPHK2 | | | | 23 | | | | | | | | |
| SPIB | | | | | | 9 | | | | | | |
| SPIC | 15 | | | | | 14.5 | 16 | | | | | |
| SPICE1 | | | | | | 15 | | | | 14.5 | | |
| SPINK5 | 23 | | | | | | | 1 | | 1.5 | | |
| SPINT1 | | | | | | | | | 22 | | | |
| SPIRE1 | | | | | | | | | 22 | | | |
| SPN | | | | | | | | 4 | | | | |
| SPNS2 | | | | | | | | 21 | | | | |
| SPOCK2 | | | | | | | | 0 | | | | |
| SPON1 | | | | | | | | 15 | 15 | 10 | | |
| SPON2 | | | | | | | | 8 | 23 | | | |
| SPOP | | | | 10 | 8.5 | 13 | | 13 | | 12 | | |
| SPP1 | | | | | | | | 21 | | | | |
| SPPL2A | | | | | | | 9 | | 20 | | | |
| SPPL2B | | | | 20 | | | 17 | | | | | |
| SPPL3 | | | 3 | | | | | | | | | |
| SPRED1 | | | | | 1 | | | | | | | |
| SPRED2 | | | | | | | | 15 | | | | |
| SPRR1A | | | | | | | | 21 | | | | |
| SPRR2A3 | | 8 | | | | | | | | 8 | | 8.5 |
| SPRY1 | 21 | | 6 | | | 3 | | 21 | | | | |
| SPRY2 | | | | | | | | 3 | | | | |
| SPRYD3 | | | | | | 7 | | 1 | | | 14 | |
| SPRYD4 | | | | | | | | 9.5 | | | | |
| SPRYD7 | | | | | | | 22.5 | | | 5.5 | | |
| SPSB1 | | | | | | | | 11 | 21.5 | | | |
| SPSB3 | 6 | | | | | | | | | | | |
| SPSB4 | | | | | | 0 | 1 | 23.5 | | | 3 | 0 |
| SPTA1 | | | | | | | | | | | 22 | |
| SPTAN1 | | | | | | | | | | | 20.5 | |
| SPTB | 7 | 9 | | | | | | 8 | | 7 | | |
| SPTLC1 | | | | | | | | | 23 | | | |
| SPTLC2 | | | | | | | | 5.5 | 3 | | | |
| SPTSSB | | | | | | | | | | | 21 | |
| SQLE | | | | | | | | | | 4 | | |
| SQSTM1 | | | 10 | | | | | | | | | |
| SRBD1 | | | | 9 | | | | | | | | |
| SRD5A1 | | | | | | 13 | | | 0 | | | |
| SRD5A2 | | | | | | | | | 23.5 | | | |
| SREBF1 | | | | | | | | | | 4 | | |
| SREBF2 | | | | | | | | 6 | | | | |
| SREK1 | | | | | | | | | | 2 | | 23.5 |
| SREK1IP1 | | | | | | | | | | 2 | | |
| SRF | | | | | | | | | 23 | | | |
| SRGAP1 | | | | | 21 | | | | 19 | | 0 | |
| SRGAP2 | | | | | | | | 7 | | | 6.5 | |
| SRGAP3 | | | 23 | | | | | | | 1 | | |
| SRGN | | | | | | | | 22 | | 7.5 | | |
| SRM | | | | | | | | | | | | 1.5 |
| SRMS | | | | | | | | | | 5.5 | | |
| SRP72 | 11 | | 9 | | | | | 12 | | | | |
| SRPK1 | | | | | | | | | | 3.5 | | |
| SRPK2 | | | | | | | | 1 | | | | |
| SRR | | | | | | | | 9 | | | | |
| SRRD | | | | | | | | | | 5 | | |
| SRRM1 | | | | | | | | | | 5.5 | | |
| SRRM2 | | | | | | | | | | | 23 | |
| SRRT | | | | | | | | | | | 5.5 | |
| SRSF1 | | 4 | | | | | | | | | | |
| SRSF10 | 16 | | 15 | | | 16.5 | | 17 | | 15 | | |
| SRSF2 | | | | | | | | 8.5 | | | | |
| SRSF3 | | | | | | | 4 | | | | | |
| SRSF6 | | | | | | | | 10 | | | | |
| SRXN1 | | | | | | | | | 2 | | | |
| SS18 | | 14 | | | | | | | | 12.5 | | |
| SS18L1 | | | | | 11.5 | | | | 23 | 13.5 | | |
| SS18L2 | | 17 | 18 | | | | | | | | | |
| SSBP2 | 18 | | | | | | 0 | 19 | | | | |
| SSBP3 | | | | | | | 20 | 20.5 | 17 | | | |
| SSBP4 | | | | | | | | 21 | 20.5 | | | |
| SSFA2 | | | | | | | | | 18 | | | |
| SSPN | | 9 | 10 | | | | | | | | | |
| SSR1 | | | | | | 13 | | 13 | 11.5 | | | |
| SSR4 | | | | | | 11.5 | 1 | 21 | | | 2 | |
| SSX2IP | | | | | | 13 | | | | | | |
| ST13 | | | | | | | | | | 5 | | |
| ST3GAL1 | | | | | | | | | 5.5 | | | |
| ST3GAL3 | | | | | | | | | 13 | | | |
| ST3GAL4 | | | | | | | | | | | 9 | |
| ST3GAL5 | | | | | 21 | | | | | | | |
| ST3GAL6 | | | | | | 21 | | | | 23 | | |
| ST5 | | | | | | | | 1 | | | | |
| ST6GAL1 | 22 | | 23 | | | | | | 22.5 | | | 23 |
| ST6GALC2 | | | | | | | | | | | | 22 |
| ST6GALC3 | | | | | | | | | 19.5 | | | |
| ST6GALC5 | | | | | | | | | 10 | | | |
| ST6GALC6 | | | | | | | | | | 11.5 | | |
| ST7 | | | | | | | | 12 | | 7.5 | | |
| ST7L | | | | | | | | | 23 | | | 23.5 |
| ST8SIA1 | | | | | | | | | 23.5 | | | |
| STAB1 | | | | | | | | | | | 6.167 | |
| STAG1 | | | | | | | 12 | | | | | |
| STAM | 10 | 9.5 | 11 | | | 10 | | | | | 9 | |
| STAM2 | | | | 22 | | | | | | | | |
| STAMBP | | | | | | | | 6 | | | | |
| STAP1 | | | | | | | | | | 15 | | |
| STAR | | | | | | | | 0 | | | | |
| STARD13 | | | | | | | | 11 | | 5 | | |
| STARD3NL | 14 | | 13 | 20.5 | | | 17 | | | 13 | | |
| STARD4 | | | 6 | | | | | | | | | |
| STARD5 | | | | | | | | 17 | | | | |
| STAT2 | | | | | | | | | | | | 9 |
| STAT3 | | | | | | | | | | | 12 | |
| STAT4 | | | | | | | | 22 | 21 | | | |
| STAT5A | | | | | | | 7 | | | | | |
| STAT5B | | 23 | | | | | | | | | 22 | 22 |
| STBD1 | | | | | | | | | 23 | | | 1.5 |
| STC1 | | | | | | | | 9 | | | | |
| STC2 | | | | | | | 4.5 | | | 2 | | |
| STEAP2 | | | | | | | | | | | | 11 |
| STEAP3 | | | | | | | | | 16 | | 12 | |
| STEAP4 | | | | | | | | | 6 | | 5 | |
| STIM2 | | | | | | | | 23.5 | | | | |
| STIP1 | | | | | | | | | | | 8 | |
| STK10 | 20.5 | | | | | | | | | | | |
| STK11 | | | | | | 10.5 | | | | | | |
| STK16 | | | | | | | | | 1 | | | |
| STK17B | | | | | | | | | | 7 | | |
| STK24 | | | | | | | | | 21 | | | |
| STK25 | 20 | | | | | | | | | | | |
| STK32A | | | | | | | | | 1 | | | |
| STK32B | | | 12 | | | | 3.5 | | 23 | | | |
| STK32C | | | | | | | | | | 6.5 | | |
| STK35 | | | | | | 10 | | 22 | | | | |
| STK36 | | | | | | | | | 23 | | | |
| STK38 | | | | | | | | | | 7 | | |
| STK39 | | | | | | | | | | | 21 | |
| STK4 | | | | | | | | | | 7 | | |
| STK40 | | | | | | | | | 19 | | | |
| STMN2 | | | | | | | | | | 22 | | |
| STOM | | | | | 7 | | | | | 7.5 | | |
| STON2 | | | | | | | | 12 | | | | |
| STOX2 | | | | | | | | | 1 | | | |
| STRA13 | | | | | 21.5 | | | 10 | 22.5 | | | |
| STRAP | | | | | | 13 | | 14 | | 11.5 | 11 | 9 |
| STRBP | | | | 17 | | 18.5 | 16.5 | | | | | |
| STRN | | | | | | | | 4 | | | | |
| STRN4 | | | | | | | | | 20 | | | |
| STT3A | | | | | | | | | 17 | | | |
| STT3B | | | | | | | | 6.5 | | | | 9 |
| STX11 | | | | | | | 23 | 17 | 21 | 17 | | |
| STX16 | | | | | | | | | | | 23 | |
| STX17 | | | | | | | | 23 | | | | |
| STX18 | | | | | | | 23 | 3.5 | 2.5 | | | |
| STX1B | | | | | | | | | | 11 | | |
| STX2 | | | | | | | | | 5.5 | | | |
| STX3 | | | | | | | | 6 | | | | |
| STX4 | | | | | | | | | 6.5 | | | |
| STX5 | | | | | | | | | | | 23 | |
| STX7 | | | | | | 20 | | | | | | |
| STXBP1 | | | 22 | | | | | | | | | |
| STXBP2 | | | | | | | | 16.5 | | | 0 | |
| STXBP4 | | | | | | | | | | | 4.833 | |
| STXBP5L | | | | | | | | | 20.5 | | 9.5 | |
| STXBP6 | | | | | | | | | | 7 | | |
| SUCNR1 | | | | | | | | | | 0 | | |
| SUDS3 | | | 22.5 | 3.5 | | | | | | | | |
| SUFU | | | | | | | | | | | 0 | 1 |
| SUGP1 | 5 | | | | | | | | 5 | | | 9 |
| SULF1 | | | | | | | | | 22 | | | |
| SULF2 | | | | | | | | 0 | | | | |
| SULT1A3 | | | | | | 10 | | | | | | |
| SULT1C2 | 23 | 1 | | | | | | | | | | 21 |
| SUMF2 | | | | | | | | | 3 | | | |
| SUMO3 | | | | | | | | | | 5 | | |
| SUN2 | | 7 | 9 | 11.5 | | | | | | | | 12 |
| SUOX | | 9 | 9 | | | | | | | | | 11 |
| SUPT16H | | | | | | | | | 9 | | | |
| SUPT4H1 | | | | | | | | | 22 | | | |
| SUPT7L | | | 10 | | | | | | | | | |
| SUSD1 | 21 | 4 | | | 3 | | | 6 | 4 | | | |
| SUSD3 | 21 | | | 22 | | | 22.5 | 21 | 23 | 18 | | |
| SUSD4 | | | | | | | | | | 14 | | |
| SUV420H1 | | | 20 | | | | | | | | | |
| SVEP1 | | | 1 | | | | | | 23 | 16 | | |
| SVIL | | | | | | | | 22 | | | 23 | |
| SVS6 | | | | | | | | 23 | | 21 | | |
| SWSAP1 | | | | | | | | | | 3 | | |
| SYAP1 | | | | | | | | 21 | | | | |
| SYDE2 | | | 7 | | | | | | | | | |
| SYK | | | | | | 10 | | | | | | |
| SYN2 | | | | | | | | | 23 | | | |
| SYNC | | | | | | | | | | 14 | | |
| SYNE2 | | | | | | | | | 3 | | | |
| SYNJ2 | | | | | | | | | 21.5 | | | |
| SYNM | | | | | 10 | | | | | 8.5 | | |
| SYNRG | | | | | | 18 | | | 13 | | | |
| SYPL1 | | | | | 9 | | | | | | | |
| SYPL2 | | | | | | | | 0 | | | | |
| SYS1 | | 3.5 | 23 | | | | | | | | | |
| SYT1 | | | | | | | | | 2 | | | |
| SYT11 | | | 3.5 | | | | | 23 | | | | |
| SYT12 | | 22 | | 22 | 21 | 23 | 22 | 22.5 | 23 | 22 | 0 | |
| SYT14 | | | | | | | | | 17 | | | |
| SYT15 | | | | | | | | | | | 11 | |
| SYT17 | | | | | | | | | | 7 | | |
| SYT2 | | | 23.5 | | | | | 21 | 22 | | | |
| SYT3 | | | 1 | | | | | 23 | 1 | | 2 | |
| SYTL2 | | | | | | | | 23 | | | | |
| SYTL5 | | | | | | 12 | | 7 | 5 | | | |
| SYVN1 | | | | | 5.5 | | | | | | | |
| SZT2 | | | | | | | | 20 | | | | |
| T10 | | | | 7.5 | | | | | | | | |
| T6 | | | | | | | | | 15 | | | |
| T8 | | | | | | | 5.5 | | | | | |
| T8L | 18 | 21 | 21 | 20 | | | | 18.5 | 5.5 | | | 20 |
| TAAR7F | 22 | | | | | | | | | | | |
| TAB2 | | | | 6 | | | | | | | | 2.833 |
| TAB3 | | | | | 12 | | | | | | | |
| TACC1 | | | | | | | | 20 | | | | |
| TACC2 | | | | | | 17 | | 9 | | | | |
| TACO1 | 20 | 22 | | | | 8 | | | | | | |
| TACR1 | | | | | | | | | | 15 | | |
| TADA2B | 22 | | | | | | | | | | | |
| TADA3 | | | | | 6 | | | | | 1.5 | | |
| TAF1 | 13 | | | | | | | | | 4.5 | | |
| TAF11 | | | | 15 | 13 | | | | 5 | 9 | | 21 |
| TAF15 | | | 8 | | | | | | 6.5 | | | |
| TAF1B | | | 4.5 | | | | | 22 | | | | |
| TAF2 | | | | | | | | 21 | | | | |
| TAF4 | | | | | | | | | 11 | | | |
| TAF4B | | | | | | | | | | | 7 | |
| TAF6 | | | | | | | | | 1 | | | |
| TAF6L | | | | | | 18 | | | | | | |
| TAF9B | | | | | | | | 18.5 | | | | |
| TAGAP | | | | | | | | 21 | | | | |
| TAGLN | | | | | | 17 | | 11 | 23 | | | |
| TAGLN2 | | | | | | | 20 | | | | | |
| TAMM41 | | | | | | | | 1.5 | | | | |
| TANC2 | | 13.5 | 12.5 | | | | | | | | | |
| TANK | | | | | | | | | 4 | | | |
| TAOK2 | | | | | | 19 | | | | | | |
| TAOK3 | | 15 | 16 | | | 18 | | 15.5 | | 14 | | 15 |
| TAP1 | | | | | | | | | 12.5 | | | |
| TAPBPL | | | | | | | | | 0 | | | |
| TAPT1 | | | | | | | 17 | | | | | |
| TARBP1 | | | | | | | 3 | | | | | |
| TARDBP | 20 | | 8 | | | | | | | | 22 | |
| TARS | | | | | | | | | 21 | | | |
| TARS2 | | | | 10.5 | | | 9 | | 22 | | | |
| TAS2R14 | | | | | | | | | 22 | | | |
| TAS2R143 | | | | | | | | | 5.5 | 15.5 | | |
| TAS2R4 | 20 | | | | | | | 20.5 | | | | |
| TASP1 | | | | | | | | | | 5.5 | | |
| TATDN1 | | | | | | | | | | 5 | | |
| TATDN2 | | | 4 | | 10 | | | | | | | |
| TATDN3 | 7.5 | | | | | | | | | | | |
| TBC1D1 | | 7 | 9 | | | | | 5.5 | 5 | | | |
| TBC1D10A | | | | | | | | 19 | 17 | | | |
| TBC1D10C | | | 5.5 | | | | | | | | | |
| TBC1D13 | | | | | | | | | 23.5 | | | |
| TBC1D14 | | | | | 12 | | | | | | | |
| TBC1D15 | | | 22 | | | 0 | | | | | | |
| TBC1D16 | | | 9 | | | | | | | | | |
| TBC1D17 | | | | | | | | 22 | | | | |
| TBC1D20 | | 8.5 | | | | | | | | 9 | | |
| TBC1D22A | | | 20 | | | 17 | | | | 17 | | |
| TBC1D22B | | | | | | | | 3.5 | | | | |
| TBC1D24 | | | 22 | | | 3 | | | | | | |
| TBC1D25 | | | | | | | | | 20.5 | | | |
| TBC1D2B | | | | | | | | 11 | | | | |
| TBC1D4 | | | 8 | | 4 | | | | | | | |
| TBC1D5 | | | | | | 18.5 | | 14 | | | | |
| TBC1D7 | | | | | | | | | 1.5 | | | |
| TBC1D8 | | | | | | | | | 1 | | | |
| TBC1D8B | | | | | | | | | 16 | | | |
| TBC1D9 | | 7.5 | 8.5 | | | | | | | | | |
| TBCD | | | | | | | | | | | 3 | |
| TBCE | | | | 21 | | | | 22.5 | | | | |
| TBCEL | 19 | | | | | | | | | | | |
| TBCK | | | | | | | | | 1 | 22 | | |
| TBL1X | | | | | | | | 11 | 22 | | | |
| TBL2 | | | | 21 | | | | | 12 | | | |
| TBL3 | | | | | | | | | | 22 | | |
| TBRG4 | | | | | | | | | 21 | | | |
| TBX10 | | | | | | | | 22 | 21.5 | | | |
| TBX5 | | | | | | | 15 | | 21 | | | |
| TBXAS1 | | | | | 21 | | | | | 17 | | |
| TCAP | | | | | | | | 6 | 2 | | | |
| TCEAL1 | | | | | | | | 22 | | | | |
| TCEAL8 | | | | | | | | 5.5 | | | | |
| TCEB1 | | | | | | | | 10 | 5 | | | |
| TCEB2 | | | | | | | | | 21 | | | |
| TCF19 | 19 | | | | | | | | 21.5 | | | |
| TCF20 | | | | | | | | | | 22.5 | | |
| TCF25 | | | | | | | | | 21 | | | |
| TCF4 | | | | | | | | 1 | | | | |
| TCF7L2 | | | 18.5 | | | | | | | | | |
| TCHHL1 | | | | | | | 11 | | | | | |
| TCIRG1 | | | | | | | | | 17 | | | |
| TCN2 | | | | 1 | | | | | | | | |
| TCOF1 | | | | | | | | 9 | | | | |
| TCP1 | | | | | | | | | 14 | | | |
| TCP11L2 | | | | | | | | 22 | | | | |
| TCTA | | | | | | | | | | 11 | | |
| TCTEX1D2 | | | | | | | | | 1 | | | |
| TCTEX1D4 | | | 21.5 | | | | | | | | | |
| TCTN2 | | | | | | | | | 4 | | | |
| TDO2 | | 1 | | | | | | | 0 | | | |
| TDRD3 | | | | | | | | 5 | 23 | | | |
| TDRD5 | | | | | | | | | | | 23 | |
| TDRD6 | | | | | | | | 1 | | | | |
| TDRKH | | | | 8.5 | | | | | | | | |
| TEAD1 | | | 16 | | | 16 | | | 1 | | | |
| TEAD4 | | | | | | | | | | 12 | | |
| TECPR1 | | | | 19 | | | 5.5 | | | 17 | | |
| TECPR2 | | | 18 | | | | | 21 | | | | |
| TECR | | | | | | 10 | | | | | | 11.5 |
| TEF | | | 6 | | | | | | | | | |
| TEK | | | | | | | | | | 12 | | 12 |
| TEKT5 | | | 21 | | | | | | | | | |
| TENC1 | | | | | 19 | | | | | | | |
| TERF1 | | | | | | | | | 23 | | | |
| TERF2IP | | | | | | | 5 | | 3 | | | |
| TES | | | | | | | | | 5.5 | 14 | | |
| TESK2 | | | | | | | | | 22 | | | |
| TET2 | | | | | | | | 23 | | | | |
| TET3 | | | | | | | | | | 19 | | |
| TEX11 | | | | | | | | | | | 2 | |
| TEX12 | | | | 22 | | | | 21 | | | | |
| TEX2 | | 21 | | | | | | | | | | |
| TEX261 | | | | | | | | | 0 | | | |
| TEX264 | | | 7 | | | | | | | | | |
| TEX9 | | | 10.5 | | | 19 | | | | | | |
| TFAP2B | | | | | | | | 21 | | | | |
| TFB1M | | | | | | | | | 22 | | | |
| TFCP2 | | | | | | 20 | | | | | | 19 |
| TFCP2L1 | | | | | | | | 10 | | | | |
| TFDP2 | | | | | | | | 0 | | 5.5 | | |
| TFE3 | | 13.5 | | | | | | | | 10 | | |
| TFEB | | | | | | | | 17 | | | | |
| TFEC | | | | | | | | 12 | | 7 | | |
| TFPI | | | | 6 | | | | | | | | |
| TFPI2 | 10 | 11 | 11 | 10 | 12 | 11 | 10 | 10 | 11 | | 9 | 10.5 |
| TFPT | | | | | | | | | 23.5 | | | |
| TFR2 | | | | | | 7 | | | | | | |
| TFRC | | | | | | | | | 16.5 | | | |
| TG | 21 | | | | | | | | | | | |
| TGFA | | | | | | | | | 23 | | | |
| TGFB3 | | | | | | | 4 | | | 15 | | |
| TGFBI | 1 | 22 | | | | 3 | 7 | | | | | |
| TGFBR1 | | | | | | | | | | 5 | | |
| TGFBR2 | | | | | | | | | 23 | | | |
| TGFBR3 | | | | | | | | | 21 | | | |
| TGIF1 | | | | | | | | | 23 | | | |
| TGM1 | | | | | | | | | | | 8 | |
| TGM2 | | | | | 21 | | | | | | | |
| TGOLN2 | | | | | | | | | | 13 | | |
| THADA | | | 23 | | | | | | | | | |
| THAP1 | | | | 1 | | | 0 | | | | | |
| THAP2 | | | | | | | | 20.5 | | | | |
| THBD | | | | | | | | 22 | | | | |
| THBS1 | 20 | | | | | | | | | | | 22 |
| THBS2 | | | | | | | | 0 | | | | |
| THEM4 | | | | | | | | 23 | | | | |
| THEM5 | | | | | | | | | 22 | | | |
| THOC3 | | | | | | | | | 22 | | | |
| THOC5 | | | | | | | | 10 | | | | |
| THOC6 | | | | | | | | | | | 2.5 | |
| THPO | | | | | | | | | 23 | | | |
| THRA | | | | | | | | | 0 | | | |
| THRAP3 | | | | | | | | | 6 | | | |
| THRB | | | 22 | | | | | | | | | |
| THRSP | 8 | | | | | | | 9 | 8.5 | | | |
| THSD4 | | | | | | 14 | | | | | | |
| THTPA | | 21 | | | | | | | 22 | 5.5 | | |
| THYN1 | | | | | 16.5 | | | | 6 | | | |
| TIA1 | | | | | | | | | 21 | | | |
| TIAM1 | | | | | | | | 7.5 | 8 | | | |
| TIE1 | | | | | | | | | 21.5 | | | |
| TIFA | | | | | | 14.5 | | | | | | |
| TIG | | | | | | | | | | 17 | | |
| TIGL1 | | 8 | | | | 13 | | | | 7.5 | | 9 |
| TIMD2 | | | | | | | | 22 | | | | |
| TIMD4 | | | | | 8 | | | | | | | |
| TIMM10 | | | | | | | | | | 12.5 | | |
| TIMM8B | | | | | | | | | | 11 | | |
| TIMM9 | 20 | | | | | | | | | | | |
| TIMMDC1 | 21 | | | | | | | | 13 | | | |
| TIMP2 | | | | | | | | 5 | | | | |
| TIMP3 | | | | | | | | | 3 | | | |
| TIMP4 | | 8 | | | | 12 | | | 7 | | 9 | |
| TINF2 | | | 6 | | | 1 | | | | | 1.5 | |
| TIPARP | | | | | | | | 5 | 0 | | | |
| TIPIN | | | 8.5 | | | 21 | | | | | | |
| TIRAP | | | | | | | | | | 18 | | |
| TJP2 | | | | | | | | | 22 | | | |
| TJP3 | | | 18.5 | | | | | | | 10.5 | | 15.5 |
| TK1 | | | | 23 | | 5 | | 3 | 2 | | | |
| TK2 | | | | | | | | | 22.5 | | | |
| TLCD1 | | | | | 9 | | | | 21 | | | |
| TLCD2 | | | | | | | | | | 17 | | |
| TLE1 | | | | | | | | | 19 | | | |
| TLE3 | 3 | | | | | | | | | | | |
| TLE4 | 6 | 7 | 7 | | | | | | 10 | | | 8 |
| TLK1 | | | | | | | | | | 10 | | |
| TLL1 | | | | | | | | | | 2.5 | | |
| TLN1 | | | | | | | | 21 | | | | |
| TLN2 | | | | | | | | 1 | | | | |
| TLR2 | | | | 21 | | | 22 | | | | | |
| TLR3 | | | | | | 9 | | | 21 | | | |
| TLR6 | | | | | | | | | 23.5 | | | |
| TM2D2 | | | | | 22.5 | | | | 21 | | | |
| TM4SF1 | | | | 17 | | | | | | | | |
| TM4SF4 | | | | | | | | | | 23 | | |
| TM6SF2 | | | | | | | 0.5 | | 22.5 | | | |
| TM9SF1 | | | | | | | | | | 9.5 | | |
| TM9SF3 | | | | | | 18 | | | 14 | | | |
| TMC1 | | | | | | | | 16 | | 16 | | |
| TMC6 | | | | | | | | 7 | | | | |
| TMC7 | | | 7 | | | | | | 22 | | | |
| TMCC2 | | | | | | | | 21 | 22.5 | | | |
| TMCO1 | | | | | | | | | | 13 | | |
| TMCO3 | | | | | | | | | 21.5 | | | |
| TMCO4 | | | | | | | | 23 | | 5.5 | | |
| TMCO6 | | | 2 | | | | | 21 | | | | 1.5 |
| TMED5 | | | | | | | | | 22 | | | |
| TMED6 | | | | | | | | | | 23 | | |
| TMED8 | | | | 21 | | 6 | | | | | | |
| TMEFF1 | | | 18 | | | | | | | | | |
| TMEFF2 | | 6 | | | | | | | | | | |
| TMEM100 | | | | | | | | | 21 | 18.5 | | |
| TMEM102 | | | | | | | | | 23 | | | |
| TMEM106A | | | | | | | | 1.5 | | | | |
| TMEM106B | | | | | | | | | 21 | 12 | | |
| TMEM107 | | | | | | | | | 2.5 | | | |
| TMEM108 | | 18.5 | | | | | | | | | | |
| TMEM109 | | | | | | | | | 22.5 | | | |
| TMEM11 | | 7 | | | | | | | | | | |
| TMEM111 | | | | | | | | | | | 8 | |
| TMEM115 | | | | | | | | 12 | | | | |
| TMEM117 | | | | | | | | | 11 | | | |
| TMEM120A | 5.5 | | | | | | | | | | | |
| TMEM120B | 19 | | | | | | | | | | | |
| TMEM123 | | | | | | 10.5 | | | | | | |
| TMEM127 | | | | | | | | | 21 | | | |
| TMEM129 | | | | | | | | 21 | | | | |
| TMEM131 | | | | | | | | | | 3 | | |
| TMEM132B | | | | | | | | | 2 | | | |
| TMEM132D | | | | | | | | 20.5 | | | | |
| TMEM135 | | | | | | | | 14 | | 12 | | |
| TMEM140 | | | 12 | | 9 | | 7 | | 7 | 5 | | |
| TMEM141 | | | 15 | | | | | | 17 | | | |
| TMEM144 | | | | | | | | 22 | | | | |
| TMEM145 | | | | | | | | | 20 | | | |
| TMEM147 | | | | | | | | 0.5 | 23 | | 0 | 22 |
| TMEM14A | | | | | | 11.5 | | 2 | | | | |
| TMEM14C | | | | | 10 | | | | | | | |
| TMEM150A | | | | | | | | | 22.5 | | | |
| TMEM159 | | | 6 | | | | | | 0.167 | | | |
| TMEM160 | | | | | | | 9 | | | | | |
| TMEM164 | | | | | | | | | | 15 | | |
| TMEM167A | | | | | 4 | | | | | | | |
| TMEM170A | | | | | | | | | 21 | 10 | | |
| TMEM170B | | | | | | | | 1 | | | | |
| TMEM171 | 5 | | | | | | | | 3 | | | |
| TMEM173 | | | | | | | | | 23 | | | |
| TMEM174 | | | | | | | | 23 | | | | |
| TMEM175 | | | | | 9.5 | | | | | | | |
| TMEM176A | | | | | 14 | | | | | 13.5 | | |
| TMEM176B | | | | | | | 21 | 22 | | | | |
| TMEM177 | | | | | | | | | 22.5 | | | |
| TMEM178 | | | | | | | | 4 | 7 | | | |
| TMEM179B | | | | | | | | | | | 23 | |
| TMEM180 | | 8 | 11 | | | | | | 10 | | | |
| TMEM182 | | | | | | | | | 22 | | | |
| TMEM184A | | | | | | | | | 2.5 | | | |
| TMEM184B | | 9 | 8.5 | | | | | | | 8.5 | | |
| TMEM184C | | 12 | | | | | | | | 12 | | |
| TMEM185A | | | | | | | 10 | | | | 13 | |
| TMEM19 | | | 8 | | | | | | | | | |
| TMEM192 | | | | | | | | 9.5 | | | | |
| TMEM194A | | | | | | | | | 3 | | | |
| TMEM196 | | | | | | | | 22 | | 17 | | 21 |
| TMEM198B | | | | | | | | | | 15.5 | | |
| TMEM200B | | | | | | | | 3.5 | | | | |
| TMEM204 | | | | | | | | 8 | | | | |
| TMEM205 | | | | | | | | 4.5 | 2 | | | |
| TMEM207 | | | | | | | | 12 | 13 | | | |
| TMEM209 | | | | | | | | 22.5 | | | | |
| TMEM212 | | 19.5 | | | | | | | | | | |
| TMEM214 | | | | | 21 | | | | | | | |
| TMEM216 | | | | | | | | 23 | | | | |
| TMEM218 | | | 13 | | | 17 | | | | 4 | | |
| TMEM220 | | | | | | | | | | | 4 | |
| TMEM229B | 13 | | | | | 14 | | | | | | |
| TMEM234 | | 15 | | | | | | | | | | |
| TMEM236 | | | | | | | | | | | 0 | |
| TMEM237 | | | | | | | | 21 | | 16.5 | | |
| TMEM242 | | | | | | | | | 21 | 8 | | |
| TMEM245 | | | | | | | | | | | | 12 |
| TMEM25 | | | | | | | | 12 | | | | |
| TMEM26 | | | | | | | | | 22.5 | | | |
| TMEM27 | | | | | | | | | 23 | | | |
| TMEM33 | | | | | | | | | | | 23.5 | |
| TMEM35 | | | | | | | | | | 22 | | |
| TMEM37 | | | | 23 | | | | | | | | |
| TMEM38B | | | | | | | | 23 | | 12 | | |
| TMEM39A | | | | | | | | | 22 | | | |
| TMEM39B | 9 | 9 | 9.5 | | 11 | 10 | 11 | 11 | 9 | 10 | 8 | 9 |
| TMEM41B | | | | | 11 | 8.5 | | 14.5 | | 11 | 7 | |
| TMEM42 | | | | | | | | | 9 | | | |
| TMEM45A | | | | | | | | | | | 9 | |
| TMEM47 | | | 9.5 | | | | | | 9 | | | |
| TMEM50A | | | | 8.5 | | | | | | | | |
| TMEM50B | | | | | | | | | 0.5 | | | |
| TMEM55A | | 7 | 8 | | | | | | | | | |
| TMEM55B | | | | | | | | 22.5 | 21 | | | |
| TMEM56 | | | | 22 | | | | | | | | |
| TMEM57 | 19.5 | | | | | | | 0 | | | | |
| TMEM62 | | | | | | | 6 | | | | | |
| TMEM63B | | | | | | | | 16 | | | | |
| TMEM63C | | | | | | | | | 14 | | | |
| TMEM64 | | | | | | | | | | 9 | | |
| TMEM67 | | | | | 9.5 | | | | 8 | | | |
| TMEM68 | | | | | | | 0.5 | | 22.5 | | | |
| TMEM71 | | | | | | | | | 21 | | | |
| TMEM80 | | | | | | | | | | 13 | | |
| TMEM85 | | | | | | | | | 0 | | | |
| TMEM86A | | | 14 | | | | | | | | | |
| TMEM86B | | | | | | | | 0 | 23 | | | |
| TMEM87A | | | | | | | | 15.5 | | | 13 | |
| TMEM8A | | | | | | 1 | | 6 | | | 4.5 | 1.5 |
| TMEM9 | | | | | | | | | | | | 17 |
| TMEM98 | | | | | | 13 | | | | | | |
| TMEM9B | | | | | | | | | | | 23 | |
| TMIE | | | | | | | 8 | 9.5 | | | | |
| TMOD1 | | | | 21 | | | | | | | | |
| TMPO | | 7 | 9.5 | | | | | | | 9 | | |
| TMPRSS11A | | | 22 | | | | | | | | | |
| TMPRSS13 | | | | | 19 | | | | | | | |
| TMPRSS5 | | | | | | | | 8 | 8 | 10 | | |
| TMTC1 | | | | | | | | | | 12 | 0 | |
| TMTC2 | | | | | | | | | 8 | | | |
| TMX1 | | | | | | | | | 5 | | | |
| TMX3 | | | | | | | | | | 18 | | |
| TNC | | | | | | | | | | 5 | | |
| TNFAIP1 | | | | | | | | | | 10 | | 11 |
| TNFAIP2 | | | 21.5 | | | | | | | | | |
| TNFAIP3 | | | | | | | | | | | 4.167 | |
| TNFAIP8 | | | | | | | | 19.5 | 21 | | | |
| TNFAIP8L1 | | | | | | | | | 23 | | | |
| TNFRSF12A | | | | | | | | | 22.5 | 7 | | |
| TNFRSF19 | 18 | | | | | | | 22 | | | | |
| TNFRSF1B | | | | | | | | | 2.5 | 4 | | |
| TNFRSF21 | | | | | | | | 0 | | | | |
| TNFRSF22 | | | | | | | | | | 5 | | |
| TNFSF10 | | | | | | | | | 23 | | | |
| TNFSF12- | | | | | | | | 20 | | | | |
| TNFSF13B | | | | | 21 | | | 3.5 | 22 | | | |
| TNFSF15 | | | | 22 | | | 19.5 | | | | | |
| TNIK | | | | | | | | | 22.5 | | | |
| TNIP1 | | | | | | | 21 | | | | | |
| TNK2 | 18 | | | 21 | | | | | | | | |
| TNKS1BP1 | | | | | | | | 10 | 10 | | | |
| TNNC1 | | | 9 | | | | | | | | | |
| TNNI1 | 15 | | | 19 | | 18 | | | | 16 | | |
| TNPO1 | | | | | | | | | | | 23 | |
| TNPO2 | | | | | | | | | | | 2.833 | |
| TNR | | | | | | | | | | 0.5 | | |
| TNRC6B | | | | | | | | | | | 4.833 | |
| TNS1 | | | | | | | | | | | 18 | |
| TNXB | | | 0 | | | | | 5 | | | 23 | |
| TOB2 | | | | | | | | | 23 | | | |
| TOMM20L | | | | | | | | 21 | 16 | | | |
| TOMM34 | | 22 | 0 | | | | | 13 | 20 | | | |
| TOMM40 | | | | | 9 | | | | | | | |
| TOMM7 | | 8 | | | | | | 9 | | | | |
| TOP1 | | | | | | | | | 6 | | | |
| TOP1MT | | | | | | 19 | | | | | | |
| TOP2B | | | | | | | | | | 4 | | |
| TOP3B | | 9 | | | | | | | | 8.5 | | |
| TOR1A | | | | | | | | | 22 | 12 | 4 | |
| TOR1AIP1 | | | | | | | | | 21 | | | |
| TOR1AIP2 | | | | | | | | | 21 | | | |
| TOR1B | | | | | 12 | | | | | | | |
| TOR2A | 22.5 | | | | | | | | | | | |
| TOR3A | | | | | | | | | 17 | | | |
| TOX | | | | | 3.5 | 23.5 | | | | | | |
| TOX2 | | | | | | | | | 13 | 10 | | |
| TP53 | | | | | | | | | | 8 | | |
| TP53BP1 | | | | | | | | | 18 | | | |
| TP53BP2 | | | | | | | | | 23 | | | |
| TP53I11 | 5 | | | | | | | | 23.5 | | 23 | |
| TP53INP1 | | 8 | 9 | | | | | | | | | |
| TP53INP2 | | | 13 | | | | | | | | | |
| TPCN1 | | | | 6 | | | | | | | | |
| TPD52L2 | | | | | | 13 | | | | | | |
| TPGS2 | | 3 | | | | | | | 0 | | | |
| TPK1 | | 23 | 23 | | | | | | | | | |
| TPM1 | | 1.5 | 1 | | | 3 | | | | | | |
| TPMT | | | | 22 | | | | | | | | |
| TPP1 | | | | | | | | 5.5 | 18.5 | | | |
| TPPP | | | | | 23 | | | | | | | |
| TPPP2 | | | | | | | | | | | 5.167 | |
| TPPP3 | | | | | | | | 22 | 21 | | | |
| TPRA1 | | | | | | | | | 12 | 22 | | |
| TPSAB1 | | | | | | 14.5 | | | | | | |
| TPST1 | | | | | | | | | | 19 | | |
| TPST2 | 14 | | | | | | | | | | | |
| TRA2A | | | | | | | | 20 | | | | |
| TRABD | | | 21 | | | | | 23 | 23 | 22.5 | | |
| TRAF3 | | | | | | 12 | | | | 9 | | |
| TRAF5 | | | | | | | | | 3 | | | |
| TRAFD1 | | | | | | | | | | 15 | | |
| TRAK1 | | | | | | 0 | | | | | 2 | |
| TRAK2 | 7 | | | | | | | | | | | |
| TRAM1 | | | | | | | | 21 | 1 | | | |
| TRAM2 | | | | | | | | | 22.5 | | | |
| TRAPPC1 | | | | | | | | | | 10 | | |
| TRAPPC12 | | | | | | | | | | | | 14 |
| TRAPPC2L | | 8 | 8 | | | | | | | | | |
| TRAPPC3 | 19 | | 7 | 22.5 | | 22 | | 23 | | | | |
| TRAPPC8 | | 16.5 | 16 | | | 17 | | 5 | 21.5 | 13 | | |
| TRAPPC9 | | | 20.5 | | | | | | | | | |
| TRDMT1 | | | | | | | | | | 8 | | |
| TRDN | | | 0 | | | 6.5 | | 23 | 1 | | | |
| TRERF1 | | | | | | | | | | 11 | | |
| TRIB1 | | | 6 | | | | | | 21 | | | |
| TRIB3 | | | | 22.5 | | | | | | | | |
| TRIL | | | | | | | | | | 22 | | |
| TRIM12A | | | | | | | | | | 12 | | |
| TRIM13 | | | | | | | | 21 | | | | |
| TRIM14 | | | | | | | | 21 | | | | |
| TRIM16 | | | | | | | | | 22 | | | |
| TRIM2 | 19 | | 19 | | | | | 18 | 5.5 | 16 | | |
| TRIM23 | | | | | | | | | | 6 | | |
| TRIM24 | | | | 21 | | | | | | | | |
| TRIM25 | | | | | | | | 2 | 1 | | | |
| TRIM3 | | | | | | | | | | 8.5 | | |
| TRIM30B | | | 11 | | | | | | 13.5 | | | |
| TRIM37 | | | 2.5 | | | | | | | | | |
| TRIM40 | | | | | | | | | 15 | | | |
| TRIM41 | | | | | | | | | 23 | | | |
| TRIM44 | | | | | | | | | 19 | | | |
| TRIM5 | | | | | | | | | | 9 | | |
| TRIM56 | | | | | | | | | 4 | | | 7.5 |
| TRIM6 | | | | | | | | | | 8 | | |
| TRIM63 | | | | | | 16 | | | | 14 | | |
| TRIM65 | 4 | | | | | | | | | | | |
| TRIM68 | | | | | | | | | 23 | | | |
| TRIM7 | | | | | | | | | 22 | | | |
| TRIM8 | | | | | | | | | 22 | | | |
| TRIM9 | | | | | | | | | 22 | | | |
| TRIP10 | | | | | | | 21 | | | | | |
| TRIP12 | | | 21 | | 9 | | | 19 | | 16 | | |
| TRIP4 | | 13 | 3.5 | | | | | | 20 | 12 | | |
| TRIT1 | | | 8 | | | | | 22 | | 12 | | |
| TRMT5 | | | | | | | | | | 10.5 | 22 | |
| TRMT6 | | | | | | | | 21 | | | | 23 |
| TRPC3 | 0 | | 22 | | | | | | | | | |
| TRPM3 | | | | | | | | 12 | 11 | | | |
| TRPM7 | | 7 | 8 | | | | | 14 | 11 | 11 | | |
| TRPS1 | | | | | 7 | | | | 14 | 9.5 | | |
| TRPV4 | | | | | | | | | | 9 | | |
| TRRAP | | | | | | | | 23 | | | | 23 |
| TRU1AP | | | | | | | | | 8 | | | |
| TSC1 | | | | | | | | | 23 | | | |
| TSC22D1 | | | | | | 14 | | | 14.5 | | | |
| TSC22D3 | | | | 6 | | | | | | | | |
| TSGA10 | | | 21 | 16 | 14 | | 15 | | | | | |
| TSHR | | | | | | | | | | 7.5 | | |
| TSHZ3 | | | | | | | 8 | | | | | |
| TSKU | | | | | | | | | | 6 | | |
| TSLP | | | | | | | | | 17 | | | |
| TSPAN1 | | | | | | | | 9 | | 6 | | |
| TSPAN11 | | | | | | | | | 2 | | | |
| TSPAN13 | | | | | | | 20.5 | 19 | | | | |
| TSPAN14 | | | | | | 6.5 | | 7 | 6 | | | |
| TSPAN17 | | | | | | | | | 22 | | | |
| TSPAN18 | | | | | | | | | | 12 | | |
| TSPAN2 | | | | | | | | | 22 | | 7 | |
| TSPAN33 | | | | | | 20 | | | 13 | 15.5 | | |
| TSPAN4 | | | | | | | | | 0 | | | |
| TSPAN5 | | | 11 | | | | | | | | | |
| TSPAN6 | | | | | | | | | 23 | | | |
| TSPAN7 | | | | | | | | | 23 | | | |
| TSPAN9 | | 8.5 | 9 | | | | | | | | | |
| TSPO | | | | | | | | | 23 | | | |
| TSPYL2 | 18 | | | | | | | | | 13.5 | | |
| TSPYL3 | | | | | | | | 23 | | | | |
| TSPYL5 | | | 1 | | 4 | | | 23 | | 0 | | |
| TSR2 | | | | | | | | | | 7.5 | | |
| TST | | | 8 | | | | | | | | | |
| TSTA3 | | 9 | | | 9 | | | | | | | |
| TSTD2 | 18 | | 20 | | | | | | | | | |
| TTC14 | | | | | | | | | 7 | | | |
| TTC26 | | | | | | | | | 23 | | | |
| TTC28 | | | | | | | | 8.5 | 17 | | | |
| TTC3 | | | 19 | | | | | | | | | 18 |
| TTC30A | | | | | | | | 10 | 16 | | | |
| TTC32 | | | | | | | | | | | 4.5 | |
| TTC38 | | | | | | | | 22 | | | | |
| TTC39B | 20 | | | | | | | 22.5 | | | | |
| TTC39C | | | | | | | | 5 | | | | |
| TTC4 | 21.5 | | | | | | | | | | 0 | |
| TTC7A | | | 23 | | | 19 | | | | | 22 | |
| TTC7B | | | | | | 4 | | | | | 6.5 | |
| TTC8 | | | | | | | | 23.5 | 22 | | | |
| TTC9 | 21 | | 23.5 | | | | | | | | | 22 |
| TTI1 | | | | | | 20 | | | | | | |
| TTL | | | | | | | | | 15 | | | |
| TTLL1 | | | | | | | | | 23 | | | 0.5 |
| TTLL12 | | | | | | | | | | 10 | | |
| TTLL3 | | | | | | | | | | 15 | | |
| TTLL5 | | | | | | | | | 20 | | | |
| TTLL7 | | | | | | | | | | 18 | | |
| TTLL8 | | | | | | | | | | 15.5 | | |
| TTPAL | 10 | | | | | 13 | | | | | | |
| TTYH2 | | | | | | 9.5 | | | | | 9 | |
| TTYH3 | | | | | | | | | | 18 | | |
| TUBA1A | | | | | | | | | | 0 | | |
| TUBA4A | 6 | | | 7 | 6 | | | 3 | 1 | | | |
| TUBA8 | | | | | | | | | | | 0 | |
| TUBB | 4 | | 11 | | | | | | 6 | | | |
| TUBB2A | | | | | | 0 | | 1.5 | | 1 | | |
| TUBB2B | | | | | | 8 | | 22 | | | 17 | |
| TUBB4A | | | | 12 | | | | | | | 12 | |
| TUBB6 | | | 3 | | | | | 1 | 21 | | 23 | |
| TUBD1 | | | | | | | | | 0 | | | |
| TUBE1 | | | | | | | | | 15 | 13 | | |
| TUBG1 | 21 | 0 | 23 | 0 | 1 | 0 | 23 | 22.5 | 22 | 23 | 23 | 23 |
| TUBG2 | | | 17 | | | | | | 19 | 14 | | |
| TUBGCP2 | 20 | | | | | | | | | | | |
| TUBGCP3 | | | | | | | | 22 | | | | |
| TUBGCP4 | | | 3 | | | | | | | | | |
| TUBGCP5 | | | | | | | | | 22.5 | | | |
| TUFT1 | 19 | | | | | | | | | | | |
| TUG1 | | | | | | | | 22 | | | | |
| TULP4 | | | 7 | | | | | | | | | |
| TUSC5 | | | | | | | | | 21 | | | |
| TUT1 | | | 9 | | | | | | | | 23 | |
| TWF2 | 8 | | | | | | | | 11 | | | |
| TWSG1 | | | | 12 | | | | | | | | |
| TXK | | | | | | | | | 23 | | | |
| TXN | | | | | | | | | 21 | | | |
| TXN2 | | 4 | 4.5 | | | | | 2 | 3 | | | 22 |
| TXNDC11 | | | | | | | | 21 | 19 | | | |
| TXNDC12 | | | | | | | | | 19 | | | |
| TXNDC15 | | | | | | | | | 8 | | | |
| TXNDC16 | | | | | | | 20.5 | | | | | |
| TXNDC2 | | | | 16 | | | | | | | 23 | |
| TXNDC5 | | | | | | 21.5 | | | | | | |
| TXNDC9 | | | | | | | | | 2 | | | |
| TXNIP | | | | | | | | | | | | 9 |
| TXNRD1 | | | | | | | | | 21.5 | | | |
| TXNRD3 | | | | | | | | | 23.5 | 16.5 | | |
| TYMP | 21 | | | | | | | | 21 | | | |
| TYROBP | | | | | 15 | | | | | | | |
| TYSND1 | | | | | | | | | | 11.5 | | |
| TYW5 | 20 | | | | | 22 | | 19.5 | 21.5 | | | |
| U1 | | | 0 | | | | | | 22 | | 23 | |
| U12 | | | | | | | | | | 9 | | |
| U2 | | | | | | | | | | 8 | | |
| U2AF1L4 | | | | 0.5 | | | | | | | | |
| U3 | | | 22.5 | | | 1 | | 23 | 1 | | | |
| U4 | | | | | | | | | | | 23 | |
| U7 | | | | | | | | | | | 23.5 | |
| UAP1 | | | | | | | | 21 | | | | |
| UAP1L1 | | | | | | | | | 23.5 | | | |
| UBA3 | | | | | | | | | 20.5 | | | |
| UBA5 | | | | | | | | | | 10 | | |
| UBA6 | | | | | | | | | | 5 | | |
| UBAC2 | | | 22 | | | 1.5 | | | | | | 0.5 |
| UBAP1 | | | | | | | | 14 | | | | |
| UBAP2 | | | 9.5 | | | 14 | | 0 | 0 | | | |
| UBC | | | | | | | | | | | 23 | |
| UBE2B | | 18 | 19 | | | 23 | | 22.5 | 23 | 17 | | 19.5 |
| UBE2E2 | | | | | | | | | | 8 | | |
| UBE2F | | | | | | | | | 12 | | | |
| UBE2K | | 23 | 23 | | | | | | | 22 | 0 | 1 |
| UBE2L3 | | | | | | 1 | | | 3 | | 3 | 0 |
| UBE2L6 | | | | | | | | 21 | | | | |
| UBE2O | | | | | | | | 23 | | | | |
| UBE2Q1 | 20.5 | | 21 | | | | | | | | | |
| UBE2QL1 | | | | | | | | | | | 23 | |
| UBE2S | | | | | | | | | | 3 | | |
| UBE2U | | 9 | | | | | | | | | | |
| UBE2V1 | | | | | | | | | | | 10 | |
| UBE3B | | 7 | | | | 21 | | | | | | |
| UBE4A | 19 | | | | | | | | | | | |
| UBFD1 | | 7 | 5.5 | | | | | | | | | |
| UBL5 | | | 12 | | | | | | | | | |
| UBQLN1 | | | 11.5 | | | | | | | | | |
| UBQLN4 | | | | | | | | | 23 | | | |
| UBR2 | | | | | | | | | | 8 | | |
| UBR4 | | | 0 | | | 0 | | | | 1.5 | | 22.5 |
| UBTF | | | | | | | | | 0.5 | 4 | | |
| UBTFL1 | | | | | | | | 10 | | | | |
| UBXN1 | | 18.5 | 21 | 22 | | | | | | | | |
| UBXN2A | | | | | | | | 22 | | | | |
| UBXN2B | | | | | | | 3 | | 7 | | | |
| UBXN4 | | | | | | 2.5 | | 1 | 23 | | | |
| UBXN8 | 6.5 | 5.5 | 7 | | | 2 | | | | | 0 | |
| UCHL3 | | | | | | | | 15 | | | | |
| UCK1 | | | | | 3.5 | 13 | | | | | | |
| UCK2 | | | | | | | | 10 | | | | |
| UCKL1 | | | | | | | | | 14 | | | |
| UCP1 | | | | | | 17 | | | | | | |
| UCP2 | | | | | | | | 8 | | | | |
| UCP3 | 7 | | | | | | | 2.5 | | 7 | | |
| UFM1 | | | | | | | | 1 | 1 | | | |
| UGGT1 | | | | | | | | | 0 | | | |
| UGGT2 | | | | | | | | | | | | 6 |
| UGT2B28 | | | 19 | | | | | | | | | |
| UGT8 | | | | | | | | | | 8 | | |
| UHRF1BP1L | | | | | | | | 16 | | | | |
| UHRF2 | | | | | | | | | 22 | | | |
| ULK1 | | | | | | | | | 22 | | | |
| ULK2 | | | | | | | | | | | 22 | |
| UMPS | | | | | | | | 9 | | | | |
| UNC119B | | | 21 | | | | | | 21 | | | |
| UNC13A | | | | | | | | | 12 | | | |
| UNC13B | | | | | | | | | 23 | | | |
| UNC45A | | | | | | 2 | | 4 | | 4 | | |
| UNC50 | | | | | | 3.5 | | 5 | | | | 19 |
| UNC5B | | 0.833 | | | | | | | | | | |
| UNC5C | | | | | | | 20 | | | | | |
| UNC79 | | | | | | | | | | 15 | | |
| UNG | 18 | | | | | | | | | 18 | | |
| UOX | | | | | | 7.5 | | | | | 7 | |
| UPF1 | | 9 | | | 9 | | | | 22 | 7 | | |
| UPK1B | | | | 11 | | | | | | 16 | | |
| UPP2 | | | | | | | | 6 | | | | |
| UPRT | | | | | | 20 | | | 22 | 16 | | |
| UQCR10 | | | | | | | | | | 7.5 | | |
| UQCRC1 | | | | | | | | | 23 | | | |
| UQCRH | | | | | | | | | | | | 21 |
| URB2 | | | | | | | | | 1 | | 10 | |
| URM1 | | | | | | | | | | 10 | | |
| UROC1 | 10 | | | | | | | 10.5 | | | 11.5 | |
| UROD | | | | | | | | | | 4 | | |
| UROS | | | | | | | | | 19 | | | |
| USE1 | 16 | | | | | | | | | | | |
| USF2 | | | | | | | | | | | | 8 |
| USH1C | | | 22 | | | | | | 11 | | | |
| USO1 | | | | | | 23 | | | 22 | | | |
| USP1 | | 9 | | | | | 8 | | | 9 | | |
| USP10 | | | 3 | | | | | 22.5 | 22 | | | |
| USP13 | | | | | | | | | | 11 | | |
| USP14 | | | | | | | | 20.5 | | | | |
| USP15 | | | | | | | | | | 10 | | |
| USP18 | | | | | | 1.5 | | | | | | |
| USP2 | | | | | 23 | | | 21 | 23 | | | |
| USP21 | | | | | | | | | 23 | | | |
| USP22 | | | | | | | | | | 4 | | |
| USP24 | | | | | | | | | 20 | | | |
| USP28 | | | | | | | | | | 7 | | |
| USP31 | | | | | | | | 11 | | 9 | | |
| USP32 | 0 | | 2 | | | 4 | | | 0 | | 2 | |
| USP33 | | 21.5 | | 19 | | | | 21 | | | | |
| USP35 | | | 0 | | | | | 21 | | | | |
| USP36 | | | 19 | | | | | | | | | |
| USP38 | 1 | | | | | | | | 22 | | | |
| USP45 | | 22 | | | | | | | | | | |
| USP46 | | | | 12 | | | | | 16 | | | |
| USP53 | 22 | | | | | | | | | | | |
| USP54 | 0 | | 1.5 | | | | | 2 | 23 | | | |
| USP6NL | | | 1 | | | | | | | | | |
| USP7 | | | | | | | | 0 | | | | |
| USP8 | | | | | | 18 | | | | | | |
| UST | | | | | 21.5 | | | | | | | |
| UTP6 | | | | 20 | 20 | | 19 | | | | | |
| UTRN | | | | 21 | | | | | | | | |
| UXS1 | | | 3 | | | | | | | | | |
| V1 | | 11 | 10 | | | 16 | | | 16 | 14.5 | | |
| V2 | | 7 | | | | | | | | 9 | | |
| V3 | | 23.5 | 0 | | | 23 | | 0 | 0 | 22 | | 23 |
| VAC14 | | 3 | 2 | | | | | | | | | 5 |
| VAMP2 | | | | | | | | 23 | | | | |
| VAMP3 | | | | | | | | | 20 | | | |
| VAMP4 | | | | | | | | | | 2.5 | 23.5 | 23 |
| VAMP5 | | | | | | | | 19 | 22 | | | 21 |
| VAMP7 | | | | 21 | | | | | | | | |
| VAMP8 | | | | | | 20 | | 18 | | | 2 | |
| VANGL2 | | | | 19.5 | | | | | | | | |
| VAPA | | | 23 | | 22 | 1 | | | 21.5 | | | |
| VARS | | | | 22 | | | | | 7 | | | |
| VARS2 | | 7 | | | | | 7 | | | 7 | | |
| VASP | | | | | | | | 21 | | | | |
| VAT1 | 17 | | | | | 16 | 14 | | 19 | 14 | | |
| VAT1L | | | | | | 12 | | | | 11 | | 9 |
| VAULT | | | | | | | | | | | | 7 |
| VAV2 | | | | | | | | | | | | 7 |
| VCAM1 | | | | | | | | 1 | 23 | | | |
| VCAN | | | | | 21 | | | | | | | |
| VCL | | | | | | | | 17 | | 16 | | 19 |
| VDAC1 | | | | | | 12 | | | | 0 | | |
| VDR | | | | 22 | | | | | | | 8 | |
| VEGFA | 20 | | | | | | | | | | | |
| VGLL3 | | 17 | | | | 17 | | 14 | | 13 | 12 | |
| VGLL4 | 3 | 2.5 | 3 | | | | | 10.5 | | | 0 | |
| VILL | 11 | 12 | 11 | | 9.5 | 12 | | 13 | | 11 | | 12.5 |
| VIM | | | 20.5 | | | | | | | | | |
| VIPR2 | | | | | | | | 2.5 | | | | 19 |
| VKORC1 | | | 22 | | | 9 | | | | | | |
| VMN1R188 | | 16 | | | | | | | 18 | 13 | | |
| VMN1R231 | | | | | | | 6 | 6 | | | | |
| VMN1R236 | | | | | | | | 2 | 1 | 19 | | |
| VMN1R24 | | | | | | | | | | 3 | | |
| VMN1R32 | | | | | | | | 13 | | | | |
| VMN1R81 | | 22 | 23 | | | 22 | | | | | | |
| VMN1R-PS144 | | | | | | | | | | | 11.17 | |
| VMN2R118 | | | | | | 16 | | | | | | |
| VMN2R83 | | | | | 11 | | | | 13 | | | |
| VMN2R9 | | 9 | | | | | | 13 | | | | |
| VMP1 | | | | | | | | 8 | 12 | 10.5 | | |
| VNN1 | | | | | | | | | 2 | | | |
| VNN3 | | | | | | | | 9 | | | 2.333 | |
| VPREB3 | | | | | | | | | 21.5 | | | |
| VPS11 | | | 0 | | | | | | | | | |
| VPS13A | | 2.5 | 3 | | | | | 8 | | | | 2 |
| VPS26A | | | | | | | | | 21 | | | |
| VPS28 | | | | | | | | 19 | 21 | | | |
| VPS33A | | | | | | | | | 17 | | | |
| VPS37B | | | | | | 1.5 | | | | | | |
| VPS41 | | 10 | | | | 10 | | 11 | | 10 | | |
| VPS45 | | | | | 9 | | | | | | | |
| VPS4B | | | | | | | | | 21.5 | | | |
| VPS52 | | | | | | | | 23 | 23 | | | |
| VPS72 | | | | | | | | | 22.5 | 20 | | |
| VSIG4 | | | 8 | | | | | | | | | |
| VSNL1 | | | | | | | | 10 | 2.5 | | | |
| VSTM4 | | | | | | | | | 21 | | | 21 |
| VTA1 | | | | | 2 | | | | | | | |
| VTCN1 | | | | | | | | | 21.5 | | | |
| VWA1 | | | | | | | | | 22 | | | |
| VWA3A | | | | | | | | 22 | | | | |
| VWA5B2 | | | | | | 23 | | 18 | | | | |
| VWC2 | | | | | | | 0 | | 14 | | | |
| VWC2L | | | | | | | | | | 12 | | |
| VWCE | | | | | | | | | 15 | | | |
| WARS | | | 21 | | | | | | 11 | | | |
| WAS | 2.5 | | | | | | | | | | | |
| WASF2 | | | | | | | | | 0 | | | |
| WBP1 | | | | | | | 23 | | 23 | | | |
| WBP11 | | | | | | | | | | | | 21 |
| WBP2 | | | | | | | 5 | | | | | |
| WBSCR25 | | | | | | | | | | 18 | 6 | |
| WBSCR27 | | | | | | | | | | 21 | | |
| WDFY3 | 8 | | | | | | | | | | | |
| WDFY4 | | | | 12 | | | | | | | | |
| WDR1 | | | | | | 16 | | | | 16 | | |
| WDR12 | | | | | | | | | | 3.5 | | |
| WDR19 | 20 | | | | | | | 22 | | | | |
| WDR27 | 10 | | | | | | | 14 | 12 | 11 | | 15 |
| WDR31 | | | 1.5 | | | | | | | | | |
| WDR33 | | | | | | | | | | 11 | | |
| WDR35 | | | | | | | | | 12 | | | |
| WDR36 | | | | | | | | | 9.5 | | | |
| WDR37 | | | | | | | | | 23 | 17 | | |
| WDR44 | | | 17 | | | 17 | | 18 | | | | |
| WDR45 | | | 10 | | 0 | | | | | | | |
| WDR46 | | 10 | 11 | | | | | | 13 | 9 | | |
| WDR47 | | | | | | | | 0 | | | | |
| WDR48 | | | | | 11.5 | | | | | | | |
| WDR54 | | | | | | | | | 18 | | | |
| WDR6 | | | | | | | | 16 | 13.5 | 9 | | |
| WDR65 | | | | | | | | | 13 | 16 | | |
| WDR67 | | | | | | 2.5 | | | | | | |
| WDR70 | 20 | | | | | | | | | | | |
| WDR75 | 21 | | | | | | | | | | | |
| WDR76 | | | 23 | | | | | | | | | |
| WDR78 | | | | | | | | 7 | | | | |
| WDR81 | 15 | | | | | | | | | | | |
| WDR82 | | | | | | 5 | | | | | | |
| WDR83 | | | | | | | | | 15 | | | |
| WDR83OS | | | | | | | | | | 11 | | |
| WDR89 | | | 21 | | | | | | | 2 | | 2 |
| WDR91 | | | | | | | | | 6 | | | |
| WDR95 | | 3 | 5 | | | 23.5 | | | | | | |
| WDSUB1 | | | | 11 | | | | | | 8 | | |
| WDTC1 | 20 | | | | | | | | | | | |
| WDYHV1 | | | | | | | | 14 | 14 | | | |
| WEE1 | | | | 17 | 17 | 18 | | 18.5 | 19.5 | 14.5 | | 19.5 |
| WFDC1 | | 9 | 11 | | | | | | 12 | | | |
| WFDC10 | | | 23.5 | | | | | | | 8 | | |
| WFDC2 | | 23 | | | 21 | 23.5 | | | | | 23 | |
| WFDC3 | | | | | | | | | 8 | | | |
| WFDC6A | | 5 | 7 | | | | | | | | | |
| WFDC8 | 21 | 6 | 6 | | | | 2 | | | 12 | | |
| WFS1 | | | | | | | 20.5 | | | | | |
| WHSC1 | | | | | | | 4 | | | | | |
| WHSC1L1 | | | | | | 12 | | | | | | |
| WHSC2 | | | | | 5.5 | | | | | | | |
| WIBG | | | 5 | | | | | | | | | |
| WIPF3 | | | | | | | | | | 21 | | |
| WIPI1 | 18 | | | 19 | | | 19 | 18 | 18 | 17 | | |
| WIPI2 | | | | | | | 21 | 18 | 18 | 17 | | |
| WISP2 | | | 21 | | | | | | | 10 | | |
| WNK1 | | | | 14 | 13 | 14 | 15.5 | | | | | |
| WNK2 | 20 | | | | | | | | | | | |
| WNK4 | 21 | | | | | | | 23 | 21 | 22 | | 22 |
| WNT2 | 13.5 | 13 | 14 | | | 15 | | 16 | 20 | 14 | | 13.5 |
| WNT5A | | | | 16 | 17 | | | 12 | | | | |
| WNT5B | | | | | | | | | 4 | | | |
| WNT7A | 20 | | | | | 7.5 | | 3 | 2 | | 4 | |
| WRAP73 | | | | | | | | 22.5 | 18 | | | |
| WRB | | | | | | | | 9 | | | | |
| WRNIP1 | | | | | | | | | 6 | 7 | | |
| WSB1 | 8.5 | 10 | 12 | | | | | | | | | |
| WSCD1 | 9 | 8 | 9.5 | | | | | | 14.5 | | | 10 |
| WWC1 | | | | | | | | | 20 | | | |
| WWC2 | | 9.5 | | | | | | | | 8.5 | | |
| WWOX | 6 | | 7 | | | | | | | 5 | | |
| WWP1 | | | | | | | | | 21 | | | |
| WWP2 | | | | | | | | 15 | 14 | | | |
| WWTR1 | | | | | | 18 | | | | 14 | | |
| XAB2 | | | | | | | | | 2 | | | |
| XAF1 | | | 22 | | | | | | 11.5 | | | |
| XBP1 | | | | | | | | 21 | | | | 18 |
| XDH | | | | | | | | | 7 | 14 | | |
| XK | | 5.5 | | | | | | | | | | |
| XKR8 | | | | | | | | | | 8.5 | | |
| XPA | 12 | | 12 | | 12 | | | | 18 | | | 13 |
| XPNPEP3 | | | | | | | | | 17 | | | |
| XPO1 | 20 | | | | | | | | | | | |
| XPO6 | | | | | | | | 22 | | | | |
| XPO7 | | | | | | | | | 21 | | | |
| XPR1 | | | | | | | | | 11 | | | |
| XRCC1 | | | | | | | | | 7 | | | |
| XRCC3 | | | | | | | | | | 12.5 | 23 | |
| XRN2 | 10 | | | | | 9 | 4 | | | | | |
| XYLB | | | 16 | | | 17 | | | | | | |
| XYLT1 | | | | | | | | | | 0 | | |
| Y_R | | | | | | 21 | | | | | | |
| YAF2 | 15 | | | 12 | | | | 19 | | | | |
| YAP1 | | | 10 | | | | | | | | | |
| YARS | | | 7 | | | | | | | | | |
| YBX1 | | | | | | | 9 | | | | | |
| YEATS4 | | 4 | 5.5 | | | | | | | 7 | | |
| YIF1B | | 2 | 0 | | | | | | | | 23 | |
| YIPF2 | | | | | | | | 10 | | 12 | 10.5 | |
| YIPF5 | | | | | | | | | | 10 | | |
| YKT6 | | | | | | | | 14.5 | | | | |
| YME1L1 | 19 | | | | | | | | | | | |
| YPEL2 | | | | | | | | | 22 | | | |
| YPEL3 | | | 6 | | | 10 | | 20.5 | | 23 | 18 | 22 |
| YPEL5 | | 7 | | | | | | | | | | |
| YWHAB | | | | | | 22.5 | | | | | | |
| YWHAG | | | 18.5 | | | | | | 17 | | | |
| YWHAH | | | | | | | | | | 18 | | |
| YWHAQ | | | | | | 22 | | | | | | |
| YWHAZ | | | 11 | | | 11.5 | | | 8.5 | | | 13 |
| YY1 | | | | | | | | | 22 | | | |
| ZAK | | | | | | | | | | | 9.5 | |
| ZBED3 | | | | | | 19 | | | | 22 | | |
| ZBTB10 | | | | 13 | 13 | | | 17 | 22 | | | |
| ZBTB16 | | | | | | | | 17 | | | | |
| ZBTB2 | | | | | | | | 13 | | | | |
| ZBTB20 | | | | | | | | 11.5 | | | | |
| ZBTB22 | | | | | | | | | | 10 | | |
| ZBTB26 | | | | | | | | | 23 | | | |
| ZBTB34 | | | | | | | | | 2 | | | |
| ZBTB38 | | | 13 | | | 13 | | 16 | 18 | | 14 | 18 |
| ZBTB40 | | | | | | 1 | | 20.5 | | | | |
| ZBTB43 | | | | | | | | | | | | 13 |
| ZBTB5 | | 9 | 10.5 | | | | | | 5 | 11 | | |
| ZBTB7A | | | | | 22 | | | | | | | |
| ZBTB7B | | | | | | | | | | 8.5 | | |
| ZBTB8A | | 7 | 9 | | | 17 | | | 9 | | | |
| ZC2HC1C | 6 | | | | | | | | | 12 | | |
| ZC3H12B | | | | | | | | 16 | | | | |
| ZC3H12C | | 9 | 10 | 3 | | | | | | | | |
| ZC3H12D | | | | | | | | | 1 | | | |
| ZC3H4 | | | | | | | | | | | | 13 |
| ZC3H6 | | | | | | | | 1 | 0 | 8 | | 10 |
| ZC3H7A | | | 5 | | | | | | | | | |
| ZC3H7B | | | | | | | | | 10 | 8.5 | | 10 |
| ZC3HC1 | | | | | | | | | 23 | | | |
| ZC4H2 | | | | 21 | | | | | | | | |
| ZCCHC10 | 10.5 | | | | | | | | 12 | 14.5 | | |
| ZCCHC11 | | 6.5 | | | | | | 7 | | | | |
| ZCCHC14 | | | | | 13 | | | | 18 | 12 | | 12 |
| ZCCHC17 | | | | | | | | 1 | | | | |
| ZCCHC24 | | 15 | | | | 20 | | | | 14 | | |
| ZCCHC5 | | | 19 | | | 11 | | | | 19 | 13 | |
| ZCCHC6 | | | | | | | | | | | | 4 |
| ZCCHC7 | | | | 4 | | | | | | | | |
| ZCCHC9 | | | | | | 1 | | | 3.5 | | | |
| ZCRB1 | | | | | | | | | | 16 | | |
| ZDHHC13 | | | | | | | | | | 6 | | |
| ZDHHC14 | | 23 | 23.5 | | | | | | | | 5 | 23 |
| ZDHHC15 | | 1 | 1 | | | | | | | | | 1 |
| ZDHHC16 | | | | | | | | | 11 | | | |
| ZDHHC17 | | | | 6 | | | | | | | | |
| ZDHHC20 | 13.5 | | | | | | | | | 23 | | |
| ZDHHC23 | | | | | | | | | | | | 3 |
| ZDHHC3 | | | 22.5 | | | | | 20 | | 21 | 22 | |
| ZDHHC4 | 4 | 4 | | | | | | | | 1.5 | 2 | |
| ZDHHC5 | | | | | | | | | | | 23 | |
| ZDHHC7 | | 12 | | | 4.5 | | | | | 8 | 7 | |
| ZDHHC8 | | | | | | | | | 10 | | | |
| ZDHHC9 | | | | | | | | | 21 | | | |
| ZEB2 | | | | | | | | | | | 5.167 | |
| ZER1 | | | | | | | 20 | | | | | |
| ZFAND3 | | | | | | | | | 9 | | | |
| ZFAND4 | | | | | | | | | | 5 | | |
| ZFAND5 | 11.5 | | | | | | | | 21 | 7 | | |
| ZFAND6 | | | | | | 14 | | | | | | |
| ZFAT | | 23.5 | 23.5 | | | 23 | | | | | 0 | |
| ZFC3H1 | | | | | | | | 22 | 19 | 23 | | |
| ZFHX4 | | | | | | | | | | | | 20.5 |
| ZFP101 | | | | | | | | 0 | | | | |
| ZFP109 | | | | | | | 21 | | | | | |
| ZFP110 | 20 | | | | | | | | | | | |
| ZFP161 | 3 | | | | | | | | | | | |
| ZFP185 | | | | | | | | 14 | | | | 7.5 |
| ZFP2 | | | | | | | | 11 | | | | |
| ZFP273 | | | | | | | | 21 | | | | |
| ZFP30 | 5 | | 1.5 | | | | | | | | | 4 |
| ZFP35 | | | | | 16 | | | | | 10 | 14 | |
| ZFP36 | 9 | | | | | | | | | | | |
| ZFP36L1 | | | | | | 18 | | | | | | 19 |
| ZFP36L2 | | | | | | | | | 6.5 | | | |
| ZFP40 | | | | | 13 | | | | | | | |
| ZFP455 | | | 4.5 | | | | | 20.5 | 22 | 16 | | |
| ZFP54 | | | | | | | | | 11 | 12 | | |
| ZFP568 | | | | | | 3.5 | 8 | | 22 | | 0 | |
| ZFP599 | 23 | | | | | | | | | | | |
| ZFP62 | | | | | | | | 21 | 21 | | | |
| ZFP64 | | | | | | | | 22 | 22 | | | |
| ZFP663 | | | | | | | | | 2 | | | |
| ZFP708 | | | | | | | | | 23 | | | |
| ZFP72 | | 14 | | | 16 | | | 9 | 9 | | | 11.5 |
| ZFP740 | | | | | | | | 1 | 23.5 | | | 23 |
| ZFP759 | | | | | | | | 19.5 | 18.5 | 17 | | |
| ZFP760 | | | | | | 23 | 22 | 20.5 | 18.5 | | | |
| ZFP809 | 9 | | 8 | | | | | 23 | 20.5 | 11 | | 12 |
| ZFP870 | | | | | | | | | 1.5 | | | |
| ZFP871 | | | | | | | | | 1.5 | | | |
| ZFP874A | | | 11 | | | | | 8.5 | 3 | | | |
| ZFP882 | | | | | | | | | 22.5 | | | |
| ZFP91 | | | | | | | | | | 3 | | |
| ZFP948 | | | | | | | 9 | 11 | | | | |
| ZFP949 | | | | | | | | | 23 | | | |
| ZFP959 | | | | | | | | | 23 | | | |
| ZFP961 | | | 2 | | | | | | | | | |
| ZFR | | 10.5 | 10 | | | | | | 14.5 | | | 12.5 |
| ZFYVE1 | | | | | | | | | 0.5 | | 8.5 | |
| ZFYVE16 | | | | | | | | 11 | 9 | | | |
| ZFYVE19 | | | 5 | | | 16 | | 14 | | 13 | | |
| ZFYVE21 | | | | | | | | 23 | 20.5 | 1 | | |
| ZFYVE26 | | | | | | | | | 15 | | | |
| ZFYVE27 | 19 | | 7 | | | | | | | | | |
| ZFYVE9 | | | | | | | | 0 | 20 | | | |
| ZGPAT | | 21.5 | 23 | | | | | 22 | 20 | | 23 | |
| ZHX2 | | | | | 15 | 21.5 | | | | | | |
| ZHX3 | | | | | | | | 12 | | | | |
| ZIC1 | | | 11 | | | 12 | 11 | | | | | |
| ZIC4 | | | | | | | | | | 14.5 | | |
| ZIM1 | | | | | | | 5 | 7 | | | | 9 |
| ZKSCAN1 | | | | | | | | | 8 | | | |
| ZKSCAN4 | | 4 | | | | | | | 21 | | | |
| ZMAT1 | | | 13 | | | | | 12 | 1 | | | |
| ZMAT5 | | | | | | | | | 22 | 15 | | |
| ZMIZ1 | | | | | | | | | | 9 | | |
| ZMPSTE24 | | | | | | | 13 | | | | | 9 |
| ZMYM3 | | 15 | | | | | | 11 | | | | |
| ZMYM6 | | | 7 | | | | | | | | | |
| ZMYM6NB | | | | | | | | | | 23 | | |
| ZMYND10 | | | | | | | | | | 6 | | |
| ZMYND11 | | | | | | | | | 2 | | | |
| ZMYND12 | | | | | | | | | | | | 16 |
| ZMYND8 | | | | | | | | | | | 10.5 | |
| ZNF142 | | 10 | 12 | | | | | | 21 | | | |
| ZNF143 | | | | | | | | 20 | 15 | 23 | | |
| ZNF157 | 8 | | | | | | | | | 9 | | |
| ZNF175 | | | | | | | | 10 | | | | |
| ZNF180 | | | | | | | | | 21 | | | |
| ZNF182 | | | | | | | | | | | 20 | |
| ZNF187 | | | | | 22 | | | | | | | |
| ZNF189 | 19 | | | | | | | | | | | |
| ZNF192 | | | 11 | | | | | | 11.5 | 11 | 8.5 | |
| ZNF207 | | | | | | | 19 | | | | | |
| ZNF226 | | | | | | | | | 7 | | | |
| ZNF23 | | | | | | | | | 11 | | | |
| ZNF236 | | | | | | | | | | 19 | | |
| ZNF238 | | | | 5.5 | | | | | | | | |
| ZNF24 | | | | | | | | | | | 12 | |
| ZNF266 | | | | | | | | | | | 5 | |
| ZNF275 | | | | | | | | | | | 4.5 | |
| ZNF277 | | | | | | | | | 11 | | | |
| ZNF280B | | | | | | | | 8.167 | | | | |
| ZNF292 | | | | | | | | | | | 4.5 | |
| ZNF295 | | | | | | | | | | | 5 | |
| ZNF317 | | | | | | | | | 10 | | | |
| ZNF32 | | | | | | | | | | | | 22 |
| ZNF329 | | | | 15.5 | | | | | | | | |
| ZNF334 | | | | | | | 8.5 | | 10.5 | | | |
| ZNF346 | | | | | | 21 | | | 21 | | | |
| ZNF35 | | | | | | | | 6 | | | | |
| ZNF367 | | | | | | | | | 6 | | | |
| ZNF384 | | | | | | 22 | | | | | | |
| ZNF397 | | 13 | | | | | | | | | | |
| ZNF407 | | | | | | | | | | 6 | | |
| ZNF41 | | | | | | | | | 21.5 | 1 | | |
| ZNF416 | | | 22 | | | | 7 | | | | 7.5 | |
| ZNF426 | | | | | | | | | 22.5 | | | |
| ZNF429 | | | | | | 16 | | | | 11 | | |
| ZNF438 | | 23 | 2.5 | | | | | | | | | |
| ZNF445 | | | 9 | | | | | | | | | |
| ZNF449 | | | | | | | 13 | | 3 | | | |
| ZNF454 | | | 20 | | | | | | 18 | 17 | | 2 |
| ZNF462 | | | | | | | | | 16.5 | | | |
| ZNF467 | | | | | | 9 | | | 2.5 | | 6 | |
| ZNF496 | 20.5 | | | | | | | | 22 | | | |
| ZNF507 | | | | | | 3 | | | | 6 | | |
| ZNF511 | | 8.5 | 10 | | | | | 11 | | | | |
| ZNF512 | 10 | | | | | | | | | | | |
| ZNF512B | 5.5 | | | | | | | | | | | |
| ZNF518A | | | | | | | | | 22 | | | |
| ZNF521 | 8.5 | | | | | | | 12 | | 10 | | |
| ZNF558 | | | | | | | | | 7 | | | |
| ZNF563 | | | | | | | | | 10 | | | |
| ZNF569 | | | | 23 | | | | | | | | 11 |
| ZNF571 | | | 15 | 13.5 | | 13 | | 2.5 | | | | 16 |
| ZNF583 | | | | | | | | 16 | | | | 9 |
| ZNF585A | | | | | | | | | | 8 | | |
| ZNF598 | 13 | 11 | | | | | | | | | | |
| ZNF616 | | 9 | | | | | | | | 11 | | |
| ZNF622 | | | | | | | | 20 | 5.5 | | | 21 |
| ZNF624 | | | | | | | | 7.5 | 21 | | | |
| ZNF627 | | | 23 | | | | | 17 | 9 | | | |
| ZNF629 | | | | | | | | | | 10.5 | | |
| ZNF652 | | | | | | | | | | 11 | 6 | |
| ZNF653 | | | 22 | 21.5 | 21.5 | | 22 | | | | | 0 |
| ZNF655 | | | 12.5 | | | | | | | 2.5 | | |
| ZNF667 | | | | | | | | 7.5 | 9.5 | | | |
| ZNF687 | | | | | | | | 11 | | | | |
| ZNF692 | | | 16 | | | 12 | 7 | | | | | |
| ZNF7 | | | | | 7 | | | 9 | | | | |
| ZNF703 | | | 22 | | | | | | | | | |
| ZNF704 | | | | | | | | | | 10 | | |
| ZNF706 | | | | | | | | | 6 | | | |
| ZNF707 | | | | | | | | | 1 | | | |
| ZNF717 | | | | | | | | | 2 | | | |
| ZNF728 | 11 | | | | | 18 | | | 13 | | | |
| ZNF770 | 18 | | 20 | 16 | | 18 | | 19 | | | | |
| ZNF773 | 17 | 16 | 19 | | | 16 | | 19 | | 16 | | 18.5 |
| ZNF799 | | | | | | | | | 3 | | | 19.5 |
| ZNF845 | | | | | 20 | | | | | | | |
| ZNF845 | | | | | | | | | 9.5 | 10 | | |
| ZNF845 | | | | | | | | | | 13 | | |
| ZNF845 | | | | | | | | 7 | | | | |
| ZNF845 | | | | | | | | | | 3 | | |
| ZNF846 | | | | | | | | | 0.5 | | | |
| ZNHIT1 | | | | | | | | | | 2 | | |
| ZNHIT2 | | | | | | | | 15 | | | | |
| ZNRD1 | | | | | | 6 | | | | | | |
| ZNRF1 | | 10.5 | 11 | | | | | | | 14 | | 10 |
| ZNRF2 | 2 | | | | | | | | | 20 | | |
| ZNRF3 | | | 10.5 | | | 10 | | 7.5 | 7 | 9 | | |
| ZRANB2 | | | | | 21 | | 20 | | | | | |
| ZRSR2 | 23 | | | | | | | | 22 | | | |
| ZSCAN20 | | | | 20 | | | | | 23 | | | |
| ZSCAN21 | | 7.5 | 8.5 | | | | | | 11 | 11 | | |
| ZSWIM1 | | | | | | 18 | | 14.5 | | | | |
| ZSWIM4 | | | | | | | | | 23 | | | |
| ZSWIM7 | | | | | 19 | | | | | | | |
| ZW10 | | | | | | | | 23 | | | | |
| ZYX | | 14.5 | 16 | | | | | | 16 | | | 5.5 |
| ZZEF1 | | | | | | | | | | 17 | | |
| 41700 | | | | | | | | | 22.5 | | | |
| 41703 | | | | | | | | | | | 23 | |
| 41704 | | | | | | | | | | | 4.167 | |
| 41705 | | | | 6 | | | | | | | | |
| 41883 | | | | 20 | | | | | | | | |
| 41886 | 13 | 16 | 16 | | | 17 | | 14 | 15.5 | 13 | | |
| 41888 | | | | 15.5 | | | | | | | | |
| 41889 | | | | | | | | | 11.5 | | | |
| 41891 | | | | | | | | | | 12 | | |
| 41893 | | | | | | | | | | 5 | | |
| 0610009B22RIK | | | | | | 22.5 | | | | | 2 | |
| 0610012H03RIK | | | | | | | | 1.5 | | | | |
| 1110002E22RIK | | | | | | | 21 | | 5 | | | |
| 1110002L01RIK | | | | | 23.5 | | | | | | | |
| 1110017F19RIK | 4 | | | | | | | 0 | 0 | | | |
| 1110035H17RIK | | | | | | | | | 21 | | | |
| 1190003J15RIK | | | | | | | | 22 | | | | |
| 1300002K09RIK | | | | | | | | 21 | | | | |
| 1520401A03RIK | | | | | | | | 0 | | | | |
| 1600029D21RIK | | | | | | | | | | | | 22.5 |
| 1700001L05RIK | | | | | | | 14.5 | | | | | |
| 1700008F21RIK | | | | 20 | | | | | | | | |
| 1700014D04RIK | | | | | | | | | | | 1 | |
| 1700048O20RIK | | | | | | | | | | 1.5 | | |
| 1700055D18RIK | | | 10 | | | | | | 4.5 | | | |
| 1700055N04RIK | | | 3.833 | | | | | | | | | |
| 1700086L19RIK | | | 13 | | | | | | | | | |
| 1810009A15RIK | | | | | | | | | 1 | | | |
| 1810058I24RIK | | | | | | | | | | | 0 | |
| 2010016I18RIK | | | | | | | 5 | | | | | |
| 2010107H07RIK | | | | | | | | | 11.5 | | | |
| 2010204K13RIK | | | 23 | | | | | | 23 | | | |
| 2010300C02RIK | | | | | | 0 | | | | | | |
| 2210407C18RIK | | | | | | | | | | | 4.167 | |
| 2310015B20RIK | | | | | | | | | | 11 | | |
| 2310016G11RIK | | | | | | | | | | 21.5 | | |
| 2310039H08RIK | | | | | | | | | | | 1.167 | |
| 2310042D19RIK | 19 | | | | | | | | | | | |
| 2310042E22RIK | | | | | | | | | 22.5 | | | 8.5 |
| 2410002F23RIK | | | | | | | | | 10 | | | |
| 2410066E13RIK | 21 | | | | | | | | | | | |
| 2610001J05RIK | | | 4 | | | | | | | | | |
| 2610002M06RIK | | | | 7.167 | | | | | | | | |
| 2610008E11RIK | | | | | | | | | | | | 3.833 |
| 2610021A01RIK | | | | | 9 | | | | | | | |
| 2610029I01RIK | | | | | | | | | 6 | | | |
| 2610044O15RIK | | | | | | | | 22 | | | | |
| 2700089E24RIK | | | 6.5 | | | | | 13 | | 13 | 13.5 | |
| 2810032G03RIK | | | 0 | | | | | | | | | |
| 2810055G20RIK | | | | | | | | | 10 | | | |
| 2810408A11RIK | | | 9 | | | | | | | | | |
| 2900026A02RIK | 0 | | | | | | | | | | | |
| 3000002C10RIK | | | | 3 | | | | | | | | |
| 3010003L21RIK | | | | | | | | 20 | | | | |
| 3110052M02RIK | | | 15 | | | | | | | | | |
| 3222401L13RIK | | | | | | | | | | 16.5 | | |
| 3300002A11RIK | 21.5 | | | | | | | | | | | |
| 4631405J19RIK | | | | | | | | | | 13 | | |
| 4632404H12RIK | | | | | | | | | 0 | | | |
| 4632427E13RIK | | | | | | | | | 23.5 | 1.5 | | |
| 4732465J04RIK | | | | | 1 | | | | | | | |
| 4832428D23RIK | | 1 | | | | | | | | | | |
| 4921513D11RIK | | | | | | | | | | 6 | | |
| 4930425F17RIK | | | | | | | | | | | | 0 |
| 4930463O16RIK | | | | | | | 22 | 23 | | | | |
| 4930503L19RIK | | | | | | | | 8 | | | | |
| 4930506C21RIK | | | | | | | | | | 3 | | |
| 4930512B01RIK | | | | | | | | | 5.5 | | | |
| 4930518I15RIK | | | | | | | | 11 | | | | |
| 4930544G11RIK | | 18 | 18 | | | | | | | | | |
| 4930544L04RIK | | | | 20 | | | | | | | | |
| 4930578G10RIK | | | | | | | | 20 | 20.5 | 16 | | 21 |
| 4930579K19RIK | | | | | 8 | | | | | | | |
| 4930581F22RIK | | | | | | | | | | | | 1 |
| 4931423N10RIK | | | | | | | | 21 | | | | |
| 4932415D10RIK | | | | | | | | 22 | 22.5 | | | |
| 4933412A08RIK | | | | | | | 1 | | | | | |
| 4933412E24RIK | | | | | | | | 0 | 0 | | | |
| 4933413J09RIK | | | | | | 16 | | 15 | | 16.5 | | |
| 4933427I04RIK | | | | | | 7 | | | | | 7 | |
| 4933431E20RIK | | | | | | | | | | | 8 | |
| 4933436C20RIK | | | | | | 21 | | | | | | |
| 5330416C01RIK | | | | | | | 8 | | | | | |
| 5430416O09RIK | | | | | | | | 12 | | | | |
| 5430435G22RIK | | | | | 3 | | | | | | 0.5 | |
| 5730522E02RIK | | 16 | | | | | | | | | | |
| 5830417I10RIK | | | | | | | | | | 13 | | |
| 5830428H23RIK | 20 | | | | | | | 3 | | | | |
| 5S_RR | | | | | | | | 22.5 | | 18 | | |
| 6330409D20RIK | 9 | | | | | | | | | | | 12 |
| 7SK | 6 | | | | | | | | 10.5 | 6 | | |
| 8430429K09RIK | | | | | | | | | 17 | | | |
| 9130017K11RIK | | | | | | 14 | | | | | | |
| 9130230L23RIK | | | | | | | | | | | | 21 |
| 9130409I23RIK | | | 10 | | | | | | | | | 7 |
| 9230104L09RIK | | | | | | | | | 20.5 | | | |
| 9230110F15RIK | | | | | | | | | | | 2.833 | |
| 9230112E08RIK | | | | | | | | | | | 6.167 | |
| 9430007A20RIK | | | | | | | | | 12 | | | |
| 9630013D21RIK | | | | | | | | | 16 | | | |
| 9830147E19RIK | 17 | | | | | | | | | | | |
| A15 | | | | | | | | 2 | 0 | | | |
| A1CF | | | | | | | | | 21 | | | |
| A25 | | | | | | | | | | 18 | | |
| A2LD1 | | | | | | | | | | 8 | | |
| A2M | | | | | | | | 20 | | | | |
| A35 | | | | | | 22 | | | | | 2.5 | |
| A38 | | | | | | | | 11.5 | 22.5 | | | |
| A40 | | | | | | 2 | | | 21 | 20 | | |
| A430078G23RIK | | | 0 | | | 0 | | 22 | 22 | 22 | | 1 |
| A4GALT | | | 10 | | | | | | 21 | | | |
| A50 | | | | | | | | 21 | | | | |
| A530053G22RIK | | | | | | | | | | | 22 | |
| A60 | | | | | | | | | | | | 22.5 |
| A730020M07RIK | | | | | | | | | | 5 | | |
| A930017M01RIK | | | | | | 8 | | | | | | |
| A930104D05RIK | | | | | | | | 9 | | | | |
| AA | | | 9 | | | | | | 9 | | | |
| AA474408 | 9 | | | | | | | 13.5 | | | | |

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Methods and Materials:

### Animal Preparation and Organ Collection

Mice were prepared as previously described (Hughes, et al., 2009, PLoS Genet., 5:e1000442). Briefly, 6-week old male C57/BL6 mice were acquired from Jackson Labs, entrained to a 12h:12h light:dark schedule for one week, then released into constant darkness. Starting at CT18 post-release, three mice were sacrificed in the darkness every 2h, for 48 hours. Specimens from the following organs were quickly excised and snap-frozen in liquid nitrogen: aorta, adrenal gland, brainstem, brown fat (anterior dorsum adipose), cerebellum, heart, hypothalamus, kidney, liver, lung, skeletal muscle (gastrocnemius) and white fat (epididymal adipose). Food and water were supplied ad libidum at all stages prior to sacrifice. All procedures were approved by the Institutional Animal Care and Use Committee.

### Microarray Data

Organ samples were homogenized in Invitrogen Trizol reagent using a Qiagen Tissuelyser. RNA was extracted using Qiagen RNeasy columns as per manufacturer's protocol, then pooled from three mice for each organ and time point. The reason for pooling was to average out both biological variance between individual animals and technical variance between individual dissections. RNA abundances were quantified using Affymetrix MoGene 1.0 ST arrays and normalized using Affymetrix Expression Console software (RMA). Probesets on the Affymetrix MoGene 1.0 ST array were cross-referenced to best-matching gene symbols using Ensembl BioMart software, then filtered for known protein-coding status. The resulting 19,788 genes formed the protein-coding background set.

### RNA-Sequencing Data

RNA samples from CT22, CT28, CT34, CT40, CT46, CT52, CT58, and CT64 were pooled for each organ, as described above (96 total pools). These RNA pools were converted into Illumina sequencing libraries using Illumina TruSeq Stranded mRNA HT Sample Preparation Kits as per manufacturer's protocol. Briefly, 1 μg of total RNA was polyA-selected, fragmented by metal-ion hydrolysis, and converted into double-stranded cDNA using Invitrogen Superscript II. The cDNA fragments were subjected to end-repair, adenylation, ligation of Illumina sequencing adapters, and PCR amplification. Libraries were pooled into groups of six and sequenced in one Illumina HiSeq 2000 lane using the 100 bp paired-end chemistry (16 lanes total). Details on alignment and quantification are included in the Supplementary Methods.

### Oscillation Detection

The JTK CYCLE (Hughes et al., J. Biol. Rhythms., 25:372-80) package for R was used, with parameters set to fit time-series data to exactly 24h periodic waveforms. Significance was bounded by q<0.05 for array data sampled at 2h and by p<0.05 for sequencing data sampled at 6h.

### Quantifying and Aligning RNA-Sequencing Data

Fastq files containing raw RNA-seq reads were aligned to the mouse genome (mm9/NCBI37) using STAR (Dobin et al., 2013, Bioinforma. Oxf. Engl., 29:15-211) (default parameters). RNA-seq quantification was performed using HTSeq®, run in stranded mode (default parameters). Protein-coding genes were quantified using the Ensembl annotation (Flicek et al., 2012, Nucleic. Acids Res., 40:D84-903). Non-coding RNAs were quantified using data from the NONCODE v3 database (Bu et al., 2012, Nucleic. Acids Res., 40:D210-2154). Quantification values were normalized using DESeq2 (Anders et al., Genome Biol., 11:R1065).

### Identifying Non-Coding RNAs Conserved Between Humans and Mice

This study began by downloading BED files listing ncRNA coordinates for humans and mice from the NONCODE v3 database. These bed files contained 33,801 human and 36,991 mouse transcripts. To prevent overlapping ncRNAs from confounding the analysis (many of these appeared to be alternative spliceforms of the same ncRNAs), all overlapping ncRNAs were merged on the same strand using the BEDTools suite (Quinlan et al., Bioinforma. Oxf. Engl., 26:841-842). This merge step resulted to 20,042 human and 27,286 mouse transcripts. By the coordinates for these merged transcripts and the UCSC Genome Browser (Meyer et al., 2013, Nucleic Acids Res., 41:D64-69), the nucleotide sequences was downloaded corresponding to each of these ncRNAs in FASTA format. Next, separate human and mouse BLAST libraries were constructed from these ncRNA sequences by running the make blastdb command with default parameters. Following this, BLAST (Altschul et al., 1990, J. Mol. Biol., 215:403-4108) was used to align the mouse ncRNA sequences against the human ncRNA BLAST library, and vice-versa. Since ncRNAs have previously been shown to have relaxed constraints on sequence conservation (Washietl et al., 2014, Genome Res., 24:616-28), blastn was run using the more permissive dc-megablast algorithm and a minimum e-value cutoff of 1E-10. These BLAST results for pairs of human and mouse ncRNAs that were each other's top BLAST hit (termed “reciprocal best hits”) were mined. Filtering for these reciprocal best hits left with 1601 human and mouse transcript pairs, termed conserved ncRNAs. Conserved ncRNAs using these relaxed BLAST parameters were found well-known, conserved ncRNAs like Xist, Tsix, Hotair, H19, and Gas5.

To assign names and annotation data to these conserved ncRNAs, BLAST was used to align their sequences to human and mouse RefSeq (Pruitt et al., 2009, Nucleic Acids Res., 37:D32-3610) transcripts. 585 of these conserved ncRNAs were mapped to protein-coding genes (i.e. RefSeq IDs beginning with NM or XM) in the sense orientation in both humans and mice. Upon visual inspection of these ncRNAs, it was found that many of these mapped along the entire length of the protein-coding transcripts. While some ncRNAs in this list might represent non-coding isoforms of these protein-coding transcripts, they were removed from further analysis as a result of conservative approach. Following the removal of these transcripts, a final list of 1016 conserved ncRNAs were left. Biotypes (defined by GENCODE (Harrow et al., 2012, Genome Res., 22:1760-177411) and Ensembl) were assigned to these transcripts using both Ensembl and manual annotation. Quantification and analysis of these transcripts was performed like all other RNA-seq transcript data.

Identifying Novel ncRNAs

Given that RNA-seq data is not limited to a specific gene annotation, novel transcripts were sought to be characterized. The study began by collecting all reads that mapped across splice junctions (i.e. reads with large gaps in their alignments). Reads falling into this class were identified by STAR during alignment and stored in files having with the SJ.out.tab extension. While this caused missing single-exon transcripts, the data came from a real, expressed transcripts if evidence of RNA splicing was found. To reduce the impact of spurious reads and noise, splice junctions were mapped by a minimum of 16 reads across entire dataset (this corresponds to 2 reads per time point in a single organ). A fairly low threshold was chosen so as not to remove junctions present in only a single organ, and those circadian transcripts expressed in a bursting patterns (like Dbp). Next, the BEDTools was used to filter out any junction mapping within 1 KB of any Ensembl or Refseq transcript, or overlapping with any NONCODE transcript. All of these steps left with 10,452 junctions from putative transcripts. All junctions within 500 bp of each other were merged to form 5,154 putative, ncRNA transcript regions. These putative transcripts were quantified and analyzed like all other RNA-seq transcripts.

### Disease-Genes, Drug Targets, and Other Data Sources

Disease-gene annotations were aggregated from the following sources: Online Mendelian Inheritance in Man (Hamosh et al., Nucleic Acids Res., 33:D514-712), Universal Protein Resource (Update on activities at the Universal Protein Resource (UniProt) in 2013, Nucleic Acids Res., 41:D43-7), Comparative Toxicogenomics Database (Davis et al., 2013, Nucleic Acids Res., 41:D1104-1414), Pharmacogenomics KnowledgeBase (Whirl-Carrillo et al., 2012, Clin. Pharmacol. Ther., 92:414-715), Literature-Derived Human Gene-Disease Network (Bundschus et al., BMC Bioinformatics, 9:207). Drug target genes were pulled from the DrugBank database (Law et al., 2014, Nucleic Acids Res., 42:D1091-109717). List of WHO essential medicines downloaded from WHO website (http://www.who.int/medicines/publications/essentialmedicines/en/,10/10/2014). MicroRNA target predictions for PTGS1 from TargetScan (Lewis et al., 2005, Cell, 120:15-20).

Tissue culture and cell maintenance. NIH3T3 cells were purchased from ATCC. These cells were maintained in growth media containing 10% FBS (Atlanta Biologicals), 1× Penicillin/Streptomycin/Glutamine (Gibco), and 1× Non-essential amino acids (NEAA; Gibco) in Dulbecco's Modified Eagle's medium (DMEM; Gibco). Cells were grown in a humidified incubator at 37° C. and 5% CO2.

### Transfections

All transfections were performed in the forward format. Briefly, cells were seeded in 6-well dishes at a density of 2.5×105 cells/well, in media containing no antibiotics (DMEM, 10% FBS, 1× Glutamine (Gibco), 1× NEAA). Cells were incubated overnight at 37° C. and 5% CO2. On the following day, cells were transfected using Opti-MEM (Gibco) and the RNAiMAX (Invitrogen) reagent, according to manufacturer's protocol. Cells were transfected with mirVana Negative Control #1, mmu-miR-22-3p mimic, or mmu-miR-22-5p mimic (Life Technologies), at a final concentration of 50 nM. Transfected cells were incubated for 72 hrs at 37° C. and 5% CO2. RNA and protein were harvested from the same well by collecting cells in ice-cold PBS, and dividing these cells suspensions into two aliquots. For each well, one aliquot was processed for protein, and the other was processed for RNA.

### Western Blot

Whole-cell protein extracts were isolated from cells using ice-cold RIPA buffer (Sigma), supplemented with Complete protease inhibitor cocktail (Roche). Protein concentrations were quantified using the DC protein assay (BioRad). 4 μg of protein was resolved on 7.5% polyacrylamide, Tris-HCL/Glycine/SDS gels (BioRad) and transferred to PVDF membranes. Membranes were blocked for 1 hr at room temperature in blocking solution (5% milk, 0.05% Tween20, 1× Tris-buffer saline), followed by overnight incubation at 4° C. with primary antibody in blocking solution. Primary antibodies used were: anti-PTGS1 (160110; Cayman Chemical), and anti-GAPDH (sc-25778; Santa Cruz). Membranes were then rinsed twice each with TBS-0.05% tween and blocking solution. Following rinses, membranes were probed with secondary antibody at room temperature for 70 min. Those membranes treated with anti-PTGS1 were incubated with anti-mouse IgG HPR-linked secondary antibodies (NA931V; GE Healthcare), while membranes treated with anti-GAPDH were incubated with anti-rabbit IgG HPR-linked secondary antibodies (NA934-1ML; GE Healthcare). Membranes were then rinsed 5 times for 10 min in TBS-0.05% tween, and then imaged using standard autoradiograph techniques after the application of Western Lightning Plus ECL (PerkinElmer) western blotting detection reagent.

### RNA Extraction and Quantitative PCR

RNA was extracted from cells using TRIzol reagent (Life Technologies) with Direct-zol RNA MiniPrep kit (Zymo research), according to manufacturer's protocol, cDNA was generated from 500 ng of RNA using the qScript cDNA Synthesis Kit (Quanta Biosciences) and qPCR was performed on the ViiA 7 Real Time PCR System (Life Technologies) using the PerfeCTa FastMix II, Low ROX reagent (Quanta Biosciences), according to the manufacturer's protocols. Relative expression quantification of the qPCR data was performed using the ΔΔCT method with the ViiA 7 analysis software v1.2 (Life Technologies). Ptgs1 (Mm00477214_m1; Life Technologies) was quantified using Gapdh (4352661; Life Technologies) as the endogenous reference.

### Example 1: Genes and Non-Coding Transcripts

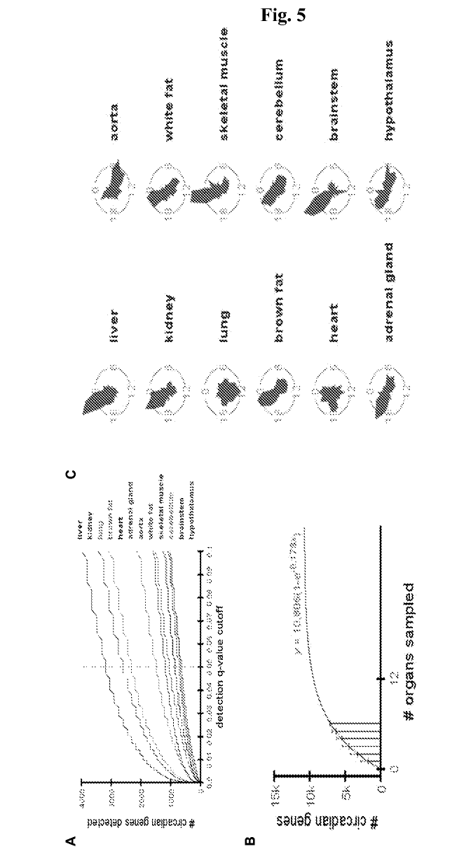

A background set of 19,788 known protein-coding mouse genes was defined and for each organ the JTK CYCLE (Hughes et al., 2010, J. Biol. Rhythms., 25:372-8011) algorithm to detect 24-hour oscillations in transcript abundance was used. For this protein-coding gene analysis, the high temporal resolution of the array data was leveraged to accurately identify circadian genes. A 5% false discovery-rate was set for detection, though the specific value of this cutoff did not affect the relative amount of rhythmic transcripts detected between organs (FIG. 5, Panel A). The base-pair level RNA-seq data was used in a complimentary fashion to identify the expressed spliceforms of these circadian genes, and for analysis of the non-coding transcriptome.

Following these analyses, it was found that liver had the most circadian genes (3,186), while hypothalamus had the fewest (642) (FIG. 1, Panel A). In fact, the three brain regions (cerebellum, brainstem and hypothalamus) had the fewest circadian genes, collectively. Due to the technical difficulty of precisely sampling brain regions, it was assumed that heterogeneous mixtures of cell types within these complex organs may express different sets of genes, or may be out of phase with each other. This transcript/phase-discrepancy within the same organ would make it difficult to accurately identify circadian genes in these brain regions. On average, 46% (s.d.=0.036%) of circadian protein-coding genes expressed multiple spliceforms detected in the RNA-seq data.

Transcript abundance for 43% of protein-coding genes oscillated in at least one organ (FIG. 1, Panel B). Only ten genes oscillated in all organs: Arntl, Dbp, Nr1d1, Nr1d2, Per1, Per2 and Per3 (core clock factors), as well as Usp2, Tsc22d3, and Tspan4. While the organs analyzed provide a broad sampling across the entire organism, there are still many more to study which may contain additional circadian genes. The average number of total circadian genes, y, detected by randomly sampling x organs was closely modeled by the exponential function y=a (1−e^{-bx}), where e is Euler's number and the coefficients a (asymptote) and b (rate of asymptotic approach) equal 10,901 and 0.123, respectively (R²>0.99; FIG. 1, Panel C). This estimate remains unchanged if we exclude the potentially noisy, heterogeneous tissues discussed above (FIG. 5, Panel B). In other words, as additional organs are sampled, without bounding to a specific theory, it is predicted that ˜10,901 mouse protein-coding genes (55% of the background set) will show circadian oscillations somewhere in the body.

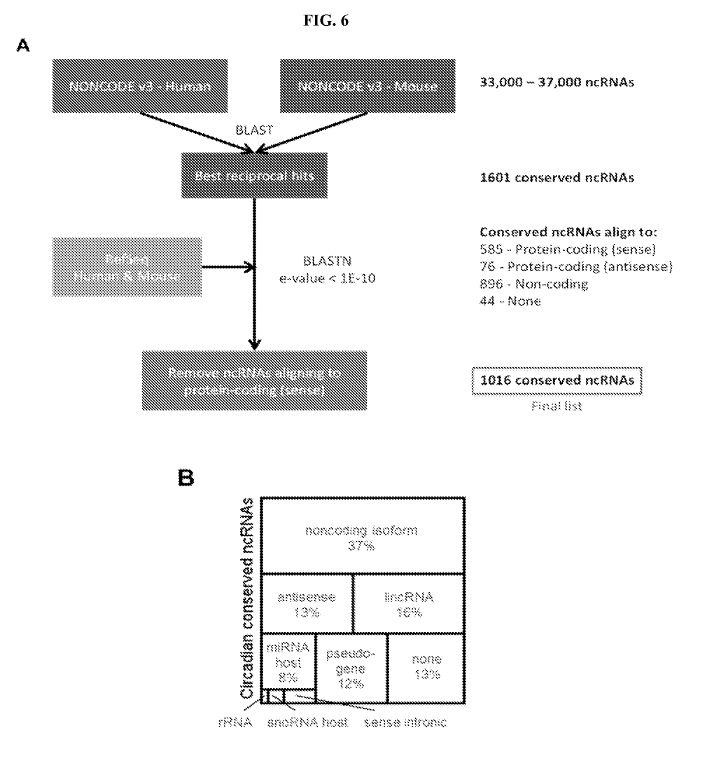

To study the non-coding transcriptome, the NONCODE was used to define a background set of 1,016 mouse-human conserved ncRNAs (FIG. 6, Panel A). It was found 32% of conserved ncRNAs oscillated (a similar proportion compared to protein-coding genes), while non-conserved ncRNAs were less likely to oscillate (FIG. 1, Panel D). This suggests the set of conserved ncRNAs may be functionally relevant. Unlike protein coding genes, no individual ncRNA oscillated in more than five organs. This is unsurprising, given that ncRNA expression is known to be organ-specific (Washietl et al., 2014, Genome Res., 24:616-28). It was also found 712 of 5,154 unannotated, spliced non-coding transcripts had rhythmic expression. 80% of these aligned to the human genome (BLASTN, E<10⁻¹⁰, sequence identity >70%), indicating they are conserved between human and mouse.

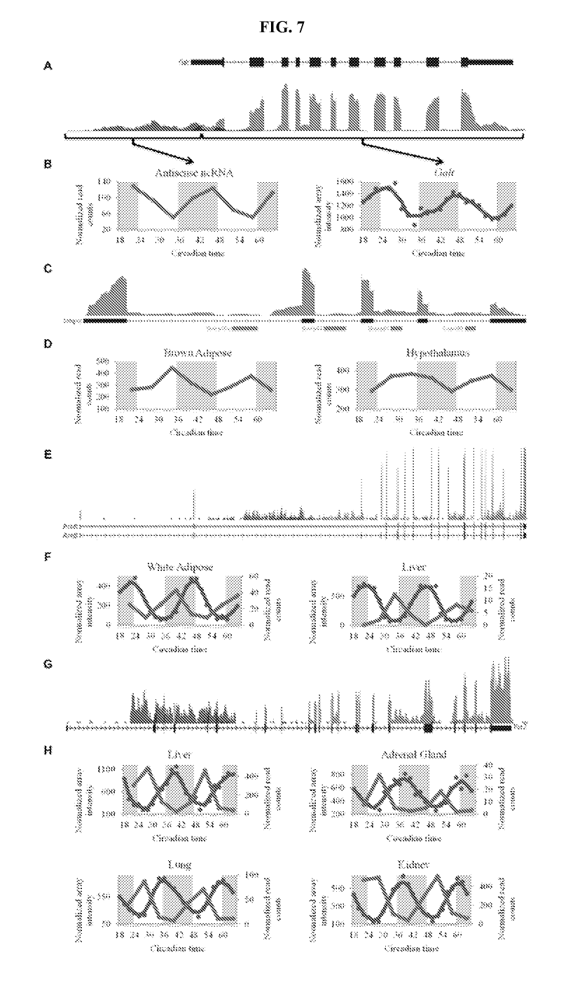

These conserved, clock-regulated ncRNAs covered a diverse set of functional classes (FIG. 6, Panel B). 30 of them were antisense to protein-coding genes, half of which were themselves circadian. There was no general phase relationship between sense and antisense ncRNAs. For example, in the liver, both Galt (galactose-1-phosphate uridylyltransferase) and an overlapping antisense ncRNA oscillated in phase with each other (FIG. 7, Panels A-D). Host genes for 39 circadian miRNAs and four snoRNA host genes were identified: Cbwd1, Snhg7, Snhg11, and Snhg12. As snoRNAs were recently shown to have light-driven oscillations in Drosophilabrains (Hughes et al., 2012, Genome Res., 22:1266-1281), these findings provide further evidence of the clock's potential to influence ribosome biogenesis (Jouffe et al., 2013, PLoS Biol., 11:e100145515). It was also found 74 conserved lincRNAs with circadian oscillations, the majority of which were Riken transcripts with no known function. Finally, it was also found 1979 genes with un-annotated antisense transcripts, 187 of which showed sense and antisense oscillations in the same organ. Of these, 43 antisense transcripts oscillated at least eight hours out of phase with their sense transcripts. Genes with antiphase, antisense oscillators included Arntl and Per2 (FIG. 7, Panels E-H). A known Per2 antisense transcript (Koike et al., 2012, Science 338:349-3549; Vollmers et al., 2012, Cell Metab., 16:833-845) oscillated in 4 organs, the most of any antisense transcript, providing further evidence of its functional relevance. Taken together, the data reflect a vast and diverse set of transcripts regulated by the clock at the organism level.

Data regarding circadian oscillations, including coding and non-coding genes, are available via the World Wide Web (www) bioinf.itmat.upenn.edu/circa, a subset of which is summarized in Table 2, supra.

### Example 2: Gene Parameters

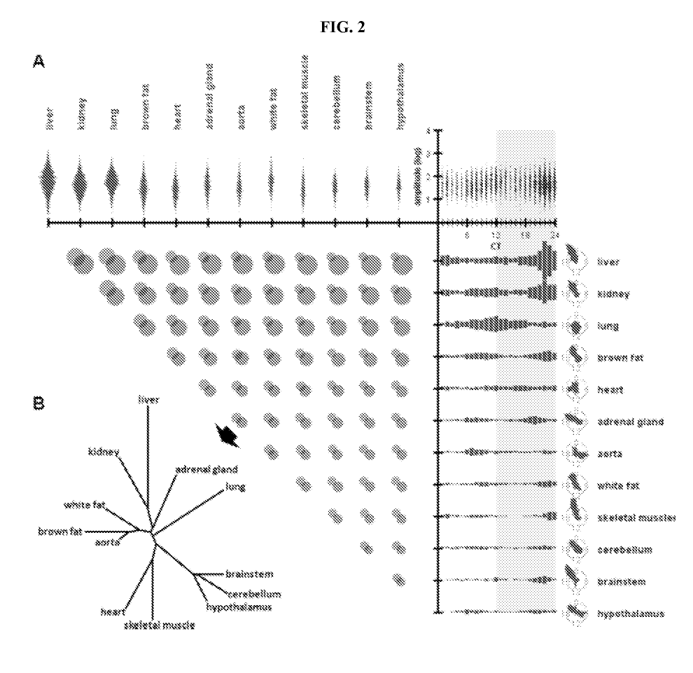

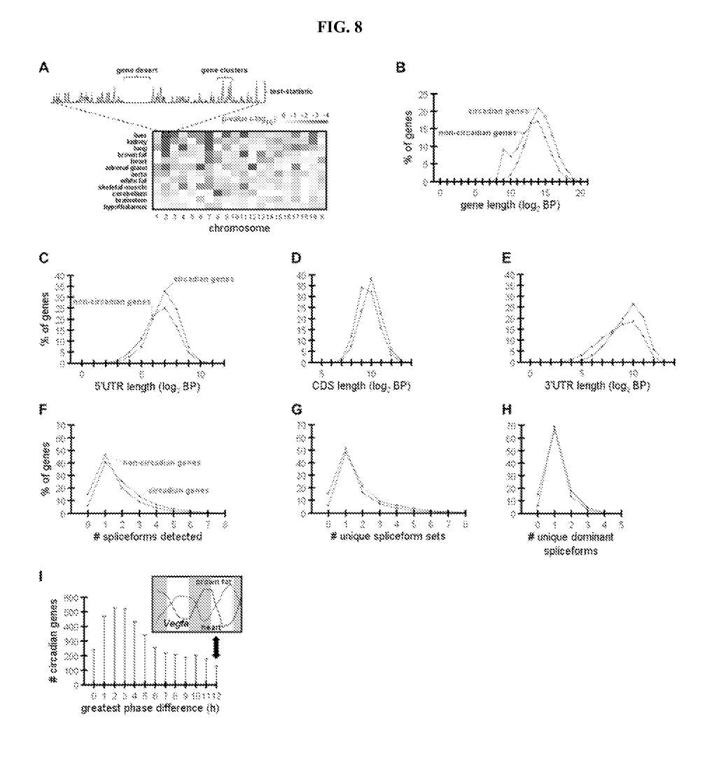

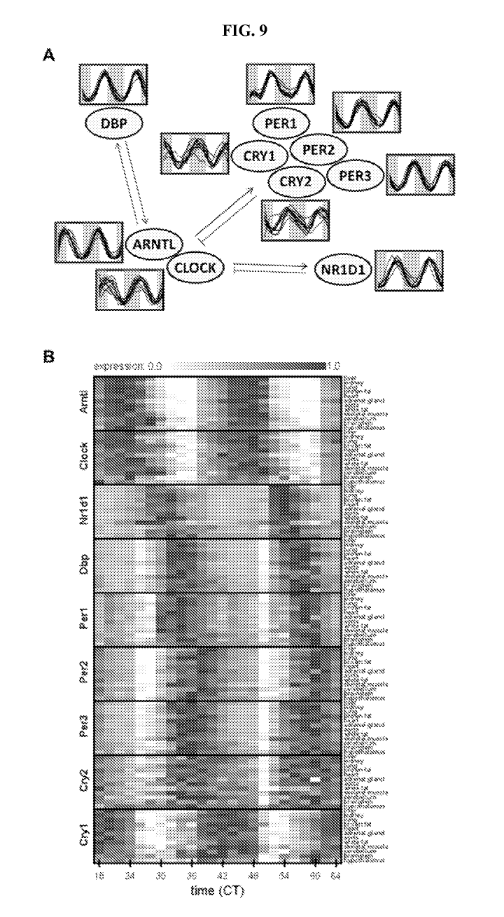

The finding from previous multi-organ studies agreed with the data generated above that the vast majority of circadian gene expression is organ-specific (Panda et al., 2002, Cell, 109:307-20: Storch et al., 2002, Nature, 417:78-837), with little overlap of circadian-gene identity between organs (FIG. 2, Panel A). In most organs, expression of circadian genes peaked in the hours preceding subjective dusk or dawn, often in a bi-modal fashion. Heart and lung were notable exceptions, with phase distributions that diverged substantially from other organs. Moreover, those circadian genes expression peaks clustered around subjective dusk or dawn also tended to have the highest average oscillation amplitude, compared to genes with expression peaks at other times of day. Taken together, these data suggest that the body may experience daily “rush hours” of transcription at these critical times. Using the average phase difference between any two organs' shared circadian genes as a distance metric, an ontogenic tree that recovered recognizable organ lineage was constructed (FIG. 2, Panel B) (Edgar et al., 2013, PloS One 8:e66629). Thus, developmentally related organs tended to share genes that oscillate synchronously. Having examined their oscillation patterns, genomic characteristics common to rhythmically-expressed genes was analyzed. Circadian genes clustered physically in the genome (FIG. 2, Panel C). Their lengths tended to be longer than non-rhythmic genes (Mann-Whitney U test p<<10⁻¹⁵; FIG. 2, Panel D). This trend was maintained at the level of 5′UTR, CDS, and 3′UTR (FIG. 8, Panels A-C). These results are in agreement with previous findings about oscillating liver transcripts (Wu et al., 2012, PloS One, 7:e46961). By using gapped, junction-spanning reads to discriminate between expressed spliceforms, it was found that circadian genes had more spliceforms than non-circadian genes (Mann-Whitney U test p<<10-15; FIG. 8, Panels D-F). Furthermore, it was found that the spliceforms expressed by circadian genes, including the identity of the dominant spliceform, tended to differ across organs more than for non-circadian genes. These findings are consistent with the idea that the circadian genes have more regulatory capacity than noncircadian genes. Remarkably, 1,400 genes were phase-shifted with respect to themselves by at least six hours between two organs, with 131 genes completely anti-phased (FIG. 2, Panel E). For example, at dusk, the transcript levels of Vegfa (vascular endothelial growth factor) peaked in brown fat but reached a nadir in heart. Such drastic phase-discrepancies of individual genes between organs have not been reported. The mechanisms for these phenomena are unclear, as the genes did not share any obvious transcription-factor or miRNA-binding motifs. The core clock genes oscillated synchronously, with the peak phases of a given gene falling within 3 hours of each other across all organs (FIG. 9). Several core clock genes did show 1-2 hour phase advances and delays in skeletal muscle and cerebellum, respectively, when compared to other organs. However, these cases were in the minority, and given the limitations in our ability to precisely resolve small (<2 hour) phase differences from data with a 2-hour resolution, their significance remains unclear. This finding indicates that the anti-phased patterns observed in genes like Vegfa are not due to phase-differences between the core clocks of each organ. Rather, these phenomena are due to additional, organ-specific levels of timing regulation positioned between the core clock and these output genes.

### Example 3: Pathways

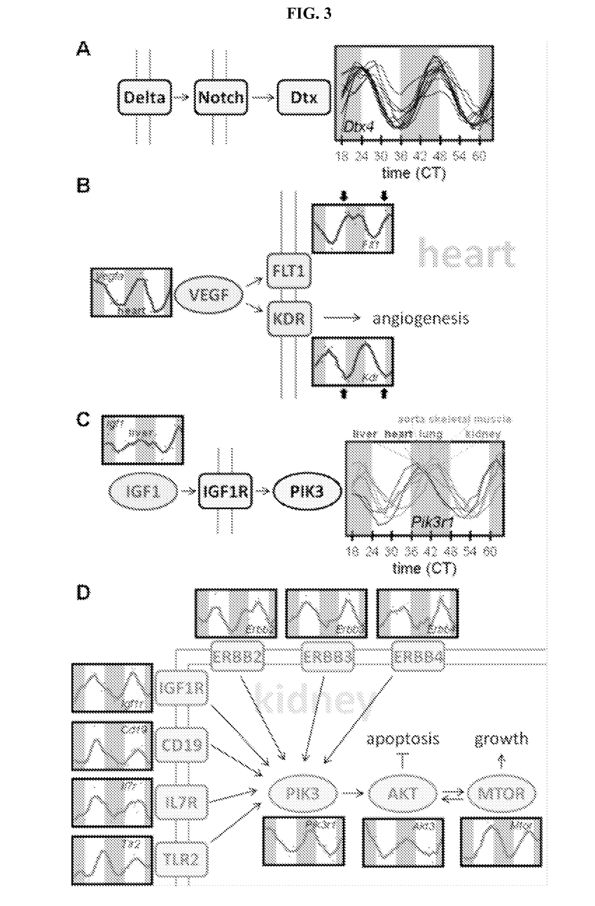

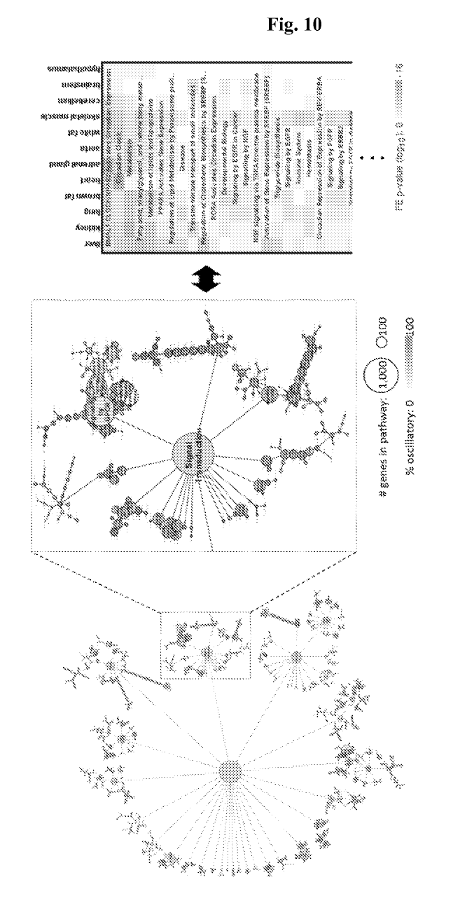

Given the high temporal and spatial resolution of the study, ways in which time and space influenced biological pathways was examined. The Reactome database (Matthews et al., 2009, Nucleic Acids Res., 37:D619-2218) was used as a basis for pathway network and found many pathways enriched for circadian genes both within and across organs (FIG. 10). Several genes oscillated synchronously across all organs, like the core clock genes. For example, Dtx4, a Notch pathway E3 ubiquitin ligase, oscillated in phase with Arntl in all organs (FIG. 3, Panel A). It was also noted that genes with “opposite” functions (e.g., activators vs. repressors) often had opposite phases. For example, members of the initial vascular endothelial growth factor (VEGF) signaling cascade oscillated in the heart (FIG. 3, Panel B). These included the primary circulating ligand, Vegfa, and its two principle membrane-bound receptors, Flt1 and Kdr. This cascade regulates angiogenesis, with critical roles in development, cancer and diabetes (Folkman et al., 2007, Nat. Rev. Drug Discov., 6:273-86). At dusk, expression of Vegfa and Kdr in the heart was low, while Flt1 was high. KDR is thought to mediate most of the known cellular responses to VEGF-signaling, while FLT1 is thought to be a decoy receptor (Zygmunt et al., 2011, Dev. Cell, 21:301-1420). Thus, the rhythmic timing of these receptors appears to reflect function, in that FLT1 (the decoy) is present when KDR is not and vice versa.

While members of some systemic pathways, such as the core circadian clock, were expressed in phase across organs, many were not. For instance, expression of the insulin-like growth factor Igf1 oscillated in the liver, peaking in the early subjective night (FIG. 3, Panel C). Since the liver produces nearly all of the circulating IGF1 (Sjögren et al., 1999, Proc. Natl. Acad. Sci. USA, 96:7088-92), IGF-signaling throughout the entire body is likely under clock influence. IGF1 is one of the most potent natural activators of the PIK3-AKT-MTOR pathway, which stimulates growth, inhibits apoptosis, and has a well-known role in cancer (Franke et al., 2008, Oncogene, 27:6473-6488). However, peak expression of Pik3r1, which encodes the regulatory subunit for PIK3, did not occur at the same time across all organs. Instead, there was a steady progression throughout the night spanning nearly ten hours, as it peaked first in liver, then heart, followed by aorta, lung, skeletal muscle, and finally in kidney (FIG. 3, Panel C). Since the core clocks of these organs were in phase with each other, as mentioned earlier, the timing differences of Pik3r1 are most likely driven by some unknown, organ-specific mechanism situated between the core clock pathway and Pik3r1. Some pathways known to function systemically were only rhythmic in a single organ. For example, IGF1's principal membrane-bound receptor, IGF1R, is present in numerous tissues. However, Igf1r expression oscillated only in kidney. In addition to Igf1r, many other membrane-bound receptors that activate the PIK3-AKT-MTOR cascade were also rhythmically expressed only in kidney (FIG. 3, Panel D). These included Erbb2, Erbb3, and Erbb4 (tyrosine kinase receptors), T1r2 (toll-like receptor), Cd19 (antigen receptor), and I17r (cytokine/interleukin receptor). These receptors were all notably in phase with one another, all having peak expression in the subjective mid-day. Thus, there is kidney-specific clock regulation of PIK3-AKT-MTOR signaling, that is distinct from and in addition to the already clock-regulated IGF1 signal coming from the liver.

### Example 4: Drug Targets and Disease

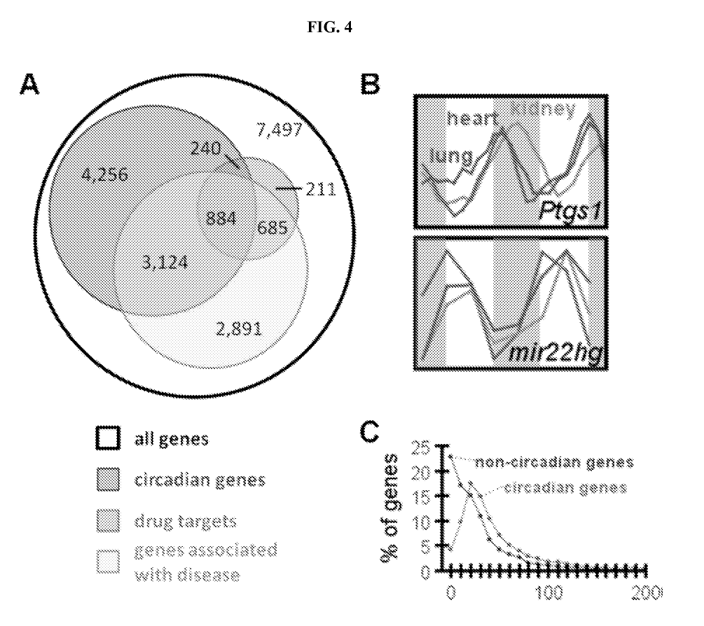

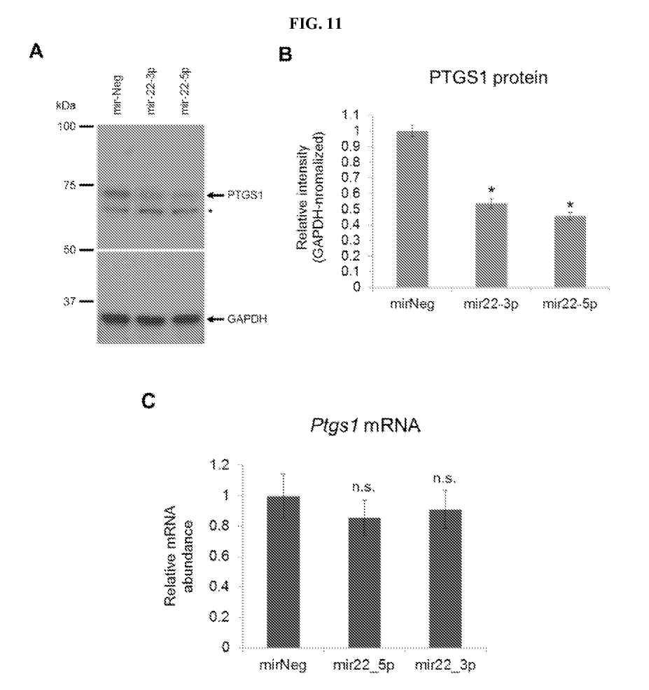

Timing is an important but underappreciated factor in drug efficacy. For example, short half-life statins work best when taken before bedtime, as cholesterol synthesis peaks when we sleep (Miettinen et al., J. Lipid. Res., 23:466-7323). The relationship between a target for a marketed drug and a circadian gene was examined. Notably, 56 of the top 100 best-selling drugs in the United States, including all top seven, target the product of a circadian gene (Table 1). Nearly half of these drugs have half-lives less than 6 hours (Table 1), suggesting the potential impact time-of-administration could have on their action. Most of these drugs are not dosed with consideration for body time and circadian rhythms. Furthermore, 119 of the World Health Organization's list of essential medicines target a circadian gene, including many of the most common and well known targets (Table 2). For example, Ptgs1 (cyclooxygenase-1, alias Cox1), the primary target of low dose aspirin therapy used in secondary prevention of heart attacks (Antithrombotic Trialists' Collaboration, 2002, BMJ, 324:71-8624), oscillated in the heart, lung, and kidney (FIG. 4, Panel B). Given that aspirin has a short half-life and that heart attacks have a circadian rhythm (Curtis et al., 2006, Ann. Med., 38:552-9.2), dosing aspirin at an optimal time of the day has great potential. Consistent with this observation, clinical reports have suggested night-time administration of low dose aspirin may be important for its cardio-protective effects (Hermida et al., 2005, Hypertension, 46:1060-8). The data suggest a mechanism for Ptgs1's circadian regulation as well. Mir22 is a micro-RNA predicted to target PTGS1, and its host transcript oscillated anti-phase to Ptgs1 in the heart, lung, and kidney. This miRNA may therefore regulate Ptgs1 function. To test this hypothesis, mir22 mimics were transfected into NIH3T3 cells and knocked down endogenous quantities of PTGS1 protein by 50% (FIG. 11). A slight, non-significant decrease was observed in Ptgs1 mRNA levels in these same samples. These data suggest that mir22 operates on PTGS1 predominantly at the posttranscriptional level, though it remains possible that Ptgs1 is a transcriptional target of the clock through other mechanisms. Beyond drug targets, circadian genes were also enriched among disease-associated genes (Pearson's Chi-square test, p<<10⁻¹⁵; FIG. 4, Panel A), and were highly studied in biomedical research. They received significantly more PubMed citations than non-oscillating genes (Mann-Whitney U test, p<<10⁻¹⁵; FIG. 4, Panel C). Furthermore, oscillating genes were also associated with nearly every major disease funded by National Institutes of Health at significantly higher rates than expected by chance (FIG. 4, Panel D). Cancer, diabetes mellitus type 2, Alzheimer's disease, schizophrenia, Down's syndrome, obesity, and coronary artery disease were most strongly associated with circadian genes. For example, many of these oscillating genes are involved in neurodegeneration, including Fus, Tdp43, alpha synuclein, gamma synuclein, Atxn1, Atxn2, Atxn3, Atxn7, Atxn10, Psen1, and Psen2. These genes are mutated in frontotemporal dementia, ALS, Parkinson's disease, spinocerebellar ataxia, and Alzheimer's disease. They were predominantly rhythmic outside of the brain in peripheral tissues (Psen2 had nearly four-fold amplitude in liver and peaked at subjective day, when mice are going to sleep). Without bounding to a specific theory, it was speculated that promoters for these genes may have evolved sensitivity to global changes in redox state, which varies between day and night (Musiek, et al., 2013, J. Clin. Invest., 123:5389-400). Lending credence to the association between clocks and neurodegeneration are two clinical observations: many patients with neurodegeneration-linked dementia display ‘sundowning’ (behavioral problems in the early evening), and most patients with neurodegeneration eventually develop circadian sleep disorders (Hastings et al., 2013, Curr. Opin. Neurobiol., 23:880-73).

### Example 5: Methods for Designing a Formulation

This example generally describes methods for designing a formulation for treating one or more diseases, conditions, or disorders associated with genes that are expressed with circadian rhythms (i.e., genes that oscillate with circadian rhythm). The formulation has regulated release of at least one therapeutic compound such that the compound's release coincides with peak or trough expression of one or more of the compound's target genes and in at least one tissue type.

Initially, a disorder, as well as the therapeutic compounds capable of treating the disorder, are identified. Examples of both disorders and therapeutic compounds are listed in Table 1, supra. Next, target gene(s) for the therapeutic compounds are ascertained. Examples of target gene(s) for various therapeutic compounds are also listed in Table 1. Likewise, the half-lives of exemplary therapeutic compounds are listed in Table 1.

Next, circadian oscillations in transcript expression (including peak expression) for the target genes in specific tissue types are determined. Data regarding circadian oscillations, including coding and non-coding genes, are available via the World Wide Web (www) bioinf.itmat.upenn.edu/circa, a subset of which is summarized in Table 2, supra.

Using the information provided in Tables 1 and 2 as well as known methods well known in the art for making appropriate immediate release and/or time-releases formulations (see, e.g., “Remington: The Science and Practice of Pharmacy” 22nd edition, Allen, Loyd V., Jr. editor, Pharmaceutical Press, Hampshire, UK (2012), which is herein incorporated by reference in its entirety), suitable formulation(s) can be devised that will be useful in treating disease(s), condition(s), or disorder(s) associated with genes that are expressed with circadian rhythms.

When a therapeutic compound has one target gene in one tissue, the formulation is designed so that release (after ingestion of the formulation) of the therapeutic compound coincides with peak or trough expression of the target gene in the target tissue. Consideration of the compound's half-life can also be made such that the compound's release period and plasma levels coincide with expression period of the target gene. For example, once release has begun, a release period may be greatly-extended for a compound having a short half-life so that the compound's activity persists. On the other hand, once release has begun, a release period for the compound may be immediate or shortly-extended for a compound having a long half-life.

Likewise, consideration of the target gene's expression period can be made when designing the formulation to ensure coincidental release of the compound with a substantial fraction of the gene's expression. For example, if a target gene is expressed over a long period, then a release period of the compound (once release has begun) could be extended. On the other hand, a release period of the compound (once release has begun) may be immediate or shortly-extended for a target gene with a short expression period.

In some cases, it may be advantageous for the formulation to release the compound in two (or more) portions such that formulation is designed to initially release a first portion of the compound and later release a second portion. This would be advantageous, for example, when the compound has a short half-life and/or the target gene has a long expression period.

A given therapeutic compound may have more than one target gene in one tissue. If the expression periods of the more than one target genes do not precisely coincide, it may be necessary to design a formulation to release the compound in two (or more) portions, with a first portion acting upon the earlier-expressed target gene and a second portion acting at the later-expressed target gene such that the formulation is designed to release a first portion of the compound before releasing a second portion. Again, as described above, consideration of the compound's half-life and/or the lengths of the target genes' expression periods can be made when designing such formulation(s).

Other therapeutic compounds may have a target gene that is differentially expressed in more than one tissue type. If the expression of the target gene do not precisely coincide between tissue types, it may be necessary to design the formulation to release the compound in two (or more) portions, with a first portion acting at the tissue type having earlier-expression of the target gene and a second portion acting at the tissue type having the later-expressed target gene. Here, the formulation is designed to release a first portion of the compound prior to releasing a second portion. Again, as described above, consideration of the compound's half-life and/or the lengths of the target genes' expression periods can be made when designing such formulation(s).

Some therapeutic compound(s) may have two (or more) target genes that are differentially expressed in more than one tissue type. If the expression periods of the target genes do not precisely coincide between tissue types, it may be necessary to design the formulation to release the compound in two (or more) portions, with a first portion affecting the target gene having earlier-expression and a second portion affecting the later-expressed target gene such that the formulation is designed to release a first portion of the compound before releasing a second portion. Again, as described above, consideration of the compound's half-life and/or the lengths of the target genes' expression periods can be made when designing such formulation(s).

Additionally, formulation(s) may be designed to include more than one therapeutic compound. The more than one therapeutic compound may have two (or more) target genes that are differently expressed, in time and/or in tissue types, such that it may be necessary to design the formulation to release the compounds sequentially with a first-released compound affecting the earlier-expressed target gene and a second-released compound affecting the later-expressed target gene. Again, as described above, consideration of the compounds' half-lives and/or the lengths of the target genes' expression periods can be made when designing such formulation(s).

Formulations may also be designed such that one therapeutic compound is released coincidental with peak or trough expression of its target gene and a second therapeutic compound is released at times that may be independent of its target gene's peak or trough expression. In such formulations, the second therapeutic compound may have effects (intended or side effects) that can be minimized by controlling the time of the compound's release. For example, a compound that has a stimulatory effect should be released when a subject is awake rather than when the subject is trying to sleep, and a compound that has a diuretic activity should likewise be released when a subject is awake. On the other hand, a compound that is soporific should not be released with the subject is awake. Additionally, release of one or more compounds may be delayed to avoid activity of an enzyme that metabolizes one or more of the compounds.

Formulations can also be designed including more than two (e.g., three, four, five, or more) therapeutic compounds. In such formulations, each therapeutic compound may have a distinct target gene or there may be overlap in target genes and/or each therapeutic compound may have a target gene expressed in a distinct tissue type or there may be overlap in tissue types. Moreover, target gene may be expressed coincidentally in each tissue type or its expression may differ between tissue types. Again, as described above, for formulations containing more than two therapeutic compounds, consideration of the compounds' half-live and/or the lengths of the target genes' expression periods can be made when designing such formulation(s).

### Example 6: Methods for Designing a Formulation to Induce Dipping in Non-Dippers Containing an Angiotensin Receptor Blocker (ARB) Plus a Beta Blocker or an Acetylcholinesterase (ACE) Inhibitor Plus a Beta Blocker

“Dipping” is defined as a 10% or more drop in nighttime blood pressure relative to daytime blood pressure. A night time dip in blood pressure is normal and desirable, and the absence of a night time dip is associated with poorer health outcomes, including increased mortality. Additionally, nocturnal hypertension is associated with end organ damage.

Worldwide, there are 300-400 million non-dippers, roughly 10% of which live in the U.S., Europe, and Japan, and these non-dippers would benefit from a treatment that induces a dip in blood pressure.

Taking an angiotensin receptor blocker (ARB) or an acetylcholinesterase (ACE) inhibitor before bedtime is known to cause a drop in blood pressure. In a trial of bedtime administered Valsartan (an ARB), a 10 mmHg better result (bedtime, −21/−14, awakening, −13/−8, net 8 mmHg/6 mmHg) than the awakening group was observed. However, these results are less than the 10% drop in blood pressure required to be considered a dip. Thus, current treatment methods are insufficient to induce a dip in non-dippers.

To address this insufficiency, a formulation is designed that combines an ARB (e.g., Valsartan and Losartan) and a beta blocker (e.g., Metoprolol and Timolol) or an ACE inhibitor (e.g., Enalapril and Ramipril) with a beta blocker (e.g., Metoprolol and Timolol) to improve blood pressure dip in non-dippers.

As shown in Table 1, the target gene for Valsartan and Losartan is Agtr1a (also known as AGTR1) and as shown in Table 2, peak expression of Agtr1a in heart and kidney tissue type (tissues relevant to blood pressure dipping) occurs at circadian time 6 and its period extends for 12 hours. The minimum reported half-lives of Valsartan and Losartan are each one hour (see Table 1). Therefore, to effectively target peak expression of Agtr1a in heart and kidney, the formulation should be designed to initially release Valsartan or Losartan 2 hours after an at-bedtime administration and release should continue for 12 hours.

As shown in Table 1, the target gene for Enalapril and Ramipril is Ace, and as shown in Table 2, peak expression of Ace in lung and heart tissue types (tissues relevant to blood pressure dipping) occurs at circadian time 12 and its period extends for 12 hours. The minimum reported half-lives of Enalapril and Ramipril are each 2 hours (see Table 1). Therefore, to effectively target peak expression of Ace in heart and lung, the formulation should be designed to initially release Enalapril and Ramipril 8 hours after an at-bedtime administration and release should continue for 12 hours.

Additionally, as shown in Table 1, the target genes for Metoprolol or Timolol is Adrb1 and Adrb2, and as shown in Table 2, peak expression of Adrb1 in the lung tissue type (tissue relevant to blood pressure dipping) occurs at circadian time 6 and its period extends for 12 hours while peak expression of Adrb2 in lung and skeletal muscle tissue types (tissues relevant to blood pressure dipping) occurs at circadian time 12 and its period extends for 12 hours. The minimum reported half-life of Metoprolol is three hours (see Table 1). Therefore, to effectively target peak expression of Adrb1 and Adrb2 in the lung and skeletal muscle, the formulation should be designed to initially release Metoprolol 2 hours after an at-bedtime administration and release should continue for 12 hours.

Specific features of suitable formulations which allow extended-release or delayed-release of Valsartan/Losartan and Metoprolol or Enalapril and Ramipril and Metoprolol are known or can readily be ascertained by a skilled artisan in the field of pharmacology and can be found in a tome relevant to this field, see, e.g., “Remington: The Science and Practice of Pharmacy” 22nd edition, Allen, Loyd V., Jr. editor, Pharmaceutical Press, Hampshire, UK (2012).

### Example 7: Methods for Designing a Formulation Containing and Angiotensin Receptor Blocker Plus an Extended-Release or Delayed-Release Diuretic

Hypertension is often treated using therapies that include more than one active agent. For example, a commonly-used hypertension therapeutic is Diovan HCT® (Novartis, Basel, CH), which is a combination of an ARB (Valsartan) and a diuretic (hydrocholorthiazide, “HCT”). However, treatment with Diovan HCT® is problematic. While there is evidence that ARBs work better at night, the side effects of a diuretic, i.e., frequent urination, make a night-time release of the diuretic from Diovan HCT® undesirable. Instead, it would be better for the ARB to work at night and the diuretic work during the day. Thus, there is a need for a single-dose formulation that includes night-time release of an ARB and a daytime release of a diuretic.

To address this need, a suitable formulation is designed that combines an ARB (e.g., Valsartan and Losartan) and a diuretic (e.g., hydrocholorthiazide) to provide night-time release of the ARB and daytime release of the diuretic.

As shown in Table 1, the target gene for Valsartan and Losartan is Agtr1a (also known as AGTR1) and as shown in Table 2, peak expression of Agtr1a in heart and lung tissue type occurs at circadian time 6 and its period extends for 12 hours. The minimum reported half-lives of Valsartan and Losartan are each one hour (see Table 1). Therefore, to effectively target peak expression of Agtr1a in heart and lung, the formulation should be designed to initially release Valsartan or Losartan 2 hours after an at-bedtime administration and release should continue for 12 hours.

Likewise, as shown in Table 1, the target genes for hydrocholorthiazide are Car4, Cart, Car12, Car9 (also known as Ca4, Ca2, Ca12, and Ca 9, respectively), and Slc12a2 and their peak expressions are at circadian times 6 to 12. Because hydrocholorthiazide is a diuretic, it is preferable to have it active when a subject is awake, when frequent urination is less troublesome. Therefore, the formulation is designed such that the hydrocholorthiazide is released independent of its target genes peak expressions. Specifically, the formulation is designed to initially release hydrocholorthiazide six to eight hours following an at-bedtime administration. Hydrocholorthiazide has a half-life of 5.6 hours (see Table 1). Therefore, the formulation can immediately release its hydrocholorthiazide or its release can continue for 12 hours using extended-release formulations, delayed-release formulations, or combination thereof.

Specific features of formulations which allow extended-release or delayed-release of Valsartan/Losartan and hydrocholorthiazide are known or can readily be ascertained by a skilled artisan in the field of pharmacology.

### Example 8: Methods for Designing a Formulation Containing an Extended-Release or Delayed-Release Fibrate

Fibrates are a class of drugs used to treat hyperlipidemia and hypertriglyceridemia. They act by activation of PPARs, principally the target gene PPARα in the liver. Fibrates are typically taken multiple times per day, usually with meals. For example, Bezafibrate is taken three times per day at 200 mg and Gemfibrozil is taken twice per day at 600 mg.

However, as shown in Table 2, PPARα exhibits a pronounced circadian rhythm, which peaks in the middle of the night. Additionally, lipoprotein lipase, a target of fibrates, also exhibits a nighttime cycling of activity. Because the target genes of fibrates have peak expression at night, it may be unnecessary to administer it during the day. Thus, a single-dose formulation which directs release of a fibrate during peak expression of PPARα is desirable.

As shown in Table 2, peak expression of PPARα in the liver occurs at circadian time 8 and its period extends for 8 hours, and as shown in Table 1, the minimum reported half-lives of Bezafibrate and Gemfibrozil are one hour and one and a half hours, respectively. Therefore, in order to effectively target peak expression of PPARα in liver, the formulation should be designed to initially release Bezafibrate or Gemfibrozil 4 hours after an at-bedtime administration and release should continue for 8 hours.

Specific features of formulations which would allow extended-release or delayed-release of Bezafibrate or Gemfibrozil are known or can readily be ascertained by a skilled artisan in the field of pharmacology.

### Example 9: Methods for Designing a Formulation Containing a Short Acting Fibrate and a Short Acting Statin

Fibrates and statins are often taken together to treat dyslipidemia. There is clinical evidence that short acting statins work better when taken at night, and, as described in Example 5, supra, fibrates may also work better at night. Despite this, current recommendations suggest that the two classes of medicines be taken separately, with fibrates taken in the morning and statins taken at night, possibly because certain commonly-prescribed fibrates (e.g., Gemfibrozil) and statins (e.g., Simvastatin) are metabolized by the same enzymes, Cyp3a4. Consequently, when taking a fibrates in combination with a statin, levels of statins can remain high, and myopathies and rhabdomyolysis (breakdown of muscle fibers) can occur more frequently. Thus, a single-dose formulation that overcomes this drug interaction is warranted. For example, a formulation containing a short acting fibrate (i.e., Gemfibrozil), whose target gene's expression peaks approximately four hours earlier at night than the target gene of a short acting hydrophilic statin (i.e., Fluvastatin).

Peak expression of Gemfibrozil's target gene, PPARα, occurs at circadian time 8 in the liver with its expression extending for 8 hours, and Gemfibrozil's half-life is one and a half hours. Therefore, to effectively target peak expression of PPARα in liver, a suitable formulation to treat dyslipidemia should be designed to initially release Gemfibrozil 2 hours after an at-bedtime administration and release should continue for 6 hours.

As shown in Table 1, the target gene for Fluvastatin in the liver is Hmgcr. Peak expression of Hmgcr occurs four hours following peak expression of PPARα. As shown in Table 2, Hmgcr expression period extends for 12 hours. Likewise, as shown in Table 1, the half-life of Fluvastatin is three hours. Therefore, to effectively target peak expression of Hmgcr in liver and avoid interactions Gemfibrozil, the formulation should be designed to initially release Fluvastatin 6 hours after an at-bedtime administration and release should continue for 12 hours.

Specific features of formulations which allow extended-release or delayed-release of Gemfibrozil and Fluvastatin are known or can readily be ascertained by a skilled artisan in the field of pharmacology.

### Example 10: Methods for Designing a Formulation Containing Delayed-Release, Immediately-Released Niacin

Niacin and extended-release formulations of niacin, e.g., Niaspan, are often taken to treat dyslipidemia. Niacin is typically given at high dosage, 500 mg (normal dietary intake is 15 mg for adults), to achieve its lipid lower effects. At these concentrations, flushing and liver function abnormalities can occur. In a Niaspan trial, half of patients taking 1000 mg dosage withdrew before the study was completed.

However, as shown in Table 2, Niacr1, a receptor for niacin as shown in Table 1, exhibit a pronounced circadian rhythm, which peaks after bedtime. Because the target genes of niacin have peak expression at night, it may be unnecessary to administer it during the day and thereby avoid niacin's side effects (e.g., flushing) during waking hours. Thus, a single-dose formulation which directs release of niacin after bedtime and/or at peak expression of Niacr1 is desirable; in particular, a delayed release, rather than extended-release, formulation of niacin, which could be taken at a reduced dosage (<500 mg).

As shown in Table 2, peak expression of Niacr1 in the adrenal tissues occurs at circadian time 4 and its period extends for 8 hours. Therefore, in order to effectively target peak expression of Niacr1 in the adrenal, the formulation should be designed to initially release niacin about 4 hours after an at-bedtime administration and immediate-released at that time.

Specific features of formulations that would allow delayed-release of niacin are known or can readily be ascertained by a skilled artisan in the field of pharmacology.

### Example 11: Methods for Designing a Formulation Containing Immediately-Released Niacin and a Short Acting Statin

Niacin and extended-release niacin formulations are often taken with a statin to treat dyslipidemia. As noted in Example 7, the high doses required to achieve niacin's lipid lower effects cause unwanted side effects. Also, as mentioned above, Niacr1 (also known as HCAR2) exhibit a pronounced circadian rhythm, which peaks after bedtime. Because the target genes of niacin have peak expression at night, administer niacin at bedtime could avoid niacin's side effects (e.g., flushing) during waking hours. As shown in Table 1, the half-life of niacin is 0.33 hours.

As shown in Table 1, the target gene for Cerivastatin, Fluvastatin and Simvastatin (three statins with half-lives of less than three hours) in the liver is Hmgcr. Peak expression of Hmgcr occurs in the liver at circadian time 12. Thus, administering a statin at bedtime and releasing the statin thereafter will allow the statin to act when its target's expression has peaked. Moreover, peak expression of Niacr1 occurs in the adrenal tissue at circadian time 4, which is 8 hours before peak expression of Hmgcr.

Therefore, to effectively target peak expression of Hmgcr in liver and avoid interactions niacin, a formulation should be designed to initially release niacin about 2 hours after an at-bedtime administration and the statin should be released 6 hours after administration.

Specific features of formulations which would allow delayed-release of niacin and/or a statin are known or can readily be ascertained by a skilled artisan in the field of pharmacology.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A formulation providing coordinated release of a therapeutic compound selected from Table 1 wherein release of the therapeutic compound from the formulation coincides with peak or trough expression of at least one target gene of the therapeutic compound.

2. The formulation of claim 1, wherein the at least one target gene of the therapeutic compound is PPARα or niacin receptor, Niacrl.

3. (canceled)

4. The formulation of claim 1, wherein the therapeutic compound is niacin.

5. The formulation of claim 4, wherein the niacin is released zero to six hours after contact with a solution having a pH of between 1 and 5 and a temperature of between 35 and 42° C.

6. The formulation of claim 1, wherein the therapeutic compound is dosed within one hour of a final meal before bedtime.

7. The formulation of claim 1, wherein the formulation provides coordinated release of a first portion of the therapeutic compound and a second portion of the therapeutic compound such that release of the first portion of the therapeutic compound coincides with peak or trough expression of the at least one target gene and release of the second portion of the therapeutic compound occurs after peak or trough expression of the at least one target gene.

8. The formulation of claim 7, wherein release of the second portion of the therapeutic compound occurs either prior to or after one half-life of the therapeutic compound following the first portion release.

9. (canceled)

10. The formulation of claim 7, wherein release of the second portion of the therapeutic compound occurs prior to or after the release of substantially the entire first portion and prior to one half-life of the therapeutic compound following the release of the first portion.

11. (canceled)

12. The formulation of claim 7, wherein release of a second portion of the therapeutic compound contained in the formulation occurs at a time independent of an expression peak or trough of its target gene in a tissue type and wherein the release of the second portion avoids an undesirable side effect.

13. The formulation of claim 7, further providing release of at least a third portion of the therapeutic compound.

14. The formulation of claim 1, wherein the therapeutic compound inhibits at least two target genes and wherein the formulation provides coordinated release such that release of a first portion of the therapeutic compound contained in the formulation coincides with peak or trough expression of a first target gene and release of a second portion of the therapeutic compound contained in the formulation coincides with peak or trough expression of a second target gene.

15. The formulation of claim 14, further providing release of at least a third portion of the therapeutic compound contained in the formulation such that release of the at least third portion coincides with peak or trough expression of at least a third target gene and wherein peak or trough expression of the at least third target gene is defined in Table 2.

16. The formulation of claim 14, wherein each of the at least two target genes is selected from the group consisting of PPARα, PPARδ, and PPARγ.

17. The formulation of claim 1 wherein the therapeutic compound is a fibrate having a half-life of less than six hours.

18. The formulation of claim 17, wherein the fibrate is released two to four hours after contact with a solution having a pH of between 1 and 5 and a temperature of between 35 and 42° C.

19. The formulation of claim 1, wherein the at least one target gene is expressed in at least two tissue types and wherein the formulation provides coordinated release of the therapeutic compound such that release of a first portion of the therapeutic compound contained in the formulation coincides with peak or trough expression of the target gene in a first tissue type and release of a second portion of the therapeutic compound contained in the formulation coincides with peak or trough expression of the target gene in a second tissue type.

20-24. (canceled)

25. The formulation of claim 19, further providing release of at least a third portion of the therapeutic compound contained in the formulation such that the release of the at least third portion coincides with peak or trough expression of the at least one target gene in an at least third tissue type and wherein peak or trough expression of the at least one target gene in the at least third tissue type is defined in Table 2.

26. The formulation of claim 14, wherein the at least two target genes are expressed in at least two tissue types and wherein the formulation provides coordinated release of the therapeutic compound such that release of the first portion of the therapeutic compound contained in the formulation coincides with peak or trough expression of the first target gene in the first tissue type and release of the second portion of the therapeutic compound contained in the formulation coincides with peak or trough expression of the second target gene in the second tissue type.

27-30. (canceled)

31. The formulation of claim 26, further providing release of at least a third portion of the therapeutic compound contained in the formulation such that the release of the at least third portion coincides with peak or trough expression of at least a third target gene and wherein peak or trough expression of the at least third target gene is defined in Table 2, optionally, wherein the at least a third target gene is expressed in a third tissue type.

32. A formulation providing coordinated release of at least two therapeutic compounds selected from Table 1, wherein each therapeutic compound inhibits at least one different target gene wherein release of a first therapeutic compound from the formulation coincides with peak or trough expression of at least one target gene of the first therapeutic compound and wherein release of a second therapeutic compound from the formulation coincides with peak or trough expression of at least one target gene of the second therapeutic compound.

33-35. (canceled)

36. The formulation of claim 32, wherein the first therapeutic compound is an angiotensin receptor blocker (ARB) having a half-life of less than six hours and wherein the second therapeutic compound is a beta blocker having a half-life of less than three hours.

37. The formulation of claim 36, wherein the ARB is released zero to two hours after contact with a solution having a pH of between 1 and 5 and a temperature of between 35 and 42° C. and the beta blocker is released two to four hours after contact with a solution having a pH of between 1 and 5 and a temperature of between 35 and 42° C.

38. The formulation of claim 36, wherein the ARB is Valsartan or Losartan and the beta blocker is Metoprolol or Timolol.

39. The formulation of claim 32, wherein the target gene of the first therapeutic compound is Agtr1a and the target gene of the second therapeutic compound is Car4, Cart, Car12, or Car9.

40. The formulation of claim 32, wherein the first therapeutic compound is an angiotensin receptor blocker (ARB) having a half-life of less than six hours and wherein the second therapeutic compound is a diuretic.

41. The formulation of claim 40, wherein the ARB is released zero to two hours after contact with a solution having a pH of between 1 and 5 and a temperature of between 35 and 42° C. and the diuretic is released six to eight hours after contact with a solution having a pH of between 1 and 5 and a temperature of between 35 and 42° C.

42. The formulation of claim 40, wherein the ARB is Valsartan or Losartan and diuretic is Hydrochlorothiazide.

43. The formulation of claim 32, wherein the target gene of the first therapeutic compound is Ace and the target gene of the second therapeutic compound is Adrb2 or Adrb1.

44. The formulation of claim 32, wherein the first therapeutic compound is an acetylcholinesterase (ACE) inhibitor having a half-life of less than six hours and wherein the second therapeutic compound is a beta blocker having a half-life of less than three hours.

45. The formulation of claim 44, wherein the ACE inhibitor is released zero to two hours after contact with a solution having a pH of between 1 and 5 and a temperature of between 35 and 42° C. and the beta blocker is released two to four hours after contact with a solution having a pH of between 1 and 5 and a temperature of between 35 and 42° C.

46. The formulation of claim 44, wherein the ACE inhibitor is Enalapril or Ramipril and the beta blocker is Metoprolol or Timolol.

47. The formulation of claim 32, wherein the target gene of the first therapeutic compound is Ace and the target gene of the second therapeutic compound is Car4, Car2, Car12, or Car9.

48. The formulation of claim 32, wherein the first therapeutic compound is an acetylcholinesterase (ACE) inhibitor having a half-life of less than six hours and wherein the second therapeutic compound is a diuretic.

49. The formulation of claim 48, wherein the ACE inhibitor is released zero to two hours after contact with a solution having a pH of between 1 and 5 and a temperature of between 35 and 42° C. and the diuretic is released six to eight hours after contact with a solution having a pH of between 1 and 5 and a temperature of between 35 and 42° C.

50. The formulation of claim 48, wherein the ACE inhibitor is Enalapril or Ramipril and diuretic is Hydrochlorothiazide.

51. The formulation of claim 32, wherein the target gene of the first therapeutic compound is PPARα and the target gene of the second therapeutic compound is Hmgcr.

52. The formulation of claim 32, wherein the first therapeutic compound is a fibrate having a half-life of less than two hours and wherein the second therapeutic compound is a statin having a half-life of less than two hours.

53. The formulation of claim 52, wherein the fibrate is released zero to two hours after contact with a solution having a pH of between 1 and 5 and a temperature of between 35 and 42° C. and the statin is released four to six hours after contact with a solution having a pH of between 1 and 5 and a temperature of between 35 and 42° C.

54. The formulation of claim 52, wherein the fibrate is principally metabolized by CYP3A4 and the statin is principally metabolized by CYP2C9.

55. The formulation of claim 52, wherein the fibrate is Gemfibrozil and the statin is Fluvastatin.

56. (canceled)

57. The formulation of claim 32, further providing release of at least a third therapeutic compound contained in the formulation such that release of the at least third therapeutic compound coincides with peak or trough expression of at least a third target gene and wherein peak or trough expression of the at least third target gene is defined in Table 2.

58. A formulation providing coordinated release of at least two different therapeutic compounds selected from Table 1, wherein the at least two therapeutic compounds have at least one common target gene, wherein release of a first therapeutic compound coincides with peak or trough expression of the common target gene and release of a second therapeutic compound coincides with peak or trough expression of the common target gene.

59-60. (canceled)

61. A method for treating a disease comprising administering an effective amount of a formulation of claim 1 at a specified time such that release of the therapeutic compound from the formulation coincides with peak or trough expression of at least one target gene of the therapeutic compound.

62. A kit comprising a formulation of claim 1 and instructions for use that specify that the formulation is provided such that release of a first therapeutic compound or a first portion of the first therapeutic compound from the formulation coincides with peak or trough expression of at least one target gene of the first therapeutic compound.

63. (canceled)

64. A method of developing an improved formulation for a therapeutic compound, the method comprising:
identifying the circadian phase of gene expression of a target for the therapeutic compound;
identifying a desired administration time;
calculating a difference between the circadian phase of the target gene expression and the desired administration time; and
developing a delayed-release formulation for the therapeutic compound corresponding to the calculated difference.

65-77. (canceled)
